(19) United States
(12) Patent Application Publication
(10) Pub. No.: US 2015/0024388 A1
(43) Pub. Date: Jan. 22, 2015

(54) Expression of SEP-like Genes for Identifying and Controlling Palm Plant Shell Phenotypes

(71) Applicant: Malaysian Palm Oil Board, Kajang (MY)

(72) Inventors: Rajinder SINGH, Kuala Lumpur (MY); Leslie Low Eng Ti, Kuala Lumpur (MY); Leslie Ooi Cheng Li, Kuala Lumpur (MY); Meilina Ong Abdullah, Seremban (MY); Rajanaidu Nookiah, Kuala Lumpur (MY); Ravigadevi Sambanthamurthi, Selangor (MY); Jared Ordway, St. Louis, MO (US); Nathan D. Lakey, Chesterfield, MO (US); Steven W. Smith, Fitchburg, WI (US); Rob Martienssen, Cold Spring Harbor, NY (US); Michael Hogan, Ballwin, MO (US)

(21) Appl. No.: 14/336,376

(22) Filed: Jul. 21, 2014

Related U.S. Application Data
(60) Provisional application No. 61/856,433, filed on Jul. 19, 2013.

(51) Int. Cl. C12Q 1/68; A01H 1/04

(52) U.S. Cl. CPC C12Q 1/6876; A01H 1/04; C12Q 2600/13
USPC 435/6.11

(57) ABSTRACT

Methods and compositions are provided for optimizing fruit morphology.

### CROSS-REFERENCE TO RELATED PATENT APPLICATION

The present application claims the benefit of priority to U.S. Provisional Application No. 61/856,433, filed on Jul. 19, 2013, the contents of which are hereby incorporated by reference in their entirety and for all purposes.

### BACKGROUND OF THE INVENTION

The oil palm (E. guineensis, E. oleifera, and hybrids thereof) can be classified into separate groups based on its fruit characteristics, and has three naturally occurring fruit forms which vary in shell thickness and oil yield. Dura type palms are homozygous for a wild type allele of the SHELL gene (Sh⁺/Sh⁺), have a thick seed coat or shell (2-8 mm) and produce approximately 5.3 tons of oil per hectare per year. Tenera type palms are heterozygous for a wild type and mutant allele of the SHELL gene (Sh⁺/sh⁻), have a relatively thin shell surrounded by a distinct fiber ring, and produce approximately 7.4 tons of oil per hectare per year. Finally pisifera type palms are homozygous for a mutant allele of the SHELL gene (sh⁻/sh⁻), have no seed coat or shell, and are usually female sterile (Hartley, 1988) (FIG. 1). Therefore the gene controlling shell thickness is a major contributor to palm oil yield.

Tenera palms are simply hybrids between the dura and pisifera palms. Whitmore (1973) described the various fruit forms as different varieties of oil palm. However, Latiff (2000) was in agreement with Purseglove (1972) that varieties or cultivars as proposed by Whitmore (1973), do not occur in the strict sense in this species. As such, Latiff (2000) proposed the term “race” to differentiate dura, pisifera and tenera. Race was considered an appropriate term as it reflects a permanent microspecies, where the different races are capable of exchanging genes with one another, which has been adequately demonstrated in the different fruit forms observed in oil palm (Latiff, 2000). In fact, the characteristics of the three different races turn out to be controlled simply by the inheritance of a single gene. Genetic studies revealed that the SHELL gene shows co-dominant monogenic inheritance, which is exploitable in breeding programs (Beirnaert and Vanderweyen, 1941).

Tenera fruit forms have a higher mesocarp to fruit ratio than dura, which directly translates to significantly higher oil yield than either the dura or pisifera palm (as illustrated in Table 1). The pisifera is usually female sterile and does not produce fruit, and the fruit bunches, if produced, rot prematurely.

TABLE 1
Comparison of dura, tenera and pisifera fruit forms

Fruit Form

| Characteristic | Dura | Tenera | Pisifera* |
|---|---|---|---|
| Shell thickness (mm) | 2-8 | 0.5-3 | Absence of shell |
| Fibre Ring ** | Absent | Present | Absent |
| Mesocarp Content | 35-55 | 60-96 | 95 |
| (% fruit weight) | | | |
| Kernel Content | 7-20 | 3-15 | — |
| (% fruit weight) | | | |
| Oil to Bunch (%) | 16 | 26 | — |
| Oil Yield (t/ha/yr) | 5.3 | 7.4 | — |

*usually female sterile, bunches rot prematurely
** fibre ring is present in the mesocarp and often used as diagnostic tool to differentiate dura and tenera palms.
(Source: Harden et al., 1985; Hartley, 1988)

Since the goal of the breeding programs in oil palm is to produce planting materials with higher oil yield, the tenera palm is the preferred choice for commercial planting. It is for this reason that substantial resources are invested by commercial seed producers to cross selected dura and pisifera palms in hybrid seed production. And despite the many advances which have been made in the production of hybrid oil palm seeds, two significant problems remain in the seed production process. First, batches of tenera seeds, which will produce the high oil yield tenera type palm, are often contaminated with dura seeds (Donough and Law, 1995). Today, it is estimated that dura contamination of tenera seeds can reach rates of approximately 5% (reduced from as high as 20-30% in the early 1990's as the result of improved quality control practices). Seed contamination is due in part to the difficulties of producing pure tenera seeds in open plantation conditions, where workers use ladders to manually pollinate tall palms, and where palm flowers for a given bunch mature over a period time, making it difficult to pollinate all flowers in a bunch with a single manual pollination event. Some flowers of the bunch may have matured prior to manual pollination and therefore may have had the opportunity to be wind pollinated from an unknown palm, thereby producing contaminant seeds in the bunch. Alternatively premature flowers may exist in the bunch at the time of manual pollination, and may mature after the pollination occurred allowing them to be wind pollinated from an unknown palm thereby producing contaminant seeds in the bunch. Notably, in the six year interval from germination to fruit production, significant land, labor, financial and energy resources are invested into what are believed to be tenera palms, some of which will ultimately be of the unwanted low yielding contaminant fruit forms. By the time these suboptimal palms are identified, it is impractical to remove them from the field and replace them with tenera palms, and thus growers achieve lower palm oil yields for the 25 to 30 year production life of the contaminant palms. Therefore, the issue of contamination of batches of tenera seeds with dura or pisifera seeds is a problem for oil palm breeding, underscoring the need for a method to predict the fruit form of seeds and nursery plantlets with high accuracy.

A second problem in the seed production process is the investment seed producers make in maintaining dura and pisifera lines, and in the other expenses incurred in the hybrid seed production process. For example, to produce lines which maintain a pisifera allele, tenera palms are often selfed or crossed with another tenera palm. In this process, at least 25% of progeny are dura, based on Mendelian inheritance, and yet are cultivated in fields designated for pisifera maintenance for up to 6 years before they bear fruit and can be phenotyped. Therefore, a molecular tool can allow for these contaminant dura palms to be discarded at the seedling stage. This has significant implications in terms of allocation of financial (including fertilizer) and land resources. The ability to identify and separate out the different fruit forms greatly improves management practice, as the different fruit forms can be planted separately in the field. In addition pisifera palms can be planted in high density to encourage male flowers and pollen production. The tenera palms planted separately also allows for better assessment of their true potential as they do not have to compete with the vigorously growing pisifera palms. Due to the co-dominant nature of the SHELL gene, traditional plant breeding techniques cannot produce a palm with an optimal shell phenotype which when crossed to itself or to another palm with optimal shell phenotype would produce seeds which would only generate optimal shell phenotypes.

Genetic mapping of the SHELL gene was initially attempted by Mayes et al. (1997). A second group in Brazil, using a combination of bulked segregation analysis (BSA) and genetic mapping, reported a random amplified polymorphic DNA (RAPD) marker closely linked to the shell thickness locus (Moretzsohn et al., 2000). More recently Billotte et al., (2005) reported a simple sequence repeat (SSR)-based high density linkage map for oil palm, involving a cross between a thin shelled E. guineensis (tenera) palm and a thick shelled E. guineensis (dura) palm. In their study, they reported an SSR marker mapping close to the SHELL locus. A patent filed by the Malaysian Palm Oil Board (MPOB) describes the identification of a marker using restriction fragment technology, in particular a Restriction Fragment Length Polymorphism (RFLP) marker linked to the SHELL gene for plant identification and breeding purposes (RAJINDER SINGH, LESLIE OOI CHENG-LI, RAHIMAH A. RAHMAN AND LESLIE LOW ENG TI. 2008. Method for identification of a molecular marker linked to the SHELL gene of oil palm. Patent Application No. PI 20084563. Patent Filed on 13 Nov. 2008). The RFLP marker (SFB 83) was identified by way of generation or construction of a genetic map for a tenera palm.

More recently, the SHELL gene has been identified as a homologue of the MADS-box gene SEEDSTICK (STK) (Singh R, et al., The oil palm SHELL gene controls oil yield and encodes a homologue of SEEDSTICK, Nature in press (2013); U.S. patent application Ser. No. 13/800,652), which controls ovule identity and seed development in Arabidopsis, (Favaro R, et al., Plant Cell, 15(11), 2602-11, 2003). The SHELL gene is responsible for the tenera phenotype in both cultivated and wild palms from sub-Saharan Africa, and the gene's identity provides a genetic explanation for the single gene heterosis attributed to SHELL, via heterodimerization. SHELL is also a homologue of the Arabidopsis gene SHATTERPROOF(SHP1), a type II MADS-box transcription factor gene of the MIKC^{c} class. The ortholog of SHP1 in tomato plays an important role in regulation of fleshy fruit expansion (Vrebalov, et al., Plant Cell, 21(10), 3041-62, 2009).

SHELL-like proteins function as transcription regulatory factors by binding to DNA as homodimers or as heterodimers with other proteins such as other MADS-box family members. In Arabidopsis, SHP1 and STK are Type II MADS-box proteins of the C and D class, respectively, and form a network of transcription factors that control differentiation of the ovule, seed and lignified endocarp (Dinneny J R, et al., Bioessays, 27, 42-49, 2005). STK and SHP bind to DNA as heteromultimers with other MADs-box proteins, and the highly conserved MADS domain is involved in both DNA binding and in dimerization.

Identification of the SHELL gene in oil palm (SHELL) allows the use of improved methods for generating oil palms with desired shell characteristics such as marker assisted selection for SHELL mutants, identification and characterization of SHELL mutants early in the lifecycle of the plant (e.g. at the seed stage, during planting, or before fruiting), and breeding of SHELL mutants.

### BRIEF SUMMARY OF THE INVENTION

Described herein are methods and compositions for modulating the morphology of fruit. In some cases, the methods and compositions can modify the thickness of a fruit shell, increase the amount of fleshy fruit, or modify the thickness of fruit mesocarp. In one aspect, methods and compositions are provided for altering the shell thickness of palm fruit, such as oil palm fruit (e.g., E. guineensis). In some cases, methods and compositions are provided for optimizing the amount of oil produced by oil palm fruit.

In some embodiments, MADS-box containing proteins, such as a protein encoded by the SHELL gene or one or more proteins encoded by a SEP-like gene can be modulated in expression or activity to alter fruit morphology. In some cases, the ratio of MADS-box containing protein expression or activity can be modulated to alter fruit morphology.

Modulation of MADS-box containing protein expression or activity can be accomplished a variety of ways. For example, SHELL can be inactivated by mutagenesis, gene knockout or replacement, posttranscriptional modulation (e.g., using RNAi or a microRNA), or the use of an interfering polypeptide to sequester SHELL, a SHELL binding partner, or a SHELL target DNA sequence. As another example, one or more SEP-like proteins can be inactivated by mutagenesis, gene knockout or replacement, posttranscriptional modulation, or the use of an interfering polypeptide to sequester one or more SEP-like proteins, a SEP-like protein binding partner, or a SEP-like protein target DNA sequence. As yet another example, SHELL or a SEP-like protein, or a fragment thereof, can be overexpressed to alter the wild-type ratio between SHELL and one or more SEP-like proteins and thus alter fruit morphology. As yet another example, naturally occurring plants with polymorphisms in a SEP-like gene or the SHELL gene can be identified that are associated with a desired fruit morphology. Similarly, such plants with polymorphisms in a SEP-like gene or the SHELL gene can be crossed with dura, tenera, or pisifera plants to produce progeny that have an altered fruit morphology. Similarly, plants with altered (e.g., increased or decreased) expression of a SEP-like gene can be identified that are associated with a desired fruit morphology. Such plants can be cultivated or crossed with dura, tenera, or pisifera plants to produce progeny with altered fruit morphology.

In some embodiments, the present invention provides a method for sorting palm seeds, seed embryos, germinated seeds and plants by predicted shell thickness and/or oil yield, the method comprising obtaining a sample from a plurality of oil palm seeds or plants, thereby providing a plurality of samples; detecting expression or genotype of a SEP-like gene in the samples; and sorting the plurality of seeds or plants based on the seed's or plant's predicted shell thickness and/or oil yield, wherein the thickness of the shell is correlated to an expression level or mutation in the SEP-like gene.

In some embodiments, the present invention provides a method for detecting a palm plant or seed with a reduced fruit shell thickness as compared to a plant with a dura fruit form, the method comprising, providing a sample from the plant; and screening the sample for a mutation in a SEP-like gene, wherein the mutation in the SEP-like gene indicates that the plant has a reduced fruit shell thickness as compared to a plant with a dura fruit form. In some cases, the method further comprises providing a plurality of samples, each from a plurality of plants; and screening for a mutation in a SEP-like gene in each of the plurality of samples. In some cases, the SEP-like gene is 80%, 90%, 95%, or 99% identical to, or identical to, a gene selected from the group consisting of SEQ ID NOs: 78-151. In some cases, the SEP-like gene encodes a polypeptide that is 80%, 90%, 95%, or 99% identical to, or identical to, a polypeptide selected from the group consisting of SEQ ID NOs: 1-74.

In some cases, the method further comprises determining the genotype of the plant or seed for one or more SEP-like genes or determining the SHELL genotype of the plant. In some cases, the plant or seed is the product of a cross that included a parent with a wild-type SHELL genotype. In some cases, the plant or seed is the product of a cross that included a parent with a wild-type SHELL allele. In some cases, the plant or seed is heterozygous for a wild-type SHELL allele. In some cases, the plant or seed is homozygous for a wild-type SHELL allele. In some cases, the plant or seed is homozygous for a mutant SHELL allele (e.g., homozygous for a SHELL allele that provides a pisifera phenotype). The plant can be less than about 6, 5, 4, 3, 2, 1, or less than about 0.5 years old.

In some cases, the method further comprises selecting the plant or seed for cultivation, breeding, or destruction if the plant or seed is heterozygous for the mutation in the SEP-like gene. In some cases, the method further comprises selecting the plant or seed for cultivation, breeding, or destruction if the plant or seed is homozygous for the mutation in the SEP-like gene. In some cases, the method further comprises selecting the plant or seed for cultivation, breeding, or destruction if the plant or seed is homozygous for the wild-type SHELL allele; or selecting the plant or seed for cultivation, breeding or destruction if the plant or seed is heterozygous for the wild-type SHELL allele.

In some embodiments, the present invention provides a method for detecting a palm plant with a reduced fruit shell thickness as compared to a plant with a dura fruit form, the method comprising, providing a sample from the plant; and screening the sample for an increase or decrease in expression (e.g., protein or mRNA expression) of a SEP-like gene, wherein the increase or decrease in expression of the SEP-like gene indicates that the plant has a reduced fruit shell thickness as compared to a plant with a dura fruit form. In some cases, the increase or decrease in expression of a SEP-like gene is increased or decreased as compared to a wild-type plant, such as a wild-type oil palm plant. In some cases, the increase or decrease in expression of a SEP-like gene is increased or decreased as compared to a typical dura, tenera, or pisifera oil palm plant. In some cases, the method further comprises providing a plurality of samples, each from a plurality of plants; and screening for an increase or decrease in expression of a SEP-like gene in each of the plurality of samples. In some cases, the SEP-like gene is 80%, 90%, 95%, or 99% identical to, or identical to, a gene selected from the group consisting of SEQ ID NOs: 78-151. In some cases, the SEP-like gene encodes a polypeptide that is 80%, 90%, 95%, or 99% identical to, or identical to, a polypeptide selected from the group consisting of SEQ ID NOs: 1-74.

In some cases, the method further comprises determining the SHELL genotype of the plant. In some cases, the plant is heterozygous for a wild-type SHELL allele. In some cases, the plant is homozygous for a wild-type SHELL allele. The plant can be less than about 6, 5, 4, 3, 2, 1, or less than about 0.5 years old.

In some cases, the method further comprises selecting the plant or seed corresponding to the sample with increased expression of a SEP-like gene for cultivation, breeding, or destruction. In some cases, the method further comprises selecting the plant or seed corresponding to the sample with decreased expression of a SEP-like gene for cultivation, breeding, or destruction. In some cases, the method further comprises selecting the plant or seed for cultivation, breeding, or destruction if the plant or seed is homozygous for the wild-type SHELL allele; or selecting the plant or seed for cultivation, breeding, or destruction if the plant or seed is heterozygous for the wild-type SHELL allele.

In some embodiments, a SEP-like protein (e.g., any one of SEQ ID NOs: 1-74 or a substantially identical sequence thereof) or SHELL can be modified to induce a protein:protein interaction failure between the modified protein and a binding partner. In some cases, SHELL can be modified (e.g., by random or directed mutation or gene replacement) to reduce or eliminate its ability to bind to another SHELL protein, or to reduce or eliminate its ability to bind to a SEP-like protein. Modifications can include a truncation, or one or more amino acid deletions or substitutions. An example modification of SHELL that reduces or eliminates protein:protein interaction is the protein encoded by the sh^{MPOB} allele of SHELL (SEQ ID NO: 76).

In some cases, a SEP-like protein can be modified (e.g., by random or directed mutation or gene replacement) to induce a protein:protein interaction failure between the modified protein and a binding partner. In some cases, a SEP-like protein can be modified to reduce or eliminate its ability to bind to SHELL, reduce or eliminate its ability to bind to another copy of itself, or reduce or eliminate its ability to bind to another SEP-like protein. Modifications can include a truncation, or one or more amino acid deletions or substitutions. An example modification of a SEP-like protein that induces a protein:protein interaction failure is a modification in the MADS-box domain.

In some cases, a protein:protein interaction failure can be induced by downregulation, or knocking out of an endogenous SHELL or an endogenous SEP-like gene. Downregulation, or knocking out SHELL or a SEP-like gene can provide a protein:protein interaction failure by limiting the number or concentration of available binding partners. Downregulation can be performed by methods such as gene knockout, gene replacement, or a mutation in a regulatory element (e.g., a promoter or enhancer). Downregulation can also be performed by regulating the SHELL or SEP-like mRNA post-transcriptionally (e.g., using a microRNA or RNA interference). Downregulation can also be performed by regulating the SHELL or SEP-like polypeptides post-translationally (e.g., by introducing destabilizing mutations or ubiquinylation sites).

In some embodiments, protein:protein interaction between SHELL and one or more binding partners can be reduced or eliminated by competitive inhibition. For example, an interfering polypeptide can be expressed in a plant that binds to SHELL and sequesters the SHELL protein from interacting with one or more endogenous binding partners. In some cases, the interfering polypeptide binds to SHELL and sequesters SHELL from interacting with another copy of SHELL (e.g., prevents homodimerization), sequesters SHELL from interacting with a SEP-like protein (e.g., prevents heterodimerization), or both. The interfering polypeptide can be heterologous. The interfering polypeptide can arise from modifying an endogenous gene. In some cases, the interfering polypeptide is expressed in the plant using an expression cassette in which a polynucleotide encoding the interfering polypeptide is operably linked to a promoter (e.g., a heterologous promoter).

In some cases, the interfering polypeptide is a SHELL-like polypeptide. SHELL-like polypeptides include polypeptides that are at least about 50%, 60%, 65%, 70%, 75%, 80%, 85%, 90%, 95%, 99%, or more identical to SHELL. SHELL-like polypeptides further include polypeptides that are at least about 50%, 60%, 65%, 70%, 75%, 80%, 85%, 90%, 95%, 99%, or more identical to a domain of SHELL, such as an M, I, K, or C (MADS-box) domain. SHELL-like polypeptides further include polypeptides that are at least about 50%, 60%, 65%, 70%, 75%, 80%, 85%, 90%, 95%, 99%, or more identical to a fragment of SHELL or a fragment of a SHELL domain that is at least about 50, 60, 70, 80, 90, or 100 amino acids or more in length. SHELL-like interfering polypeptides can bind to endogenous SEP-like proteins, wild-type SHELL, or both. An example of a SHELL-like interfering polypeptide that can be overexpressed to sequester SHELL is the protein encoded by the sh^{AVROS} allele (SEQ ID NO: 77).

In some cases, the interfering polypeptide is a similar to a SEP-like protein. Polypeptides similar to SEP-like proteins include polypeptides that are at least about 50%, 60%, 65%, 70%, 75%, 80%, 85%, 90%, 95%, 99%, or more identical to one or more SEP-like proteins (e.g., one or more of SEQ. ID NOs: 1-74). Polypeptides similar to SEP-like proteins further include polypeptides that are at least about 50%, 60%, 65%, 70%, 75%, 80%, 85%, 90%, 95%, 99%, or more identical to or similar to a domain of one or more SEP-like proteins, such as an M, I, K, or C (MADS-box) domain. Polypeptides similar to SEP-like proteins further include polypeptides that are at least about 50%, 60%, 65%, 70%, 75%, 80%, 85%, 90%, 95%, 99%, or more identical to or similar to a fragment of a SEP-like protein or a fragment of a SEP-like protein domain that is at least about 50, 60, 70, 80, 90, or 100 amino acids or more in length. Interfering polypeptides similar to SEP-like proteins can bind to endogenous SEP-like proteins, wild-type SHELL, or both.

In some embodiments, a SEP-like protein or SHELL (e.g., any one of SEQ ID NOs: 1-74, or any one of SEQ ID NOs: 75-77) can be modified (e.g., by random or directed mutation or gene replacement) to induce a protein:DNA binding failure. For example, the protein can be modified to reduce or eliminate binding to target promoter regions or to increase binding to non-target promoter regions (e.g., reduce target sequence fidelity). In some cases, the modified SHELL or SEP-like protein can form protein:protein complexes, but such complexes have a reduced ability to bind to target promoter regions. In some cases, the modification is in a conserved DNA binding domain, such as the MADS-box domain. An example modification that induces a protein:DNA binding failure is the protein encoded by the sh^{AVROS} allele (SEQ ID NO: 77).

In some embodiments, SHELL or a SEP-like polypeptide (e.g., any one of SEQ ID NOs: 1-77) can be modified to reduce or eliminate the ability of the polypeptide to transcriptionally regulate target genes. Such modifications can include a truncation, or one or more amino acid deletions or substitutions. In some cases, such modifications include modifications that reduce or eliminate tetramer formation (e.g., formation of tetramers containing one or more of SHELL or a SEP-like protein). In other cases, such modifications reduce or eliminate the ability of SHELL or SEP-like containing tetramers, or other higher order protein complexes, to recruit additional transcriptional machinery.

In some cases, the modifications reduce or eliminate binding of such tetramers, or other higher order protein complexes, to RNA polymerase II. In some cases, the modifications reduce or eliminate the RNA polymerase II activity of complexes containing such tetramers, or other higher order protein complexes. The modifications can also reduce or eliminate binding of protein complexes containing SHELL to a SEP-like protein, to an APETALA-like protein, to a PISTILLATA-like protein, or to an AGAMOUS-like protein.

In some embodiments, the ability of SHELL-containing protein complexes, or protein complexes containing a SEP-like protein (e.g., tetramers or higher order protein complexes) to activate transcription of target genes can be disrupted by an interfering polypeptide. The interfering polypeptide can be heterologous, or it can arise from modifying an endogenous gene. In some cases, the interfering polypeptide is expressed in the plant using an expression cassette in which a polynucleotide encoding the interfering polypeptide is operably linked to a promoter (e.g., a heterologous promoter).

For example, an interfering polypeptide can be expressed in a plant that binds to SHELL and forms a non-productive tetramer or higher order protein complex. For example, the non-productive protein complex can be incapable of activating transcription of target genes, or activate transcription of target genes at a reduced level. In some cases, the interfering polypeptide sequesters other components of the protein complex (e.g., SHELL) from forming productive protein complexes. In some cases, the non-productive protein complex containing the interfering polypeptide can bind to a target sequence and occupy the site, thus blocking endogenous transcriptional regulation machinery from binding to and activating transcription of the target gene.

Alternatively, an interfering polypeptide can be expressed in a plant that binds to a SEP-like protein and forms a non-productive tetramer or higher order protein complex. For example, the non-productive protein complex can be incapable of activating transcription of target genes, or activate transcription of target genes at a reduced level. In some cases, the interfering polypeptide sequesters other components of the protein complex (e.g., a SEP-like protein) from forming productive protein complexes. In some cases, the non-productive protein complex containing the interfering polypeptide can bind to a target sequence and occupy the site, thus blocking endogenous transcriptional regulation machinery from binding to and activating transcription of the target gene.

In some cases, the interfering polypeptide is a SHELL-like polypeptide. SHELL-like polypeptides include polypeptides that are at least about 50%, 60%, 65%, 70%, 75%, 80%, 85%, 90%, 95%, 99%, or more identical to or similar to SHELL. SHELL-like polypeptides further include polypeptides that are at least about 50%, 60%, 65%, 70%, 75%, 80%, 85%, 90%, 95%, 99%, or more identical to or similar to a domain of SHELL, such as an M, I, K, or C (MADS-box) domain. SHELL-like polypeptides further include polypeptides that are at least about 50%, 60%, 65%, 70%, 75%, 80%, 85%, 90%, 95%, 99%, or more identical to or similar to a fragment of SHELL or a fragment of a SHELL domain that is at least about 50, 60, 70, 80, 90, or 100 amino acids or more in length.

In some cases, the interfering polypeptide is similar to a SEP-like protein. Polypeptides similar to SEP-like proteins include polypeptides that are at least about 50%, 60%, 65%, 70%, 75%, 80%, 85%, 90%, 95%, 99%, or more identical to or similar to one or more SEP-like proteins (e.g., one or more of SEQ. ID NOs: 1-74). Polypeptides similar to SEP-like proteins further include polypeptides that are at least about 50%, 60%, 65%, 70%, 75%, 80%, 85%, 90%, 95%, 99%, or more identical to or similar to a domain of one or more SEP-like proteins, such as an M, I, K, or C (MADS-box) domain. Polypeptides similar to SEP-like proteins further include polypeptides that are at least about 50%, 60%, 65%, 70%, 75%, 80%, 85%, 90%, 95%, 99%, or more identical to or similar to a fragment of a SEP-like protein or a fragment of a SEP-like protein domain that is at least about 50, 60, 70, 80, 90, or 100 amino acids or more in length.

In one embodiment, the present invention provides an isolated nucleic acid comprising an expression cassette, the expression cassette comprising a promoter (e.g., a heterologous promoter) operably linked to a polynucleotide, which polynucleotide, when expressed in the plant, reduces expression of a SEPALLATA (SEP)-like polypeptide in the plant (compared to a control plant lacking the expression cassette). The nucleic acid promoter can be constitutive, tissue-specific, or inducible.

In one aspect, the nucleic acid comprises at least 10, 15, 20, 30, 40, 50, or 100 contiguous nucleotides, or the complement thereof, of an endogenous nucleic acid encoding a SEP-like polypeptide substantially (e.g., a least 80, 85, 90, 95, 97, 98, 99%) identical or identical to one of SEQ ID NOs: 1-74, such that expression of the polynucleotide in an oil palm plant inhibits expression of the endogenous SEP-like gene.

In some cases, the nucleic acid encodes a siRNA, antisense polynucleotide, a microRNA, or a sense suppression nucleic acid, thereby suppressing expression of the endogenous SEP-like gene.

In another embodiment, the present invention provides an expression vector comprising any of the foregoing nucleic acids.

In another embodiment, the present invention provides a transgenic palm plant comprising an expression cassette comprising any of the foregoing nucleic acids, wherein expression of the polynucleotide reduces expression of an endogenous SEP-like polypeptide in the plant (compared to a control plant lacking the expression cassette), and wherein reduced expression of the SEP-like polypeptide results reduced shell thickness in the plant.

In one aspect, the present invention provides a transgenic palm plant comprising an expression cassette comprising any of the foregoing nucleic acids wherein the nucleic acid comprises at least 10, 15, 20, 30, 40, 50, or 100 contiguous nucleotides, or a complement thereof, of an endogenous nucleic acid encoding a SEP-like polypeptide substantially (e.g., at least 80, 85, 90, 95, 97, 98, 99%) identical or identical to one of SEQ ID NOs: 1-74, such that expression of the polynucleotide inhibits expression of the endogenous SEP-like gene.

In another aspect, the present invention provides a transgenic palm plant comprising an expression cassette comprising any of the foregoing nucleic acids, wherein the nucleic acid encodes a siRNA, antisense polynucleotide, a microRNA, or a sense suppression nucleic acid, thereby suppressing expression of an endogenous SEP-like gene.

In another aspect, the present invention provides any of the foregoing transgenic palm plants, wherein the plant makes mature shells that are on average less than 2 mm thick. In some cases, the palm plant is an oil palm plant.

In one embodiment, the present invention provides an isolated nucleic acid comprising an expression cassette, the expression cassette comprising a promoter operably linked to a polynucleotide encoding an interfering polypeptide comprising a MADS-box domain of a SEP-like polypeptide, wherein, when expressed in a palm plant, the interfering polypeptide binds an endogenous SHELL polypeptide in the plant, thereby resulting in reduced shell thickness compared to shells of a control plant lacking the interfering polypeptide.

In one aspect, the MADS-box domain of the isolated nucleic acid is a MADS-box domain from an endogenous palm plant SEP-like polypeptide substantially (e.g., at least 80, 85, 90, 95, 97, 98, 99%) identical or identical to a MADS-box domain of one of SEQ ID NOs: 1-74. In some cases, the interfering polypeptide is not a full-length SEP-like polypeptide. In some cases, the interfering SEP-like polypeptide is a fragment of a MADS-box domain that contains about 10, 11, 12, 13, 14, 15, 20, 25, 30, 35, 40, 45, 50, 75, 100, 125, 150, 175, 200, 225, 250, 300, or about 400 or 500 continuous amino acids or more that are at least 80, 85, 90, 95, 97, 98, 99% identical or identical to a MADS-box domain fragment in one of SEQ ID NOs: 1-74.

In one embodiment, the present invention provides an isolated nucleic acid comprising an expression cassette, the expression cassette comprising a promoter operably linked to a polynucleotide encoding an interfering polypeptide comprising a MADS-box domain of a SHELL polypeptide, wherein, when expressed in a palm plant, the interfering polypeptide binds an endogenous polypeptide encoded by a SEP-like gene in the plant, thereby resulting in reduced shell thickness compared to shells of a control plant lacking the interfering polypeptide.

In one aspect, the MADS-box domain of the isolated nucleic acid is a MADS-box domain from an endogenous palm plant SHELL polypeptide substantially (e.g., at least 80, 85, 90, 95, 97, 98, 99%) identical or identical to a MADS-box domain of one of SEQ ID NOs: 75-77. In some cases, the interfering polypeptide is not a full-length SHELL polypeptide. In some cases, the interfering SHELL polypeptide is a fragment of a MADS-box domain that contains about 10, 11, 12, 13, 14, 15, 20, 25, 30, 35, 40, 45, 50, 75, 100, 125, 150, 175, 200, 225, 250, 300, or about 400 or 500 continuous amino acids or more that are at least 80, 85, 90, 95, 97, 98, 99% identical or identical to a MADS-box domain fragment in one of SEQ ID NOs: 75-77.

In some embodiments, the present invention provides a palm plant comprising any one of the foregoing expression cassettes and transgenically expressing an interfering polypeptide, wherein the interfering polypeptide binds an endogenous SHELL polypeptide in the plant, thereby resulting in reduced shell thickness compared to shells of a control plant lacking the interfering polypeptide. In some aspects, wherein the expression cassette comprises a nucleic acid comprising a MADS-box domain from an endogenous palm plant SEP-like polypeptide substantially (e.g., at least 80, 85, 90, 95, 97, 98, 99%) identical or identical to a MADS-box domain of one of SEQ ID NOs: 1-74. In some cases, the interfering polypeptide is a truncated SEP-like polypeptide. In some cases, the transgenic palm plant is an oil palm plant.

In some embodiments, the present invention provides a palm plant comprising any one of the foregoing expression cassettes and transgenically expressing an interfering polypeptide, wherein the interfering polypeptide binds an endogenous SEP-like polypeptide in the plant, thereby resulting in reduced shell thickness compared to shells of a control plant lacking the interfering polypeptide. In some aspects, wherein the expression cassette comprises a nucleic acid comprising a MADS-box domain from an endogenous palm plant SHELL polypeptide substantially (e.g., at least 80, 85, 90, 95, 97, 98, 99%) identical or identical to a MADS-box domain of one of SEQ ID NOs: 75-77. In some cases, the interfering polypeptide is a truncated SHELL polypeptide. In some cases, the transgenic palm plant is an oil palm plant.

In another embodiment, the invention provides a method of making any of the foregoing palm plants, the method comprising introducing an expression cassette into a palm plant via crossing with a transgenic palm plant comprising the expression cassette or transforming the plant with a nucleic acid comprising the expression cassette. In one aspect, the present invention provides a method comprising cultivating any of the foregoing plants.

In one embodiment, the present invention provides a method of making an oil palm plant with reduced shell thickness compared to a shell of a control plant comprising: generating a plurality of mutant oil palm plant cells; and screening the oil palm plant cells for reduced SEP-like gene mRNA expression, reduced SEP-like protein activity, reduced SHELL gene mRNA expression, or reduced SHELL protein activity.

In one aspect, the plurality of mutant oil palm plant cells are generated via random mutagenesis of oil palm plant cells. In some cases, the random mutagenesis comprises contacting the plant cells with a chemical mutagen (e.g., ethylmethane sulphonate (EMS), ethylene imine (EI), nitrosoethyl urea, nitrosoethyl urethane, N-Methyl-N′-nitro-N-nitrosoguanidine (MNNG), or sodium azide); irradiating the plant cells (e.g., by fast neutron bombardment, X-ray, or gamma ray irradiation), mobilization of transposable elements in the genome of the plant cells, or random insertion of transposable elements or T-DNA into the genome of the plant cells (e.g., using Agrobacterium spp. or Ensifer spp.).

In another aspect, the plurality of mutant oil palm plant cells are generated via site directed mutagenesis. In some cases, the site directed mutagenesis comprises contacting the plant cells with a transcription activator-like effector nuclease (TALEN), a zinc finger nuclease, or a chimeraplast. In some cases, the TALEN or zinc finger nuclease specifically cleaves a sequence within 1 kb of a SEP-like gene in the oil palm genome, or within 1 kb of the SHELL gene in the oil palm genome. In some cases, the chimeraplast specifically binds to a sequence within 1 kb of a SEP-like gene in the oil palm genome, or within 1 kb of the SHELL gene in the oil palm genome. In some cases, the site directed mutagenesis comprises contacting the plant cells with a nucleic acid that contains at least 15 continuous nucleotides that are homologous to a sequence within 1 kb of the SEP-like gene in the oil palm genome, or within 1 kb of the SHELL gene in the oil palm genome.

In another embodiment, the present invention provides a plant produced by any of the foregoing methods, wherein the plant has an enhanced oil yield compared to a control plant in which mRNA expression of a SEP-like gene is not reduced and SEP-like protein activity is not reduced.

In yet another embodiment, the present invention provides a plant produced by any of the foregoing methods, wherein the plant has an enhanced oil yield compared to a control plant in which mRNA expression of SHELL gene is not reduced and SHELL protein activity is not reduced.

### BRIEF DESCRIPTION OF THE DRAWINGS

FIG. 1 Illustrates transcriptional activation of target genes by MADS-box genes. A. In Arabidopsis MADS-box gene products can interact to form dimers and tetramers. The different tetramer complexes illustrated initiate different developmental programs. B. Wild-type SHELL can bind OSMADS24, a SEP-like protein to form a dimer as illustrated. This dimer can form higher order complexes such as a tetramer and can also bind DNA to regulate transcription. C. The sh^{MPOB} allele has a mutation in the MADS-box domain that inhibits dimer formation and leads to loss of transcriptional regulation. D. The sh^{AVROS} allele has a mutation in the MADS-box domain that inhibits DNA binding and thus leads to a loss of transcriptional regulation.

FIG. 2 Illustrates different steps at which compositions and methods described herein can be utilized to alter fruit morphology. In step 1, binding of MADS-box containing proteins such as SHELL and the SEP-like proteins can be modulated via mutations that disrupt the protein:protein interaction, down regulation of the MADS-box containing protein or its binding partner, or competitive inhibition with an interfering polypeptide. Interfering polypeptides include MADS-box domain containing polypeptides. In step 2, binding of MADS-box containing proteins such as SHELL and the SEP-like proteins to DNA can be modulated via mutations that disrupt DNA binding. In step 3, transcriptional regulation of target genes can be modulated by introducing mutations that disrupt tetramer formation or disrupt binding to RNA polymerase II or other transcription factors. Transcriptional regulation of target genes can also be modulated by expressing interfering peptides that bind to endogenous SHELL or a SEP-like protein and fail to properly regulate transcription of target genes.

FIG. 3 Depicts the results from a yeast two-hybrid assay to identify SHELL binding partners. a, Legend for plating layout. Auto-activation controls: 1, shAVROS (BD)+pGADT7; 2, shMPOB (BD)+pGADT7; 3, OsMADS24 (BD)+pGADT7; 4 ShDeliDura+pGADT7. Interaction tests: 5, shAVROS (AD)+shAVROS (BD); 6, shAVROS (AD)+shMPOB (BD); 7, shAVROS (AD)+OsMADS24 (BD); 8, OsMADS24 (AD)+shAVROS (BD); 9, shMPOB (AD)+shAVROS (BD); 10, shMPOB (AD)+shMPOB (BD); 11, shMPOB (AD)+OsMADS24 (BD); 12, OsMADS24 (AD)+shMPOB (BD); 13, shAVROS (AD)+ShDeliDura (BD); 14, shMPOB (AD)+ShDeliDura (BD); 15, ShDeliDura (AD)+ShDeliDura (BD); 16, OsMADS24 (AD)+ShDeliDura (BD); 17, ShDeliDura (AD)+shAVROS (BD); 18, ShDeliDura (AD)+shMPOB (BD); 19, ShDeliDura (AD)+OsMADS24 (BD); 20, OsMADS24 (AD)+OsMADS24 (BD); A, pGBKT7-53+pGADT7-T (positive control); B, pGBKT7-lam+pGADT7-T (negative control). Co-transformants were plated on selective media, as labeled (b-d) and on X-gal media (e). Interaction assay results are summarized in Table 1 and Supplementary Table 1. Abbreviations: AD, construct made in activation domain fusion plasmid pGADT7; BD, construct made in DNA binding domain fusion plasmid pGBKT7.

FIG. 4 Pairwise co-transformations of the indicated MADS-box peptides expressed as activation domain fusions (AD) and as DNA binding domain fusions (BD) were performed in yeast strain AH109 as described (Methods). Heterodimerization with OsMADS24 occurred only when the peptide was fused to the activation domain. Auto-activation column/row indicates the lack of auto-activation by all fusion constructs.

FIG. 5 Depicts SEPALLATA (SEP) sequences recovered from GenBank from rice (O. sativa) and oil palm (E. guineensis) and aligned using Clustal X. Conserved residues are highlighted. Gaps are denoted by “-.”

FIG. 6 Depicts a parsimony tree from the aligned sequences of FIG. 3. Clades are classified as A, B, C, D, and E class MADS-box proteins.

### DETAILED DESCRIPTION OF THE INVENTION

### I. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, N.Y. 1989); Raven et al. PLANT BIOLOGY (7th ed. 2004). Any methods, devices and materials similar or equivalent to those described herein can be used in the practice of this invention.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. In some embodiments, the plant is of the genus Elaeis. In some cases, the plant is an oil palm plant (e.g., Elaeis guineensis, Elaeis oleifera, or a hybrid thereof).

An “expression cassette” refers to a nucleic acid construct, which when introduced into a host cell (e.g., a plant cell), results in transcription and/or translation of a RNA or polypeptide, respectively. An expression cassette typically includes a sequence to be expressed, and sequences necessary for expression of the sequence to be expressed. The sequence to be expressed can be a coding sequence or a non-coding sequence (e.g., an inhibitory sequence). The sequence to be expressed is generally operably linked to a promoter. The promoter can be a heterologous promoter. Generally, an expression cassette is inserted into an expression vector to be introduced into a host cell. The expression vector can be viral or non-viral.

“Recombinant” refers to a human manipulated polynucleotide or a copy or complement of a human manipulated polynucleotide. For instance, a recombinant expression cassette comprising a promoter operably linked to a second polynucleotide may include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). A recombinant expression cassette may comprise polynucleotides combined in such a way that the polynucleotides are extremely unlikely to be found in nature. For instance, human manipulated restriction sites or plasmid vector sequences may flank or separate the promoter from the second polynucleotide. One of skill will recognize that polynucleotides can be manipulated in many ways and are not limited to the examples above. A recombinant protein is one that is expressed from a recombinant polynucleotide, and recombinant cells, tissues, and organisms are those that comprise recombinant sequences (polynucleotide and/or polypeptide).

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is different from any naturally-occurring allelic variants. As another example a heterologous promoter can be a promoter operably linked to a polynucleotide encoding an RNA or protein, wherein the promoter is not found operably linked to that polynucleotide in a wild-type organism. Similarly, an expression cassette can be heterologous. A heterologous expression cassette can be an expression cassette that differs in at least one aspect from endogenous expression cassettes. For example, the expression cassette can contain a heterologous promoter. As another example, the expression cassette can contain genomic sequences normally found in a chromosome of an organism, yet the expression cassette can be heterologous because it replicates as an extrachromasomal nucleic acid.

The term “exogenous,” in reference to a polypeptide or polynucleotide, refers to polypeptide or polynucleotide which is introduced into a cell or organism (e.g., plant) by any means other than by a sexual cross.

The term “transgenic,” e.g., a transgenic plant or plant tissue, refers to a recombinantly modified organism with at least one introduced genetic element. The term is typically used in a positive sense, so that the specified gene is expressed in the transgenic organism. However, a transgenic organism can be transgenic for an inhibitory nucleic acid, i.e., a sequence encoding an inhibitory nucleic acid is introduced. The introduced polynucleotide can be from the same species or a different species, can be endogenous or exogenous to the organism, can include a non-native or mutant sequence, or can include a non-coding sequence.

In the case of both expression of transgenes and inhibition of endogenous genes (e.g., by antisense, or sense suppression) one of skill will recognize that a polynucleotide sequence need not be identical and can be “substantially identical” to a sequence of the gene from which it was derived.

The term “promoter” refers to regions or sequence located upstream and/or downstream from the start of transcription and which are involved in recognition and binding of RNA polymerase and other proteins to initiate transcription. A “plant promoter” is a promoter capable of initiating transcription in plant cells. In some cases, a plant promoter used in the present invention may originally derive from the same species or variety of plant into which it is introduced, e.g., methods and compositions using a canola promoter in a canola plant. In other cases, a plant promoter used in the present invention may originally derive from a different plant, e.g., methods using methods and compositions using a petunia promoter in a canola plant. In yet other cases, the plant promoters of the present invention may not derive from a plant, e.g. a bacterial or fungal promoter in a plant that is capable of initiating transcription in plant cells.

A “constitutive promoter” in the context of this invention refers to a promoter that is capable of initiating transcription in nearly all cell types, whereas a “cell type-specific promoter” or “tissue-specific promoter” initiates transcription only in one or a few particular cell types or groups of cells forming a tissue. In some embodiments, a promoter is tissue-specific if the transcription levels initiated by the promoter in a specific cell-type or tissue are at least 2-fold, 3-fold, 4-fold, 5-fold, 6-fold, 7-fold, 8-fold, 9-fold, 10-fold, 50-fold, 100-fold, 500-fold, 1000-fold higher or more as compared to the transcription levels initiated by the promoter in non-specific tissues. In some embodiments, the promoter is vessel-specific, root-specific, flower-specific, shoot-specific, or meristem-specific.

An “inducible promoter” refers to a promoter which can respond to a signal to increase or decrease transcription. For example, an inducible promoter may be silent, i.e., does not substantially initiate transcription, in the absence of a signal and active, i.e., initiates transcription, in the presence of the signal. Examples of inducible promoters include promoters are provided herein. In some cases inducible promoters may initiate transcription in response to biotic stress or abiotic stress (i.e., stress-inducible promoters), temperature (e.g. heat shock promoters), drought, hypoxia, the level of a particular hormone, or the presence of a small-molecule or chemical such as tetracycline, dexamethasone, copper, salicyclic acid herbicide safeners, or cis-Jasmone. In some embodiments of the invention, tissue specific promoters are inducible. In some embodiments, a promoter is inducible if the transcription levels initiated by the promoter under inducing conditions is at least 2-fold, 3-fold, 4-fold, 5-fold, 6-fold, 7-fold, 8-fold, 9-fold, 10-fold, 50-fold, 100-fold, 500-fold, 1000-fold higher or more as compared to the transcription levels initiated by the promoter in a non-induced state.

The term “inactivate,” with reference to a particular gene, refers to methods or compositions in which one or more genes are rendered partially, substantially, or completely unable to perform their function. For example, a gene may be inhibited, mutated, knocked-out, or modulated such that it no longer effectively performs its function.

The term “modulate” as in to “modulate a gene,” “modulate expression” of a gene, “or “modulate the activity” of a gene or protein, refers to increasing or decreasing the expression, activity, or stability of a gene or gene product (e.g., a protein or RNA product of a gene). For example, a gene may be modulated by increasing or decreasing the amount of RNA that is transcribed from the gene or altering the rate of such transcription. Decreased expression may include expression that is reduced by 5%, 10%, 15%, 20%, 25%, 30%, 50%, 75%, 80%, 90%, 95%, 99% or more. Increased expression includes expression that is increased by 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, 100%, or more. In some cases expression may be increased by at least 2-fold, 3-fold, 4-fold, 5-fold, 6-fold, 7-fold, 8-fold, 9-fold, 10-fold, 50-fold, 100-fold, 500-fold, 1000-fold or higher. Expression may be modulated in a tissue specific or inducible manner as provided herein. In some cases, increased or decreased expression can be identified by measuring mRNA or protein levels in a tissue (e.g., root, shoot, stem, leaf, sepal, petal, seed, etc.) of a plant. Modulation of a gene can also include altering a gene by targeted gene editing, gene replacement, or gene knockout.

Modulation of the activity of gene products that are involved in protein:protein or protein:DNA interactions can include altering the binding or enzymatic activity of the gene product, sequestering a gene product from participating in protein:protein interactions (e.g., sequestering a protein so that it does not bind to its binding partner), sequestering a gene product from binding to target DNA, or sequestering a target DNA from being bound by a gene product.

In some cases, the gene product is a transcription factor and modulating the activity of the transcription factor gene product includes altering the transcriptional activation of target genes. For example, transcriptional activation of target genes can be increased or decreased. Transcriptional activation can be increased, and thus increase expression of one or more target genes by 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, 100%, or more. Transcriptional activation may also be increased, and thus increase expression of one or more target genes by at least 2-fold, 3-fold, 4-fold, 5-fold, 6-fold, 7-fold, 8-fold, 9-fold, 10-fold, 50-fold, 100-fold, 500-fold, 1000-fold or higher. Decreased transcriptional activation may include expression that is reduced by 5%, 10%, 15%, 20%, 25%, 30%, 50%, 75%, 80%, 90%, 95%, 99% or more.

The term “knockdown” or “knockout,” with reference to a particular gene, describes an organism that is genetically modified to delete the gene, reduce expression of the gene (e.g., to less than 1, 5, 10, or 20% of wild type expression), or to express a non-functional gene product. The term gene knockdown is used synonymously with gene knockout or gene deficient.

The terms “antisense,” “inhibitory nucleic acid,” “inhibitory polynucleotide,” “interfering polynucleotide,” and “interfering nucleic acid” are used generally herein to refer to RNA targeting strategies for reducing gene expression. These strategies include RNAi, siRNA, shRNA, dsRNA, etc. Typically, the antisense sequence is identical to the targeted sequence (or a fragment thereof), but this is not necessary for effective reduction of expression. For example, the antisense sequence can have 85, 90, 95, 98, or 99% identity to the complement of a target RNA or fragment thereof. The targeted fragment can be about 10, 20, 30, 40, 50, 10-50, 20-40, 20-100, 40-200 or more nucleotides in length.

The term “interfering polypeptide” is generally used herein to refer to a polypeptide which binds to an endogenous target polypeptide thereby reducing the ability of the target polypeptide to 1) bind to its normal cellular protein partner, 2) to bind to a DNA target, and/or 3) to transactivate its normal cellular target genes. The interfering polypeptide can be identical, substantially identical, or substantially similar to the amino acid sequence of the endogenous binding partner of the endogenous target protein. Alternatively, the interfering polypeptide can be or identical, substantially identical or substantially similar to a fragment of the endogenous binding partner. For example, the interfering polypeptide sequence can have 85, 90, 95, 98, 99% identity, or be identical to the endogenous binding partner of the endogenous target polypeptide, or to a fragment thereof. The interfering polypeptide can be a polypeptide fragment of about 10, 20, 30, 40, 50, 60, 75, 100, 125, 150, 200, 250, or more amino acids in length that is 85, 90, 95, 98, 99% identical, or identical to a polypeptide fragment of about 10, 20, 30, 40, 50, 60, 75, 100, 125, 150, 200, 250, or more amino acids in length of an endogenous binding partner of the endogenous target gene.

Interfering polypeptides can act to “sequester” MADS-box proteins from binding to endogenous binding partners, forming dimers or tetramers, or transcriptionally regulating target genes (e.g., activating transcription). As used herein, “sequester,” “sequestering,” and the like refers to binding to and interfering with the wild-type function of a gene. Sequestering can include binding to an endogenous protein (e.g., a MADS-box protein such as SHELL or a SEP-like protein) and removing its ability to interact with other endogenous proteins.

The term “RNAi” refers to RNA interference strategies of reducing expression of a targeted gene. RNAi technique employs genetic constructs within which sense and anti-sense sequences are placed in regions flanking an intron sequence in proper splicing orientation with donor and acceptor splicing sites. Alternatively, spacer sequences of various lengths can be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases then bind to and cleave the double-stranded RNA, thereby initiating the cascade of events leading to degradation of specific mRNA gene sequences, and silencing specific genes. The phenomenon of RNA interference is described and discussed in Bass, Nature 411: 428-29 (2001); Elbahir et al., Nature 411: 494-98 (2001); and Fire et al., Nature 391: 806-11 (1998); and WO 01/75164, where methods of making interfering RNA also are discussed.

The term “siRNA” refers to small interfering RNAs, that are capable of causing interference with gene expression and can cause post-transcriptional silencing of specific genes in cells, e.g., in plant cells. The siRNAs based upon the sequences and nucleic acids encoding the gene products disclosed herein typically have fewer than 100 base pairs and can be, e.g., about 30 bps or shorter, and can be made by approaches known in the art, including the use of complementary DNA strands or synthetic approaches. Typical siRNAs have up to 40 bps, 35 bps, 29 bps, 25 bps, 22 bps, 21 bps, 20 bps, 15 bps, 10 bps, 5 bps or any integer thereabout or there between. Tools for designing optimal inhibitory siRNAs include that available from DNAengine Inc. (Seattle, Wash.) and Ambion, Inc. (Austin, Tex.).

A “short hairpin RNA” or “small hairpin RNA” is a ribonucleotide sequence forming a hairpin turn which can be used to silence gene expression. After processing by cellular factors the short hairpin RNA interacts with a complementary RNA thereby interfering with the expression of the complementary RNA.

“Co-suppression” as used herein refers to the introduction of nucleic acid configured in the sense orientation to block the transcription of target genes. For an example of the use of this method to modulate expression of endogenous genes see Assaad et al., Plant Mol. Bio. 22: 1067-1085 (1993); Flavell, Proc. Natl. Acad. Sci. USA 91: 3490-3496 (1994); Stam et al., Annals Bot. 79: 3-12 (1997); Napoli et al., The Plant Cell 2:279-289 (1990); and U.S. Pat. Nos. 5,034,323, 5,231,020, and 5,283,184.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The term “substantial identity” of polynucleotide sequences means that a polynucleotide comprises a sequence that has at least 25% sequence identity. Alternatively, percent identity can be any integer from at least 25% to 100% (e.g., at least 25%, 26%, 27%, 28%, . . . , 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%), preferably calculated with BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from at least 40% to 100% (e.g., at least 40%, 41%, 42%, 43%, . . . , 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%). More preferred embodiments include at least 60%, 65%, 70%, 75%, 80%, 85%, 90%, 95%, or 99%.

The present invention provides palm SEPALLATA (SEP)-like polypeptides (and polynucleotides encoding such polypeptides) substantially identical to the sequences exemplified herein (e.g., any of SEQ ID NOs: 1-74), polynucleotides and expression cassettes encoding such SEP-like polypeptides or a mutation or fragment thereof, and vectors or other constructs for reducing SEP-like polypeptide expression in a palm plant. The present invention also provides palm SHELL polypeptides (and polynucleotides encoding such polypeptides) substantially identical to the sequences exemplified herein (e.g., any of SEQ ID NOs: 75-77), polynucleotides and expression cassettes encoding such SHELL polypeptides or a mutation or fragment thereof, and vectors or other constructs for reducing SHELL polypeptide expression in a palm plant.

Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Unless otherwise indicated, the comparison window extends the entire length of a reference sequence. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

One example of a useful algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length “W” in the query sequence, which either match or satisfy some positive-valued threshold score “T” when aligned with a word of the same length in a database sequence. “T” is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters “W”, “T”, and “X” determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

(see, e.g., Creighton, Proteins (1984)).

An indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

The present invention provides polynucleotides that selectively hybridize to one of SEQ ID NOs:78-154. The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, highly stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 time background hybridization. Polynucleotides that selectively hybridize to any one of SEQ ID NOs:78-154 can be of any length, e.g., at least 10, 15, 20, 25, 30, 50, 100, 200 500 or more nucleotides or having fewer than 500, 200, 100, or 50 nucleotides, etc.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cased, the nucleic acids typically hybridize under moderately stringent hybridization conditions.

In some embodiments, genomic DNA or cDNA comprising nucleic acids of the invention can often be identified in standard Southern blots under stringent conditions using the nucleic acid sequences disclosed here. For the purposes of this disclosure, suitable stringent conditions for such hybridizations are those which include a hybridization in a buffer of 40% formamide, 1 M NaCl, 1% SDS at 37° C., and at least one wash in 0.2×SSC at a temperature of at least about 50° C., usually about 55° C. to about 60° C., for 20 minutes, or equivalent conditions. A positive hybridization is at least twice background. Those of ordinary skill will readily recognize that alternative hybridization and wash conditions can be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., a northern or Southern blot.

As used herein, the term “SEP-like” refers to genes and gene products that comprise type-II MADS-box proteins and that are identified as having significant homology to SEP genes and gene products respectively. Consequently, SEP-like genes and gene products include SEP genes and gene-products. As explained above, SEP-like genes and gene products can be identified by use of a weighted sequence homology algorithm such as BLAST. SEP-like genes can also be identified by use of hybridization. For example, genes that hybridize under stringent conditions to known SEP genes can be identified as SEP-like. SEP-like genes and gene products can also be identified searching a database with a probabilistic hidden markov model. Exemplary SEP-like proteins include SEQ ID NOs: 1-74. Exemplary SEP-like genes include SEQ ID NOs: 78-151.

As used herein, the term “SHELL” refers to the oil palm ortholog of Arabidopsis thaliana SEEDSTICK (STK). SHELL, in combination with one or more SEP-like proteins, is believed to control the shell thickness phenotype in oil palm plants. SHELL protein (SEQ ID NOs: 75-77) and gene (SEQ ID NOs: 152-154) sequences are provided herein.

### II. Introduction

The present disclosure describes the identification of binding partners of the gene product responsible for the development of the oil palm fruit shell, SHELL (a homologue of the Arabidopsis gene SEEDSTICK (STK)). It is believed that such gene products can bind SHELL and alter SHELL activity. Accordingly, nucleic acids, proteins, and mutations thereof that affect the activity or expression of these SHELL-binding proteins can affect the activity of SHELL itself and are thus useful in the oil palm industry. For example, such nucleic acids, proteins, and mutations thereof that affect the activity or expression of SHELL-binding proteins can be used for breeding of optimized oil palm plant varieties, commercial seed production of oil palm plants with desired fruit phenotypes, and production of oil palm fruit with enhanced oil yield.

### II. Protein:Protein Interactors

### A. Binding Partners of SHELL

The inventors have surprisingly discovered that the protein encoded by the SHELL gene allele found in thick shelled oil palm fruits, or dura, (Sh^{DeliDura}) allele, binds to SEPALLATA (SEP) orthologs from rice (Oryza sativa) in a yeast two-hybrid system. The inventors have further discovered that inactive SHELL protein variants, encoded by the Sh^{MPOB} allele, which are associated with the no-shell phenotype (pisifera), do not bind to SEP orthologs in rice in a yeast two-hybrid system. It is believed that SHELL activity can be regulated by altering expression or activity of SHELL binding partners in oil palm. Accordingly, it is believed that oil palm fruit phenotypes associated with SHELL genotypes, such as shell thickness, the absence or presence of a shell, and oil yield can be optimized by modulating the expression or activity of SHELL binding partners in oil palm.

SHELL binding partners include oil palm SEP and SEP-like proteins. The inventors have therefore identified SEP-like oil palm genes. SEP-like oil palm genes were identified by searching RefSeq (Pruitt K D, Tatusova T, Klimke W, Maglott D R. NCBI Reference Sequences: current status, policy and new initiatives. Nucleic Acids Res. 2009 January; 37 (Database issue):D32-36.) for SEP protein sequences. The SEP protein sequences were then utilized to generate a profile hidden markov model (HMM) of SEP proteins. The HMM which was then used to search the oil palm genome, containing approximately 34,000 genes, for genes encoding SEP-like proteins. SEQ ID NOs: 1-74 were identified as SEP-like proteins. SEQ ID NOs: 1-74 are representative SEP-like sequences and individual oil palms may have a substantially identical amino acid sequence (e.g., having one, two, three, or more amino acid changes) relative to SEQ ID NOs: 1-74 due, for example, to natural variation.

It is believed that inactivating, knocking out, or downregulating SEP-like proteins (e.g., one or more of SEQ ID NOs: 1-74) or genes encoding SEP-like proteins can reduce the level of SHELL/SEP protein complexes in an oil palm plant. Thus, for example, one can inactivate, knockout, or downregulate a SHELL binding partner (e.g., a SEP-like protein) and thus affect oil palm fruit shell thickness or oil palm fruit oil yield. In some cases, inactivating, knocking out, or downregulating a SHELL binding partner (e.g., a SEP-like protein) can provide an oil palm plant with a reduced shell thickness or an enhanced oil yield. For example, induced or naturally occurring mutations in one or more SEP-like genes that reduce expression or activity of a SEP-like protein (e.g., one or more of SEQ ID NOs: 1-74) can provide an oil palm plant that has a reduced shell thickness or enhanced oil yield.

In some embodiments, mutations in one or more SEP-like genes that reduce the activity of, or interfere with SHELL can provide an oil palm plant that has a reduced shell thickness or enhanced oil yield. Thus, expression of one or more SEP-like genes in oil palm that interfere with, or reduce the activity of SHELL can provide reduced shell thickness or enhanced oil yield phenotype compared to a wild-type palm plant and/or a wild-type SEP allele.

SEP-like genes encode MADS-box type transcription factors. Such transcription factors generally bind to DNA as homodimers or as heterodimers (Huang et al., Plant Cell. 8(1): 81-94, 1996), and the highly conserved C-(MADS-box) domain is involved in both DNA binding and in protein-protein interaction (Immink et al., Semin Cell Dev Biol. 21(1):87-93 2010). SEP-like proteins also contain additional domains, such as M, I, and K domains. The structure and function of these domains is described in, e.g. Gramzow and Theissen, 2010 Genome Biology 11: 214-334 and corresponding domains can be identified in the oil palm sequences provided herein.

In some embodiments, expression of a SEP-like protein having active protein:protein interaction activity but a non-functional DNA binding activity can remove proteins that interact with the modified SEP-like protein from biological action. Thus, for example, one can express a SEP-like protein with a non-functional DNA binding activity under control of a heterologous promoter in the plant (e.g., a palm plant, e.g., a dura or tenera background), thereby resulting in a reduced shell thickness or enhanced oil yield.

As another example, by expressing a SEP-like protein having a non-functional protein:protein interaction domain but an active DNA binding domain, DNA binding sites may be titrated or sequestered away from functional SHELL-containing protein complexes. Thus, for example, one can express a SEP-like protein with a functional DNA binding activity and a non-functional protein:protein interaction activity under control of a heterologous promoter in the plant (e.g., an oil palm plant, e.g., a dura or tenera background), thereby resulting in a reduced shell thickness or enhanced oil yield.

In some cases, one or more endogenous or wild-type SEP-like proteins negatively regulate SHELL activity. In such cases, overexpression of one or more of these SEP-like proteins can be used to alter oil palm fruit shell thickness. Thus for example, one can express a SEP-like protein herein under control of a heterologous promoter in the plant (e.g., an oil palm plant, e.g., a dura background), thereby resulting in a reduced shell thickness or enhanced oil yield. Alternatively, overexpression of one or more SEP-like proteins can alter the ratio of the SEP-like protein and one or more binding partners (e.g., SHELL) such that the transcriptional activation of SEP/SHELL target genes is altered. Thus, optimization of fruit shell thickness or oil yield can result from overexpression of one or more SEP-like proteins. As explained herein, overexpression can be performed, for example, via an expression cassette containing a polynucleotide encoding a SEP-like protein operably linked to a promoter, such as a heterologous promoter.

In some cases, one or more SEP-like proteins can be heterologously overexpressed in order to enhance SHELL activity. For example, in a tenera or pisifera background, one or more SEP-like proteins can be overexpressed to provide an altered (e.g., increased or decreased) shell thickness or enhanced oil yield as compared to a wild-type tenera or pisifera oil palm plant.

In some embodiments, SEP-like alleles can be partially inactivated. In some cases, one or more SEP-like alleles can be partially defective in protein:protein interaction. For example, the SEP-like allele can interact with SHELL with a reduced affinity. In other cases, one or more SEP-like alleles can be partially defective in DNA binding. For example, the SEP-like allele can bind to SEP transcription factor binding sites with a reduced affinity or reduced fidelity. In other cases, one or more SEP-like alleles can be partially defective in transcriptional regulation. For example, the SEP-like allele does not provide the same type or level of transcriptional regulation as a wild-type allele. As another example, the SEP-like allele can be reduced in expression as compared to a wild-type plant, but not inactivated or knocked out.

In such embodiments, oil palm plants with partially defective SEP-like alleles can provide additional shell phenotype diversity. For example a SEP-like allele with reduced expression or activity (e.g. reduced binding to SHELL, reduced DNA binding activity, or reduced transcriptional regulation) in a dura background can provide a shell phenotype that is reduced in thickness as compared to a dura plant. In some cases, the thickness is not reduced as compared to a tenera plant (e.g., has a thicker shell than a tenera plant). Similarly, a SEP-like allele with reduced expression or activity (e.g. reduced binding to SHELL, reduced DNA binding activity, or reduced transcriptional regulation) in a tenera background can provide a shell phenotype that is reduced in thickness as compared to a tenera plant, but not as compared to a pisifera plant. One of skill in the art will recognize that shell thickness and oil yields can thus be optimized by altering expression levels and activities of the various SEP genes provided herein in various SHELL genotypic backgrounds.

### B. Binding Partners of SEP-Like Proteins

SEP orthologs in Arabidopsis and rice often form dimeric and tetrameric protein complexes with other MADS-box proteins, including SEPALLATA, SHATTERPROOF, AGAMOUS, APETALA, and PISTILLATA. The interplay between the various combinations of possible MADS-box dimers, tetramers, and the like among SEPALLATA, SHATTERPROOF, AGAMOUS, APETALA, and PISTILLATA genes, homologs, and orthologs can be altered in order to modulate fruit morphology. Consequently, it is believed that the activity of one or more SEP-like proteins, and thus oil palm fruit phenotypes such as shell thickness and oil yield, can be optimized by modulating the expression or activity of one or more SEP-like protein binding partners. SEP-like protein binding partners are encoded, for example, by SHELL genes (SEQ ID NOs: 152-154) or gene products (SEQ ID NOs: 75-77), or fragments thereof SEQ ID NOs: 75-77 are representative SHELL sequences and individual oil palms may have a substantially identical amino acid sequence (e.g., having one, two, three, or more amino acid changes) relative to SEQ ID NOs: 75-77 due, for example, to natural variation.

It is believed that inactivating, knocking out, or downregulating SHELL proteins (e.g., one or more of SEQ ID NOs: 75-77) or genes encoding SHELL proteins can reduce the level of SHELL/SEP-like protein complexes in an oil palm plant. Thus, for example, one can inactivate, knockout, or downregulate SHELL and thus affect oil palm fruit shell thickness or oil palm fruit oil yield. In some cases, inactivating, knocking out, or downregulating SHELL can provide an oil palm plant with a reduced shell thickness or an enhanced oil yield. For example, induced or naturally occurring mutations in SHELL that reduce expression or activity of a SHELL protein (e.g., one or more of SEQ ID NOs: 75-77) can provide an oil palm plant that has a reduced shell thickness or enhanced oil yield.

In some embodiments, mutations in SHELL that reduce the activity of, or interfere with, a SEP-like gene can provide an oil palm plant that has a reduced shell thickness or enhanced oil yield. Thus, expression of one or more SHELL genes in oil palm that interfere with, or reduce the activity of, a SEP-like gene can provide reduced shell thickness or enhanced oil yield phenotype compared to a wild-type palm plant and/or a wild-type SHELL allele.

SHELL encodes a MADS-box type transcription factor. Such transcription factors generally bind to DNA as homodimers or as heterodimers (Huang et al., Plant Cell. 8(1): 81-94, 1996), and the highly conserved C-(MADS-box) domain is involved in both DNA binding and in protein-protein interaction (Immink et al., Semin Cell Dev Biol. 21(1):87-93 2010). SHELL also contains additional domains, such as M, I, and K domains. The structure and function of these domains is described in, e.g. Gramzow and Theissen, 2010 Genome Biology 11: 214-334 and corresponding domains can be identified in the oil palm sequences provided herein.

In some embodiments, expression of a SHELL polypeptide having protein:protein interaction activity but a non-functional DNA binding activity can remove proteins that interact with the modified SHELL polypeptide from biological action. Thus, for example, one can express a SHELL polypeptide with a non-functional DNA binding activity under control of a heterologous promoter in the plant (e.g., a palm plant, e.g., a dura or tenera background), thereby resulting in a reduced shell thickness or enhanced oil yield.

As another example, by expressing a SHELL polypeptide having a non-functional protein:protein interaction domain but an active DNA binding domain, DNA binding sites may be titrated or sequestered away from functional protein complexes that contain SEP-like proteins. Thus, for example, one can express a SHELL polypeptide with a functional DNA binding activity and a non-functional protein:protein interaction activity under control of a heterologous promoter in the plant (e.g., an oil palm plant, e.g., a dura or tenera background), thereby resulting in a reduced shell thickness or enhanced oil yield.

As yet another example, overexpression of SHELL can alter the ratio of SHELL and one or more SHELL binding partners (e.g., one or more SEP-like proteins). In some cases, this alteration of the ratio of SHELL to SHELL binding partners via SHELL overexpression can thus optimize fruit shell thickness or provide enhanced oil yield. As explained herein, overexpression can be performed, for example, via an expression cassette containing a polynucleotide encoding a SHELL protein operably linked to a promoter, such as a heterologous promoter.

In some embodiments, SHELL alleles can be partially inactivated. In some cases, one or more SHELL alleles can be partially defective in that they encode for proteins which are defective in the protein:protein interaction. For example, the resulting SHELL protein can interact with SEP-like proteins with a reduced affinity. In other cases, one or more SHELL alleles can encode proteins that are partially defective in DNA binding. For example, such a SHELL protein can bind to SHELL transcription factor binding sites with a reduced affinity or reduced fidelity. In other cases, one or more SHELL alleles can encode proteins that are partially defective in transcriptional regulation. For example, the SHELL protein does not provide the same type or level of transcriptional regulation as a wild-type protein. As another example, the SHELL allele can be reduced in expression as compared to a wild-type plant, but not inactivated or knocked out.

In such embodiments, oil palm plants with partially defective SHELL alleles can provide additional fruit shell phenotype diversity. For example a SHELL allele with reduced expression or activity (e.g. reduced binding to a SEP-like protein, reduced DNA binding activity, or reduced transcriptional regulation) in a dura background can provide a shell phenotype that is reduced in thickness as compared to a dura plant. In some cases, the fruit shell thickness is not reduced as compared to a tenera plant (e.g., has a thicker shell than a tenera plant). Similarly, a SHELL allele with reduced expression or activity (e.g. reduced binding to a SEP-like protein, reduced DNA binding activity, or reduced transcriptional regulation) in a tenera background can provide a shell phenotype that is reduced in thickness as compared to a tenera plant, but not as compared to a pisifera plant. One of skill in the art will recognize that shell thickness and oil yields can thus be optimized by altering expression level and activities of SHELL in various genotypic backgrounds.

### III. Transgenic Plants

Any of a number of methods can be used to express SHELL genes, SEP-like genes, or nucleic acids derived therefrom in plants. Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. Alternatively, a SHELL gene, a SEP-like gene, or a nucleic acid derived therefrom can be expressed constitutively (e.g., using the CaMV 35S promoter).

As discussed above, the SHELL gene of palm has been discovered to control shell phenotype. Moreover, the SHELL gene product is thought to interact with one or more SEP-like genes. Thus in some embodiments, plants having modulated expression or activity of a SHELL gene or polypeptide, or a SEP-like gene or polypeptide are provided. Such plants can provide fruit with enhanced oil yield, reduced shell thickness, or a combination thereof. Such plants can also provide fruit with additional phenotypic diversity as compared to the natural dura, tenera, and pisifera phenotypes.

It has been discovered that pisifera SHELL alleles contain missense mutations in portions of the gene encoding the MADS box domain of the protein, which plays a role in transcription regulation. Moreover, it has been discovered that, in a yeast two-hybrid screen, proteins encoded by such pisifera SHELL alleles do not interact with SEP gene products. In contrast, proteins encoded by dura alleles do have the ability to interact with one or more SEP gene products. Therefore, it is believed that SHELL activity can require interaction with a SEP-like gene product (e.g., heterodimerization) to bind DNA and induce a thick shell phenotype in oil palm plants.

Thus, plants with a reduced level of SHELL or one or more SEP-like proteins compared to wild-type plants can provide fruit with reduced shell thickness, enhanced oil yield, or a combination thereof as compared to dura plants or as compared to tenera plants. Accordingly, in some embodiments, plants having reduced level of SHELL or one or more SEP-like proteins as compared to a wild-type plant are provided. Such plants can be generated, for example, using gene inhibition technology, including but not limited to siRNA technology, to reduce, but not eliminate, gene expression of endogenous SHELL or an endogenous SEP-like gene (e.g., in a dura or tenera background).

In some cases, a recombinant SHELL or SEP-like expression cassette (i.e., a transgene) can be introduced into an oil palm plant in which one or more SHELL or SEP-like genes have been knocked out or inactivated. Such an expression cassette can be configured to control expression of a SHELL or SEP-like gene at a reduced level or an increased level compared to the native promoter. This can be achieved, for example, by operably linking a mutated SHELL or SEP-like gene promoter to a polynucleotide encoding a SHELL or SEP-like polypeptide, thereby weakening the “strength” of the promoter, or by operably linking a heterologous promoter that is weaker than the native promoter to a polynucleotide encoding a SHELL or SEP-like polypeptide.

Alternatively, some embodiments provide SHELL proteins (e.g., one or more of SEQ ID NOs: 75-77) or SEP-like proteins (e.g., one or more of SEQ ID NOs: 1-74) that have been altered to have reduced protein:protein binding activity. For example, plants that heterologously express one or more SEP-like proteins, or a fragment thereof, with one or more M, I, K or C domains that are non-functional with respect to SHELL binding but functional with respect to DNA binding are provided. Similarly, plants that heterologously express a SHELL protein, or a fragment thereof, with one or more M, I, K or C domains that are non-functional with respect to binding to a SEP-like protein but functional with respect to DNA binding are provided. M, I, K, and C-domains are described in, e.g., Gramzow and Theissen, 2010 Genome Biology 11: 214-224 and the corresponding domains can be identified in the oil palm sequences described herein. By expressing such a protein (having active DNA binding activity but a reduced or defective SHELL binding activity), genomic transcription factor binding sites can be sequestered from SHELL/SEP binding and transcriptional regulation. In some cases, such plants can provide fruit with an altered (e.g., reduced) shell thickness or enhanced oil yield as compared to a tenera or dura oil palm plant.

In other embodiments, plants that heterologously express one or more SEP-like proteins (e.g. any one of SEQ ID NOs: 1-74 or a sequence substantially identical thereto) are provided. Expression of such a protein can alter the wild-type ratio of MADS-box proteins present in the cell. In some cases such alteration can disrupt wild-type transcriptional regulation of MADS-box target genes. For example, overexpression of a SEP-like gene can disrupt transcriptional activation of SHELL target genes.

In other embodiments, plants that heterologously express one or more SEP-like proteins with one or more M, I, K, or C domains that bind SHELL but do not bind DNA or have a reduced or altered DNA binding activity are provided. Expression of such a protein (having protein:protein interaction activity but a non-functional, reduced or altered DNA binding activity), will lead to binding with SHELL, but the resulting SHELL/SEP-like heterodimer can have a reduced DNA binding activity. Thus SHELL can be removed from biological action, thereby resulting in a reduced shell thickness or enhanced oil yield. Thus, for example, one can express a SEP-like protein of one or more of SEQ ID NOs: 1-74, or a fragment thereof, in which the C-domain is missing or inactive under control of a heterologous promoter in the plant (e.g., a palm plant, e.g., a dura or tenera background), thereby resulting in the reduced shell thickness or enhanced oil yield.

Similarly, plants that heterologously express a SHELL protein with an M, I, K, or C domain that binds a SEP-like protein but does not bind DNA or has a reduced or altered DNA binding activity are provided. Expression of such a protein (having protein:protein interaction activity but a non-functional, reduced or altered DNA binding activity), will lead to binding with a SEP-like protein, but the resulting SHELL/SEP-like heterodimer can have a reduced DNA binding activity. Thus the endogenous SEP-like protein can be removed from biological action, thereby resulting in a reduced shell thickness or enhanced oil yield. Thus, for example, one can express a SHELL protein of one or more of SEQ ID NOs: 75-77, or a fragment thereof, in which the C-domain is missing or inactive under control of a heterologous promoter in the plant (e.g., a palm plant, e.g., a dura or tenera background), thereby resulting in the reduced shell thickness or enhanced oil yield.

### a. Inhibition or Suppression of SEP-Like Gene Expression

Also provided herein are methods for controlling shell thickness in a palm or other plant by reducing expression of an endogenous nucleic acid molecule encoding a SEP-like polypeptide that binds with SHELL such as one or more of SEQ ID NOs: 1-74. Exemplary gene sequences that encode SEP-like proteins include SEQ ID NOs: 78-151. For example, in a transgenic plant, a nucleic acid molecule, or antisense, siRNA, microRNA, or dsRNA constructs thereof, targeting a SEP-like gene, or fragment thereof, or a SEP mRNA, or fragment thereof can be operatively linked to an exogenous regulatory element, wherein expression of the construct suppresses endogenous SEP-like gene expression. In any case, suppression includes gene expression that is less than about 75%, 60%, 50%, 40%, 30%, 20%, 10%, 5%, 1%, 0.5%, 0.1%, 0.05%, or 0.01% of the gene expression found in a wild-type plant or control plant.

A number of methods can be used to inhibit gene expression in plants. For instance, antisense technology can be conveniently used. To accomplish this, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the enzyme of interest, see, e.g., Sheehy et al., Proc. Nat. Acad. Sci. USA, 85:8805-8809 (1988); Pnueli et al., The Plant Cell 6:175-186 (1994); and Hiatt et al., U.S. Pat. No. 4,801,340.

The antisense nucleic acid sequence transformed into plants will be substantially identical to at least a portion of the endogenous gene or genes to be repressed. The sequence, however, does not have to be perfectly identical to inhibit expression. Thus, an antisense or sense nucleic acid molecule encoding only a portion of a SEP-like encoding sequence can be useful for producing a plant in which expression of one or more SEP-like genes is suppressed. The vectors can be designed such that the inhibitory effect applies to other proteins within a family of genes exhibiting homology or substantial homology to the target gene, or alternatively such that other family members are not substantially inhibited. For example, a vector can be designed to express a nucleic acid encoding a sequence corresponding to a conserved region with substantially shared homology between 2 or more, 3 or more, 4 or more, 5 or more, or 6 or more SEP-like genes such as 2, 3, 4, 5, 6 or more of a gene encoding any 2, 3, 4, 5, 6, or more of SEQ ID NOs: 1-74, or a polypeptide substantially identical thereto. Such a vector can thus suppress expression of 2, 3, 4, 5, 6 or more SEP-like genes such as 2, 3, 4, 5, 6 or more of SEQ ID NOs: 78-151, or a polynucleotide substantially identical thereto. Alternatively, a vector can be designed to express a nucleic acid encoding a sequence corresponding to a relatively non-conserved region such that expression of 7 or fewer, 6 or fewer, 5 or fewer, 4 or fewer, 3 or fewer, 2 or 1 SEP-like gene is substantially suppressed.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. In some embodiments, a sequence of at least, e.g., 15, 20, 25 30, 50, 100, 200, or more continuous nucleotides (up to mRNA full length) substantially identical to an endogenous SEP mRNA, or a complement thereof, can be used.

Catalytic RNA molecules or ribozymes can also be used to inhibit expression of a SEP gene. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al. Nature, 334:585-591 (1988).

Another method of suppression is sense suppression (also known as co-suppression). Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al., The Plant Cell 2:279-289 (1990); Flavell, Proc. Natl. Acad. Sci., USA 91:3490-3496 (1994); Kooter and Mol, Current Opin. Biol. 4:166-171 (1993); and U.S. Pat. Nos. 5,034,323, 5,231,020, and 5,283,184. In some cases, co-suppression can be performed by introducing into a plant cell an expression cassette in which a nucleic acid encoding one or more of SEQ ID NOs: 1-74, or a substantially identical polypeptide or fragment thereof, is operably linked to a suitable promoter.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be suppressed. This minimal identity will typically be greater than about 65%, but a higher identity might exert a more effective suppression of expression of the endogenous sequences. In some embodiments, the level of identity is more than about 80% or about 95%. As with antisense regulation, the effect can apply to any other proteins within a similar family of genes exhibiting homology or substantial homology and thus which area of the endogenous gene is targeted will depend whether one wished to inhibit, or avoid inhibition, of other gene family members.

For sense suppression, the introduced sequence in the expression cassette, needing less than absolute identity, also need not be full length, relative to either the primary transcription product or fully processed mRNA. This may be preferred to avoid concurrent production of some plants that are over expressers. A higher identity in the introduced nucleic acid sequence relative to the gene to be suppressed can compensate for a short introduced nucleic acid sequence length. Furthermore, the introduced sequence need not have the same intron or exon pattern, and identity of non-coding segments will be equally effective. In some cases, a sequence of the size ranges noted above for antisense regulation is used.

Endogenous gene expression may also be suppressed by way of RNA interference (RNAi), which uses a double-stranded RNA having a sequence identical or similar to the sequence of the target gene. RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA. In some cases, the introduced double-stranded RNA is initially cleaved into small fragments, which then serve as indexes of the target gene, thereby degrading the target gene. RNAi is known to be also effective in plants (see, e.g., Chuang, C. F. & Meyerowitz, E. M., Proc. Natl. Acad. Sci. USA 97: 4985 (2000); Waterhouse et al., Proc. Natl. Acad. Sci. USA 95:13959-13964 (1998); Tabara et al. Science 282:430-431 (1998)). For example, to achieve suppression of the expression of a DNA encoding a protein using RNAi, a double-stranded RNA having the sequence of a DNA encoding the protein, or a substantially similar sequence thereof (including those engineered not to translate the protein) or fragment thereof, is introduced into a plant of interest. The resulting plants may then be screened for a phenotype associated with the target protein and/or by monitoring steady-state RNA levels for transcripts encoding the protein. Although the genes used for RNAi need not be completely identical to the target gene, they may be at least 70%, 80%, 90%, 95% or more identical to the target gene sequence. See, e.g., U.S., Patent Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Publication No. 2003/0221211.

The RNAi polynucleotides may encompass the full-length target RNA or may correspond to a fragment of the target RNA. In some cases, the fragment will have fewer than 100, 200, 300, 400, 500 600, 700, 800, 900 or 1,000 nucleotides corresponding to the target sequence. In addition, in some embodiments, these fragments are at least, e.g., 50, 100, 150, 200, or more nucleotides in length. In some cases, fragments for use in RNAi will be at least substantially similar to regions of a target protein that do not occur in other proteins in the organism or may be selected to have as little similarity to other organism transcripts as possible, e.g., selected by comparison to sequences in analyzing publicly-available sequence databases.

Expression vectors that continually express nucleic acids in transiently- and stably-transfected plants have been engineered to express small hairpin RNAs, which get processed in vivo into siRNA molecules capable of carrying out gene-specific silencing (Brummelkamp et al., Science 296:550-553 (2002), and Paddison, et al., Genes & Dev. 16:948-958 (2002)). Post-transcriptional gene silencing by double-stranded RNA is discussed in further detail by Hammond et al. Nature Rev Gen 2: 110-119 (2001), Fire et al. Nature 391: 806-811 (1998) and Timmons and Fire Nature 395: 854 (1998).

By using technology based on specific nucleotide sequences (e.g., antisense or sense suppression, siRNA, microRNA technology, etc.), families of homologous genes can be suppressed with a single sense or antisense transcript, if desired. For instance, if a sense or antisense transcript is designed to have a sequence that is conserved among a family of genes (e.g., the SEP-like genes or a family of SEP-like genes such as the class A, B, C, D, E, F or G SEP genes; AGL12-type, ANR1-type, or T(SVP)-type SEP genes; or SEP1, SEP2, or SEP3 genes), then multiple members of a gene family can be suppressed. Conversely, if the goal is to only suppress one member of a homologous gene family, then the sense or antisense transcript should be targeted to sequences with the most variance between family members. In some cases, sequences with the most variance can be found in non-coding sequences, sequences found between conserved domains, or sequences that encode variable loops or linker regions, e.g., linker sequences between different domains, of the SEP-like proteins.

Yet another way to suppress expression of an endogenous plant gene is by recombinant expression of a microRNA that suppresses a target (e.g., a SEP-like gene). Artificial microRNAs are single-stranded RNAs (e.g., between 18-25 mers, generally 21 mers), that are not normally found in plants and that are processed from endogenous miRNA precursors. Their sequences are designed according to the determinants of plant miRNA target selection, such that the artificial microRNA specifically silences its intended target gene(s) and are generally described in Schwab et al, The Plant Cell 18:1121-1133 (2006) as well as the internet-based methods of designing such microRNAs as described therein. See also, US Patent Publication No. 2008/0313773.

### B. Use of Nucleic Acids of the Invention to Express SEP-Like Polypeptides

Nucleic acid sequences encoding SEP-like proteins that interfere with SHELL activity can be heterologously expressed in an oil palm plant to, for example, alter shell thickness or enhance oil yield. In some cases, nucleic acid sequences encoding wild-type SEP-like protein sequences, or alternatively SEP-like proteins sequences containing mutations (e.g., one or more substitutions, additions, or deletions) can be heterologously expressed in an oil palm plant to, for example, alter shell thickness or enhance oil yield. For example, nucleic acid sequences encoding all or a portion of a SEP-like polypeptide (including but not limited to (i) a polypeptide substantially identical to a portion of one of SEQ ID NOs: 1-74; (ii) a SEP-like polypeptide having a functional M, I, and K domain and a non-functional C-domain; or (iii) a SEP-like polypeptide having a non-functional M, I, or K domain and a functional C-domain), can be used to prepare expression cassettes that enhance oil yield or reduce shell thickness when introduced into an oil palm plant. Where overexpression of a gene is desired, the desired SEP-like gene from a different species may be used to decrease potential co-suppression effects.

The SEP-like polypeptides described herein, like other proteins, have different domains which perform different functions. Thus, the gene sequences need not be full length, so long as the desired functional domain of the protein is expressed as a desired functional or non-functional variant. For example, a nucleotide sequence encoding a C-domain from a SEP-like polypeptide without one or more of the corresponding M, I, or K domains can be expressed in an oil palm plant. In some cases, the C-domain is non-functional with respect to protein:protein interaction (e.g., SHELL binding). In other cases, the C-domain is non-functional with respect to DNA binding. Such a C-domain can then sequester SHELL or SHELL DNA binding sites and alter shell thickness or enhance oil yield from oil palm fruit. Similarly, in some cases, a nucleotide sequence encoding an M domain, an I domain, or a K domain of a SEP-like protein can be overexpressed in an oil palm plant. In some cases, other combinations of domains, including but not limited to M and I, M and K, M and C, I and K, or I and C can be overexpressed. In some cases, the SEP-like polypeptide is functional with respect to binding to SHELL, binding to other SEP-like proteins, or binding to DNA, but non-functional with respect to activating transcription of target genes.

### C. Use of Nucleic Acids of the Invention to Express SHELL Polypeptides

Nucleic acid sequences encoding SHELL polypeptides that interfere with the activity of one or more SEP-like proteins can be heterologously expressed in an oil palm plant to alter shell thickness or enhance oil yield. For example, nucleic acid sequences encoding all or a portion of a SHELL polypeptide (including but not limited to (i) a polypeptides substantially identical to a portion of one of SEQ ID NOs: 75-77; (ii) a SHELL polypeptide having a functional M, I, and K domain and a non-functional C-domain; or (iii) a SHELL polypeptide having a non-functional M, I, or K domain and a functional C-domain), can be used to prepare expression cassettes that enhance oil yield or reduce shell thickness when introduced into an oil palm plant. Where overexpression of a gene is desired, a SHELL homolog from a different species may be used to decrease potential co-suppression effects.

The SHELL polypeptides described herein, like other proteins, have different domains which perform different functions. Thus, the gene sequences need not be full length, so long as the desired functional domain of the protein is expressed as a desired functional or non-functional variant. For example, a nucleotide sequence encoding a C-domain from a SHELL polypeptide without one or more of the corresponding M, I, or K domains can be expressed in an oil palm plant. In some cases, the C-domain is non-functional with respect to protein:protein interaction (e.g., binding to a SEP-like protein). In other cases, the C-domain is non-functional with respect to DNA binding. Such a C-domain can then sequester SHELL or SHELL DNA binding sites and alter shell thickness or enhance oil yield from oil palm fruit. Similarly, in some cases, a nucleotide sequence encoding an M domain, an I domain, or a K domain of a SEP-like protein can be overexpressed in an oil palm plant. In some cases, other combinations of domains, including but not limited to M and I, M and K, M and C, I and K, or I and C can be overexpressed. In some cases, the SHELL polypeptide is functional with respect to binding to a SEP-like protein, binding to another copy of SHELL, or binding to DNA, but non-functional with respect to activating transcription of target genes.

### D. Use of Nucleic Acids of the Invention to Inactivate One or More Endogenous SHELL or SEP-Like Genes

Nucleic acid sequences encoding reagents that inactivate, replace, or knockout endogenous SHELL or SEP-like genes are also provided herein. For example, a TALEN, zinc finger nuclease, or chimeraplast can be constructed that recognizes a sequence within or near a SHELL gene (e.g., one or more of SEQ ID NOs: 152-154) or a SEP-like gene (e.g., one or more of SEQ ID NOs: 78-151). In some cases, the reagent is directed to a sequence conserved amongst more than one genes, such as a SHELL gene and one or more SEP-like genes, or more than one SEP-like gene such that 1, 2, 3, 4, 5, 6 or more genes are inactivated, replaced, or knocked out. In other cases, the reagent is directed to a sequence that is unique to SHELL or unique to a subset of SEP-like genes, such that only SHELL, less than 6, 5, 4, 3, or 2 SEP-like genes, or only 1 SEP-like gene is specifically targeted. Methods and compositions for designing and using TALENS, zinc finger nucleases, and chimeraplasts are known in the art, see, e.g., U.S. Patent Application Publication Nos. 2011/0145940; 2012/0329067; 2010/0257638; and U.S. Pat. No. 8,106,259.

In some cases, the TALEN, zinc finger nuclease, or chimeraplast can be used to target SHELL one or more SEP genes, or a sequence in proximity to SHELL or one or more SEP-like genes (e.g., within about 500 bp, 1 kb, 5 kb, 10 kb, 50 kb, 100 kb, or 1000 kb). Such targeting can induce single or double stranded breaks in the targeted sequence. In some cases, the single or double stranded breaks are repaired by the endogenous repair machinery such that the sequence is altered. The altered sequence can reduce expression of SHELL or one or more SEP-like genes, or reduce activity (e.g., reduce competency for homodimerization, heterodimerization, tetramer formation, DNA binding, or transcriptional activation of one or more target genes) of SHELL or one or more SEP-like gene products. The altered sequence can produce a SEP-like gene product that interferes with SHELL activity. Alternatively, the altered sequence can produce a SHELL gene product that interferes with activity of one or more SEP-like gene products. In some cases, oil palm plants containing the altered sequence can provide fruit with a reduced shell thickness or enhanced oil yield.

Methods are also provided in which a TALEN, zinc finger nuclease, or chimeraplast is used to target SHELL or one or more SEP genes, or a sequence in proximity to SHELL or one or more SEP genes, and a sequence homologous to the targeted sequence is introduced into the plant cell. Thus, single or double stranded breaks are induced in the targeted sequence, and the homologous sequence can be inserted at the targeted sequence by homologous recombination or endogenous repair machinery. Accordingly, targeted sequence replacement or knockout can be induced. The altered sequence can reduce expression of SHELL or one or more SEP genes, or reduce activity of SHELL or one or more SEP gene products. The altered sequence can produce a SEP-like gene product that interferes with SHELL activity, or produce a SHELL gene product that interferes with activity of one or more SEP-like genes.

### IV. Preparation of Recombinant Vectors

In some embodiments, recombinant DNA vectors containing isolated nucleic acid sequences suitable for transformation of plant cells are prepared. Techniques for transforming a wide variety of higher plant species are well known and described in the technical and scientific literature. See, for example, Weising et al. Ann. Rev. Genet. 22:421-477 (1988). Transformation of oil palm is also known in the art. See, for example, Izawati, et al. Methods Mol. Biol.; 847:177-88 (2012). A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant.

For example, for overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill.

Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers. Examples of environmental conditions that may affect transcription by inducible promoters include anaerobic conditions, elevated temperature, or the presence of light.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention can optionally comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

Nucleic acid encoding all or a portion of a wild-type SEP-like gene, or all or a portion of a mutant SEP-like gene operably linked to a promoter is provided that is capable of driving the transcription of the nucleic acid in plants. Nucleic acid encoding all or a portion of a wild-type SHELL gene, or all or a portion of a mutant SHELL gene operably linked to a promoter that is capable of driving transcription of the nucleic acid in plants is also provided. The promoter can be, e.g., derived from plant or viral sources. The promoter can be, e.g., constitutively active, inducible, or tissue specific. In some cases, the promoter can be a native or modified SHELL or SEP-like gene promoter. In construction of recombinant expression cassettes, vectors, and transgenics, of the invention, a different promoters can be chosen and employed to differentially direct gene expression, e.g., in some or all tissues of a plant or animal. In some embodiments, as discussed above, desired promoters are identified by analyzing the 5′ sequences of a genomic clone corresponding to a SHELL gene or a SEP-like gene as described herein.

### V. Production of Transgenic Plants

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Various palm transformation methods have been described. See, e.g., Masani and Parveez, Electronic Journal of Biotechnology Vol. 11 No. 3, Jul. 15, 2008; Chowdhury et al., Plant Cell Reports, Volume 16, Number 5, 277-281 (1997).

Microinjection techniques are known in the art and well described in the scientific and patent literature. The introduction of DNA constructs using polyethylene glycol precipitation is described in Paszkowski et al. EMBO J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983). Agrobacterium-mediated transformation of oil palm is also described in the scientific literature. See, for example, Iwazata et al., Methods Mol. Biol.; 847:177-88 (2012).

Transformed plant cells that are derived from any transformation technique can be cultured to regenerate a whole plant that possesses the transformed genotype and thus the desired phenotype. Such regeneration techniques rely on manipulation of certain phytohormones in a tissue culture growth medium, optionally relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillan Publishing Company, New York, 1983; and Binding, Regeneration of Plants, Plant Protoplasts, pp. 21-73, CRC Press, Boca Raton, 1985. Regeneration of oil palm plants from protoplasts has been described in Masani et al., Plant Science 210, 118-127 (2013). Regeneration can also be obtained from plant callus, explants, organs, or parts thereof. Such regeneration techniques are described generally in Klee et al. Ann. Rev. of Plant Phys. 38:467-486 (1987).

The nucleic acids described herein can be used to confer desired traits on species from the genera Elaeis, such as the oil palm plant Elaeis guineensis, Elaeis oleifera, or a hybrid thereof.

### VI. Identification or Production of Non-Transgenic Plants with Altered SHELL or SEP-Like Gene Expression or Activity

In some embodiments, methods and compositions for altered shell thickness or enhanced oil yield of oil palm fruits are provided that do not involve making or using transgenic plants, do not include the introduction of recombinant DNA into a plant, or do not involve the expression of a heterologous gene in the plant. Methods and compositions for identifying and/or sorting plants with altered shell thickness or enhanced oil yield that do not involve making, using, or screening transgenic plants are also provided. Such methods include, but are not limited to, marker assisted breeding. Marker assisted breeding involves the identification of a marker associated with a natural or induced variant and using that marker to assist the introduction of the variant into a commercially useful plant genetic background. Other non-transgenic methods for optimizing fruit morphology via alteration of SHELL or SEP-like genes or activity can include TILLING, and/or random mutagenesis. TILLING and/or random mutagenesis for production of non-transgenic plants with desired characteristics is generally described in, e.g., International Patent Publication No. WO/2006/032504; and U.S. Patent Publication Nos. 2010/0212043; and 2004/0053236. Still other methods can include identifying naturally occurring SEP-like gene mutations that confer an enhanced oil yield or altered shell thickness phenotype in a homozygous or heterozygous wild-type SHELL plant.

In some embodiments, a natural or induced genetic variation that alters SEP-like gene expression or activity can be identified by examining plants that have an altered fruit form phenotype as compared to the expected phenotype based on the genotype at the SHELL locus. In some cases, a natural or induced genetic variation that alters SEP-like gene expression or activity can be identified by examining plants that have a dura genotype (Sh⁺/Sh⁺) at the SHELL locus and a reduced shell thickness or enhanced oil yield phenotype as compared to most dura oil palm plants. Alternatively, a natural or induced genetic variation that alters SEP-like gene expression or activity can be identified by examining plants that have a tenera genotype (Sh⁺/sh⁻) and an altered shell thickness or enhanced oil yield phenotype as compared to the vast majority of tenera oil palm plants. In other cases, a natural or induced genetic variation that alters SEP-like gene expression or activity can be identified by examining plants that have a dura or tenera genotype at the SHELL locus and a pisifera phenotype. In still other cases, a plant with a natural or induced variation that alters the expression or activity of a SEP-like gene and provides a desired shell thickness or enhanced oil yield phenotype is identified, sorted or screened and the genotype at the SHELL locus is not known, not determined, or is determined after the identification, sorting or screening.

In some cases, the SEP-like variant can be confirmed, e.g., by sequencing one or more SEP-like genes or, e.g., by sequencing a region that includes, or is in proximity to, one or more SEP-like genes. Alternative methods for determining the sequence of the genome within or in proximity to one or more SEP-like genes are known in the art, and include DNA amplification with one or more primers that are sensitive to changes in the target genome sequence.

In some cases, a SEP-like variant can be identified, e.g., by sequencing, SNP analysis, or amplification, prior to, or in lieu of, determination of fruit phenotype. Markers can then be identified that co-segregate, or are expected to co-segregate, with the desired phenotype. In some cases, the markers include one or more polymorphisms that lie within, or in proximity to, a SEP-like gene, such as one or more of the SEP-like genes encoded by SEQ ID NOs:78-151. Thus, the phenotype of plants generated by breeding or crossing of parent lines can be predicted with high probability prior to fruit production.

In some cases, naturally occurring SEP-like gene variants can be identified, e.g., by sequencing, SNP analysis, or amplification, and their corresponding fruit form phenotype (e.g., shell thickness, mesocarp ratio, or oil yield) determined. For example, naturally occurring oil palm plants, e.g. plants with a wild-type SHELL genotype, with a reduced shell thickness as compared to a typical dura plant can be assayed for mutations in one or more SEP-like genes. Similarly, palm plants, e.g. plants heterozygous for the wild-type SHELL allele, with an enhanced oil yield as compared to a typical tenera plant can be assayed for mutations in one or more SEP-like genes. Alternatively, SEP-like variants can be identified and then their fruit form phenotype determined. Variants that are correlated with a desired fruit form phenotype can then be cultivated to produce oil palm plants with the desired fruit form phenotype and/or bred with traditional oil palm plant varietals to produce oil palm plants with the desired fruit form phenotype. Oil palm plants or seeds with the desired fruit form phenotype can then be identified prior to maturity (e.g., bearing fruit) by assaying for the presence of the mutation in the SEP-like gene that is correlated with the desired fruit form phenotype.

In some cases, naturally occurring oil palm plants that have an increased or decreased expression of a SEP-like gene, e.g., by ELISA, mass-spectrometry, dPCR, qPCR, RT-PCR, northern blot, microarray, SAGE, etc., and their corresponding fruit form phenotype (e.g., shell thickness, mesocarp ratio, or oil yield) determined. For example, naturally occurring oil palm plants, e.g. plants with a wild-type SHELL genotype, with a reduced shell thickness as compared to a typical dura plant can be assayed for increased or decreased expression of one or more SEP-like genes. Similarly, palm plants, e.g. plants heterozygous for the wild-type SHELL allele, with an enhanced oil yield as compared to a typical tenera plant can be assayed for increased or decreased expression of one or more SEP-like genes. Alternatively, plants with increased or decreased expression of one or more SEP-like genes can be identified and then their fruit form phenotype determined. Variants that are correlated with a desired fruit form phenotype can then be cultivated to produce oil palm plants with the desired fruit form phenotype and/or bred with traditional oil palm plant varietals to produce oil palm plants with the desired fruit form phenotype. Oil palm plants or seeds with the desired fruit form phenotype can then be identified prior to maturity (e.g., bearing fruit) by assaying for the increased or decreased expression of one or more SEP-like genes that is correlated with the desired fruit form phenotype. Alternatively, the genetic basis (e.g., mutation) for the increased or decreased expression of the one or more SEP-like genes correlated with the desired fruit form phenotype can be determined and detected to identify plants or seeds with the desired fruit form phenotype prior to maturity (e.g., bearing fruit).

In some cases, SHELL or SEP-like variants can be generated by random mutagenesis. For example, plants or seeds can be subjected to chemical mutagenesis, irradiation, random T-DNA insertion, or transposon mobilization. In other cases, variants are obtained by directed mutagenesis using recombinant DNA techniques as described above, e.g., using TALENS, zinc finger nucleases, or chimeraplasts. Methods for T-DNA insertion and transposon mobilization are well known in the art, see e.g.; Altmann et al., Mol. Gen. Genet. 247:646-652 (1995); Smith et al., Plant J. 10:721-732 (1996); Azpiroz-Leehan, et al., Trends Genet. 13:152-156 (1997); Long et al., Methods Mol. Biol. 82:315-328 (1998); Martienssen, R. A. Proc. Natl. Acad. Sci. USA 95:2021-2026 (1998); Pereira et al., Methods Mol. Biol. 82:329-338, (1998); van Houwelingen et al., Plant J. 13: 39-50 (1998); and Speulman et al., Plant Cell 11:1853-1866 (1999).

Chemical mutagens suitable for generation of SEP mutants include DNA alkylating agents, ethylmethane sulphonate (EMS), methylmethane sulfonate, ethylene imine (EI), nitrosoethyl urea, nitrosoethyl urethane, N-Methyl-N′-nitro-N-nitrosoguanidine (MNNG), triethylenemelamine, diepoxyalkanes (diepoxyoctane, diepoxybutane, and the like), 2-methoxy-6-chloro-9[3-(ethyl-2-chloro-ethyl)aminopropylamino]acridine dihydrochloride, procarbazine, chlorambucil, cyclophosphamide, diethyl sulfate, acrylamide monomer, melphalan, nitrogen mustard, vincristine, dimethylnitrosamine, nitrosoguanidine, 2-aminopurine, 7, 12 dimethyl-benz(a)anthracene (DMBA), ethylene oxide, hexamethylphosphoramide, bisulfan formaldehyde, and sodium azide. Irradiation includes subjecting a plant or seed to ultraviolet light, X-rays, gamma radiation, alpha radiation, or fast neutron bombardment. One of skill in the art will appreciate that other chemical or physical mutagenesis techniques are suitable for generating variants for marker assisted breeding.

The use of EMS, nitrosoguanidine or 2-aminopurine, and the like, in certain embodiments allows one to predict what mutation has taken place because these mutagens result in a high (95% or greater) frequency of specific base substitutions (transitions or transversions such as GC to AT transitions). Thus upon identification of the location of the mutation, one can determine from the known sequence, what the identity of the mutated sequence is with a probability equal to the specificity of the base substitution of the mutagen.

Random T-DNA insertion includes the use of Agrobacterium or Ensiferadhaerens organisms to introduce heterologous T-DNA into the plant cell genome. In some cases, the T-DNA inserts randomly into the genome and can interrupt or alter the genomic sequence at the site of insertion. Plants in which the T-DNA has inserted into, or in proximity to, one or more SEP-like genes can be identified by fruit phenotype or using molecular techniques (e.g., DNA amplification or sequencing). In some cases, the T-DNA can contain a marker such that organisms with the inserted T-DNA can be identified during breeding. In some cases, the T-DNA can contain sequences that suppress or activate nearby genes. For example, the T-DNA can contain one or more KPRE elements. KPRE elements can suppress expression of genes up to 3 kb or farther away (Lai C, et al. Plant Cell Rep. 28(5): 851-60 (2009)). Other suppression elements are known in the art.

Similarly, transposon mobilization includes the mobilization, or activation, of a transposable element in the genome of a plant cell. The mobilized transposable element will re-insert into the genome at random. In some cases, the transposon can insert in or near SHELL or in or near one or more SEP-like genes. The insertion of a transposon in or near SHELL or in or near a SEP-like gene can be identified by fruit phenotype and/or molecular techniques. The transposon can contain additional sequences such as markers or suppressor elements. Plants subject to such random mutagenesis protocols can then be screened for fruit phenotype or SHELL or one or more SEP-like genes can be directly assayed (e.g., by sequencing or DNA amplification) to determine the presence of desirable mutations.

TILLING (Targeting Induced Local Lesions In Genomes) is a reverse genetic strategy that combines the high density of mutations offered by traditional mutagenesis methods with rapid mutational screening to discover induced lesions. The method, combines the efficiency of mutagenesis methods, e.g., chemical-induced (for example, using ethyl methanesulfonate (EMS) (Koornneef et al., Mutat. Res. 93:109-123 (1982))), or radiation with the ability of mutational analysis tools, such as the detection of single base pair changes by heteroduplex analysis (Underhill et al., Genome Res. 7:996-1005 (1997)) to identify, concurrent with screening, the location of the mutation thus eliminating needless follow-up in areas such as introns, and non-conserved sequences. The TILLING method generates a wide range of mutant alleles, is fast and automatable, and is applicable to any organism that can be mutagenized, stored and propagated. Methods and compositions for TILLING are described in U.S. Patent Publication No. 2004/0053236. In some cases, TILLING methods can be combined with marker assisted breeding. For example, one of skill in the art can identify mutations within, or in proximity to, SHELL or one or more SEP genes and introduce desired mutations into commercial plants without the generation of transgenic plants. Such methods can allow the production of oil palm plants non-transgenic plants that have a reduced shell thickness or enhanced oil yield relative to dura or tenera plants.

VII. Sequences

SEQ ID NO: 1 >EG4P29517
MGRGRVELKRIENKINRQVTFAKRRNGLLKKAYELSVLCDAEVALIIFSNRGKLYEF
CSSSRVKLDDKSAKEGNAKETHMVTITQIMMKTLERYQKCNYGAPETNIISRETQSS
QQEYLKLKARAEALQRSQRNLLGEDLGPLSSKELEQLERQLDASLKQIRSTRTQYML
DQLADLQRKLEESNQAGQQQVWDPTAHAVGYGRQPPQPQSDGFYQQIDSEPTLQIG
YPPEQITIAAAPGPSVNTYMPGWLA*
SEQ ID NO: 2 >EG4P81074
MGRGRVELKRIENKINRQVTFAKRRNGLLKKAFELSVLCDAEVALIIFSSRGRLFEFC
SSSRTNAGTITKKKGKLVTVQIFTREYLKNKWVPDFELEPYSTHLKLILQPFSQELFIM
LKTLERYQRCNYSASEAAAPSSEIQNTYQEYVRLKARVEFLQHSQRNLLGEDLDPLS
TNELDQLENQLEKSLKQIRSAKTQSMLDQLCDLKRRLREAASQNPLQLTWANGSGD
HAAGSSNGPCNREAALSRGFFQPLACHPPEQIGTRAVLAKLKSTFINSLHFQLIEHWL
KVFT*
SEQ ID NO: 3 >EG4P15412
MGRGKVELKRIENKINRQVTFAKRRNGLLKKANELSVLCDAEVALIIFSSSGRRFEFC
SCSSVLKTIERYQTYNYAASEVVAPPSETQQNTYQEYAKLKARVEFLQRSHRNLLGE
DLDPLSTNELEQLENQVEKSLKQISSAKDSKWPYLKVSQITILPNFTLEGDQSCCHLT
HLMLDQLYDLKRKLQEAIPYNPLQWSWINGGGNGAGGASDGPCNHESALSEEFFQP
LACHPLQVGNSCDLVMGFKQNKDKFMQIFLATPRTHFPLYLEETTRCWVIDRAG*
SEQ ID NO: 4 >EG4P57231
MGRGKIEIKRIENSTNRQVTFSKRRNGIIKKAREISVLCDAQVSVVIFSSSGKMSEYCS
PSTTLSRILERYQHNSGKKLWDAKHESLSAEIDRIKKENDNMQIELRHLKGEDLNSLS
PKELIPIEDALQNGLISVRDKQHQQELAMDANVRELELGYPSKDRDFASHMPLAFHN
SVMERFTLRRET*
SEQ ID NO: 5 >EG4P67349
MGRGRVELKRIENKINRQVTFAKRRNGLLKKAYELSVLCDAEVALIVFSNRGKLYEF
CSSSSMLKTLERYQKCNYGAPETNIVSRETQEDRRPYLIYEMKENKSWT*
SEQ ID NO: 6 >EG4P109263
MGRGKIEIKRIENSTNRQVTFSKRRNGIIKKAREISVLCDAQVSLVIFSSSGKMSEYCSP
STTLSRLLEKYQVNSGKKLWDVKHENLSVEIDRIKKENDNMQIELRHLKGGDLNSLN
PKELILIEDVLQNGLTSVRGKQHHQELAMNGNVRELELGDPLKARDFACQIPIAFRE
WEEVA*
SEQ ID NO: 7 >EG4P29529
MGRGRVELKRIENKINRQVTFAKRRNGLLKKAYELSVLCDAEVALIIFSNRGKLYEF
CSSSRRNIELNV*
SEQ ID NO: 8 >EG4P115489
MGRGKIEIKKIENPTNRQVTYSKRRTGIMKKAKELTVLCDAEVSLIMFSSTGKFSEYC
SPLSEQRMGEDLDSLGIHELRGLEQNLDEALKVVRHRKILYPEGPLDLADIEYPFMEK
EIHDTVRKVVMLGDEKI*
SEQ ID NO: 9 >EG4P6889
MGRGKIEIKRIENTTNRQVTFCKRRNGLLKKAYELSVLCDAEVALIVFSSRGRLYEYA
NNRLLASTNLWREPFTRSPHVKATIERYKRACTDTSNSGSVSEADSQLNSSFLE*
SEQ ID NO: 10 >EG4P39137
MGRGKVELKRIENKINRQVTFSKRRNGLLKKAYELSVLCDAEVALIIFSSRGKLYEFG
SVGGSLVS*
SEQ ID NO: 11 >EG4P44072
MGRGRVELKRIENKINRQVTFSKRRNGLVKKANELSVLCDAEVALIIFSNRGRITEFC
SSSSGGTSQKLITSKAWKALELTTPYSIHEILSVVAIYPHLKSHTNLQQPEHSEFDDGS*
SEQ ID NO: 12 >EG4P62915
MGRGKVELKRIENKINRQVTFSKRRNGLLKKAYELSILCDAEVALIIFSGRGKLYEFG
SVGHLGNRIGVGRTPFRLSD*
SEQ ID NO: 13 >EG4P64304
MGRGKIEIKRIENTTNRQVTFCKRRNGLLKKAYELSVLCDAEIALIIFSGRGRLYEYSN
NRSVFIDLHPKDEGCFSQILYREL*
SEQ ID NO: 14 >EG4P104954
MKKIVKSKEIMGRGKIEIKRIENTTNRQVTFCKRRNGLLKKAYELSVLCDAEIALIVFS
SRGRLYEYSNNRCVYVDVR*
SEQ ID NO: 15 >EG4P82414
MGRGRVELKRIENKINRQVTFSKRRSGLLKKAYELSVLCDAEIALIIFSSRGKLYEFGS
VGSRANYNPAKETVTNVAINPLPPPPIKGEPIYTRDESQPFGKHTARKPILSRAFYLDL
VPNIENKTSISRLEILLPYSKACPQRKSERSVKLIMDRIISNMIRFLLSDIPLS*
SEQ ID NO: 16 >EG4P39130
MVRGKTEMKLIENATSRQVTFSKRRNGLLKKAFELSVLCDAEVAVIVFSPRGKLYEF
SSTSLSMPDTQQKSGSSQEPCSELLEDEELEGVDNVCDGVVGSGWTYDPYAKGNPL
QKEEHAKKLFFSLRLGKRNPTWVRSAVVTWNQLLEEQIATLKEQEQTLMEENALLR
EKCKLQSQLRPAAAPEETVPCSQDGENMEVETELYIGWPGRGRTNCRSQG*
SEQ ID NO: 17 >EG4P44048
MGRGRVQLKRIENKINRQVTFSKRRSGLLKKAHEISVLCDAEVALIVFSTKGKLYEYS
TNARLRSVFGGAGGGQPKSKLENGIFLQRTSKVSLWGYPPLLGQSRISAMLILGRGAF
FAHGCLSLLESSLDRNK*
SEQ ID NO: 18 >EG4P2672
MGRGRVQLKRIENEINRQVTFSKRRSGLLKKAHEISVLCDAEVAVVVFSTKGKLYEY
STDSRMDQGGLGGLASVRGGGLAGCPAVTVDDGEARDGWRQVKANERKAFNSQG
KPKNKKWSAPSWRWHPNLDAPLWH*
SEQ ID NO: 19 >EG4P15413
MGRGRVQLRRIENKINRQVTFSKRRSGLLKKAHEISVLCDAEVALIIFSTKGKLYEYA
TDSWLQAATTAWKTHWDLTISCWLADRQCNWHEATVGRRRGDPAARGRPSRWPV
AATDAHTFKKARIPFSKKSDDSGRRRSCTRARGERRRREEGEEAHLRRRRGFSGEQK
KDGTGTVSAVVFQRLPPTESRIFGERERGGFSLNRAGGGALSDSDWEPLLSSRTIELG
RPDLHGSLVAITGISAELCDCNR*
SEQ ID NO: 20 >EG4P155269
MEGIGELRGLIEKRTPAIWSKGRGHAAFPLSLPPLGIHGNGVPLKVRRKLEEKRVRISI
WKWISGELEVIPPLLKSKEIMGRGKIEIKRIENTTNRQVTFCKRRNGLLKKAYELSVL
CDAEIALIIFSGRGRLYEYSNNRN*
SEQ ID NO: 21 >EG4P11519
MARGKVQMRRIENPVQRQVTFCKRRAGLLKKARELSVLCGADIGIIIFSTHGKLYEL
ATNGDMQSLIERYKSIGAEAQIEGGEVNQPQVSEQEISMLKQEINLLQKGIRKCNLPE
SNSESHYYGEEEIEDNNKPRRLRHATGEGDERGREKVSREATGVEGRPSSGSAALAL
SPVSTDLRATDLGGVVANAAACVLGEAGWTSRPEGEVVAGRTLVEGLRKRNASKA*
SEQ ID NO: 22 >EG4P14715
MLMHLTLKDKCVGDELELEVGDGLTFGEVCVHKISYAALYTSPGVASLVLERGRCI
CFWCCEKRTMVRGRREIKRIENPIQRQSTFYKRRDGLFKKARELSILCDADLLLLLFS
SSGKLYEYHTPSVPSAEELVKRYEVATQNKIWRDLHLERNAEMEKVQKLCELLERD
LRFMKVDASQHYSLPVLDVLEGNLEAAINKVRSEKDRKIVGEINHLENMVRDRQQE
RYDLGDKVARAQGLKDMAVPLNRLDLKLGTCVS*
SEQ ID NO: 23 >EG4P82401
MVRGKTEIKRIENATSRQVTFSKRRNGLLKKAFELSVLCDAEVALIVFSPRGKLYEFS
STRYTGYLGKINVKIMQDKNKTLRACLVFVNILITLMPGNALSLQCHALLTPSQYNQ
NLSSTNDEGLRFKSDSSFNKMGEWPDSVLVK*
SEQ ID NO: 24 >EG4P37080
MVRGKTEVRRIENATSRQVTFSKRRNGLLKKAFELSVLCDAEVALIVFSPRGKLYEFS
SSSRLIVMAVTTSLADHVDRISENLNDRIVDNISEALRLLAPKPLHDFLHMCVSPRLD
RGVLRGVSSCWRVEAVVNPMT*
SEQ ID NO: 25 >EG4P63104
MRGPCEEHRAGRATRARLSLGRAPCAPAHWATCSQPSRMLPRAPAQAAYRKTQVR
RIENATSRQVTFSKRRNGLLKKAFELSVLCDAEVALIVFSPRGKLYEFSSSRATVSFGS
RKVWIIQATMDAEANDCGRASSTKMLSACNSCCVQAVGEWVYTAFNRGGSESKTR
EVSQDLGTESCAIEELHDLELQLEQSLSSIRNRKLNAEPRLQLCAPAVSDDYDSQNTD
VETELVIGRPGTCKVK*
SEQ ID NO: 26 >EG4P37079
MVRGKTEVRRIENATSRQVTFSKRRNGLLKKAFELSVLCDAEVALIVFSPKGKLYEF
SSSSRDGVEDQYSGGERTYSSLVSFSKYMLRNCTEDPLGMMIKPKLYHLVTKSYAGT
ILLQYRIQKTVDRYLMHTKDVNINIRATEQNMQCKTEPPVQLITQASSNGDACQNME
VETELIIGRPGTCEAKQQDHVSLNKQWSQENGAFGMESRQNP*
SEQ ID NO: 27 >EG4P29559
MVRGRVELRRIEDKTSRQVSFSKRRSGLLKKAHELAVLCDAEVGLIIFSAKGKLYDF
ASTSSVYRYNIIMDNRPELLEEKRIECYVALMHDLYIKIWCKIALSNVDYKLAAEFAL
LRCKPLTRPFNERHPTMSWKLLVEQRKAQTGYTPLNSTPHLYGGNWPGHSCTPLGS
G*
SEQ ID NO: 28 >EG4P43162
MGRGKIVIRRIENSTSRQVTFSKRRKGLLKKAKELAILCDAEVGFVIFSSTGRLYDFAS
SSEAELGHHKTKVYISATEWWQRIEFESDQIWVGSKNLQRPLHQYKDKTFFLRQHRG
KTFGSSLLQWMEDADNLWG*
SEQ ID NO: 29 >EG4P31052
MRLRLSSFTLHLPRPHPIIVYVASIVRVVFGFDGTKPSPLSDPDAPRATRPAPFAASPH
RHPLSFSLTTPMNPSPCGFIATYTVPESQEGGTVQNGGTNFRRESVWCILGSMVREKI
QIRKIDNATARQVTFSKRRRGLLKKAEELSILCDAEVALIVFSSTGKLYEYSSSSAPLP
FAAPLPSPIVSPYRRPSHAGGLLVPAMLVASLCCGLPARQHQLPPLAVCPLFTWAGV
GLPLDRPLPLPPLLSPIASIMKEIIEKHSMHSKNLQKPDQPPLDLNGEWLLHAIVTPKY
LHQVLTSNDEYFSPDET*
SEQ ID NO: 30 >EG4P86343
MVRGKVQMKRIENPVHRQVTFCKRRAGLLKKAKELSVLCDAEIGIIIFSTHGKLYEL
ATKGSYN*
SEQ ID NO: 31 >EG4P39902
MGRVKLQIKRIENNTNRQVTFSKRRNGLIKKAYELSVLCDIDIALIMFSPSGRLSHFSG
RRRFFEPDPLSITSMDELESCEKFLMEALRRVAERKHGGSWVKLVQLPRGWYQNELP
HLAVFTNDTKFLIPMLLKNTVICIVYRQKLL*
SEQ ID NO: 32 >EG4P48307
MDKLEARSFRTRFIGYPKKIMRYYFYLPENHNRRSDLITFNLPWRRCASLMRRHGSG
SHNTYLSCGQGMPLRAARVITRGSETITRTRKPNRPITTTPTCRVPRGEIRVPNGVWN
PRWASPLPVHLPRSSRPPAHSNGLSLGFRRPTAAAMRRGKVQIRRIEDKASRQVTFSK
RRGGLFKKARELAVLCDAEVGLIVFSPSGKPYEFCSSSRCVSILLLRLRSSDPSRSIDSL
RDQPGSVRQTLRSSSFLRRW*
SEQ ID NO: 33 >EG4P23857
MGRGKIEIKRIENPTNRQVTFSKRRGGLLKKANELAILCDVQASMRQYTGEDLSSMT
MNDLNQLEQQLEYSVNKVRTRKLSEHQAAMEHQQAAMEHKVPDVPMLEPFGLFY
QDEPSRNLLQLSPQLHAFRLQPAQPNLQEASLPGHSLQLW*
SEQ ID NO: 34 >EG4P29533
MVTLLLAQSSQQEYLKLKARVEALQRSQRNLLGEDLGPLSSKELEQLERQLDASLKQ
IRSTRTQYMLDQLADLQRRLEESNQAGQQQVWDPTAHAVGYGRQPPQPQSDGFYQ
QIDGEPTLQISVEGEEDEGELVEEDMEKRASDVKEELEYTLVYVMRYPPEQITIAAAP
GSSWAIISNKLDDEKEEEEGSFSDDDWRLTWDSEWVISMRLVMGSFPCFVKED*
SEQ ID NO: 35 >EG4P70708
MGEEHLSDGKTASPIQLSEESRRGMAREKIQIRKIDNATARQVTFSKRRRGLFKKAEE
LAILCDADVALIIFSSTGKLFEFSSSRVFMVIRVKLRTGLARWVLLQMITTLPKSGHSS
VGIPLISFKAIVVEMARAGRRVLTDSENVMYEDGQSSESVTNASQLVVPPNYDDSSD
TSLKLGSTDCGLTEVCVDYDLYVTTSCTLFEGYTAVRKQALSLFLYDRSTHAAQIDR
KRRQQVRIQEWRRLSKLTGLLAGALNLFGAVSGPKYDGKFLHSKVKELLGDTKLHQ
TLTNIVIPAFDIKLLQPVIFSTFEDDTLEGDTASVDVSTSENLRKLVQVGQDLLKKPVS
RVNLETGVSEACDVEGTNEDALIRFAKMLSNERKSRNAKMSAA*
SEQ ID NO: 36 >EG4P67350
MDKFEIAIKTSQQEYLKLKARVEALQRSQRNLLGDDLGPLSSKELEQLERQLDASLK
QIRSTRLEESNQATQQQVWDPNAPAVGYGRQPPQPQGDGFYQQIECDPTLHIGYPPE
QITIAAAPGPSVSNYMPGWLA*
SEQ ID NO: 37 >EG4P44069
MAEDRWRLAAGRRRAAQKWQRPAWVRRVRPSTCVRDAAQALAQACMRVQPRPT
RARAGNLMLKTIERYQRCSYNATDAIVPPKETQDLGPLSVKELEQLENQIEISLKHIRS
KKTQLMLDQLCDLERKEQMLQEANKALRRRLEEDTINSLQLSWQNGANVVGNAPC
DGEPPQTEGFFQPLGCEPSLQIG*
SEQ ID NO: 38 >EG4P67198
MSERGSREHWWWTEDVELKRIENKINRQVTFSKRCNGLLKKAYEVSILCDVEVALII
FSSRGKL*
SEQ ID NO: 39 >EG4P130373
MVRKPSMGRQKIDIKRIESEEARQVCFSKRRAGLFKKANELSILCGAEIGVIVFSPAGK
PFSFGHPSVDSIIDRFLFGSPSPTTLPSADPRMPVAREMMVVHEFNQQYTVLTALLETE
KRKKAVLEEAVRVKQAGEAALWGANIEELSLGELESLHKSFERLRRDVAMRADQL
VIEAAHTRSSSVAAAGSFVPPPPLGVNLGFGRGVEGSMALPPPTFFGYGRGPF*
SEQ ID NO: 40 >EG4P128041
MDRGDVDLQKIDGKENLANPFTKALTIKEFDNHKKKEEEALRTTPTEDDDDMILLDE
GVDIASSSKRDNSDHACNMVRKPSMGRQKIDIKRIESEEARQVCFSKRRAGLFKKAN
ELSILCGAEIGVIVFSPAGKPFSFGHPSVDSIIDRFLSGSPSPMTLPSADPRMPAAREMM
VVHEFNQQYTVLTALLETERRKKAVLEEAVRVKRAGEAALWGANIEELGLGELESL
YNSFERLRRDVAMRADQLVIEAAHTRSSSVAAAGSTVPPPPPGVNLGFGRGVEGSM
ALPPPTSFGYGRGPF*
SEQ ID NO: 41 >EG4P147209
MGRQKIEIKRIQNEEARQVCFSKRRTGLFKKASELSILCGAEIGVVVFSPAGKAFSFGH
PSVDAVFDRFLTGNPHHGNSGGPAADSRRGAVVRELNRQYMELHGLVDAERKRRE
ALEEAMKGEQGGRPYWWDNNVDSLALEDLEEYEKKLLELRNNVAKVADQLLHEA
MARKQQQHHHHHHQQQQQQFPMVGAAVALPGPFAIKNEDAIHPSLGGGLGFGHGF
F*
SEQ ID NO: 42 >EG4P37712
MGRQKIEIKRIESEEARQVCFSKRRVGLFKKANELSILCGAEIGVIVFSPAGQPFSFGHP
SVDSIIDRFLSGGPSPPTLASADRRMPAAREMMVVRELNRQYTELAALLETERRRKV
VLEEAVRVKRAGEAALWGANVDELGLGELERLHKSLERLRRDVARCADQLVIEAA
HARSSSIAAASRSTAPPPPPGIHLGFGRGLEGSMALILPPPPTPTAFGYGRGLF*
SEQ ID NO: 43 >EG4P153108
MVKAEVELMGIVEDKTLERYQKCNYGAPETNIISRETQILELVEWIRYKWLDEDIDK
NLLGEDLGPLSSKELEQLERQLDASLKQIRSTREQMLCEANKSLRRRLEESNQAGQQ
QVWDPTAHAVGYGRQPPQPQSDGFYQQIDGEPTLQISVEGEEDEGELVEEDMEKRA
SDVKEELEYTLVSSRTNNNRSSTRDTDESIEIKGLKLQKFDKDQGEGQHTAL*
SEQ ID NO: 44 >EG4P108259
MGRQKIEIKRIESEEARQVCFSKRRAGLFKKAIELSILCGAEIGVIVFSPAGKPFSFGHP
SVDSIIDRFISGSPSPTTIPSANPRMPAAREMMVVRELNRQYTDLAALLETERRKKVV
LEEAVRVMRAGKAVSWEANIEELGLGELEGLQKSFERLRMDMAMRADQLVIEAAH
AQSSSMAAASSAAPPPSGVSLGFGRELEGSMALPPPTFFGHGRGLF*
SEQ ID NO: 45 >EG4P71703
MARRTSHGRRKIEIKRIEDEQTRQVTFSKRRGGLFKKASELSTLCGAQVGILVYSPGG
RPYSFGQPGFVEVSDRFLPCVPTPIGSDPPPMPPPAYLSVSQPSKHYLEVVNVLEAAR
AKGAVLKERLAMVLEEEGRAYESENDDLTVEELGDLVARLEALKMRVFSRFSTILN
QQQASSSSAALTVTPLNVINPYATNGPQAYPGGGFVLGNNGHGAGGFLGTGGHGTP
SGFMGNDGNGPLGFIA*
SEQ ID NO: 46 >EG4P2959
MVRKTSNGHRKIEIKRIENEQIRQVTFSKRRQGLFKKASELSTLCGAQVGILVYSPAG
RPYSFGQPGFEVVSNQLIAHNSFMTSPNPIEGPQGNAIVQQLNCHCMEIMSLLDTAKT
KGAVLKERLEITPKGKEKAFETELEGFGMDELERLVKSYNDLKLKADSRIYKIMSGG
ASSSGGPLPVNPKLARDRELLFQPNICLEIFSIIKDRSMQRGAE*
SEQ ID NO: 47 >EG4P82416
MAKLKAKFESLQRSQRHLLGEDLGPLSVKELQQLERQLESALSQARQRKAQIMLDQ
MEELRKKVSMLDEGQGSEHLEARFPCSIEEIAIVGFSRVV*
SEQ ID NO: 48 >EG4P14105
MGRVKLKIKKLENSSGRQVTYSKRRAGILKKAKELSILCDIDLVLLMFSPTGKPTLCV
GDRSTIEEVVAKFAQLTPQERAKSYWTDPDKINNVDHIGAMEQSLQESLSRIQVHKE
NLGKQLMSLDCSGQVKALLGKQAEANDQLQEDSLHEFSQNACLRLQLGGQYPYQS
YCQNLIGENAFKPDTENSLPESTIDYQVDHFEPPRPGYDASFQNWASTSGTCDVAIYD
DQSYSRRSAFRHSIDPVAYRGSYDWCPSTCVPQCFPYPPTSAVPAPNHDRSFPKRRLI
NIHPVNLRDPLLKPHLFLGSLKNHVPKWRSQKDLARANPASGLPTRASRGTHTLTPP
KREQIKSTHTCQRHNILL*
SEQ ID NO: 49 >EG4P37867
MSKEIVGKKTPYPHEEALAGSQGQGVSKNSQQDCTLAKGTAISWKPWNAPPQSHHY
SAIETARAQNSTATTSKLVKTSGRLSAEMARGKVQMRRIENPVHRQVTFCKRRAGLL
KKAKELSVLTDADIGDISSKARDQHTTEVFEIVEQNGHFDVAPMMVQQNGHFGVSP
MIVQQNEHFTAAPAMEDIPYPLTIQNDYSSFTSLDMG*
SEQ ID NO: 50 >EG4P71708
MATMPKKTMGRQKVKLKRIENEDALYVTFSKRKSSLFQKAAELATLCGSEIALVVFS
PAGRPYSLGLPTVDKVFHRVLSSGPAQMGSGHSVVSHSAKQCSEITKHLEQEKSRKA
ILVERLQKEAPPRWEDGLHGLGWDDLLILAKEVEELKSKVDSRVCEILLQGASSSTA
NADAWPVGSSEGSYGVGPRGPLDNNI*
SEQ ID NO: 51 >EG4P37348
MPRKTRTTRGKQKIEIKRIEKEEARQICFSKRRSGVFTKASDLSTLCGPDVAVLAFSPR
GKPFSFGSPAVNPVIDRFVLDISSSPGSGHHCGPPSNTVQQLSKLCLDLTNQLHACKA
KSAVLEEKLSSPGYDILELDWFENVDDLELDKLGKLAEALKRVKVNADAHVDARLL
HGRGALSSSTTPVMTANQVEGASSSNRVMAAASSKGVMAAGNVPVAFLTISMLAM
FGNMIKKNHLDNVEVSPYWTRLDAK*
SEQ ID NO: 52 >EG4P71707
MAERTFRGRQKIEIKKIEKKAARDVTFSKRRVGVFGKASELATLCGVDIGVVAFSPA
GRPYTFGHPDANVVFNRFLGLVQPEGSSGSVGAMARHRAEMLRQLTLHCSQMMDR
LAAEREKRAVLEERLRKVSEDPQERAWPEDLEGLGLERLARMVRGFEEQRAKARAR
LHQIRELGESSSGPSATVEFKKSVV*
SEQ ID NO: 53 >EG4P104943
MNGENDAASRIIFSSLKERLVQSGVSYAKAVKKHPIPSPVVRKSTETVKDLMSSNSG
NVHHHPRSRGHRVKLLSKGTCFRCGDRDHTRESCRNPIKCFLCKGYGHVQKSTASPF
WKGVLSTHGLFQQLFSITIGNGKWVSCWTFIKSTIERYKKACANTSNSGSIVDVDSQQ
YYQQESAKLRHQIQILQNANRHLMGDSLGSLTVKELKQLENRLERGITRIRSKKIAET
ERAQQVSIIEAGHEFDALPGFDSRNYYHPHISQQKSMMALVNEKEQSQNQSQLLQEL
GQSE*
SEQ ID NO: 54 >EG4P35645
MGRSKVKLKFIEEQHRRSATYRRRIAGLKKKASELAILCDIPVLVISFGPREQVETWP
EDNQAARHIIDRYRELSIDIRNKNKLDLPGYMKAEIIRHQASFNRRCRDLADMPLLPL
DGLFYALLKSLRELAHQLDSRMEVIKERIQLLKDRKHFNLGETMNMGSQLLEITPRD
GMMGIQNTASAYDMMFSDPYLTMNASLQDPPQPTSFSSGQISPDAFLQYLYGPMGM
DEVPLAMVPSIPSNMDEVPLAMMPSIPMNMNEPPGAQLAKLCD*
SEQ ID NO: 55 >EG4P37749
MARKKVNLAWIANDSTRRATFKKRRKGLMKKVSELATLCDVKACVIVYGPQEPQP
EVWPSVPEVTRVLARFKSMPEMEQCKKMMNQEGFLRQRVAKQQEQLRKQERENRE
LETMLLMYQGLAGRSLHSLRIEDATSLAWMVEMKVKAVQERMGLVRAQMASSSQ
QVVLEAPIEAPAPMAVMKEKTPLEAAMEALQRQNWLMEVMNPNDNLMFGGGEEM
VQPYMDHTNNPWLDPCYFPLN*
SEQ ID NO: 56 >EG4P154153
MARNKVKLAWIANDATRRATLKKRRKGLLKKVQELSILCGVEACAIVYGPNDRVPE
VWPSPPEAARIVGRFKSMPEMEQTRKMVNQEGFLRQRAVKLLEQLRKQERENREME
MKLLIREGLKGRSFDNLGIEDVTCLSWMLERKIKEIYDKMDEIKNKVTVNQVAGGPS
ALPLQVMAPPPAAPIGPVVPKEKTTVEQAMEALQRQNWFMDMMSPWPEDFYQPAQ
PMDPYQPPPPAPLDHTIPWPDPSFPFN*
SEQ ID NO: 57 >EG4P45603
MARNKVKLAWIANDATRRATLKKRRKGLLKKVQELSILCGVEACAIVYGPNDRVPE
VWPSPPEAARIVGRFKSMPEMEQTRKMVNQEGFLRQRAVKLLEQLRKQERENREME
MKLLIREGLKGRSFDNLGIEDVTCLSWMLERKIKEIYDKMDEIKNKVTVNQVAGGPS
ALPLQVMAPPPAAPIGPVVPKEKTTVEQAMEALQRQNWFMDMMSPWPEDFYQPAQ
PMDPYQPPPPAPLDHTIPWPDPSFPFN*
SEQ ID NO: 58 >EG4P140076
MARRRRRWQFIENQRQRLATYRKRRGGLRKKASQLSSLCGVPIAVISFGPNGRLDT
WPDDQGAIHDLLLTYRSFDPEKRRKHDLDLPTLLEAQEGSQNLLWDPRLDAMPTES
LRNLTNSLDSKVKAIDERIQQLLEENSKCSNQDNNNSSREQGVNSKCNDQDNNNTGS
EQRDDSKSSNQAKQIKRVRK*
SEQ ID NO: 59 >EG4P41944
MGKIEKKEALHICFTKRRQGIFKKAGELAVLCGAQITVITLSPGGKPFSFGQPSTDAVI
ARYLDPGRHQVPIPITTSLEIRLRYYLKYCKLGEQSGGGLWWWEAPIDGLDLEELVV
MKGAIEELYKAILKKANQPTSAGEAVQGMPQKPSLAMLNGLDSCDWLIQLLANCSQ
WLRDLKRVCGSLLSIFPNITIKAEVRGSVDRRLATHIIRDEDKQQVHRSTAIMRINV*
SEQ ID NO: 60 >EG4P3001
MRRSQVKRILLKCPVKKAKEGEEPLEAVANKIWPNDDLEFQSGKSMIQKVKGMLRV
RSMDTAIYSSKVMYLPKITLPYQKFTNTWCLGWFGPIIQQLPIGSAPGTLTFVTCRSES
QTHPRTWLTTSPTWDTSMKSVIERYNKTKEENHLVMNASSETKPIRFRLASTAKSHN
SDGADERGKDSNLMLVDAHERQELLTDLGRNQPHKHHFYRNREADHIQPQGGAAIS
YEVKDVFVQEDGIFWQREAASLRQQLHNLQESHRQLLGEELSGLSVKDLQNLENQL
EMSLRGIRMKKVYAMRGVNGIDKGPITPYGFNVTEDANISIHLELSQPQLQTDATLA
QGQGNKEVDQGHSHQPTNEDIMPSGFTIEYVLAIEQVVAGAPTAPFPRGQRGPTLDP
RRANLGRRHVGVVGGGNLFAKRYDFLEENVGFRRVTIISLQKYGTSTESISRLRSNLF
QNNKKS*
SEQ ID NO: 61 >EG4P60802
MTNRGRGLQLIENRTQCLVTYRKRRESLKKKANQLSSLCGVLIAVISFDPDGRLHTW
PDDQGALPDLLLTYRSLDPKKRQKHDLDLPTLLGAMPAGSLRTGPAKGHLCLRKLA
NSLHSKVEAIDERIQQLLDKNSKCTNQDNNSTSREQDDDSKCNKKGKNNNTSSEKG
DDDSKGSNQGNNNNNTSSEQGDYSKSNNEGNDKNKVCLLVVTRWSFIPSL*
SEQ ID NO: 62 >EG4P14015
MSRSSMKLELIADDAARKTSLKKRKKGLLKKVQELSILCDVDACAIIYEPDDRHPEL
WPSSEEATRMLVRLRSMPEMEQKQKMMNQEEFLYQKMRKLVDQLHKQEFENKEL
EKKLKMYEALRTGDFSELDMEQAMNLSMMIEQMLKKIYEKMDAIKKHQAAMARV
DGVVQEGGNAAGLNTPRENTPTEKDNEILQRQKQMLDMMIPRSSKTYQPSAGPTNP
WPANSLFPFN*
SEQ ID NO: 63 >EG4P21371
MTNPDDGEVGGGGGSERCVASEKVTGKKARRATFKKRKKGLMKKVSELSTLCDVK
ACLIVYGPNEPEAEVWPSVPDAMRVLTKLKKMPEMEQSKKMMNQEGFMRQRIMKL
QEQLRKQDRENRELETILLMYQGLAGRSLHTVTIEDMTSLAWLIEMKVNKVQERIEH
SKGEIASKMVEGMKEEKKKVEGPSNIKEKISLEVAMEELQRQEWFTEIMNPHDLMIC
GNEVVQPYIDHNNPWLDAYFP*
SEQ ID NO: 64 >EG4P122402
MGRHKIPVKMIDKKDESNICFSKQKKGLFSKAKQIARAGSEVAIIVFSRVGNIFTFCHP
SIESVASRFLSQQNIKHRSSNDDNFHGNADFVYPGSDAARGGLTGPSEEGETSNKGD
NKLDGGNTIMQDKGFESDHEEEEVESKTSSKAEGSDVAGSSQEEHALMHDGEEHAT
GEKETSSDETLHSGRFWWNNRIDNRELHELLEFESALVELREKVRDQANQILVQKPV
MGYYLDFSNYKFKFDEQASQD*
SEQ ID NO: 65 >EG4P42750
MVPRAELWAVWAGIAYARLALTVDRLIIEGDSGTMVKWIQMRDTEDAAHPLLRDIA
MLLRGATITAVTIRMENLSIRASSFSLTNGRSELSGLVCGGVPKIQSSIFTERVSSCISR
VDSPFVPVCSNVPEKLMGEQLSGLNVKELQNLEIQLERSLHCVQKKKGYLLHNENIE
LYKKVNLIRQENMELRKKPRNILSRTDKA*
SEQ ID NO: 66 >EG4P157194
MNGENDAASRIIFSSLKERLVQSGVSYAKAVKKHPIPSPVVRKSTETVKDLMSSNSG
NVHHHPRSRGHRVKLLSKGTCFRCGDRDHTRESCRNPIKCFLCKGYGHVQKGFATL
STKIETGATSCPVSLVVLESKTSLPLSLCRFLRGPYWKVILGYIARDTSELSYDDCFER
RERTFGWRGLFFGPSAITSLSSLWCRLPICNLRRPYLVLFSFRQNLNLVDKHLMGDSL
GSLTVKELKQLENRLERGITRIRSKKIAETERAQQVSIIEAGHEFDALPGFDSRNYYHV
SMLEAAPHYSHQQDQTALHLGI*
SEQ ID NO: 67 >EG4P6887
MGLRNKPPNQRRYGISYERNFKGIPRNLMGESLGSMSPRDLKQLEGRLEKGINKIRT
KKIAENERAQQQMNMLPQTTEYEVMAPYDSRNFLQVNLMQSNQHYSHQQQTTLQL
GKKIVDRVASSTDRSDVGIIQDLPNQRGPEGRRPWSDGLQQHGRWFGSGD*
SEQ ID NO: 68 >EG4P91665
MSIVDNSDMSMASCRLQLIESRRQRLATYRKRRESLKKKANQLSSLCGVPIAVISFGP
NG*
SEQ ID NO: 69 >EG4P126213
MEVLPIIDLHPTVILGSVLELPQREGKPQRRIEEAKKNWFFHPWMDDRRSRRALLFPL
RDANDPTPAHDSDLSQQGLWQPPTATPSQPRSVTDIWLCKWIESDFRNSFGSWEELF
FLKINFQPVFSRHLMGDALSSLSVKELKQLENRLERGITRIRSKKIAENEQAALQVSIA
QEGPQFDALPAFDSRNYYHVNLLEAATHYSHQQDQTALHLGYEARSDHAA*
SEQ ID NO: 70 >EG4P36286
MPRRKVVLEPHPTEQARMQCYLTRRNGIKKKVRELSILCDADIAHLSIPPAGEPSLFL
GAHTSCGGLVVLAGSVYSTIALHP*
SEQ ID NO: 71 >EG4P3542
MAPPLGSGAATSGGNGDGRGERYRWKSIEKRTWGLCKKAYELATLCDVDVALICY
LPSVDTPTIWPPYRHKVEQVVHRYVDIPADKKLPKNQITLHIPNSTAGNTKDAGEAA
AVADADRIRVPFPYDEDKLIAIVRYLDSKIVEVRRMIAARRMERRSEPALAVASGGD
GDPGTADWDRGKRVARDCGPVWGRGRPDFSALAAAAAAAARGGGSGGAPNSSRS
CLCCYCPHHGHWFTGFDGRNASRDGSDGI*
SEQ ID NO: 72 >EG4P71936
MAPPRGDGRSDKSLRLSIKNRTKGLCKKAYELATLCDVELALVSYPSDGAEPTTWPP
DRSKIEDAFHRYFETPAHKKLPKNQITLDNPNPGAVEKKDAAKAAASKAPKETDRLR
IPFPDDEDKLIALRGILDSRLEAVRKMIAIRRAEERRDPRPSARDTEKELAVAVANAG
GGDPTPSAGDPGKRLAQGQGGPLPAAAAVAAASAGREDPRPSVRDVEKMVAGDCG
PVSGRGNPDCSAAPAAAGSGGGGAPNSWLQPSAHGGRSHWSYRLQTEPTFSPQKEA
AGNGRYPPGTRESVAYPVIQPKLQWHSSSLAPPQRHLLREAASPITPPFTVTWHRRRF
THFLRRRNATYDTVHGKWKHHDIKVKDSKTLLFGEKQVTVFGIRNPEEIPWGETGA
EYVVESTGVFTDKEKASAHLKGGAKKVIISAASKDVPMFVVGVNEHEYKSDIDIVSN
ASCTTNCLAVLAKVINDKFGIIEGLMSTVHSITATQKTVDGPSSKDWRGGRAASFNII
PSSTGAAKVGRSFGVLTTTYKDAAEDKADRCRNQTVRGEEEADVWDRTLTTAEETL
NSSADRRRIGGRSVGAGNCTFGSDSASGRAASGGSGRRNIGDFTD*
SEQ ID NO: 73 >EG4P29531
MEGVEKIEEIIARELNMMKTLERYQKCNYGAPETNIISRETQEDVDALYGQVCDIFLK
YPNELAVEWSEGLD*
SEQ ID NO: 74 >EG4P44436
MREAIGGSQPRAQGGERRSRDRGDGRRSRARGGRLGGQGGRRQAGARGRELEEVG
GSQGLEEASRGLREAEGGRGLTVGGSESRETAWILGRRSDAHSRGLEEVRDGRMLTI
GGSRRRRQRKEGVGKNKGGWQGTGLGLSSTAINKASYPSQEPEAWSKPMVGKKLN
VEFIKHRKKRLATYRRRKEALKQAAYELSTLCGTPTAVIYFGPDGQPESWPEDEGAV
RDIIGRHPGLGAKKRSTRPFDLRDLPPFDDTSEEFLREMLCSMESGMEAVKERIQLLK
KDSRCNQGDFHGDTGGVQQQGCQCNNPAFMEECFDVPMVSKAAMDDGPGQGHGA
FAPMELKQVEGVAADAFLPCSSNASMDFNDELAAFSMPLIFMPPPFTGATSEHDIACI
WQ*
SEQ ID NO: 75 >EG4P37875; SHELL (encoded by the DeliDura
Allele; Sh^{DeliDura}; Sh⁺)
MGRGKIEIKRIENTTSRQVTFCKRRNGLLKKAYELSVLCDAEVALIVFSSRGRLYEYA
NNSIRSTIDRYKKACANSSNSGATIEINSQQYYQQESAKLRHQIQILQNANRHLMGEA
LSTLTVKELKQLENRLERGITRIRSKKHELLFAEIEYMQKREVELQNDNMYLRAKIAE
NERAQQAGIVPAGPDFDALPTFDTRNYYHVNMLEAAQHYSHHQDQTTLHLGYEMK
ADPAAKNLL*
SEQ ID NO: 76 >SHELL (encoded by the MPOB Allele; sh^{MPOB};
sh⁻) (amino acid change italicized and underlined
in the following listing)
MGRGKIEIKRIENTTSRQVTFCKRRNGL [symbol] KKAYELSVLCDAEVALIVFSSRGRLYEYA
NNSIRSTIDRYKKACANSSNSGATIEINSQQYYQQESAKLRHQIQILQNANRHLMGEA
LSTLTVKELKQLENRLERGITRIRSKKHELLFAEIEYMQKREVELQNDNMYLRAKIAE
NERAQQAGIVPAGPDFDALPTFDTRNYYHVNMLEAAQHYSHHQDQTTLHLGYEMK
ADPAAKNLL*
SEQ ID NO: 77 >SHELL (encoded by the AVROS Allele; sh^{AVROS};
sh⁻) (amino acid change italicized and underlined
in the following listing)
MGRGKIEIKRIENTTSRQVTFCKRRNGLLK [symbol] AYELSVLCDAEVALIVFSSRGRLYEYA
NNSIRSTIDRYKKACANSSNSGATIEINSQQYYQQESAKLRHQIQILQNANRHLMGEA
LSTLTVKELKQLENRLERGITRIRSKKHELLFAEIEYMQKREVELQNDNMYLRAKIAE
NERAQQAGIVPAGPDFDALPTFDTRNYYHVNMLEAAQHYSHHQDQTTLHLGYEMK
ADPAAKNLL*
SEQ ID NO: 78 >EG4N29517
ATGGGGAGGGGAAGGGTGGAGCTGAAGAGAATCGAGAACAAGATCAATCGCCA
GGTGACCTTCGCGAAGCGGCGGAATGGGCTCCTCAAGAAGGCCTACGAGCTCTC
CGTGCTCTGCGACGCCGAGGTTGCTCTCATCATCTTCTCCAACCGCGGGAAGCTT
TACGAGTTCTGCAGCAGCTCCAGAGTTAAGCTTGATGATAAGAGTGCCAAAGAA
GGTAATGCAAAAGAGACACATATGGTCACCATCACTCAAATTATGATGAAGACA
CTTGAAAGGTATCAAAAATGCAACTATGGTGCTCCGGAGACTAATATTATATCAA
GAGAGACTCAGAGTAGTCAGCAGGAGTACTTGAAaCTAAAAGCACGTGCTGAAG
CCTTACAGAGATCGCAAAGAAATCTCCTCGGTGAGGACTTGGGCCCACTCAGCA
GCAAGGAGCTTGAGCAGCTTGAGCGGCAACTTGATGCATCGTTAAAGCAAATCA
GATCAACACGGACCCAATACATGCTTGATCAGCTTGCAGATCTTCAACGAAAGTT
GGAGGAAAGTAACCAGGCTGGTCAGCAGCAAGTTTGGGATCCCACTGCTCATGC
AGTAGGCTATGGCCGGCAGCCACCTCAACCACAGAGCGATGGATTCTACCAACA
GATAGATAGTGAACCTACTCTCCAAATCGGGTATCCTCCAGAACAAATAACAATC
GCAGCAGCACCCGGGCCAAGTGTGAATACTTATATGCCAGGATGGCTTGCATAA
SEQ ID NO: 79 >EG4N81074
ATGGGGAGGGGAAGGGTGGAGCTGAAGAGGATCGAGAACAAGATAAACAGGCA
GGTGACGTTCGCCAAGCGGCGGAACGGGTTGCTGAAGAAGGCCTTCGAGCTCTC
CGTCCTCTGCGACGCCGAGGTCGCCCTCATCATTTTCTCCAGCCGCGGCCGCCTCT
TCGAATTCTGCAGCAGCTCCAGGACCAATGCGGGAACAATAACTAAAAAGAAGG
GAAAACTTGTAACTGTTCAAATCTTTACTCGAGAATATCTGAAAAATAAGTGGGT
GCCCGACTTCGAACTCGAGCCATATAGTACACACCTGAAGCTGATTCTCCAACCT
TTCTCTCAAGAACTTTTCATCATGCTTAAGACACTCGAAAGGTACCAAAGATGCA
ATTATAGTGCATCAGAAGCTGCTGCTCCGTCAAGTGAGATACAGAACACTTACCA
AGAGTACGTGAGGCTGAAGGCAAGAGTTGAGTTTCTGCAGCACTCACAGAGAAA
TCTCCTTGGTGAGGACTTGGACCCACTAAGTACAAATGAACTTGATCAACTTGAG
AATCAACTAGAGAAATCTTTAAAGCAGATCAGATCAGCAAAGACACAATCAATG
CTCGATCAGCTTTGTGATCTTAAAAGAAGGTTGCGAGAAGCAGCTTCACAAAATC
CCCTCCAATTGACATGGGCAAATGGTAGTGGTGATCATGCTGCTGGTTCATCAAA
TGGCCCTTGTAATCGTGAGGCTGCTCTATCAAGGGGATTCTTCCAGCCATTGGCA
TGTCACCCTCCTGAGCAAATTGGAACACGGGCTGTACTCGCCAAGCTGAAGTCCA
CTTTCATCAACAGCCTCCATTTTCAGTTAATAGAGCATTGGCTCAAGGTGTTCAC
ATGA
SEQ ID NO: 80 >EG4N15412
ATGGGGAGGGGGAAGGTGGAGCTGAAAAGGATTGAGAACAAGATAAACAGGCA
GGTTACCTTTGCAAAGCGACGGAACGGATTGCTGAAGAAGGCTAACGAGCTCTC
TGTCCTCTGCGACGCCGAGGTCGCCCTCATCATCTTCTCCAGCAGCGGCCGCCGC
TTCGAGTTCTGCAGCTGCTCCAGCGTGCTTAAGACAATCGAGAGGTACCAAACAT
ACAACTATGCTGCATCAGAAGTTGTTGCCCCACCAAGCGAGACACAGCAGAACA
CTTATCAGGAATATGCGAAGCTGAAGGCAAGAGTTGAGTTTCTGCAACGTTCGCA
TAGAAATCTCCTAGGTGAGGACTTGGACCCATTAAGTACAAATGAACTTGAGCA
ACTTGAGAATCAAGTAGAGAAGTCTTTAAAGCAGATCAGTTCAGCAAAGGATTC
CAAATGGCCATATCTCAAGGTGTCTCAGATCACCATTCTTCCCAACTTCACCTTA
GAGGGTGACCAATCATGCTGTCATCTTACGCATTTAATGCTTGATCAACTTTATG
ATCTTAAGAGAAAGTTACAAGAAGCCATTCCATATAATCCCCTCCAGTGGTCATG
GATAAATGGTGGTGGCAATGGTGCTGGTGGTGCATCCGATGGCCCTTGTAATCAC
GAGTCTGCTCTATCAGAGGAATTCTTCCAGCCATTGGCATGCCACCCTCTACAAG
TTGGTAATAGTTGTGATCTGGTTATGGGATTCAAGCAGAATAAGGATAAATTTAT
GCAGATTTTTCTTGCAACGCCTCGTACACATTTCCCGCTTTACCTGGAGGAGACT
ACGAGATGTTGGGTGATTGACCGGGCCGGGTAG
SEQ ID NO: 81 >EG4N57231
ATGGGGCGAGGGAAGATTGAGATTAAGCGGATCGAGAACTCCACCAACCGGCAA
GTGACCTTCTCCAAGCGGCGGAATGGGATCATCAAGAAGGCACGGGAGATCAGC
GTCCTCTGCGATGCCCAGGTCTCCGTCGTCATCTTCTCCAGCTCCGGCAAGATGTC
CGAGTACTGCAGCCCCTCCACCACGCTGTCGAGGATTCTCGAGAGGTACCAGCAT
AACTCTGGCAAGAAGCTCTGGGATGCCAAGCACGAGAGTCTTAGTGCTGAGATC
GACCGGATCAAGAAAGAGAATGACAACATGCAGATCGAGCTGAGGCATTTGAAG
GGTGAGGATCTGAACTCACTGAGCCCAAAGGAACTCATTCCAATTGAAGATGCC
CTCCAGAATGGTCTCATCAGTGTTCGGGACAAGCAGCACCAGCAGGAATTGGCA
ATGGATGCAAATGTAAGGGAACTGGAGCTTGGATATCCTTCGAAAGATAGGGAT
TTTGCTTCCCACATGCCACTAGCCTTCCATAACTCCGTAATGGAAAGGTTCACAC
TCAGGCGGGAGACTTAG
SEQ ID NO: 82 >EG4N67349
ATGGGGAGAGGAAGGGTGGAGCTGAAGAGGATCGAGAACAAGATCAATCGCCA
GGTAACCTTCGCGAAGCGGCGGAACGGGCTTCTCAAGAAAGCCTACGAGCTCTC
CGTGCTCTGCGACGCCGAGGTCGCCCTTATCGTCTTCTCCAACCGCGGGAAGCTC
TATGAGTTCTGCAGCAGCTCCAGTATGTTGAAGACACTAGAAAGGTACCAAAAA
TGCAACTATGGTGCACCAGAGACTAATATTGTGTCAAGGGAAACTCAGGAGGAC
AGAaGACCCTACTTAATCTATGAGATGAAGGAGAaCAAATCATGGAcAtAA
SEQ ID NO: 83 >EG4N109263
ATGGGGCGAGGGAAGATTGAGATCAAGCGGATCGAGAACTCCACCAACCGGCA
GGTAACCTTCTCCAAGCGGCGGAATGGGATCATCAAGAAGGCCCGGGAGATAAG
CGTGCTCTGCGATGCCCAGGTCTCCCTCGTCATCTTCTCCAGCTCCGGGAAGATG
TCCGAGTACTGCAGCCCCTCCACCACGTTGTCGAGGTTGCTGGAGAAGTACCAGG
TGAACTCTGGCAAGAAGCTCTGGGATGTCAAGCACGAGAATCTGAGTGTTGAGA
TTGACCGAATCAAGAAGGAGAATGACAACATGCAGATTGAGCTGAGGCATTTGA
AGGGTGGCGATCTGAACTCGCTGAACCCAAAGGAACTCATTCTAATTGAGGATG
TCCTCCAGAATGGTCTCACCAGTGTTAGGGGCAAGCAGCATCACCAGGAATTGG
CAATGAATGGAAATGTAAGGGAATTGGAGCTTGGGGATCCTCTGAAAGCTAGGG
ATTTTGCATGCCAGATTCCAATAGCCTTCCGTGAGTGGGAGGAAGTTGCTTAG
SEQ ID NO: 84 >EG4N29529
ATGGGgAGGGgAAGGGTGGAGCTGAAGAGAATCGAGAACAAGATCAATCGCCAG
GTGACTTTCGCGAAGCGGCGGAATGGGCTCCTCAAGAAGGCCTACGAGCTCTCC
GTCCTCTGCGACGCCGAGGTCGCTCTCATCATCTTCTCCAACCGCGGGAAGCTTT
ACGAGTTCTGCAGCAGCTCCAGGAGGAACATCGAACTAAATGTCTAG
SEQ ID NO: 85 >EG4N115489
ATGGGGAGGGGGAAGATAGAGATCAAGAAGATAGAGAATCCTACcAACAGGCA
GGTGACCTACTCCAAGAGGAGGACGGGGATCATGAAGAAGGCTAAGGAgCTGAC
GGTGCTTTGCGATGCTGAGGTCTCGCTTATCATGTTCTCCAGCACCGGCAAGTTCT
CCGAGTATTGCAGCCCCCTTTCCGAGCAGCGGATGGGTGAAGATCTCGACAGTTT
GGGCATCCATGAACTGCGCGGTCTTGAGCAAAATTTAGATGAGGCTTTGAAGGTT
GTTCGTCACAGAAAAATTCTTTATCCAGAAGGACCTCTGGATCTTGCTGACATTG
AGTATCCATTTATGGAGAAAGAAATCCATGATACAGTGCGGAAAGTGGTGATGC
TTGGCGATGAGAAGATTTGA
SEQ ID NO: 86 >EG4N6889
ATGGGTCGAGGAAAGATCGAGATCAAGAGGATAGAGAACACGACCAACCGGCA
GGTGACCTTCTGCAAGCGCCGCAACGGCCTGCTCAAAAAGGCCTACGAGTTGTCC
GTGCTCTGCGACGCGGAGGTCGCCCTCATCGTCTTCTCGAGCCGCGGCCGCCTCT
ACGAATACGCCAACAACAGGTTGCTAGCTTCTACGAATCTTTGGAGGGAACCGTT
CACGAGATCTCCCCATGTGAAAGCTACCATCGAGAGGTATAAAAGAGCATGCAC
TGATACCTCCAACTCTGGATCTGTTTCTGAAGCTGATTCTCAGCTTAATTCTTCCT
TTCTTGAGTGA
SEQ ID NO: 87 >EG4N39137
ATGGGgAGGGgAAAAGTTGAGCTGAAGAGGATCGAGAACAAGATCAACCGCCAG
GTTACCTTCTCCAAGCGCCGCAACGGCCTGCTCAAGAAGGCCTACGAACTCTCCG
TCCTCTGCGATGCCGAGGTTGCACTCATCATCTTCTCCAGCCGCGGCAAGCTCTA
CGAGTTCGGCAGCGTTGGGGGTTCTCTAGTTAGTTAG
SEQ ID NO: 88 >EG4N44072
ATGGGGAgGGGGAGGGTGGAGCTGAAGAGGATCGAGAACAAGATAAACCGGCA
GGTGACGTTCTCCAAGCGGAGGAACGGGCTGGTGAAGAAGGCGAACGAGCTGTC
GGTGCTCTGCGATGCGGAGGTCGCCCTCATCATCTTCTCCAACCGCGGCAGGATC
ACCGAGTTCTGCAGCAGCTCCAGCGGAGGAACTTCCCAGAAATTGATAACTTCA
AAGGCGTGGAAGGCTTTAGAGCTGACCACCCCCTATTCCATACATGAGATCCTAT
CGGTGGTAGCAATTTATCCCCAcCTCAAGAGTCACACCAACCTCCAACAGCCTGA
GCATAGCGAGTTTGACGACGGCAGCTAG
SEQ ID NO: 89 >EG4N62915
ATGGGGAGGGGGAAAGTGGAGCTGAAGAGGATTGAGAACAAGATCAACCGCCA
GGTGACCTTCTCCAAGAGAAGAAATGGGCTCCTAAAGAAGGCTTATGAGTTGTC
GATTCTTTGCGATGCCGAGGTCGCCCTCATCATCTTCTCCGGTCGTGGAAAGCTCT
ATGAGTTCGGCAGCGTCGGCCACTTGGGCAATAGAATAGGCGTTGGACGCACTC
CATTCAGGCTGTCTGACTGA
SEQ ID NO: 90 >EG4N64304
ATGGGGAGGGGgAAGATTGAGATCAAGAGAATTGAGAACACTACAAACCGCCAA
GTGACCTTCTGCAAGCGGAGGAATGGTTTGCTGAAGAAAGCCTATGAATTATCG
GTTCTTTGTGATGCAGAGATCGCGCTCATCATCTTCTCaGgCCGTGGCCGGCTCTA
TGAGTACTCCAATAACAGATCTGTCTTTATAGATCTTCATCCCAAGGATGAAGGA
TGCTTCTCCCAAATCCTTTATAGAGAACTGTGA
SEQ ID NO: 91 >EG4N104954
ATGAAAAAGATAGTGAAGAGTAAGGAGATCATGGGGAGGGGTAAGATTGAGAT
CAAGAGAATTGAGAACACTACAAATCGCCAAGTGACCTTCTGCAAGCGGAGGAA
TGGTTTGCTGAAGAAAGCCTATGAACTTTCGGTTCTTTGTGATGCAGAGATCGCC
CTCATCGTCTTCTCAAGCCGTGGCCGCCTCTACGAGTACTCCAATAACAGGTGTG
TTTATGTGGATGTGAGGTGA
SEQ ID NO: 92 >EG4N82414
ATGGGgAGGGGgAGAGTTGAACTGAAGAGGATCGAAAACAAGATCAACCGCCAG
GTAACCTTCTCCAAGCGCCGCAGCGGCCTGCTCAAGAAGGCCTATGAGCTCTCCG
TCCTCTGCGACGCCGAGATTGCACTCATCATCTTCTCCAGCCGCGGCAAGCTCTA
CGAGTTCGGCAGCGTTGGGTCCAGAGCAAATTATAATCCTGCCAAAGAAACGGT
TACAAACGTCGCCATCAATCCATTACCTCCTCCACCTATAAAAGGAGAACCCATA
TACACCAGAGATGAATCCCAGCCTTTTGGGAAGCACACAGCTCGGAAGCCTATCT
TAAGCAGGGCATTCTATTTGGATTTGGTCCCCAATATCGAGAACAAGACATCAAT
CTCTCGCTTGGAAATTCTTCTTCCTTACAGCAAAGCATGTCCTCAAAGAAAGTCA
GAAAGATCTGTGAAGCTCATCATGGATCGAATCATATCCAATATGATTCGATTCC
TTCTCTCGGATATCCCATTAAGTTGA
SEQ ID NO: 93 >EG4N39130
ATGGTGAGGGGGAAGACGGAGATGAAGCTGATAGAGAACGCGACGAGCAGGCA
GGTGACGTTCTCGAAGCGGAGGAATGGGCTTCTGAAGAAGGCGTTCGAGCTCTC
GGTCCTTTGCGACGCCGAGGTCGCCGTCATCGTCTTCTCTCCCCGTGGAAAGCTC
TACGAGTTCTCCAGCACCAGCTTGTCAATGCCAGATACACAACAGAAAAGTGGA
TCTTCTCAGGAACCTTGTTCAGAGCTACTTGAAGATGAAGAACTGGAAGGAGTTG
ATAATGTTTGTGATGGAGTCGTTGGCAGTGGATGGACATATGACCCATATGCCAA
GGGGAATCCACTTCAAAAAGAAGAGCATGCAAAGAAATTATTCTTTTCCTTAAG
ATTAGGCAAGAGAAATCCTACATGGGTGAGGTCAGCTGTGGTGACATGGAATCA
GTTACTTGAAGAGCAAATTGCAACGCTCAAAGAACAGGAGCAGACACTTATGGA
GGAGAATGCATTACTACGAGAGAAGTGCAAGCTACAATCTCAACTACGGCCAGC
CGCTGCTCCAGAGGAAACTGTTCCATGCaGCCAGGACGGTGAGAATATGGAGGT
AGAGACAGAGCTGTACATTGGATGGCCAGGAAGGGGAAGGACCAATTGCAGGTC
GCAAGGTTGA
SEQ ID NO: 94 >EG4N44048
ATGGGGAGAGGTAGGGTGCAGCTGAAGAGGATCGAGAACAAGATAAACCGGCA
GGTGACGTTCTCCAAGCGGCGGTCGGGGCTGTTGAAGAAGGCGCACGAGATCTC
GGTGCTCTGCGACGCGGAGGTCGCTCTCATCGTCTTCTCCACCAAGGGCAAGCTC
TACGAGTACTCCACCAACGCCAGGTTGAGGTCAGTGTTTGGCGGAGCTGGAGGT
GGTCAGCCAAAATCCAAACTAGAGAATGGCATCTTCCTTCAAAGGACTTCAAAG
GTTTCCTTATGGGGTTATCCCCCACTTCTCGGACAATCAAGGATTTCTGCTATGCT
CATCTTGGGACGAGGGGCATTCTTTGCTCATGGTTGTTTGAGTCTTCTTGAATCAT
CTCTCGATCGGAACAAGTAA
SEQ ID NO: 95 >EG4N2672
ATGGGGAGAGGGAGGGTGCAGCTGAAGAGGATCGAGAACGAGATAAACAGGCA
GGTGACGTTCTCGAAACGCCGGTCGGGGCTGCTGAAGAAGGCGCACGAGATCTC
GGTGCTCTGTGACGCCGAGGTCGCCGTCGTCGTCTTCTCTACCAAGggCAAGCTCT
ACGAGTACTCCACCGACTCCAGGATGGACCAAGGGGGACTTGGTGGCTTGGCTT
CGGTGAGGGGCGGCGGCTTGGCCGGATGTCCGGCAGTGACGGTCGACGATGGTG
AGGCAAGGGATGGCTGGCGGCAAGTAAAAGCAAATGAGAGAAAAGCTTTCAAT
AGTCAAGGTAAACCAAAGAATAAAAAGTGGAGCGCCCCTTCGTGGAGGTGGCAT
CCTAACTTGGATGCCCCTCTTTGGCACTAG
SEQ ID NO: 96 >EG4N15413
ATGGGGAGAGGGAGGGTGCAGCTGAGGCGGATCGAGAACAAGaTAAACCGGCA
GGTGACGTTCTCGAAGCGCCGgTCGGGGCTCCTGAAGAAAGCCCACGAGATCTCC
GTCCTCTGCGACGCCGAGGTCGCCCTCATCATCTTCTCGACCAAGGGCAAGCTCT
ACGAGTACGCCACCGACTCCTGGCTCCAAGCAGCTACAACTGCTTGGAAAACCC
ATTGGGATCTCACAATCTCCTGTTGGCTGGCCGACCGACAGTGCAACTGGCATGA
GGCGACTGTCGGCAGGAGGAGGGGTGACCCAGCGGCAAGAGGAAGGCCAAGCC
GGTGGCCGGTGGCGGCCACCGACGCCCACACATTCAAAAAGGCCCGAATCCCTT
TCTCAAAGAAATCCGACGACTCCGGTCGCCGGCGATCGTGCACACGGGCACGGG
GAGAAAGGAGGAGAAGAGAGGAAGGGGAGGAGGCTCACCTTCGACGTCGGCGA
GGCTTTTCCGGCGAGCAAAAAAAaGATGGCACAGGGACGGTCTCCGCGGTGGTTT
TCCAACGATTGCCGCCGACTGAGTCTCGAATCTTCGGTGAGAGGGAGAGAGGAG
GATTCTCCTTAAATAGAGCCGGAGGGGGGgCTCTTTCCGACTCCGATTGGGAGCC
GCTTCTATCATCAAGGACTATTGAGCTTGGGAGACCCGACCTCCATGGCTCTTTG
GTGGCCATTACAGGCATCTCCGCTGAGCTATGTGATTGCAATCGCTGA
SEQ ID NO: 97 >EG4N155269
ATGGAAGGGATAGGAGAGCTTCGGGGGCTCATTGAAAAGAGAACACCGGCCATC
TGGTCCAAGGGCCGCGGCCATGCAGCTTTTCCTCTCTCACTTCCTCCCCTCGGAAT
CCACGGAAATGGAGTTCCTCTGAAAGTTAGAAGGAAACTAGAAGAAAAAAGGGT
GAGAATCTCGATTTGGAAGTGGATTTCCGGGGAGTTGGAGGTCATTCCTCCACTT
CTAAAGAGCAAGGAGATCATGGGGAGGGGgAAGATTGAGATCAAGAGAATTGA
GAACACTACAAACCGCCAAGTGACCTTCTGCAAGCGGAGGAATGGTTTGCTGAA
GAAAGCCTATGAATTATCGGTTCTTTGTGATGCAGAGATCGCGCTCATCATCTTC
TCaGgCCGTGGCCGGCTCTATGAGTACTCCAATAACAGGAACTGA
SEQ ID NO: 98 >EG4N11519
ATGGCACGCGGAAAGGTGCAGATGAGACGGATTGAGAACCCTGTCCAGCGGCAG
GTCACCTTCTGCAAGCGCCGAGCCGGACTGCTCAAAAAGGCTAGGGAGTTGTCA
GTGTTGTGTGGTGCTGATATTGGCATCATTATATTCTCCACCCATGGCAAGCTTTA
TGAGCTAGCCACTAACGGGGACATGCAAAGTTTGATTGAGAGATACAAGAGCAT
TGGTGCAGAAGCTCAAATTGAAGGTGGTGAAGTGAATCAACCTCAGGTCTCAGA
ACAGGAGATATCCATGTTGAAGCAAGAGATCAATCTGCTGCAGAAGGGCATAAG
GAAGTGCAACCTTCCCGAATCAAACAGTGAGAGTCACTACTATGGAGAAGAGGA
GATCGAAGACAACAACAAACCAAGGAGGCTCCGGCATGCGACGGGAGAAGGCG
ACGAGAGGGGGCGCGAGAAGGTCTCCAGAGAGGCCACTGGGGTGGAGGGGAGG
CCGTCAAGCGGCAGCGCCGCCTTGGCCTTGTCACCCGTCTCCACGGACTTGAGAG
CCACGGATTTGGGAGGAGTGGTGGCAAACGCCGCCGCCTGCGTGTTAGGGGAGG
CCGGCTGGACGTCGAGGCCCGAAGGCGAGGTCGTGGCCGGACGGACTCTCGTCG
AGGGACTGCGAAAAaGAAaTGCTTCAAAGGCCTAG
SEQ ID NO: 99 >EG4N14715
ATGTTGATGCATTTGACACTGAAGGACAAATGTGTTGGAGATGAGCTTGAGCTTG
AAGTTGGTGATGGACTTACATTTGGAGAAGTTTGTGTACATAAGATCTCTTATGC
AGCTCTTTATACAAGCCCAGGGGTGGCAAGCCTTGTTTTGGAGAGGGGGCGGTG
CATTTGTTTCTGGTGTTGTGAGAAGAGAACGATGGTGAGAGGAAGAAGGGAGAT
AAAAAGAATCGAGAACCCCATCCAGAGGCAGTCCACTTTCTATAAAAGAAGGGA
TGGCTTGTTTAAAAAAGCCAGGGAGCTCTCCATTCTCTGCGACGCCGACCTCCTC
CTCCTCCTCTTTTCCTCTTCCGGAAAGCTCTACGAGTATCACACCCCTTCTGTGCC
CAGTGCCGAGGAGCTTGTCAAGAGGTACGAGGTTGCCACCCAAAATAAGATTTG
GAGGGACCTCCACTTGGAACGAAATGCTGAGATGGAGAAGGTCCAGAAGTTGTG
CGAGCTCTTAGAAAGAGATCTAAGATTCATGAAGGTTGACGCAAGCCAACACTA
CTCGCTGCCAGTTCTCGACGTTTTAGAGGGCAATCTGGAGGCAGCCATCAACAAG
GTCCGGTCGGAGAAGGATCGGAAGATAGTAGGAGAGATCAACCACTTGGAAAAC
ATGGTAAGAGATCGCCAGCAAGAGAGGTACGATTTGGGCGACAAGGTTGCCCGT
GCACAGGGTCTTAAAGACATGGCAGTACCACTCAACCGACTGGATCTGAAATTG
GGTACTTGTGTTTCCTAA
SEQ ID NO: 100 >EG4N82401
ATGGTGAGGGGAAAGACGGAGATAAAGCGGATAGAGAACGCGACGAGCAGGCA
GGTGACGTTCTCGAAGCGGAGGAATGGGCTTCTGAAGAAGGCGTTCGAGCTTTC
GGTCCTCTGCGACGCCGAGGTCGCCCTCATCGTCTTCTCCCCCcGGGGgAAGCTCT
ACGAATTCTCCAGCACCAGATATACTGGCTATTTGGGAAAAATCAATGTCAAAAT
AATGCAGGACAAGAACAAGACTTTGAGAGCTTGTTTGGTGTTTGTCAACATCTTA
ATCACCTTGATGCCAGGGAaCGCATTATCATTGCAATGCCATGCTCTACTCACCCc
TTCGCAATACAACCAGAATCTTTCGAGTACGAATGATGAAGGCCTTCGTTTCAAA
TCAGATTCATCTTTTAACAAAATGGGGGAGTGGCCCGATTCAGTTTTGGTGAAAT
GA
SEQ ID NO: 101 >EG4N37080
ATGGTTCGAGGGAAGACGGAGGTGAGACGGATCGAGAACGCGACCAGCCGGCA
GGTAACGTTCTCCAAGCGCCGGAATGGTCTCCTGAAGAAGGCCTTCGAGCTCTCC
GTCCTCTGCGACGCCGAGGTGGCTCTCATCGTCTTCTCTCCCCGAGGAAAATTGT
ACGAGTTCTCGAGCTCCAGCAGACTTATTGTGATGGCTGTGACCACAAGCTTAGC
TGATCACGTAGATAGGATCTCAGAGAATCTCAACGATCGTATCGTGGACAATATC
TCAGAAGCTTTAAGGTTGCTGGCTCCAAAGCCTCTGCATGACTTCCTCCACATGT
GCGTTAGCCCACGTTTGGATCGTGGAGTCTTGAGAGGAGTATCGAGTTGCTGGAG
GGTCGAAGCTGTGGTGAATCCTATGACCTAG
SEQ ID NO: 102 >EG4N63104
ATGCGTGGACCGTGTGAGGAGCATCGCGCTGGCCGTGCAACGCGCGCCCGCCTG
AGCCTGGGCCGCGCACCTTGTGCGCCCGCACATTGGGCCACATGCTCACAGCCAT
CCCGCATGCTGCCACGTGCACCCGCTCAGGCGGCCTACAGGAAGACACAGGTGA
GACGGATCGAGAACGCCACCAGCCGGCAGGTAACGTTTTCCAAGCGCCGGAATG
GGCTTCTTAAGAAGGCCTTCGAGCTCTCCGTCCTCTGCGACGCCGAGGTCGCCCT
TATCGTCTTCTCCCCTAGAGGGAAGCTCTACGAGTTCTCCAGCTCCAGAGCTACT
GTGAGTTTTGGTTCCAGGAAGGTATGGATTATTCAAGCTACAATGGATGCAGAAG
CCAATGACTGTGGTAGAGCATCCTCCACGAAGATGCTCTCTGCATGCAACTCTTG
CTGTGTGCAGGCTGTAGGGGAGTGGGTCTATACTGCCTTCAATAGAGGAGGTTCT
GAGAGTAAAACTCGAGAGGTTTCCCAAGATCTGGGCACAGAATCATGTGCAATT
GAGGAACTGCATGATCTAGAGCTCCAGTTAGAGCAAAGCCTAAGCAGCATCAGA
AATCGGAAATTAAATGCAGAACCTCGGCTACAGCTATGTGCTCCTGCTGTTTCTG
ATGATTATGATAGTCAGAATACAGATGTAGAGACAGAGCTGGTAATTGGTAGGC
CAGGGACTTGCAAGGTCAAGTGA
SEQ ID NO: 103 >EG4N37079
ATGGTTCGGGGGAAGACGGAGGTGAGACGGATCGAGAACGCGACCAGCCGGCA
GGTGACGTTCTCCAAGCGCCGGAATGGTCTCCTGAAGAAGGCCTTCGAGCTCTCC
GTCCTCTGCGACGCCGAGGTGGCTCTTATCGTCTTCTCCCCCAAGGGAAAGCTCT
ACGAGTTCTCCAGCTCCAGCAGGGATGGAGTCGAAGATCAATACTCAGGAGGTG
AGCGAACCTATAGCTCCTTAGTCTCGTTTTCCAAATATATGTTAAGAAACTGTAC
TGAGGATCCATTAGGAATGATGATTAAGCCCAAGCTTTACCATCTCGTTACCAAA
TCCTATGCGGGTACTATCTTATTACAGTATCGCATTCAAAAGACAGTTGATCGTT
ATTTAATGCACACAAAAGATGTCAACATCAACATCAGAGCAACGGAACAAAATA
TGCAGTGCAAGACAGAACCTCCAGTACAACTGATAACTCAGGCATCTTCAAATG
GTGATGCTTGTCAAAATATGGAGGTAGAGACTGAGCTGATTATTGGAAGGCCAG
GAACCTGTGAGGCTAAACAACAGGATCATGTTAGCCTCAACAAGCAGTGGTCGC
AGGAAAATGGGGCATTCGGAATGGAGAGCAGACAAAACCCATAA
SEQ ID NO: 104 >EG4N29559
ATGGTGAGGGGGAGGGTGGAGCTCCGGCGGATCGAGGACAAGACGAGCCGCCA
GGTGAGCTTCTCCAAGCGGCGGAGTGGCCTACTCAAGAAGGCGCACGAGCTCGC
CGTCCTCTGCGACGCCGAGGTCGGCCTCATCATCTTCTCTGCCAAGGGCAAGCTC
TACGACTTCGCGAGCACCTCCAGTGTGTACAGATACAACATCATCATGGACAATA
GGCCAGAATTGTTGGAAGAAAAAAGGATCGAATGTTATGTGGCCCTGATGCATG
ATTTGTACATAAAGATTTGGTGCAAAATTGCACTGAGTAATGTGGATTATAAACT
TGCTGCCGAGTTTGCCCTTCTAAGATGCAAGCCTTTAACACGTCCTTTCAATGAA
AGGCATCCAACAATGTCTTGGAAGCTTCTTGTGGAGCAAAGGAAGGCCCAAACA
GGCTATACACCCTTGAACAGCACCCCTCACCTCTATGGAGGAAATTGGCCAGGCC
ATTCCTGCACTCCGCTTGGAAGTGGTTGA
SEQ ID NO: 105 >EG4N43162
ATGGGCAGAGGGAAGATCGTGATCCGAAGGATTGAGAACTCGACCAGCCGGCAG
GTGACCTTCTCTAAGCGGCGCAAGGGTCTGTTGAAGAAGGCCAAGGAGCTCGCC
ATCCTTTGCGATGCCGAGGTCGGCTTTGTCATCTTCTCCAGCACTGGCAGGCTCTA
CGATTTTGCCAGCTCcAGCGAGGCTGAACTTGGGCATCACAAAACCAAAGTCTAT
ATAAGCGCAACGGAATGGTGGCAAAGGATTGAGTTTGAGTCGGATCAAATATGG
GTTGGGTCAAAGAATCTTCAACGACCACTCCATCAATATAAAGATAAGACCTTTT
TCTTAAGGCAACATAGAGGCAAGACTTTCGGCTCAAGTCTCCTCCAATGGATGGA
GGATGCTGATAACTTGTGGGGATAA
SEQ ID NO: 106 >EG4N31052
ATGAGGCTCAGGTTGTCGTCGTTCACACTACACCTACCGcGGCCCCACCCTATTAT
TGTCTACGTCGCATCCATCGTTCGTGTAGTATTCGGCTTTGACGGCACCAAGCCTT
CTCCCCTTTCCGATCCtGATGCACCCCGTGCGACCCGcCCCGCACCCTTTGCGGCC
TCGCCCCACCGCCATCCCCTTTCCTTCTCTCTTACGACcCCGATGAATCCGAGCCC
TTGTGGCTTTATAGCGACATACACGGTTCCCGAGAGCCAGGAAGGCGGAACCGT
CCAAAACGGGGGCACCAACTTTCGACGAGAAAGCGTCTGGTGCATATTAGGATC
AATGGTGAGGGAGAAAATCCAGATAAGGAAGATAGACAACGCGACAGCGAGGC
AGGTGACGTTTTCCAAGAGGAGGAGGGGACTGCTGAAGAAGGCGGAGGAGCTCT
CGATCCTCTGCGATGCCGAGGTCGCCCTTATCGTCTTCTCGTCCACCGGCAAGCT
CTACGAGTACTCGAGCTCCAGTGCCCCACTTCCATTCGCCGcCCCCCTCCCCTCGC
CCATAGTATCTCCATACCGGCGGCCTTCCCACGCCGGCGGCCTCCTTGTGcCGGC
AATGCTGGTAGCGTCCCTGTGCTGTGGCCTCCCTGCGAgGCAGCATCAGCTGcCCC
CTCTTGCTGTCTGTCCCCTCTTCACGTGGGCAGGCGTTGGCCTTCCACTTGATCGc
CCCCTCCCTTTGcCCCCCCTCCTCTCACCCATAGCATCCATCATGAAGGAGATCAT
TGAAAAGCACAGCATGCATTCAAAGAACCTACAGAAACCAGACCAACCCCCCCT
TGACTTAAATGGAGAATGGCTTCTACATGCAATTGTAACCCCGAAGTATTTACAT
CAAGTTCTAACATCAAATGATGAATACTTCTCCCCTGATGAAACTTAA
SEQ ID NO: 107 >EG4N86343
ATGGTGCGTGGCAAGGTGCAGATGAAGAGGATCGAGAACCCCGTCCACCGGCAA
GTCACCTTCTGCAAACGCCGGGCAGGGCTGCTGAAGAAGGCCAAGGAGCTGTCT
GTGTTGTGTGATGCCGAAATCGGAATCATAATCTTCTCCACGCATGGCAAGTTGT
ATGAGCTAGCTACTAAGGGGTCTTACAACTGA
SEQ ID NO: 108 >EG4N39902
ATGGGGCGTGTTAAGCTCCAGATAAAGAGAATAGAGAACAACACCAATCGCCAG
GTGACCTTCTCCAAGCGTCGCAATGGGCTCATCAAGAAAGCCTACGAGCTCTCGG
TTCTTTGTGACATTGATATCGCCCTCATCATGTTCTCTCCCTCCGGGAGGCTCAGC
CATTTCTCCGGCaGACGGAGATTTTTTGAGCCAGACCCCCTCAGCATCACTTCTAT
GGATGAGCTTGAATCATGTGAGAAATTTCTCATGGAGGCCTTAAGGCGcGTGGCA
GAGAGAAAGCATGGAGGATCATGGGTCAAATTAGTACAATTACCGCGAGGATGG
TACCAAAATGAACTGCCACATCTAGCGGTATTCACCAACGACACAAAGTTCTTAA
TTCCCATGCTGCTGAAGAACACCGTGATTTGTATTGTGTATCGCCAAAAGCTTTT
GTGA
SEQ ID NO: 109 >EG4N48307
ATGGATAAATTAGAGGCTAGaTCCTTTAGGACTCGCTTTATAGGGTATCCTAAGA
AAATCATGAGATACTACTTCTATCTTCCTGAGAATCACAATAGGCGATCAGACTT
GATAACTTTCAATTTGCCATGGAGAAGATGTGCTAGTTTGATGAGACGGCATGGC
AGTGGCTCACACAACACCTACCTGAGTTGTGGTCAAGGCATGCCTTTGCGGGCCG
CTAGGGTGATAACTAGAGGAAGCGAAACCATCACTCGGACGCGAAAACCGAACC
GCCCCATCACCACCACGCCAACGTGTCGCGTCCCGAGAGGGGAGATTCGGGTGC
CGAATGGAGTCTGGAATCCTCGGTGGGCCTCCCCTCTCCCCGTTCATCTTCCTCGG
TCCTCAAGACCGCCAGCCCACTCTAACGGCTTAAGCTTGGGGTTCCGGCGTCCAA
CGGCGGCGGCGATGAGAAGGGGGAAGGTCCAGATTCGGCGAATCGAGGACAAG
GCCAGCCGCCAGGTGACCTTTTCCAAGCGGCGGGGCGGCCTCTTCAAGAAAGCC
CGCGAGCTCGCCGTCCTCTGCGACGCGGAGGTCGGCCTGATCGTCTTCTCCCCCA
GCGGCAAGCCCTACGAATTCTGCAGCTCCTCCAGGTGCGTTTCCATTCTCCTCCTT
CGGCTTAGGTCGTCGGATCCCTCGAGATCCATCGATTCCCTCAGAGACCAGCCCG
GCTCAGTTCGTCAAACACTTCGCTCGTCTTCGTTCTTGAGACGGTGGTGA
SEQ ID NO: 110 >EG4N23857
ATGGGTCGTGGAAAGATAGAGATCAAGAGGATCGAGAACCCAACTAACCGTCAG
GTCACCTTCTCCAAGAGGCGGGGAGGGCTCCTCAAGAAGGCAAATGAGCTTGCG
ATACTGTGTGATGTGCAGGCTAGCATGAGGCAGTACACTGGGGAAGACTTGAGC
TCTATGACCATGAATGACTTGAATCAGCTCGAACAACAGCTGGAGTACTCGGTTA
ACAAGGTTCGAACAAGGAAGCTATCAGAGCACCAGGCAGCAATGGAGCATCAGC
AGGCTGCCATGGAGCACAAGGTGCCGGACGTGCCCATGCTGGAGCCATTCGGGT
TGTTCTATCAGGATGAGCCATCGAGGAATTTGCTGCAGCTTTCGCCCCAACTGCA
TGCATTCCGTCTCCAGCCGGCGCAACCCAATCTGCAAGAGGCCAGCCTCCCAGGT
CATAGTCTGCAGCTGTGGTAA
SEQ ID NO: 111 >EG4N29533
ATGGTTACTCTTTTGCTAGCACAGAGTAGTCAGCAAGAGTACTTGAAATTAAAAG
CACGTGTTGAAGCCTTACAGAGATCGCAAAGAAATCTCCTCGGTGAGGACTTGG
GTCCACTCAGCAGCAAGGAGCTTGAGCAGCTCGAGCGGCAACTTGATGCATCGT
TAAAGCAAATCAGATcAACACGGACCCAATACATGCTTGATCAGCTTGCAGATCT
TCAACGAAGGTTGGAAGAAAGTAACCAGGCTGGTCAGCAGCAAGTTTGGGATCC
CACTGCTCATGCAGTAGGCTATGGCCGGCAGCCACCTCAACCACAGAGCGATGG
ATTCTACCAACAGATAGATGGTGAACCTACTCTCCAAATCAGTGTTGAAGGAGA
GGAGGATGAGGGTGAATTAGTAGAGGAGGACATGGAGAAAAGAGCAAGTGATG
TAAAAGAGGAATTGGAGTACACCCTTGTATATGTGATGAGGTATCCTCCAGAAC
AAATAACAATCGCAGCAGCACCCGGGTCAAGTTGGGCCATAATTTCTAACAAAC
TCGATGATGAAAAAGAAGAAGAAGAGGGGTCCTTTTCCGATGATGATTGGAGGC
TGACGGTGGTTGATTCGGAGTGGGTCATATCGATGAGGTTGGTGATGGGTTCTTT
TCCATGCTTTGTCAAGGAAGACTAA
SEQ ID NO: 112 >EG4N70708
ATGGGGGAGGAACATCTTTCCGACGGAAAGACTGCCTCGCCGATCCAGTTGAGT
GAGGAGTCTAGGAGAGGGATGGCGAGGGAGAAGATTCAGATAAGGAAGATAGA
CAACGCGACGGCGAGGCAGGTGACCTTCTCCAAGAGGAGGAGGGGGCTCTTCAA
GAAGGCCGAGGAACTCGCCATCCTCTGCGACGCCGACGTCGCCCTCATCATCTTC
TCCTCCACCGGCAAGCTTTTTGAGTTCTCGAGCTCAAGGGTTTTTATGGtGATCAG
AGTGAAGCTCCGTACGGGTTTAGCTAGGTGGGTTTTGTTGCAGATGATTACAACT
CTACCAAAATCTGGACACTCAAGTGTTGGAATTCCATTGATTAGCTTCAAGGCTA
TTGTGGTGGAGATGGCCAGAGCAGGGAGACGTGTGCTGACTGATTCGGAAAATG
TTATGTATGAGGATGGGCAGTCATCGGAGTCGGTTACTAATGCTTCACAATTGGT
AGTGCCACCGAACTATGACGACAGCTCCGACACATCCCTCAAATTGGGGTCCACT
GATTGTGGGCTCACTGAGGTCTGTGTGGATTATGATCTGTATGTCACAACCTCCT
GCACTTTGTTTGAGGGATATACTGCTGTGAGAAAACAGGCACTGTCTTTGTTCTT
ATATGATCGGAGTACGCATGCAGCACAAATTGATAGAAAACGGCGCCAGCAAGT
ACGGATCCAGGAATGGCGCCGGTTGAGCAAATTGACTGGTCTCTTAGCTGGAGC
ACTTAATTTGTTTGGCGCCGTATCAGGGCCAAAATATGATGGCAAATTTCTGCAC
TCTAAAGTGAAAGAACTGCTTGGTGATACAAAGCTTCATCAAACTTTAACTAACa
TTGTGATTCCCGCTTTCGACATCAAGCTTCTTCAACCTGTCATATTCTCAACCTTT
GAGGATGACACCTTGGAAGGAGACACGGCATCCGTGGACGTCTCGACGAGTgAG
AACTTGCGAAAGTTGGTGCAAGTTGGCCAGGATCTCCTTAAGAAGCCGGTATCG
AGGGTCAATCTAGAGACTGGCGTGTCTGAGGCCTGCGATGTTGAAGGAACCAAC
GAAGATGCCCTCATCCGCTTTGCGAAGATGCTCTCCAACGAAAGAAAGTCTAGG
AATGCAAAAATGTCAGCTGCTTGA
SEQ ID NO: 113 >EG4N67350
ATGGACAAATTTGAAATAGCTATCAAGACTAGTCAGCAAGAGTACTTAAAACTT
AAAGCACGTGTTGAAGCATTACAGAGATCACAGAGAAATCTCCTTGGTGATGAC
TTAGGGCCACTCAGCAGCAAGGAGCTTGAGCAGCTTGAGCGGCAACTAGATGCA
TCATTGAAGCAAATCAGATCCACAAGGTTGGAGGAAAGCAACCAGGCTACTCAG
CAGCAAGTTTGGGATCCCAATGCTCCTGCAGTGGGCTATGGCCGGCAGCCACCTC
AACCACAGGGAGATGGATTCTACCAACAGATAGAGTGCGATCCAACTCTCCATA
TCGGGTATCCTCCAGAACAAATAACGATTGCTGCAGCGCCTGGGCCTAGCGTGA
GTAATTACATGCCAGGATGGCTTGCGTGA
SEQ ID NO: 114 >EG4N44069
ATGGCGGAGGACCGCTGGCGGCTTGCGGCGGGCCGGCGGCGCGCGGCCCAGAAG
TGGCAGCGCCCGGCTTGGGTGCGCAGGGTGCGGCCTAGTACATGCGTGCGGGAT
GCGGCCCAGGCCCTGGCCCAGGCGTGCATGCGGGTGCAGCCTAGGCCCACGCGA
GCCCGTGCTGGAAACCTCATGCTCAAGACAATCGAGAGGTACCAGAGGTGCAGC
TATAATGCAACAGATGCAATAGTTCCTCCAAAGGAGACACAGGACCTTGGTCCA
TTAAGTGTAAAGGAGCTCGAGCAACTTGAGAATCAAATAGAGATATCTCTCAAG
CACATCAGATCAAAAAAGACCCAATTAATGCTTGATCAGCTATGTGATCTTGAGC
GCAAGGAACAAATGTTGCAGGAAGCTAACAAAGCCTTGAGAAGAAGGTTGGAA
GAAGATACAATTAATTCCCTCCAACTTTCATGGCAAAATGGAGCCAATGTTGTGG
GGAATGCCCCATGTGATGGTGAACCTCCTCAAACAGAGGGATTCTTTCAACCGCT
GGGATGTGAACCTTCTCTGCAAATTGGGTAA
SEQ ID NO: 115 >EG4N67198
ATGAGTGAGCGGGGgAGCAGGGAGCATTGGTGGTGGACGGAAGACGTTGAGCTG
AAGAGGATCGAGAACAAGATCAACCGCCAGGTTACCTTCTCCAAGCGCTGCAAC
GGCCTGCTCAAGAAGGCCTACGAGGTCTCCATCCTTTGCGATGTCGAGGTTGCAC
TCATCATCTTCTCCAGCCGTGGCAAGCTCTAG
SEQ ID NO: 116 >EG4N130373
ATGGTGAGGAAGCCGAGCATGGGCCGTCAGAAGATCGACATCAAAAGGATTGAG
AGTGAGGAGGCCCGCCAGGTGTGCTTCTCGAAGCGCCGCGCCGGGCTCTTCAAG
AAGGCCAACGAGCTGTCCATCTTGTGTGGCGCCGAGATCGGTGTCATCGTCTTTT
CCCCCGCAGGCAAGCCGTTCTCCTTCGGCCACCCCTCCGTCGACTCCATCATCGA
CCGCTTCCTCTTTGGCAGCCCCTCCCCTACGACTCTGCCGTCCGCCGACCCCCGCA
TGCCGGTGGCGCGCGAGATGATGGTCGTCCACGAGTTCAATCAACAGTACACGG
TGCTCACGGCCTTGCTGGAGACCGAGAAGAGGAAGAAAGCGGTGCTCGAGGAGG
CCGTGAGGGTGAAGCAGGCTGGGGAGGCCGCCTTGTGGGGCGCAAACATTGAGG
AACTCAGCCTGGGGGAGCTCGAAAGTCTGCACAAGTCCTTTGAGAGGCTGAGGA
GGGACGTGGCGATGCGCGCCGACCAGCTCGTCATAGAGGCCGCGCATACTCGCA
GCTCCAGCGTCGCAGCGGCAGGTAGTTTTGTTCCTCCTCCTCCCCTTGGTGTCAAT
CTAGGCTTTGGTCGTGGGGTGGAGGGGAGCATGGCGCTTCCTCCTCCCACTTTCT
TTGGTTATGGCCGTGGGCCCTTTTAG
SEQ ID NO: 117 >EG4N128041
ATGGATCGAGGTGACGTCGACCTTCAAAAGATCGATGGAAAGGAGAACCTGGCT
AACCCCTTCACTAAAGCCCTGACGATAAAGGAGTTCGACAACCACAAGAAGAAG
GAAGAAGAGGCATTAAGGACCACACCCACGGAAGATGATGATGATATGATATTG
TTGGATGAAGGTGTTGATATAGCATCCTCTAGTAAGAGAGATAATAGTGATCATG
CGTGCAATATGGTGAGGAAGCCGAGCATGGGCCGTCAGAAGATCGACATCAAAA
GGATTGAGAGTGAGGAGGCCCGCCAGGTGTGCTTCTCGAAGCGCCGCGCCGGGC
TCTTCAAGAAGGCCAACGAGCTGTCCATCTTGTGTGGCGCCGAGATCGGTGTCAT
CGTCTTTTCCCCCGCGGGTAAGCCGTTCTCCTTCGGCCACCCCTCCGTCGACTCCA
TCATCGACCGCTTCCTCTCTGGCAGCCCCTCCCCTATGACTCTGCCGTCCGCCGAC
CCCCGCATGCCGGCGGCGCGTGAGATGATGGTCGTCCACGAGTTCAACCAACAG
TACACGGTGCTCACGGCCTTGCTGGAGACCGAGAGGAGGAAGAAAGCTGTGCTC
GAGGAGGCCGTGAGGGTGAAGCGGGCTGGGGAGGCCGCCTTGTGGGGCGCAAA
CATTGAGGAACTCGGCCTGGGGGAGCTCGAAAGTCTGTACAATTCCTTTGAGAG
GCTGAGGAGGGACGTGGCGATGCGCGCCGACCAGCTCGTCATAGAGGCCGCGCA
TACTCGCAGCTCCAGCGTCGCTGCGGCAGGTAGTACTGTTCCTCCTCCTCCTCCTG
GTGTCAATCTAGGCTTTGGTCGTGGGGTGGAGGGGAGCATGGCGCTTCCTCCTCC
CACTTCCTTTGGTTATGGCCGTGGGCCCTTTTAG
SEQ ID NO: 118 >EG4N147209
ATGGGTCGCCAGAAGATCGAGATCAAGCGGATCCAGAACGAGGAGGCCCGCCA
GGTGTGCTTCTCGAAGCGCCGGACCGGCCTTTTCAAGAAGGCGAGCGAGCTGTCC
ATCCTCTGCGGCGCCGAGATCGGGGTCGTCGTATTCTCCCCcGCCGGCAAGGCCT
TCTCCTTCGGCCACCCGTCGGTCGACGCGGTCTTCGACCGCTTCCTCACGGGcAAC
CCCCACCACGGCAACAgCGGGGGgCCCGCGGCGGACTCGCGGCGCGGGGCGGTC
GTGCGCGAGCTGAACCGCCAGTACATGGAGCTGCATGGGCTGGTGGACGCGGAG
AGGAAGCGGCGGGAGGCCCTGGAGGAGGCCATGAAGGGGGAGCAGGGGGGCCG
CCCCTACTGGTGGGACAACAACGTGGACTCCCTCGCCCTGGAGGATCTGGAGGA
GTACGAGAAGAAGCTGCTGGAGCTGAGGAACAATGTCGCCAAGGTTGCTGATCA
GCTGCTGCATGAGGCCATGGCTCGCAAGCAGCAGCAGCACCATCACCACCACCA
CCAGCAGCAGCAGCAGCAGTTTCCGATGGTCGGCGCTGCCGTCGCTCTCCCTGGG
CCCTTCGCCATTAAGAACGAGGATGCCATCCATCCTTCTCTTGGTGGCGGGTTGG
GTTTCGGGCATGGCTTCTTCTGA
SEQ ID NO: 119 >EG4N37712
ATGGGCCGTCAGAAGATTGAGATCAAGCGAATCGAGAGCGAGGAAGCCCGCCA
GGTGTGCTTCTCGAAGCGCCGCGTCGGGCTCTTCAAGAAGGCCAACGAGCTCTCC
ATCCTGTGCGGCGCCGAGATCGGCGTCATCGTCTTCTCCCCCGCCGGCCAGCCTT
TCTCCTTCGGCCACCCCTCCGTCGACTCCATCATCGACCGCTTCCTCTCCGGCGGC
CCCTCCCCTCCGACTCTAGCCTCCGCCGACCGCCGCATGCCGGCGGCGCGCGAGA
TGATGGTCGTCCGCGAGCTCAACCGCCAGTACACGGAGCTCGCGGCCTTGCTGGA
GACGGAGAGGAGGAGGAAGGTGGTGCTGGAGGAGGCCGTGAGGGTGAAGCGGG
CGGGGgAGGCCGCCTTGTGGGGTGCGAACGTGGACGAGCTCGGCCTGGGGGAGC
TCGAGAGGCTGCACAAGTCCTTGGAGAGGCTGAGGAGGGACGTGGCGAGGTGCG
CCGACCAGCTCGTCATCGAGGCCGCGCATGCTCGGAGCTCCAGCATCGCAGCGG
CGAGTCGCAGTACTGCTCCTCCTCCTCCTCCTGGTATCCATCTGGgCTTTGGTCGT
GGATTGGAGGGGAGCATGGCGTTAATTCTTCCTCCTCCTCCCACTCCCACTGCCTT
TGGTTAcGGCCGTGGGCTCTTTTAG
SEQ ID NO: 120 >EG4N153108
ATGGTCAAAGCTGAAGTGGAGCTAATGGGCATAGTCGAGGATAAGACACTCGAA
AGGTACCAAAAATGTAACTATGGTGCTCCGGAGACTAATATTATATCAAGAGAG
ACTCAGATTCTTGAGCTTGTAGAATGGATCCGCTATAAGTGGCTTGATGAAGATA
TCGACAAAAATCTCCTCGGTGAGGACTTGGGTCCACTCAGCAGCAAGGAGCTTG
AGCAGCTCGAGCGGCAACTTGATGCATCGTTAAAGCAAATCAGATcAACACGGG
AACAAATGCTATGTGAGGCCAACAAAAGTCTAAGGCGAAGGTTGGAAGAAAGTA
ACCAGGCTGGTCAGCAGCAAGTTTGGGATCCCACTGCTCATGCAGTAGGCTATGG
CCGGCAGCCACCTCAACCACAGAGCGATGGATTCTACCAACAGATAGATGGTGA
ACCTACTCTCCAAATCAGTGTTGAAGGAGAGGAGGATGAGGGTGAATTAGTAGA
GGAGGACATGGAGAAAAGAGCAAGTGATGTAAAAGAGGAATTGGAGTACACCC
TTGTATCCTCCAGAACAAATAACAATCGCAGCAGCACCCGGGATACAGATGAGT
CAATAGAAATCAAGGGGCTCAAACTTCAAAAGTTCGACAAGGACCAAGGGGAG
GGCCAGCACACTGCCCTATAA
SEQ ID NO: 121 >EG4N108259
ATGGGCCGTCAGAAGATCGAAATCAAGAGGATCGAGAGTGAAGAGGCCCGCCA
GGTATGCTTCTCGAAGCGCCGCGCCGGGCTGTTCAAGAAGGCCATCGAGCTGTCC
ATCCTGTGCGGCGCCGAGATCGGTGTCATCGTCTTCTCCCCCGCCGGCAAGCCGT
TCTCCTTCGGCCACCCCTCGGTCGACTCCATCATCGACCGCTTCATCTCTGGCAGC
CCCTCCCCTACGACTATTCCATCCGCCAACCCCCGCATGCCGGCGGCGCGCGAGA
TGATGGTCGTCCGCGAGCTCAACCGCCAATACACGGATCTCGCGGCCTTGCTGGA
GACTGAAAGGAGGAAGAAGGTGGTGCTCGAGGAGGCCGTGAGGGTGATGCGGG
CGGGGAAGGCCGTCTCGTGGGAAGCGAACATCGAGGAGCTCGGCCTGGGGGAGC
TCGAAGGACTGCAGAAGTCCTTTGAGAGGCTGAGGATGGACATGGCGATGCGCG
CCGACCAGCTCGTCATCGAGGCCGCGCATGCTCAGAGCTCCAGCATGGCAGCGG
CAAGCAGTGCTGCTCCTCCTCCTTCTGGTGTCAGTCTAGGCTTTGGTCGTGAATTG
GAGGGGAGCATGGCGCTTCCTCCTCCCACTTTCTTTGGTCATGGCCGTGGGCTCTT
TTAG
SEQ ID NO: 122 >EG4N71703
ATGGCCAGGAGAACCAGCCACGGCCGGCGAAAGATCGAGATCAAGAGGATAGA
AGATGAACAAACTCGGCAAGTGACGTTCTCAAAACGTCGAGGTGGGTTGTTCAA
GAAGGCCAGCGAGCTTTCCACCCTGTGTGGGGCTCAGGTCGGGATCTTGGTGTAC
TCCCCAGGAGGAAGGCCCTACTCCTTCGGCCAACCTGGCTTCGTGGAGGTCTCTG
ATCGATTCCTCCCATGCGTCCCCACGCCGATCGGCTCAGACCCTCCTCCTATGCC
ACCTCCAGCCTACTTGTCGGTGTCCCAGCCCAGCAAGCACTACCTGGAGGTCGTG
AACGTGCTGGAGGCCGCGCGGGCCAAGGGTGCAGTGCTTAAGGAGAGACTTGCC
ATGGTTCTCGAGGAGGAGGGGCGGGCCTATGAGTCTGAAAATGATGACCTCACC
GTGGAGGAGCTTGGAGACCTCGTCGCGCGATTGGAGGCGCTTAAAATGCGGGTG
TTTTCCAGATTCTCTACGATCCTGAATCAACAACAAGCTTCTTCATCGAGTGCTGC
TTTGACTGTCACCCCGCTGAATGTGATCAACCCTTATGCCACCAATGGACCCCAG
GCTTATCCAGGTGGTGGGTTCGTCCTGGGGAATAATGGCCATGGTGCCGGTGGGT
TCCTGGGAACCGGTGGCCATGGTACTCCCAGTGGATTCATGGGGAACGATGGTA
ATGGTCCTCTTGGGTTCATTGCTTGA
SEQ ID NO: 123 >EG4N2959
ATGGTTAGAAAGACAAGCAATGGTCACCGGAAAATTGAGATCAAGAGGATAGA
AAATGAACAAATCCGGCAAGTCACATTCTCAAAGCGACGACAGGGCCTGTTCAA
GAAGGCCAGCGAGCTTTCAACCCTATGTGGTGCTCAAGTTGGAATTTTGGTCTAT
TCTCCTGCTGGAAGGCCCTATTCATTCGGCCAACCTGGGTTCGAAGTGGTATCGA
ATCAATTAATCGCTCACAACTCCTTCATGACCAGCCCAAACCCTATAGAGGGACC
TCAGGGCAATGCAATTGTGCAACAACTGAATTGTCACTGTATGGAGATCATGAGT
CTACTCGACACCGCGAAGACCAAAGGTGCAGTGCTGAAAGAAAGACTTGAAATA
ACTCCAAAGGGGAaGGAGAAGGCTTTCGAGACCGAGCTTGAAGGCTTTGGTATG
GATGAGCTTGAAAGGTTGGTgAAGTCCTACAATGATTTGAAACTAAAGGCGGATT
CAAGAATTTATAAGATAATGAGTGGAGGAGCTTCTTCATCAGGTGGCCCTTTGCC
CGTTAACCCTAAGCTTGCTAGAGATAGAGAGTTACTCTTCCAACCTAATATCTGC
TTGGAGATCTTTTCAATCATAAAAGACCGATCTATGCAGCGAGGAGCGGAGTGA
SEQ ID NO: 124 >EG4N82416
ATGGCGAAGTTGAAGGCAAAGTTTGAGTCTCTGCAGCGCTCCCAGAGGCATTTGC
TGGGGGAAGACCTTGGACCATTGAGTGTGAAAGAACTGCAACAACTTGAACGTC
AACTTGAGTCTGCTCTGTCACAAGCTAGGCAAAGAAaGGCTCAGATAATGCTGGA
CCAGATGGAAGAACTTCGGAAAAAAGTAAGCAtGCTGGATGAAGGCCAAGGTTC
AGAACATTTGGAGGCACGATTTCCATGTTCGATAGAAGAGATTGCCATCGTTGGC
TTCAGCAGAGTGGTGTAG
SEQ ID NO: 125 >EG4N14105
ATGGGGAGGgTGAAGCTAAAGATCAAGAAATTGGAGAATAGCAGTGGTCGGCAG
GTCACCTACTCGAAACGGAGGGCTGGAATATTGAAAAAGGCTAAGGAGCTATCC
ATATTGTGTGACATAGATCTCGTCCTTCTCATGTTCTCACCCACTGGAAAGCCGA
CATTATGCGTTGGAGACCGGAGCACCATTGAGGAGGTTGTTGCAAAGTTTGCCCA
ACTAACTCCACAAGAAAGAGCAAAAAGTTATTGGACCGATCCTGATAAGATTAA
TAACGTAGACCATATTGGGGCTATGGAACAATCTCTCCAGGAATCTCTCAGCCGC
ATTCAGGTGCATAAGGAAAACCTTGGAAAACAACTTATGTCTCTAGATTGCAGTG
GCCAGGTAAAAGCACTTCTTGGTAAGCAAGCAGAGGCCAATGACCAATTACAAG
AGGATTCTTTGCATGAGTTTAGCCAAAACGCATGCTTGAGGTTGCAGCTAGGAGG
CCAGTACCCTTACCAGTCCTATTGTCAGAATTTAATTGGCGAGAATGCATTCAAG
CCTGATACAGAGAATAGCTTACCGGAAAGCACTATAGATTACCAAGTTGACCAC
TTTGAGCCACCTAGACCTGGATACGATGCAAGCTTTCAGAATTGGGCTTCGACAT
CTGGGACATGTGATGTTGCTATATATGATGACCAGTCGTACTCCCGACGCTCCGC
GTTCCGTCATTCCATCGACCCTGTAGCATACCGTGGATCTTACGATTGGTGTCCGT
CAACCTGTGTTCCCCAATGCTTCCCCTATCCACCCACATCTGCTGTACCAGCACCG
AATCATGACCGTTCCTTCCCCAAACGTAGGCTCATTAATATTCATCCAGTCAACC
TACGCGACCCGTTGCTTAAGCCCCACCTTTTCCTTGGATCACTCAAAAACCATGTT
CCAAAATGGAGAAGTCAGAAGGATCTCGCACGTGCCAACCCGGCCTCGGGCCTC
CCAACACGTGCCAGTCGCGGTACCCACACGTTGACGCCACCCAAAAGGGAACAA
aTAAAAAGTACTCACACGTGTCAGCGTCATAACATCCTCCTGTAA
SEQ ID NO: 126 >EG4N37867
ATGTCGAAAGAAATAGTGGGGAAAAAAACTCCTTATCCTCATGAAGAAGCCTTG
GCAGGTTCTCAAGGCCAAGGAGTGTCCAAAAATTCTCAACAAGACTGCACATTA
GCTAAAGGAACAGCAATTAGTTGGAAGCCATGGAATGCCCCTCCCCAGAGTCAT
CACTATAGTGCAATAGAGACAGCTAGAGCTCAGAACAGTACTGCAACAACCTCG
AAGCTAGTCAAAACTAGTGGGAGGTTGTCTGCGGAGATGGCACGCGGCAAGGTG
CAGATGAGGAGGATTGAGAACCCCGTCCACCGGCAGGTCACGTTCTGCAAACGC
CGGGCAGGGCTGCTCAAGAAGGCGAAGGAGCTATCAGTGTTAACCGATGCCGAT
ATTGGAGATATCAGTTCTAAAGCAAGAGATCAACATACTACAGAAGTGTTTGAG
ATAGTGGAGCAAAATGGGCATTTTGATGTAGCTCCAATGATGGTACAACAAAAT
GGGCATTTTGGTGTATCCCCAATGATAGTACAGCAAAATGAGCATTTTACTGCAG
CTCCAGCGATGGAAGACATTCCATATCCACTAACCATACAGAATGACTATTCCAG
TTTTACGAGCTTAGACATGGGCTAA
SEQ ID NO: 127 >EG4N71708
ATGGCCACCATGCCCAAGAAGACCATGGGCCGTCAAAAGGTTAAGCTCAAGAGG
ATAGAAAATGAGGATGCTCTcTATGTGACCTTCTCCAAGAGAAAGTCGAGTCTCT
TCCAGAAAGCTGCCGAGCTTGCCACCCTGTGCGGGTCCGAGATTGCACTGGTGGT
GTTCTCCCCGGCAGGCCGGCCGTACTCTCTCGGCCTCCCCACCGTCGACAaGGTCT
TCCACCGAGTCCTCTCGAGTGGACCTGCCCAAATGGGCTCCGGCCACAGCGTGGT
GAGCCACTCCGCCAAGCAGTGCTCCGAGATAACCAAACACTTGGAACAAGAGAA
GAGCAGGAAGGCCATTCTCGTGGAGAGGCTCCAGAAGGAGGCACCACCCAGGTG
GGAGGATGGGCTCCATGGACTCGGGTGGGACGACcTCCTGaTACTGGCTAAAGAG
GTGGAGGAGCTCAAGTCCAAGgTGGATTCCAGGGTctGCGAGATCCTTCTCCAAGG
GGCTTCATCATCcACGGCTAATGCTGATGCTTGGCCCGTCGGAAGCTCTGAGGGTt
cGTATGGGGTTGGACCACGGGGGCCGCTGGATAATAACATCTAA
SEQ ID NO: 128 >EG4N37348
ATGCCTAGGAAAACCAGGACCACGCGGGGCAAACAAAAGATAGAGATCAAGAG
GATCGAGAAGGAGGAAGCTCGCCAAATTTGCTTCTCCAAAAGAAGATCTGGCGT
CTTTACGAAGGCTAGCGATCTCTCCACCCTCTGTGGCCCGGATGTTGCAGTGCTG
GCATTCTCCCCTCGAGGTAAGCCtTTTTCTTTTGGCAGCCCGGCCGTCAACCCGGT
GATCGACCGGTTCGTGTTGGATATTTCTTCCTCCCCCGGTTCAGGCCACCATTGTG
GACCGCCGAGCAATACGGTCCAACAACTCAGCAAGCTATGCCTGGACCTCACCA
ATCAGCTACATGCTTGTAAGGCCAAGAGTGCAGTGCTGGAGGAGAAGCTCAGCT
CCCCCGGTTATGATATCTTGGAGCTCGATTGGTTCGAGAACGTGGATGACTTGGA
GCTGGACAAACTGGGGAAGCTGGCAGAGGCTCTGAAGCGAGTGAAGGTGAACG
CTGATGCACACGTTGACGCACGCCTCCTGCATGGTAGGGGGGCCTTGTCCTCCTC
TACTACTCCTGTTATGACCGCCAACCAAGTTGAGGGAGCTTCGTCTTCTAATAGG
GTGATGGCTGCTGCATCTTCTAAAGGGGTCATGGCTGCAGGAAATGTGCCGGTGG
CATTCTTGACGATCTCCATGTTAGCGATGTTCGGGAATATGATCAAGAAGAACCA
CTTGGATAATGTGGAGGTTAGTCCATATTGGACAAGGTTGGATGCCAAGTGA
SEQ ID NO: 129 >EG4N71707
ATGGCTGAGAGGACCTTCAGAGGCCGCCAGAAGATCGAGATAAAAAaGATAGAG
AAAAaGGCTGCTCGAGATGTGACATTCTCCAAGCGTAGGGTTGGGGTGTTCGGCA
AGGCGAGCGAGCTGGCAACCCTGTGCGGTGTGGACATTGGGGTGGTGGCCTTCT
CGCCCGCTGGCCGGCCATATACGTTCGGCCATCCGGATGCCAATGTGGTGTTCAA
TCGTTTtCTCGGGCTGGTCCAACCAGAAGGCTCTAGCGGCTCCGTAGGCGCGATG
GCAAGGCATCGGGCTGAGATGCTTCGCCAGCTGACCCTACACTGCTCGCAGATG
ATGGACCGCCTCGCGGCGGAAAGAGAGAAGAGAGCTGTCCTGGAAGAGAGGCTT
CGCAAGGTGAGCGAAGATCCCCAGGAACGCGCATGGCCCGAGGACCTCGAGGG
GTTGGGGCTCGAGAGACTTGCCAGGATGGTGAGGGGCTTCGAGGAGCAGAGGGC
GAAGGCTCGAGCGAGGCTGCATCAGATACGGGAGTTGGGGGAATCATCTTCGGG
GCCTTCGGCCACTGTGGAATTTAAGAAGAGTGTTGTATGa
SEQ ID NO: 130 >EG4N104943
ATGAACGGCGAGAACGACGCTGCTAGCAGGATCATCTTTTCTTCTCTGAAAGAAC
GGCTGGTACAATCCGGTGTTTCCTATGCAAAAGCGGTCAAAAAGCACCCCATCCC
ATCCCCAGTGGTCAGGAAATCTACCGAAACAGTCAAGGATCTCATGAGTTCCAAT
TCAGGAAATGTACATCATCATCCCCGTTCTCGAGGGCACCGGGTGAAGCTCTTGA
GTAAAGGAACTTGTTTTCGCTGTGGAGATCGTGATCACACCCGAGAATCTTGCAG
AAATCCGATTAAATGCTTTCTTTGCAAGGGTTATGGGCATGTTCAAAAGAGCACA
GCATCACCCTTCTGGAAAGGTGTCTTAAGCACGCATGGACTTTTTCAGCAGCTCT
TCTCAATCACCATAGGCAATGGAAAATGGGTCTCATGCTGGACTTTCATCAAATC
AACCATTGAGAGATACAAGAAGGCATGTGCTAATACTTCAAATTCAGGTTCTATT
GTTGACGTTGATTCTCAACAATATTATCAGCAAGAATCAGCAAAACTGCGCCACC
AGATCCAAATATTACAAAATGCAAATCGGCACTTAATGGGTGATTCTCTGGGTTC
TTTGACTGTGAAGGAGCTTAAGCAACTCGAAAACCGACTTGAAAGAGGCATCAC
AAGGATCAGATCAAAGAAGATTGCAGAGACTGAGCGAGCACAGCAAGTAAGCA
TCATTGAAGCAGGACATGAGTTTGATGCTCTTCCAGGATTTGATTCTAGGAACTA
CTACCATCCGCATATATCGCAACAAAAATCTATGATGGCTCTTGTAAATGAAAAA
GAACAGTCACAAAATCAATCACAgCTCCTCCAAGAGCTTGGTCAGTCAGAATGA
SEQ ID NO: 131 >EG4N35645
ATGGGCCGGTCCAAGGTGAAaCTAAAGTTCATTGAAGAACAGCATCGACGTTCGG
CAACCTATAGGAGAAGAATAGCAGGGCTAAAGAAGAAGGCTAGTGAATTGGCC
ATTCTTTGTGACATCCCGGTCTTGGTGATAAGCTTTGGACCCCGAGAACAAgTAG
AGACATGGCCTGAGGACAATCAAGCAGCTCGACACATTATTGACAGGTAtCGAGA
GCTTAGTATCGATATCCGAAACAAGAACAAACTTGACTTACCAGGTTACATGAA
GGCTGAAATCATCAGACATCAAGCATCATTCAATAGGAGGTGCAGGGATTTAGC
TGATATGCCATTGTTGCCTTTGGATGGTTTGTTttATGCCCTGCTCAAGTCACTAAG
GGAGCTTGCTCATCAACTGGACTCAAGAATGGAGGTGATCAAAGAGAGAATCCA
ATTGCTTAAAGATAGAAAGCACTTCAATTTAGGAGAGACCATGAACATGGGAAG
CCAATTGCTAGAAATCACTCCCCGTGATGGGATGATGGGTATTCAAAATACAGCT
TCTGCTTATGATaTGATGTTTTCGGATCCATATCTCACCATGAACGCTTCTTTGCA
AGACCCTCCACAGCCAACGAGCTTCAGTAGCGGACAGATTTCTCCAGATGCTTTC
TTGCAGTATcTTTaTGGGCCAATGGGCATGGATGAGGTACCCTTAGCTATGGTGCC
TTCAATTCCATCGAACATGGATGAGGTACCCTTGGCTATGATGCCTTCGATTCCA
ATGAACATGAATGAGCCTCCAGGGGCACAATTGGCAAAATTATGTGACTAA
SEQ ID NO: 132 >EG4N37749
ATGGCAAGGAAGAAGGTGAACCTGGCATGGATCGCCAACGACTCGACGAGGAG
GGCGACGTTCAAGAAGAGGAGGAAGGGGTTGATGAAGAAGGTGAGCGAGCTGG
CGACGCTGTGCGACGTGAAGGCGTGCGTGATCGTGTACGGCCCTCAGGAGCCGC
AGCCGGAGGTGTGGCCGTCGGTGCCGGAGGTGACGAGGGTGCTGGCGCGGTTCA
AGAGCATGCCGGAGATGGAGCAGTGCAAGAAGATGATGAACCAGGAAGGATTC
CTCCGCCAGCGCGTCGCCAAGCAGCAGGAGCAGCTGCGGAAGCAGGAGCGCGA
GAACCGGGAGTTGGAGACGATGCTGCTCATGTACCAAGGCCTGGCGGGGAGGAG
CCTGCACAGCCTCCGCATCGAGGATGCgACCAGCCTGGCGTGGATGGTGGAGATG
AAGGTGAAGGCGGTGCAGGAGAGGATGGGGCTGGTGAGGGCACAGATGGCGTC
CAGCAGCCAGCAGGTGGTGCTGGAGGCGCCGATCGAGGCACCGGCACCGATGGC
GGTGATGAAGGAGAAGACGCCGCTGGAGGCGGCCATGGAGGCGCTCCAGAGGC
AGAACTGGCTCATGGAGGTGATGAACCCCAATGACAACTTGATGTTTGGTGGTG
GAGAGGAGATGGTGCAGCCCTACATGGACCATACCAACAACCCATGGCTTGACC
CCTGCTACTTCCCTTTGAACTGA
SEQ ID NO: 133 >EG4N154153
ATGGCCCGTAACAAGGTGAAGCTCGCCTGGATCGCCAACGACGCTACCCGCCGC
GCGACCCTGAAGAAGAGACGAAAGGGTCTGCTGAAGAAGGTGCAGGAGCTGAG
CATCCTGTGCGGTGTTGAAGCATGCGCGATCGTGTACGGGCCGAACGACCGGGT
GCCGGAGGTGTGGCCGTCGCCCCCGGAGGCGGCTCGGATCGTGGGGCGGTTCAA
GAGCATGCCGGAGATGGAGCAGACGCGCAAGATGGTCAACCAGGAAGGGTTCCT
CCGCCAGCGCGCCGTGAAGCTGTTGGAGCAGCTCCGCAAGCAGGAGCGCGAGAA
TAGAGAGATGGAAATGAAGCTGCTGATCCGCGAGGGGCTCAAGGGACGGAGCTT
CGACAACCTCGGCATCGAGGATGTCACCTGCCTCTCCTGGATGCTTGAACGaAAA
ATaAAAGAAATTTATGATAAAATGGATGAGATAAAGAATAAGGTGACTGTTAAC
CAAGTCGCCGGCGGCCCGTCGGCACTGCCACTGCAGGTCATGGCTCCTCCTCCTG
CTGCTCCGATCGGGCCGGTCGTGCCCAAGGAGAAGACTACAGTGGAGCAGGCGA
TGGAGGCCCTCCAAAGGCAGAACTGGTTCATGGATATGATGAGTCCATGGCCTG
AGGACTTCTACCAGCCTGCTCAGCCGATGGATCCTTACCAGCCTCCTCCTCCTGC
ACCTCTGGACCACACCATCCCATGGCCGGATCCATCGTTCCCGTTCAACTGA
SEQ ID NO: 134 >EG4N45603
ATGGCCCGTAACAAGGTGAAGCTCGCCTGGATCGCCAACGACGCTACCCGCCGC
GCGACCCTGAAGAAGAGACGAAAGGGTCTGCTGAAGAAGGTGCAGGAGCTGAG
CATCCTGTGCGGTGTTGAAGCATGCGCGATCGTGTACGGGCCGAACGACCGGGT
GCCGGAGGTGTGGCCGTCGCCCCCGGAGGCGGCTCGGATCGTGGGGCGGTTCAA
GAGCATGCCGGAGATGGAGCAGACGCGCAAGATGGTCAACCAGGAAGGGTTCCT
CCGCCAGCGCGCCGTGAAGCTGTTGGAGCAGCTCCGCAAGCAGGAGCGCGAGAA
TAGAGAGATGGAAATGAAGCTGCTGATCCGCGAGGGGCTCAAGGGACGGAGCTT
CGACAACCTCGGCATCGAGGATGTCACCTGCCTCTCCTGGATGCTTGAACGaAAA
ATaAAAGAAATTTATGATAAAATGGATGAGATAAAGAATAAGGTGACTGTTAAC
CAAGTCGCCGGCGGCCCGTCGGCACTGCCACTGCAGGTCATGGCTCCTCCTCCTG
CTGCTCCGATCGGGCCGGTCGTGCCCAAGGAGAAGACTACAGTGGAGCAGGCGA
TGGAGGCCCTCCAAAGGCAGAACTGGTTCATGGATATGATGAGTCCATGGCCTG
AGGACTTCTACCAGCCTGCTCAGCCGATGGATCCTTACCAGCCTCCTCCTCCTGC
ACCTCTGGACCACACCATCCCATGGCCGGATCCATCGTTCCCGTTCAACTGA
SEQ ID NO: 135 >EG4N140076
ATGGCCCGTCGTCGGCGTCGATGGCAGTTCATAGAAAACCAGAGACAACGTTTG
GCCACCTACAGGAAGAGGAGAGGAGGCCTCAGGAAGAAGGCCAGCCAGCTCTC
CTCCCTCTGCGGCGTCCCCATCGCCGTCATCTCTTTCGGTCCCAACGGCCGGCTCG
ACACATGGCCGGACGACCAAGGAGCCATCCACGACCTCCTCCTCACCTATCGAA
GCTTCGACCCCGAGAAGCGGCGGAAGCACGACCTCGACCTACCGACCCTCCTCG
AAGCCCAAGAAGGCAGCCAAAACCTCCTGTGGGATCCTCGCCTCGACGCCATGC
CCACGGAGTCCCTTCGAAACCTCACCAACTCACTCGACTCCAAGGTGAAGGCTAT
CGACGAGAGAATCCAACAGCTGCTCGAGGAAAATTCCAAGTGCAGCAACCAAGA
CAACAATAATTCCAGCAGAGAACAAGGTGTTAATTCCAAGTGCAACGACCAGGA
TAACAATAACACCgGCAGTGAACAGCGTGATGATTCCAAGAGCAGCAACCAAGC
TAAGCAGATAAAAAGGGTGAGAAAATAA
SEQ ID NO: 136 >EG4N41944
ATGGGCAAGATCGAAAAGAAGGAAGCACTCCATATTTGTTTCACCAAGCGCCGC
CAGGGGATCTTCAAAAAGGCCGGAGAGCTCGCCGTCCTCTGCGGTGCCCAGATT
ACCGTCATCACACTCTCTCCTGGTGGGAAGCCCTTCTCCTTCGGCCAACCCTCCAC
TGATGCCGTCATCGCCCGATACCTTGACCCAGGACGCCACCAGGTCCCAATCCCC
ATCACTACTTCACTTGAGATCCGACTGAGATATTATCTAAAGTACTGCAAACTGG
GGGAGCAGTCCGGCGGTGGGTTATGGTGGTGGGAAGCGCCCATAGATGGGCTCG
ACCTCGAAGAACTTGTGGTGATGAAAGGTGCAATAGAGGAGCTCTACAAGGCCA
TCCTGAAGAAGGCCAACCAGCCTACGAGTGCAGGCGAAGCAGTACAAGGCATGC
CACAAAAACCATCGCTAGCAATGCTGAATGGATTAGACAGTTGTGATTGGCTTAT
CCAGCTTTTGGCCAACTGCTCCCAGTGGTTGCGTGATTTGAAAAGAGTGTGTGGG
AGTCTGCTGTCAATCTTTCCGAATATAACGATCAAAGCGGAAGTCAGAGGAAGT
GTGGATCGACGGCTTGCCACGCATATTATTAGAGATGAGGATAAACAGCAGGTG
CACAGGTCGACAGCCATCATGAGGATCAATGTTTGA
SEQ ID NO: 137 >EG4N3001
ATGAGAAGGTCTCAAGTCAAGCGGATACTTTTAAAATGTCCTGTAAAGAAAGCT
AAGGAGGGCGAGGAGCCTTTGGAGGCTGTTGCCAACAAAATCTGGCCTAATGAT
GATCTGGAGTTTCAAAGTGGAAAGTCGATGATTCAGAAAGTGAAGgggATGCTGA
GGGTTAGAAGCATGGATACGGCTATATATTCTTCCAAAGTTATGTACCTTCCAAA
AATTACTCTTCCTTATCAAAAATTCACAAACACTTGGTGCTTGGGGTGGTTTGGA
CCAATTATCCAGCAGCTGCCAATCGGTTCAGCACCAGGAACACTTACTTTTGTGA
CTTGTCGCTCAGAGTCACAAACCCATCCTAGGACTTGGTTGACCACCAGCCCGAC
CTGGGACACTAGCATGAAGTCAGTGATAGAACGCTACAACAAGACCAAAGAGGA
GAATCATCTAGTTATGAATGCAAGTTCAGAGACTAAGCCTATCAGGTTCCGCCTA
GCTTCAACTGCCAAAAGTCATAATTCTGATGGGGCAGATGAAAGGGGAAAGGAC
TCAAATTTAATGCTTGTAGATGCTCATGAGCGACAAGAATTACTGACAGATTTAG
GACGGAATCAACCTCACAAACATCACTTCTACAGAAATAGAGAGGCAGATCACA
TTCAGCCTCAAGGTGGAGCAGCAATTTCCTATGAGGTGAAGGATGTTTTTGTCCA
AGAGGATGGAATTTTTTGGCAAAGGGAGGCAGCAAGCTTGAGGCAGCAACTGCA
TAACTTGCAAGAAAGTCACCGGCAGTTGTTGGGAGAAGAGCTTTCTGGCCTAAGT
GTGAAAGATCTACAAAATCTAGAGAACCAACTTGAGATGAGCTTACGTGGTATC
CGAATGAAGAAGGTTTATGCAATGAGGGGTGTAAATGGCATTGATAAAGGTCCG
ATTACTCCATATGGTTTTAATGTCACCGAGGATGCAAACATATCCATTCATCTTG
AACTCAGCCAGCCACAACTGCAAACAGATGCAACGCTTGCTCAAGGCCAAGGAA
ACAAGGAAGTTGACCAAGGTCATTCTCATCAACCTACCAATGAAGATATAATGC
CTTCCGGGTTCACCATAGAATACGTGTTGGCCATTGAACAGGTAGTAGCGGGTGC
CCCCACTGCTCCCTTTCCACGTGGACAGAGAGGCCCGACGCTGGACCCCCGACGT
GCCAACTTAGGTCGTCGACACGTGGGTGTTGTCGGCGGTGGGAACCTCTTTGCGA
AGAGATATGACTTTTTGGAAGAGAATGTTGGTTTCCGAAGAGTTACAATCATATC
TCTTCAAAAATATGGCACTTCGACAGAGTCTATAAGTAGGCTTCGATCCAATTTG
TTTCAAAATAATAAAAAATCTTAA
SEQ ID NO: 138 >EG4N60802
ATGACAAATCGTGGGCGTGGATTGCAGTTGATAGAAAATCGGACACAATGTTTG
GTCACCTACAGGAAGAGGAGAGAAAGCCTCAAGAAGAAGGCCAACCAGCTTTCC
TCCCTCTGTGGCGTCCTCATCGCCGTCATCTCTTTCGATCCCGATGGCCGGCTCCA
CACATGGCCAGATGACCAAGGAGCTCTCCCCGACCTCCTCCTCACCTATCGAAGC
CTCGACCCCAAGAAGCGGCAGAAACACGACCTCGACCTACCGACCCTCCTCGGT
GCCATGCCCGCGGGATCCCTTCGAACAGGACCGGCTAAAGGCCATCTCTGCCTTC
GAAAGCTCGCCAACTCACTCCACTCCAAGGTGGAGGCTATCGACGAGAGAATCC
AACAACTGCTCGACAAGAATTCCAAGTGCACCAACCAAGACAATAATAGTACCA
GCAGAGAACAAGACGATGATTCCAAGTGTAACAAGAAAGGTaAAAATAATAATA
CCAGCAGTGAAAAAGGTGATGATGACTCCAAGGGCAGCAACCAAGGTAATAATA
ACAATAATACCAGCAGTGAACAAGGTGATTATTCCAAGAGTAACAACGAGGGTA
ATGATAAGAACAAGGTTTGCCTCCTTGTAGTAACCCGGTGGTCTTTCATCCCTTCC
CTATAA
SEQ ID NO: 139 >EG4N14015
ATGTCGAGGAGCAGCATGAAGCTcGAGTTGATTGCCGATGATGCTGCTCGGAAGA
CATCCCTGAAGAAGAGAAAGAAGGGCTTGTTGAAGAAGGTGCAGGAACTCAGCA
TCCTATGCGATGTCGATGCATGTGCGATAATTTACGAGCCAGATGATCGCCACCC
AGAGTTATGGCCCTCATCCGAAGAGGCTACCCGGATGCTCGTGCGGCTCCGAAG
CATGCCAGAAATGGAACAGAAGCAGAAGATGATGAACCAAGAGGAGTTCCTCTA
CCAGAAGATGAGGAAATTGGTAGACCAACTTCATAAGCAGGAGTTCGAGAATAA
GGAGCTGGAGAAGAAGCTAAAGATGTATGAGGCACTGAGGACGGGGGACTTCA
GTGAATTGGACATGGAGCAAGCCATGAACCTGTCGATGATGATCGAGCAGATGT
TGAAGAAAATCTATGAGAAGATGGACGCGATCAAGAAGCATCAAGCAGCAATG
GCACGGGTTGACGGAGTAGTGCAAGAGGGTGGGAATGCGGCTGGACTGAACACT
CCGAGGGAGAACACCCCAACGGAGAAGGATAACGAGATACTCCAGAGGCAGAA
GCAGATGCTGGATATGATGATCCCGAGGTCAAGTAAAACCTATCAGCCTTCTGCG
GGTCCGACCAACCCATGGCCGGCTAATTCCTTGTTCCCCTTCAATTGA
SEQ ID NO: 140 >EG4N21371
ATGACGAATCCGGACGATGGAGAGGTGGGCGGAGGAGGAGGAAGCGAGCGATG
TGTAGCATCAGAGAAAGTTACAGGGAAGAAGGCTAGGAGAGCTACATTTAAGAA
GAGAAAGAAGGGTTTGATGAAGAAGGTAAGTGAATTGAGCACTTTATGTGATGT
CAAAGCATGTTTGATTGTCTATGGGCCAAATGAACCAGAAGCGGAGGTATGGCC
ATCAGTGCCAGATGCTATGCGTGTGCTTACAAAGCTAAAGAAAATGCCCGAGAT
GGAGCAAAGCAAAAAAATGATGAACCAAGAAGGCTTCATGCGTCAGAGGATCAT
GAAGCTACAAGAACAACTCAGGAAGCAAGATAGAGAGAACAGAGAGCTCGAGA
CAATCCTATTGATGTATCAAGGCTTGGCAGGGAGGAGCTTACACACCGTGACTAT
TGAAGATATGACAAGCCTCGCATGGCTTATTGAGATGAAGGTAAATAAAGTACA
AGAGAGGATAGAGCATTCAAAAGGAGAGATCGCATCAAAGATGGTGGAGGGGA
TGAAAGAGGAGAAGAAGAAAGTCGAAGGGCCATCAAATATCAAAGAAAAAATA
TCTTTGGAGGTTGCCATGGAGGAACTTCAGAGGCAAGAATGGTTCACTGAAATA
ATGAATCCACATGACCTAATGATTTGTGGAAATGAAGTCGTGCAACCCTACATAG
ATCATAATAACCCATGGTTGGATGCTTACTTTCCTTGA
SEQ ID NO: 141 >EG4N122402
ATGGGTCGCCACAAGATCCCCGTCAAGATGATCGACAAAAAAGACGAGAGCAAC
ATCTGCTTCTCGAAGCAAAAGAAGGGTCTCTTCTCCAAGGCGAAGCAAATCGCTC
GTGCAGGCAGTGAAGTCGCCATCATCGTCTTCTCCCGTGTCGGTAACATATTCAC
TTTCTGCCACCCTAGCATAGAATCTGTTGCTAGTCGCTTCCTCAGCCAGCAAAAC
ATCAAACACAGATCATCCAATGATGATAATTTTCATGGCAATGCCGACTTCGTGT
ATCCGGGGTCCGACGCTGCAAGAGGAGGTCTTACCGGACCATCCGAAGAAGGTG
AAACATCAAATAAAGGAGATAATAAATTAGATGGAGGAAACACCATCATGCAGG
ATAAGGGGTTCGAGTCTGACCATGAAGAAGAAGAAGTGGAAAGTAAGACCAGCT
CGAAGGCTGAAGGGTCGGACGTCGCCGGCAGTTCGCAAGAGGAACATGCATTGA
TGCATGATGGAGAAGAACATGCAACAGGAGAAAAAGAGACTTCTTCTGACGAGA
CACTGCATAGCGGTCGATTTTGGTGGAACAACCGAATTGATAATCGTGAGTTACA
TGAGCTGTTAGAGTTTGAGAGCGCGCTCGTGGAGCTGCGGGAGAAGGTGCGAGA
CCAAGCAAATCAGATCCTGGTTCAGAAACCAGTGATGGGATATTATTTAGATTTT
AGTAATTACAAGTTCAAGTTTGATGAGCAGGCGTCACAGGATTAG
SEQ ID NO: 142 >EG4N42750
ATGGTCCCGAGGGCAGAGCTGTGGGCAGTGTGGGCTGGTATTGCCTATGCGAGG
CTGGCTCTTACAGTAGACCGACTCATCATTGAGGGTGACTCAGGCACTATGGTTA
AATGGATTCAAATGCGGGATACAGAGGATGCTGCTCACCCACTTCTGAGGGATA
TCGCGATGCTGCTGAGGGGGGCCACCATCACTGCAGTCACAATCCGGATGGAAA
ATCTCTCAATAAGAGCATCCTCGTTCAGTCTAACAAATGGTCGATCTGAGCTCTC
TGGACTAGTCTGTGGAGGGGTGCCAAAAATTCAGTCTTCTATCTTCACTGAGAGA
GTCAGCTCTTGCATCTCAAGAGTCGACTCGCCATTCGTGCCAGTGTGTTCCAATG
TGCCAGAGAAATTGATGGGCGAACAGTTGTCTGGCTTAAATGTCAAAGAACTGC
AAAATCTAGAGATCCAACTTGAAAGGAGTCTTCATTGTGTCCAAAAGAAGAAGG
GGTACCTTCTTCACAATGAAAATATTGAACTCTACAAGAAGGTAAACCTTATACG
TCAAGAAAACATGGAGTTGCGTAAGAAGCCTCGCAATATACTCAGTCGCACTGA
CAAAGCATAG
SEQ ID NO: 143 >EG4N157194
ATGAACGGCGAGAACGACGCTGCTAGCAGGATCATCTTTTCTTCTCTGAAAGAAC
GGCTGGTACAATCCGGTGTTTCCTATGCAAAAGCGGTCAAAAAGCACCCCATCCC
ATCCCCAGTGGTCAGGAAATCTACCGAAACAGTCAAGGATCTCATGAGTTCCAAT
TCAGGAAATGTACATCATCATCCCCGTTCTCGAGGGCACCGGGTGAAGCTCTTGA
GTAAAGGAACTTGTTTTCGCTGTGGAGATCGTGATCACACCCGAGAATCTTGCAG
AAATCCGATTAAATGCTTTCTTTGCAAGGGTTATGGGCATGTTCAAAAGGGTTTC
GCCACTCTTAGCACCAAGATAGAAACTGGGGCCACCTCCTGCCCGGTTTCCCTTG
TGGTGCTAGAGTCTAAAACCTCTCTCCCTCTCTCCCTTTGTCGTTTCCTCCGGGGC
CCTTATTGGAAAGTAATATTGGGTTACATTGCTCGTGACACATCTGAGCTTAGTT
ATGATGATTGCTTTGAACGGAGAGAGAGAACTTTTGGcTGGCGTGGATTGTTTTTT
GGACCGAGCGCCATCACGTCGCTTTCAAGCTTGTGGTGTCGTCTGCCCATTTGTA
ATCTCCGAAGGCCGTACCTTGTCTTGTTTTCCTTTCGCCAGAACCTTAACCTCGTC
GATAAGCACTTAATGGGTGATTCTCTGGGTTCTTTGACTGTGAAGGAGCTTAAGC
AACTCGAAAACCGACTTGAAAGAGGCATCACAAGGATCAGATCAAAGAAGATTG
CAGAGACTGAGCGAGCACAGCAAGTAAGCATCATTGAAGCAGGACATGAGTTTG
ATGCTCTTCCAGGATTTGATTCTAGGAACTACTACCATGTCAGTATGTTGGAGGC
AGCACCCCACTACTCACACCAACAAGATCAGACAGCCCTTCATCTCGGTATATAA
SEQ ID NO: 144 >EG4N6887
ATGGGTCTACGAAACAAGCCACCAAATCAAAGGAGATATGGGATATCTTACGAG
AGAAATTTCAAGGGAATACCAAGGAATTTGATGGGAGAGTCTCTTGGCTCTATG
AGCCCTAGGGACCTGAAGCAACTGGAGGGTAGGTTGGAAAAGGGCATAAACAA
AATAAGGACAAAAAAGATTGCTGAGAATGAGAGAGCACAGCAACAGATGAATA
TGTTACCCCAGACAACTGAATATGAGGTCATGGCTCCGTACGATTCAAGGAACTT
CCTTCAAGTGAATCTCATGCAAAGCAATCAGCATTACTCTCATCAGCAGCAGACG
ACTCTCCAACTAGGAAAGAAGATCGTAGATCGGGTGGCTAGTTCAACTGACAGA
TCGGATGTTGGGATAATTCAGGATCTTCCTAACCAAAGGGGACCAGAGGGGCGT
CGCCCGTGGTCCGACGGGCTACAGCAGCATGGTCGCTGGTTCGGCAGTGGTGACT
GA
SEQ ID NO: 145 >EG4N91665
ATGAGCATCGTCGATAACTCTGATATGTCGATGGCATCGTGTCGATTGCAATTGA
TAGAAAGCCGGAGACAACGTTTGGCCACCTACAGGAAGAGGAGGGAAAGCCTC
AAGAAGAAGGCCAACCAGCTCTCCTCCCTCTGCGGCGTCCCCATCGCCGTCATCT
CTTTCGGTCCCAATGGTTGA
SEQ ID NO: 146 >EG4N126213
ATGGAAGTCCTCCCGATCATTGACCTCCACCCGACTGTTATCTTGGGATCAGTTCT
TGAATTGCCCCAGCGAGAAGGAAAGCCCCAAAGAAGAATAGAAGAAGCaAAAA
AGAACTGGTTCTTCCAcCCATGGATGGATGATAGAAGATCGAGGAGAGCTCTTCT
CtTTCCGCTTCGAGATGCCAATGACCCAACACCAGCACACGACAGTGACCTCTCgC
AGCAGGGGCTGTGGCAACCTCCTACGGCAACCCCATCACAGCCACGTTCAGTGA
CAGATATTTGGTTGTGCAAGTGGATTGAAAGTGACTTTCGGAACTCGTTTGGTTC
ATGGGAAGAACTTTTCTTCCTAAAAATTAACTTTCAACCAGTTTTTTCCAGGCACT
TGATGGGTGATGCTCTGAGTTCTTTGAGTGTGAAGGAACTTAAGCAACTTGAAAA
CCGACTTGAAAGAGGCATCACAAGGATCAGATCAAAGAAGATTGCAGAGAATGA
GCAAGCAGCACTGCAGGTAAGCATTGCACAAGAAGGACCTCAGTTTGATGCTCT
TCCAGCATTTGATTCTAGAAACTACTACCATGTCAATCTGTTGGAGGCTGCAACC
CATTACTCCCACCAACAAGATCAAACAGCTCTCCATCTTGGGTATGAAGCAAGAT
CTGATCATGCTGCATAG
SEQ ID NO: 147 >EG4N36286
ATGCCaCGGAGGAAGGTCGTGTTAGAGCCCCACCCCACCGAGCAAGCTCGGATG
CAGTGCTACTTGACTCGAAGGAATGGTATTAAGAAGAAGGTGAGGGAGCTCTCC
ATCCTCTGCGATGCCGATATTGCCCACCTCTCCATCCCTCCTGCAGGAGAGCCTTC
GCTGTTCCTCGGCGCcCACACGTCATGTGGAGGCCTTGTGGTGCTCGCTGGCTCG
GTGTACTCCACCATAGCCTTGCACCCCTAG
SEQ ID NO: 148 >EG4N3542
ATGGCTCCTCCTCTCGGAAGCGGCGCCGCCACCTCCGGCGGCAACGGCGACGGT
CGCGGCGAGAGATACCGGTGGAAATCCATCGAGAAGCGGACGTGGGGCCTCTGC
AAGAAAGCGTACGAGCTCGCCACCCTCTGCGACGTCGACGTCGCCCTCATCTGCT
ACCTCCCCAGCGTCGACACGCCCACCATCTGGCCGCCGTACCGCCATAAAGTCGA
ACAAGTCGTCCACCGCTACGTCGACATCCCCGCCGACAAGAAGCTCCCCAAGAA
CCAGATCACCCTCCACATCCCCAACTCCACGGCCGGGAACACGAAGGACGCAGG
CGAGGCGGCGGCAGTGGCGGACGCCGACCGCATCCGTGTcCCCTTcCCCTACGAT
GAAGACAAGCTGATAGCTATCGTGAGGTATTTGGATTCGAAGATCGTGGAGGTG
CGGAGGATGATCGCGGCCCGTcGGATGGAGCGGAGGAGCGAGCCGGCGCTGGCG
GTGGCGAGCGGCGGTGATGGGGATCCTGGGACGGCCGATTGGGATAGGGGGAA
GAGGGTAGCCCGGGATTGCGGTCCGGTTTGGGGACGGGGGCGTCCGGATTTCTC
GGCTCTGGCGGCGGCGGCGGCGGCGGCGGCGAGGGGCGGTGGCAGCGGGGGAG
CACCGAATTCTTCGCGCTCCTGCCTGTGCTGTTACTGCCCCCATCACGGGCACTG
GTTCACTGGATTCGACGGtAGAAATGCTTCGAGAGATGGATCGGACGGCATTTGA
SEQ ID NO: 149 >EG4N71936
ATGGCTCCTCCCCGAGGCGACGGTCGAAGCGATAAATCCCTCCGCCTATCCATCA
AGAATCGGACGAAGGGCCTCTGCAAGAAGGCGTACGAGCTCGCCACTCTCTGCG
ACGTCGAGCTCGCCCTCGTCTCCTACCCCTCCGACGGCGCCGAACCCACCACATG
GCCGCCCGACCGATCCAAGATCGAAGACGCCTTCCACCGCTACTTCGAAACCCCC
GCCCACAAGAAGCTCCCCAAGAACCAGATCACCCTCGACAACCCCAACCCCGGT
GCCGTCGAGAAGAAAGACGCCGCCAAAGCGGCCGCGTCGAAGGCGCCGAAGGA
GACCGACCGCCTCCGCATCCCCTTTCCTGACGACGAGGACAAGCTGATAGCGCTG
CGAGGGATCTTGGATTCGAGGCTCGAGGCGGTGCGGAAGATGATCGCGATCCGT
CGGGCGGAGGAGAGGAGGGATCCGAGACCGTCCGCTCGGGATACGGAGAAGGA
GCTTGCCGTCGCAGTGGCGAATGCCGGTGGTGGTGATCCGACGCCGTCCGCTGGA
GATCCGGGGAAAAGGCTTGCCCAGGGTCAAGGTGGGCCGCTGCCAGCAGCGGCG
GCGGTCGCGGCGGCGAGCGCCGGTCGAGAGGATCCGCGGCCGTCCGTTCGAGAT
GTGGAGAAGATGGTGGCCGGGGATTGCGGTCCGGTTTCTGGACGGGGGAATCCG
GATTGCTCGGCCGCGCCGGCTGCGGCGGGCAGCGGAGGCGGCGGGGCACCAAAT
TCTTGGCTTCAACCATCTGCTCATGGTGGAAGAAGCCATTGGAGCTACAGGCTCC
AAACCGAACCCACCTTCTCACCCCAGAAAGAAGCCGCCGGAAACGGAAGATACC
CCCCCGGAACGCGGGAATCAGTGGCATATCCCGTAATTCAACCCAAACTCCAGT
GGCATTCTTCTTCCCTGGCCCCACCTCAACGTCACCTCTTGCGTGAAGCGGCGTC
ACCGATCACGCCCCCCTTCACGGTGACGTGGCACCGGCGGCGGTTTACCCATTTC
CTGCGCCGCCGGAACGCCACTTATGATACCGTGCATGGGAAGTGGAAGCACCAC
GATATCAAGGTCAAGGACTCGAAGACCCTTCTCTTTGGCGAGAAGCAAGTCACT
GTCTTTGGCATTAGGAACCCTGAGGAGATCCCATGGGGTGAAACTGGTGCAGAG
TATGTTGTGGAGTCTACTGGTGTCTTTACTGACAAGGAGAAGGCTTCTGCTCACC
TGAAGGGTGGTGCCAAGAAGGTCATCATCTCTGCTGCTAGCAAAGATGTTCCTAT
GTTTGTGGTGGGTGTGAACGAGCATGAATACAAGTCTGACATTGATATCGTCTCC
AATGCTAGCTGCACCACAAACTGTCTAGCTGTTCTGGCCAAGGTCATCAATGATA
AATTTGGCATCATTGAGGGTTTGATGAGCACAGTGCATTCCATCACTGCTACTCA
GAAGACTGTTGATGGGCCATCCAGCAAGGACTGGAGGGGTGGACGAGCTGCCAG
CTTTAACATCATTCCTAGCAGCACTGGTGCTGCCAAGGTTGGAAGGAGTTTTGGG
GTACTTACCACTAcGTACAAGGATGCCGCTGAGGATAAGGCCGACCGATGCCGA
AATCAGACAGTACGCGGCGAGGAAGAGGCCGACGTCTGGGACCGGACCCTCACG
ACCGCCGAAGAAACCCTCAACAGCAGTGCCGACCGTCGTCGCATCGGCGGCCGA
TCAGTCGGAGCCGGTAATTGCACTTTCGGCTCCGACAGCGCCTCCGGAAGAGCG
GCCAGCGGAGGAAGTGGCCGAAGGAACATCGGTGATTTCACCGATTGA
SEQ ID NO: 150 >EG4N29531
ATGGAAGGGGTGGAAAAAATTGAGGAAATAATTGCTCGTGAGCTAAATATGATG
AAGACACTCGAAAGGTACCAAAAATGTAACTATGGTGCTCCGGAGACTAATATT
ATATCAAGAGAGACTCAGGAAGATGTGGATGCTTTGTATGGCCAAGTTTGTGATA
TTTTtCTTAAATATCCTAACGAACTAGCAGTTGAATGGTCTGAAGGTCTAGATTAG
SEQ ID NO: 151 >EG4N44436
ATGCGgGAGGCGaTCGGGGGCTCGCAGCCAAGGGCTCAGGGAGGCGAGAGGCggT
CAAGGGaTCGAGGAGATGGGAGGcGATCGAGGGCTAGGGGAGGCAGATTGGGGG
gTCAGGGAGGTAGGAGGcAGGCAGGGGCTCGCGGTCGGGAGCTCGAGGAGGtGG
GAGGCAGCcAGGGGCTCgAggAGGCAAGCCGGGGGCttAGAGAGGcGgAaGGCGgTC
GGGGGCTCACAGTCGGGGGCtcGGAGAGTCGGGAGAcAGCCTGGaTCTTAGGGAG
GcGGTCGgATGCTCAtAGtcGAGGGCTTGAGGAGGTCAGAGACGGTCGGATGCTTA
CGATCGGGGgCTCGAGGAGGCGGAGGCaGAGGAAAGAGGGGGTGGGGAAAAaTA
AGGGGGGgTGGCAGGGCACGGGACTGGGACTCTCCTCAACCGCtATAAATAAagC
AAGCTACCCCTCACAAGAACCAGAAGCTtGGAGCAAACCAATGGTTGGTAAAAA
ATTGAACGTAGAATTCATAAAACACCGGAAAAAGCGTTTGGCCACCTACCGGAG
GAGGAAAGAAGCCCTCAAGCAGGCGGCCTACGAGCTCTCGACGCTCTGCGGCAC
CCCCACCGCCGTCATATACTTCGGTCCCGATGGCCAGCCCGAATCATGGCCGGAG
GACGAAGGAGCCGTCCGCGACATCATCGGAAGGCATCCAGGCCTCGGCGCAAAG
AAGCGGAgCACGCGTCCCTTCGACTTACGGGATCTTCCTCCGTTTGACGACACGT
CGGAGGAGTTTTTGAGAGAGATGCTTTGTTCAATGGAGTCGGGTATGGAGGCTGT
CAAGGAGAGGATCCAACTTCTCAAAAAGGATTCCAGGTGCAACCAAGGCGACTT
CCATGGTGATACTGGCGGTGTACAACAACAAGGTTGCCAATGTAATAATCCTGCT
TTCATGGAGGAGTGCTTTGATGTGCCAATGGTGTCCAAGGCAGCCATGGATGATG
GACCAGGCCAAGGCCATGGTGCTTTCGCGCCGATGGAGCTAAAACAAGTGGAAG
GAGTTGCTGCCGATGCTTTCTTGCCATGTTCTTCTAATGCATCGATGGACTTCAAT
GATGAACTGGCGGCGTTCTCCATGCCGTTAATTTTCATGCCACCACCATTCACCG
GAGCTACTTCAGAGCATGACATTGCATGCATCTGGCAGTGA
SEQ ID NO: 152 >EG4N37875; SHELL (DeliDura Allele;
Sh^{DeliDura}; Sh⁺)
ATGGGTAGAGGAAAGATTGAGATCAAGAGGATCGAGAACACCACAAGCCGGCA
GGTCACTTTCTGCAAACGCCGAAATGGACTGCtGAAGAAaGCTTATGAGTTGTCTG
TCCTTTGTGATGCTGAGGTTGCCCTTATTGTCTTCTCCAGCCGGGGCCGCCTCTAT
GAGTACGCCAATAACAGCATAAGATCAACAATTGATAGGTACAAGAAGGCATGT
GCCAACAGTTCAAACTCAGGTGCCACCATAGAGATTAATTCTCAACAATACTATC
AGCAGGAATCAGCAAAGTTGCGCCACCAGATACAGATTTTACAAAATGCAAACA
GGCACTTAATGGGTGAAGCTTTGAGCACTCTGACTGTAAAGGAGCTCAAGCAAC
TCGAAAACAGACTTGAAAGAGGTATCACACGGATCAGATCGAAGAAGCATGAGC
TGTTGTTTGCAGAGATCGAGTATATGCAGAAAAGGGAAGTAGAACTCCAAAATG
ACAATATGTACCTCAGAGCTAAGATAGCAGAGAATGAGCGAGCACAGCAAGCAG
GTATTGTGCCGGCAGGGCCTGATTTTGATGCTCTTCCAACGTTTGATACCAGAAA
CTATTACCATGTCAATATGCTGGAGGCAGCACAACACTATTCACACCATCAAGAC
CAGACAACCCTTCATCTTGGATATGAAATGAAAGCTGATCCAGCTGCAAAAAATT
TACTTTAAGTATGTCGCTGCTTGT
SEQ ID NO: 153 >SHELL(MPOB Allele; sh^{MPOB}; sh⁻)
(base mutation italicized and underlined in the following
listing)
ATGGGTAGAGGAAAGATTGAGATCAAGAGGATCGAGAACACCACAAGCCGGCA
GGTCACTTTCTGCAAACGCCGAAATGGACTGC [symbol] GAAGAAAGCTTATGAGTTGTCT
GTCCTTTGTGATGCTGAGGTTGCCCTTATTGTCTTCTCCAGCCGGGGCCGCCTCTA
TGAGTACGCCAATAACAGCATAAGATCAACAATTGATAGGTACAAGAAGGCATG
TGCCAACAGTTCAAACTCAGGTGCCACCATAGAGATTAATTCTCAACAATACTAT
CAGCAGGAATCAGCAAAGTTGCGCCACCAGATACAGATTTTACAAAATGCAAAC
AGGCACTTAATGGGTGAAGCTTTGAGCACTCTGACTGTAAAGGAGCTCAAGCAA
CTCGAAAACAGACTTGAAAGAGGTATCACACGGATCAGATCGAAGAAGCATGAG
CTGTTGTTTGCAGAGATCGAGTATATGCAGAAAAGGGAAGTAGAACTCCAAAAT
GACAATATGTACCTCAGAGCTAAGATAGCAGAGAATGAGCGAGCACAGCAAGCA
GGTATTGTGCCGGCAGGGCCTGATTTTGATGCTCTTCCAACGTTTGATACCAGAA
ACTATTACCATGTCAATATGCTGGAGGCAGCACAACACTATTCACACCATCAAGA
CCAGACAACCCTTCATCTTGGATATGAAATGAAAGCTGATCCAGCTGCAAAAAA
TTTACTTTAAGTATGTCGCTGCTTGT
SEQ ID NO: 154 >SHELL(AVROS Allele; sh^{AVROS}; sh⁻)
(base mutation italicized and underlined in the following
listing))
ATGGGTAGAGGAAAGATTGAGATCAAGAGGATCGAGAACACCACAAGCCGGCA
GGTCACTTTCTGCAAACGCCGAAATGGACTGCTGAAGAA [symbol] GCTTATGAGTTGTCT
GTCCTTTGTGATGCTGAGGTTGCCCTTATTGTCTTCTCCAGCCGGGGCCGCCTCTA
TGAGTACGCCAATAACAGCATAAGATCAACAATTGATAGGTACAAGAAGGCATG
TGCCAACAGTTCAAACTCAGGTGCCACCATAGAGATTAATTCTCAACAATACTAT
CAGCAGGAATCAGCAAAGTTGCGCCACCAGATACAGATTTTACAAAATGCAAAC
AGGCACTTAATGGGTGAAGCTTTGAGCACTCTGACTGTAAAGGAGCTCAAGCAA
CTCGAAAACAGACTTGAAAGAGGTATCACACGGATCAGATCGAAGAAGCATGAG
CTGTTGTTTGCAGAGATCGAGTATATGCAGAAAAGGGAAGTAGAACTCCAAAAT
GACAATATGTACCTCAGAGCTAAGATAGCAGAGAATGAGCGAGCACAGCAAGCA
GGTATTGTGCCGGCAGGGCCTGATTTTGATGCTCTTCCAACGTTTGATACCAGAA
ACTATTACCATGTCAATATGCTGGAGGCAGCACAACACTATTCACACCATCAAGA
CCAGACAACCCTTCATCTTGGATATGAAATGAAAGCTGATCCAGCTGCAAAAAA
TTTACTTTAAGTATGTCGCTGCTTGT

### EXAMPLES

The following examples are offered to illustrate, but not to limit the claimed invention.

### Example 1

### Identification of SHELL Binding Partners

The coding sequences for oil palm Sh^{DeliDura}, Sh^{MPOB}, Sh^{AVROS} and rice OsMADS24 were synthesized as two ˜300 bp gBlocks each that overlapped by 30 bp (Integrated DNA Technologies). Gibson assembly of the two fragments was performed using kit manufacturer's protocols (NEB). EcoRI and BamHI sites were added to the gBlock sequences for simple ligation into MatchMaker Gold Yeast Two-Hybrid vectors. Each sequence was cloned into both the binding domain vector, pGBKT7, and the activation domain vector, pGADT7. SHELL sequences encoded amino acids 2 to 175, including the entire MADS-box, I and K domains. The C domain was excluded from yeast two-hybrid constructs to avoid auto-activation of selection genes in the yeast two-hybrid system. The Sh^{DeliDura} peptide sequence encoded by the vectors was:

(SEQ ID NO: 155)

GRGKIEIKRIENTTSRQVTFCKRRNGL [symbol] K [symbol] AYELSVLCDAEVALIVFSSR
GRLYEYANNSIRSTIDRYKKACANSSNSGATIEINSQQYYQQESAKLRH
QIQILQNANRHLMGEALSTLTVKELKQLENRLERGITRIRSKKHELLFAE
IEYMQKREVELQNDNMYLRAKIAEN.

The Sh^{MPOB} peptide sequence encoded by the vectors was identical to the above sequence, with the exception that the underlined leucine residue (L) was converted to proline (P). The Sh^{AVROS} peptide sequence encoded by the vectors was identical to the above sequence, with the exception that the underlined lysine residue (K) was converted to asparagine (N). OsMADS24 sequences encoded amino acids 2 to 177, including the entire MADS-box, I and K domains, but excluding the C domain. The OsMADS24 peptide sequence encoded by the vectors was:

(SEQ ID NO: 156)

GRGRVELKRIENKINRQVTFAKRRNGLLKKAYELSVLCDAEVALIIFSN
RGKLYEFCSGQSMTRTLERYQKFSYGGPDTAIQNKENELVQSSRNEYL
KLKARVENLQRTQRNLLGEDLGTLGIKELEQLEKQLDSSLRHIRSTRT
QHMLDQLTDLQRREQMLCEANKCLRRKLEES.

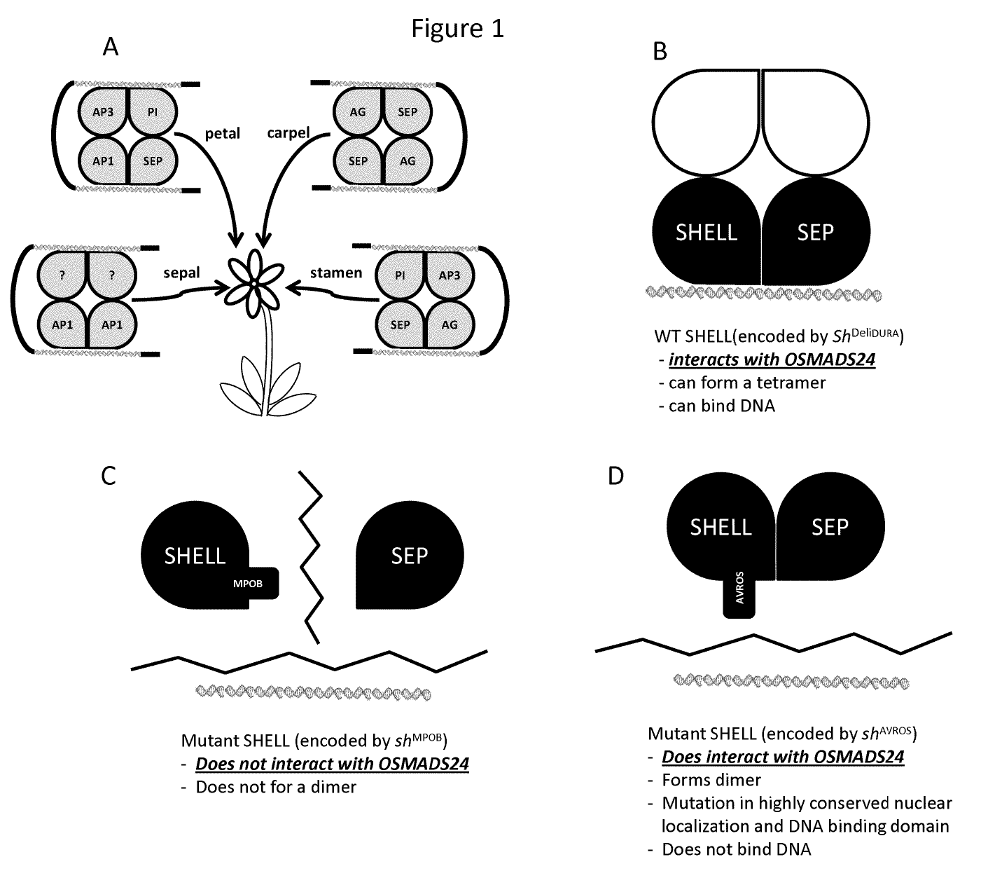

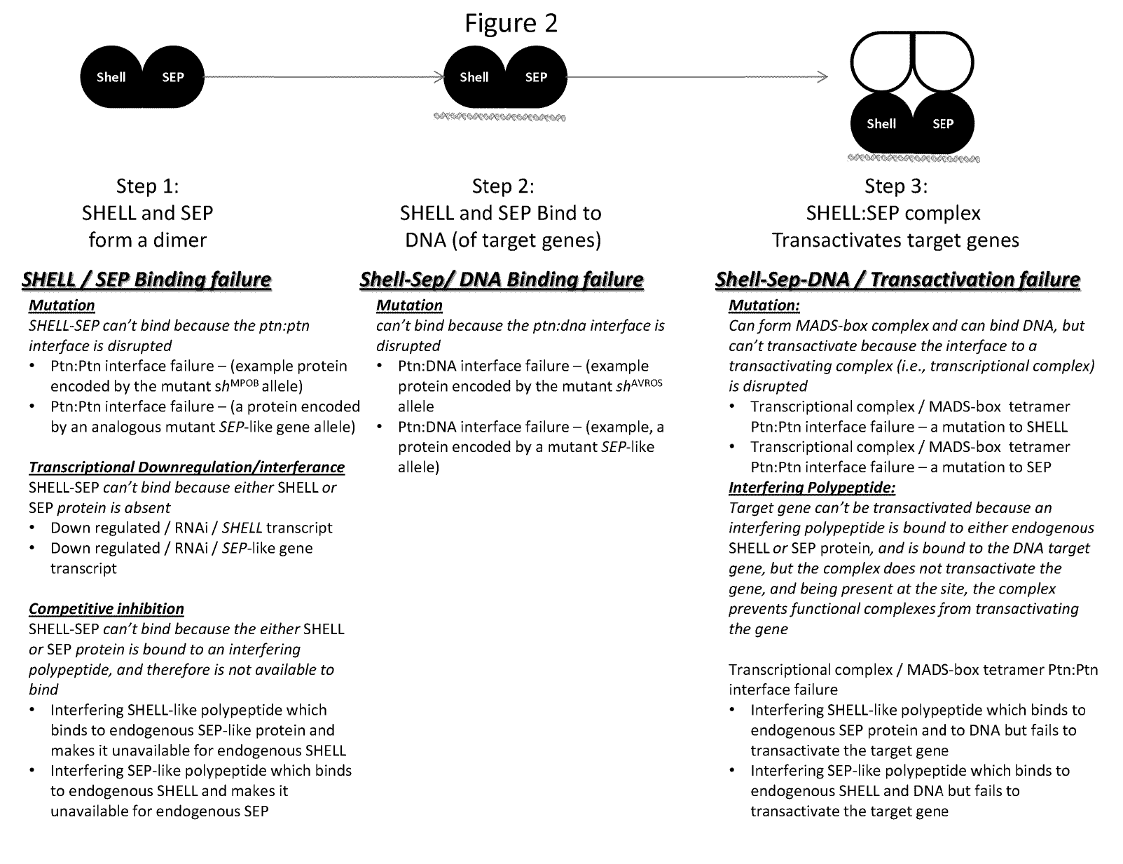

Auto-activation control tests were performed by transforming each BD fusion vector into yeast alone, and each vector showed no auto-activation of selection reporter genes. Co-transformations were performed for all 16 pairwise combinations of BD and AD vectors and scored for growth on SD-Leu-Trp, SD-Leu-Trp-His, SD-Leu-Trp-His-Ade and X-gal media plates. Positive interactions were scored as blue co-tranformants (on X-gal plate) that were able to grow on SD-Leu-Trp-His-Ade selection plates (FIGS. 1 and 2).

It was observed that in the yeast two-hybrid experiment, SHELL encoded by the allele associated with thick shelled dura palms (Sh^{DeliDura}) interacts with the SEP protein family member OsMADS24. It was also observed that in the yeast two-hybrid experiment, SHELL encoded by one allele associated with shell-less pisifera palms (sh^{MPOB}) does not interact with the SEP protein OsMADS24. This suggests that the sh^{MPOB} mutation disrupts the interaction of the protein encoded by the sh^{MPOB} allele with its endogenous oil palm SEP-like protein binding partner, and this disruption alters the normal function of SHELL in controlling shell thickness and subsequently the oil yield phenotype of the palm. Finally it was observed that in the yeast two-hybrid experiment, that the SHELL protein encoded by a second allele associated with shell-less pisifera palms (sh^{AVROS}) does interact with the SEP protein family member OsMADS24. It is important to note that the sh^{AVROS} mutation encodes for a residue change at a position within the MADS box domain that is highly conserved in plants, which has been shown to be involved in nuclear localization and DNA binding. This suggests that while the sh^{AVROS} mutation does allow for the successful interaction of the encoded SHELL protein with its endogenous oil palm SEP-like protein binding partner, the sh^{AVROS} mutation likely prevents the encoded protein from successful nuclear localization and/or DNA binding, and as a result, this disruption alters the shell thickness and subsequently the oil yield phenotype of the palm. Therefore, the yeast two hybrid results indicate that i) the successful binding of SHELL protein to an endogenous SEP-like protein, and ii) the successful binding of SHELL containing protein complexes to target DNA, are both required for the normal function of SHELL. Therefore, since an interaction with an endogenous SEP-like binding partner is required for normal SHELL function, then it is evident that the mutation, inactivation, interference or reduced expression of the SEP-like gene which encodes for the protein binding partner of SHELL can lead to a reduced shell thickness or enhanced oil yield phenotype.

### Example 2

### Identification of MADS-Box Proteins in Rice (0. Sativa) and Oil Palm (E. guineensis)

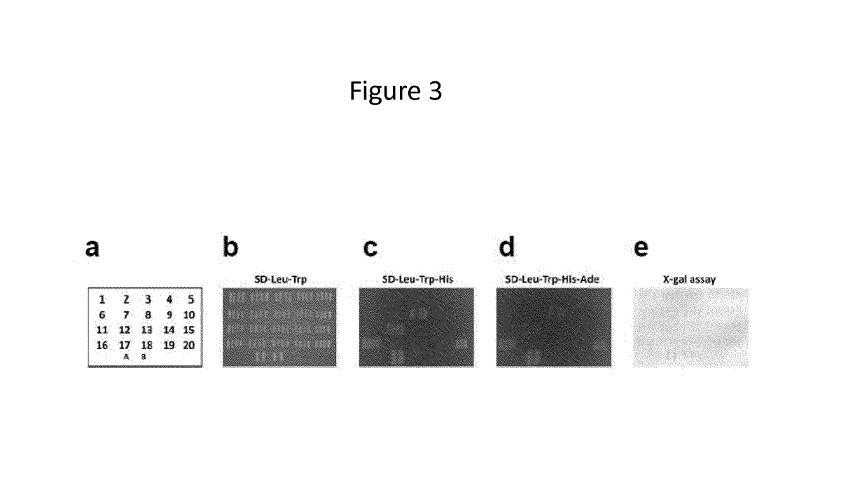

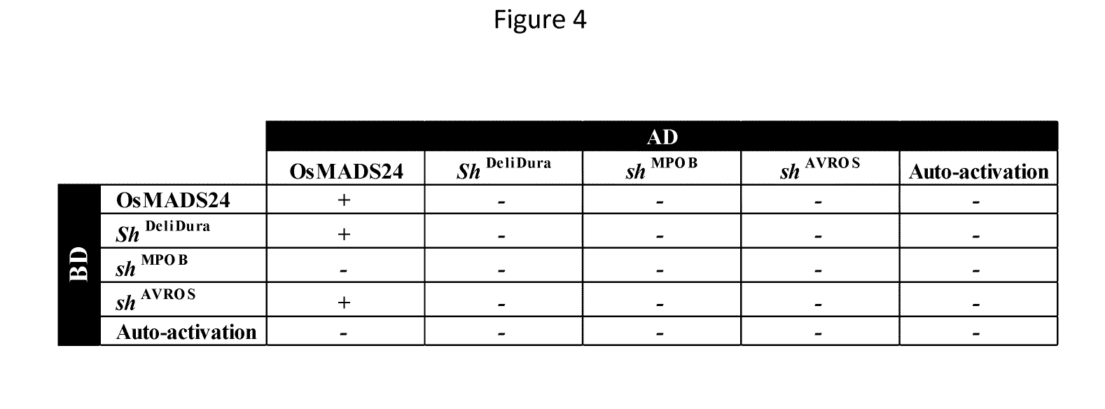

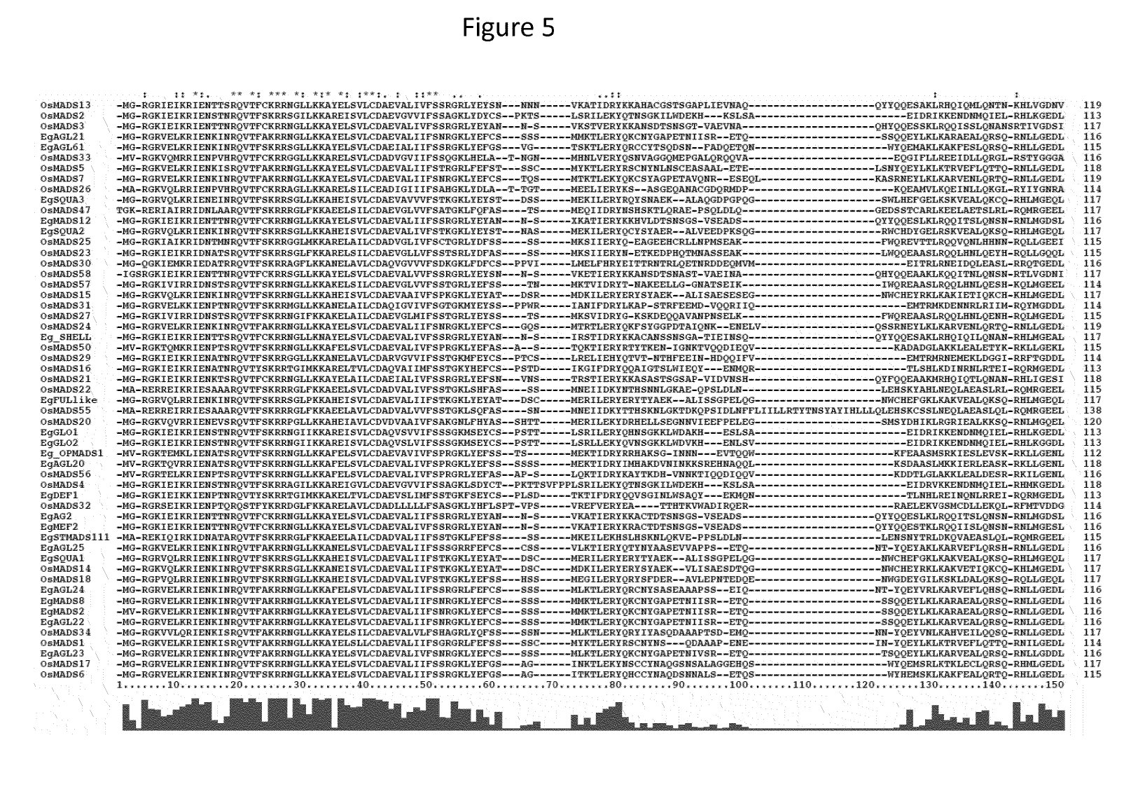

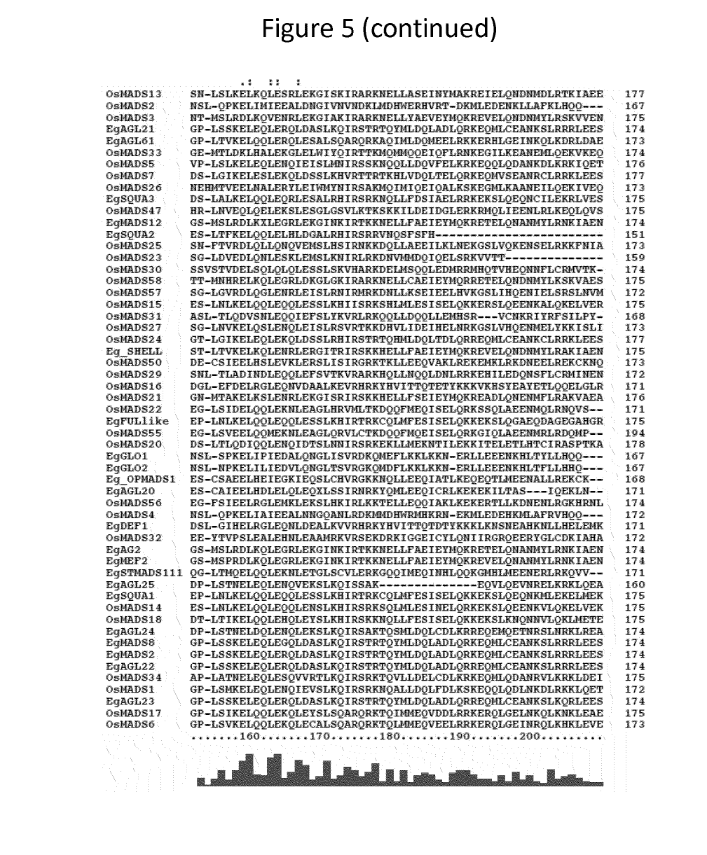

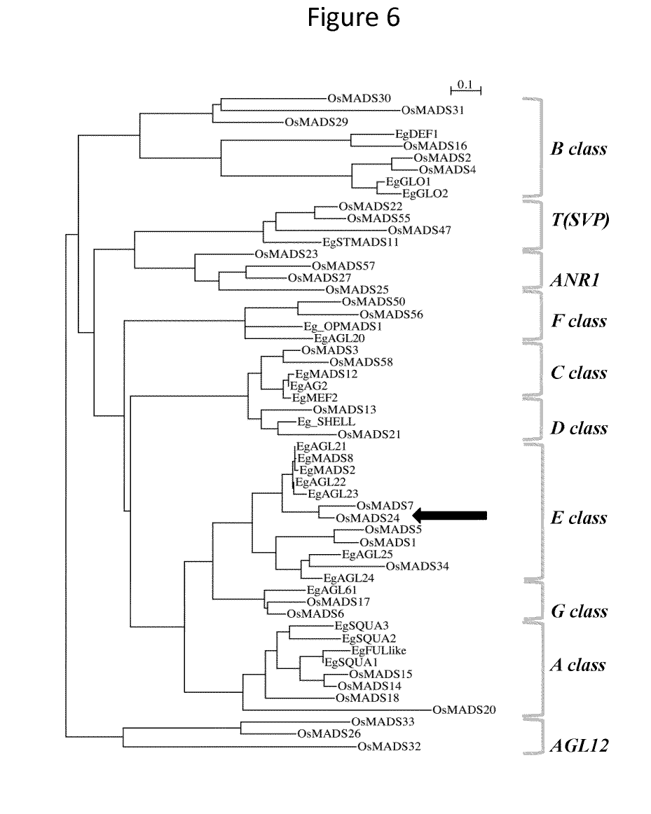

Sequences were recovered from GenBank and aligned using ClustalX (gap extension penalty=2.0). Conserved residues are highlighted (FIG. 3). A parsimony tree was constructed from the alignment using Phylip Promlk with default parameters. Clades were classified as A, B, C, D and E Class MADS-box proteins according to placement of the rice proteins according to Nam J et al., PNAS 2004 and Kramer et al., Genetics, 2004 (FIG. 4). Note that Zahn et al., Evol. Dev., 2006 place OsMADS13, the functional homologue to Shell, in the C (AG/SHP) rather than D (STK) lineages. Gene numbers are similar in Classes A-D, but the E (SEP) class genes have been duplicated in oil palm. The remaining rice genes are involved in transition to flowering and are included as an outgroup.

The identified MADS-box proteins provide candidate SHELL protein binding partners. Moreover, inactivation or downregulation of one or more of these genes are predicted to result in reduced shell thickness or enhanced oil yield.

### Example 3

### Identification of SEP-Like Proteins in Oil Palm (E. guineensis)

In order to identify the candidate set of SEP genes, a set of known SEP-like proteins was collected from the RefSeq database (NCBI), and a multiple sequence alignment was generated with ClustalX program (Clustal W and Clustal X version 2.0. Larkin M A et al, Bioinformatics, 23, 2947-2948. 2007). The resulting sequence alignment was next used as the input to the hmmbuild program (Accelerated profile HMM searches. S. R. Eddy. PLoS Comp. Biol., 7:e1002195, 2011.) to create a generalized Hidden Markov Model (HMM) (ibid) for the SEP-like protein family. The resulting HMM was used to search all predicted proteins from E. guineensis using the hmmsearch program, and a list of SEP-like genes was produced.

This provided a ranked listing of the 75 genes most similar to the SEP gene family (Table 1). Of these 75 genes, one encodes the SHELL protein (SEQ ID NO. 152) Accordingly, SEQ ID NOs: 1-74 were identified as encoded by SEP-like genes in oil palm.

TABLE 1
Score = Hmmersearch score; E-value = number of times
one would expect a similar match at random; Sequence =
the protein sequence (replace ‘P’ with ‘N’ for the DNA identifier)

| Rank | score | E-value | Sequence |
|---|---|---|---|
| 1 | 311.1 | 1.20E−92 | EG4P29517 |
| 2 | 283.2 | 3.90E−84 | EG4P81074 |
| 3 | 270.0 | 4.40E−80 | EG4P15412 |
| 4 | 252.8 | 7.80E−75 | EG4P37875 |
| 5 | 208.0 | 3.60E−61 | EG4P57231 |
| 6 | 196.6 | 1.10E−57 | EG4P67349 |
| 7 | 196.2 | 1.50E−57 | EG4P109263 |
| 8 | 158.6 | 4.40E−46 | EG4P29529 |
| 9 | 156.4 | 2.20E−45 | EG4P115489 |
| 10 | 151.6 | 6.20E−44 | EG4P6889 |
| 11 | 150.0 | 1.90E−43 | EG4P39137 |
| 12 | 149.3 | 3.20E−43 | EG4P44072 |
| 13 | 146.4 | 2.40E−42 | EG4P62915 |
| 14 | 144.4 | 1.00E−41 | EG4P64304 |
| 15 | 144.0 | 1.30E−41 | EG4P104954 |
| 16 | 144.0 | 1.30E−41 | EG4P82414 |
| 17 | 142.7 | 3.10E−41 | EG4P39130 |
| 18 | 142.1 | 5.00E−41 | EG4P44048 |
| 19 | 141.2 | 9.40E−41 | EG4P2672 |
| 20 | 140.0 | 2.10E−40 | EG4P15413 |
| 21 | 139.2 | 3.80E−40 | EG4P155269 |
| 22 | 138.2 | 7.40E−40 | EG4P11519 |
| 23 | 134.3 | 1.20E−38 | EG4P14715 |
| 24 | 131.0 | 1.20E−37 | EG4P82401 |
| 25 | 130.9 | 1.30E−37 | EG4P37080 |
| 26 | 129.9 | 2.60E−37 | EG4P63104 |
| 27 | 129.6 | 3.10E−37 | EG4P37079 |
| 28 | 125.5 | 5.60E−36 | EG4P29559 |
| 29 | 125.0 | 8.30E−36 | EG4P43162 |
| 30 | 120.6 | 1.90E−34 | EG4P31052 |
| 31 | 120.5 | 2.00E−34 | EG4P86343 |
| 32 | 118.5 | 8.00E−34 | EG4P39902 |
| 33 | 117.9 | 1.20E−33 | EG4P48307 |
| 34 | 114.9 | 9.80E−33 | EG4P23857 |
| 35 | 114.8 | 1.10E−32 | EG4P29533 |
| 36 | 113.7 | 2.30E−32 | EG4P70708 |
| 37 | 110.7 | 1.90E−31 | EG4P67350 |
| 38 | 110.4 | 2.40E−31 | EG4P44069 |
| 39 | 110.1 | 2.80E−31 | EG4P67198 |
| 40 | 105.5 | 7.30E−30 | EG4P130373 |
| 41 | 104.6 | 1.30E−29 | EG4P128041 |
| 42 | 104.0 | 2.10E−29 | EG4P147209 |
| 43 | 101.7 | 1.10E−28 | EG4P37712 |
| 44 | 100.6 | 2.30E−28 | EG4P153108 |
| 45 | 99.9 | 3.90E−28 | EG4P108259 |
| 46 | 89.0 | 8.30E−25 | EG4P71703 |
| 47 | 87.2 | 2.90E−24 | EG4P2959 |
| 48 | 86.3 | 5.50E−24 | EG4P82416 |
| 49 | 84.9 | 1.50E−23 | EG4P14105 |
| 50 | 78.0 | 1.80E−21 | EG4P37867 |
| 51 | 77.3 | 2.90E−21 | EG4P71708 |
| 52 | 73.6 | 4.10E−20 | EG4P37348 |
| 53 | 69.2 | 9.10E−19 | EG4P71707 |
| 54 | 67.9 | 2.20E−18 | EG4P104943 |
| 55 | 61.5 | 2.00E−16 | EG4P35645 |
| 56 | 61.5 | 2.00E−16 | EG4P37749 |
| 57 | 59.2 | 1.00E−15 | EG4P154153 |
| 58 | 59.2 | 1.00E−15 | EG4P45603 |
| 59 | 55.4 | 1.50E−14 | EG4P140076 |
| 60 | 53.2 | 6.80E−14 | EG4P41944 |
| 61 | 50.8 | 3.70E−13 | EG4P3001 |
| 62 | 46.0 | 1.10E−11 | EG4P60802 |
| 63 | 44.8 | 2.50E−11 | EG4P14015 |
| 64 | 43.7 | 5.70E−11 | EG4P21371 |
| 65 | 42.4 | 1.40E−10 | EG4P122402 |
| 66 | 37.3 | 5.00E−09 | EG4P42750 |
| 67 | 34.6 | 3.20E−08 | EG4P157194 |
| 68 | 33.4 | 7.40E−08 | EG4P6887 |
| 69 | 33.2 | 8.70E−08 | EG4P91665 |
| 70 | 32.7 | 1.30E−07 | EG4P126213 |
| 71 | 31.7 | 2.50E−07 | EG4P36286 |
| 72 | 27.0 | 7.20E−06 | EG4P3542 |
| 73 | 24.1 | 5.40E−05 | EG4P71936 |
| 74 | 22.0 | 0.00023 | EG4P29531 |
| 75 | 17.9 | 0.0041 | EG4P44436 |

### Example 4

### Altering the Shell Thickness and Oil Yield Phenotypes of a Plant, or Identifying Plants with Altered Shell Thickness or Oil Yield Phenotypes

The shell thickness and oil yield phenotypes of a plant, is altered by introducing a mutation in the SHELL gene such that the mutation disrupts the binding interface between the encoded SHELL protein and its SEP-like protein binding partner, thereby inhibiting dimer formation. The sh^{MPOB} allele is one example of such a mutation. It is observed that the protein encoded by sh^{MPOB} does not interact with OSMADS24, a rice SEP family member, in a yeast two hybrid screen, while the wild type SHELL protein encoded by the Sh^{DURA} allele does interact with OSMADS24 in the yeast two hybrid screen. Given that palms which are homozygous for the sh^{MPOB} allele are pisifera type and lack altogether a shell, while palms which are heterozygous for Sh^{DeliDura}/sh^{MPOB} are tenera type and have a shell with an intermediate thickness, it is evident that the protein encoded by the sh^{MPOB} allele likely modulates the shell thickness phenotype by disrupting the SHELL/SEP-like protein binding interface. It follows therefore that the introduction of an analogous mutation to the SEP-like gene, will likewise disrupt the binding interface between the encoded SEP-like protein and its SHELL protein binding partner, and will inhibit dimer formation thereby modulating the shell thickness and oil yield phenotypes of a plant.

It also follows that identifying naturally occurring mutations in a SEP-like gene, which are analogous to the sh^{MPOB} mutation in the SHELL gene, in a plant of seed, will enable the selection of plants or seeds with a disrupted binding interface between the encoded SEP-like protein and its SHELL protein binding partner, which will have inhibited dimer formation, thereby identifying plants with altered shell thickness and oil yield phenotypes. Other naturally occurring mutations can be identified which increase or reduce expression of a SEP-like gene, thereby identifying plants with altered shell thickness or oil yield phenotypes. Other naturally occurring mutations can be identified in a SEP-like gene that encode a protein that binds to SHELL but does not form a complex competent in transactivation of downstream targets, thereby identifying plants with altered shell thickness or oil yield phenotypes. A wide range of naturally occurring mutations that affect the expression or activity of a SEP-like gene or gene product can alter fruit shell thickness or oil yield. Once seeds or plants are identified as having analogous mutation in SEP-like genes, these plants can be selected for planting or for breeding trials, or for removal from the field.

The shell thickness and oil yield phenotypes of a plant, can also be altered by down regulating the expression of genes encoding for SHELL or SEP-like proteins such that the amount of functional SHELL or SEP-like protein in the cell is reduced. This reduction decreases the number of SHELL:SEP-like dimers in a cell, which ultimately can reduce target gene transactivation, thereby modulating the shell thickness phenotype of a plant. Reduced expression can be achieved by transforming plants with an expression cassette that reduces the expression of SHELL or its SEP-like binding partner, or an expression cassette that expresses an RNA that interferes with SHELL or SEP-like transcripts.

The shell thickness and oil yield phenotypes of a plant, can also be optimized by expressing a transgene encoding an interfering polypeptide, which can form a dimer with SHELL or alternatively with SEP-like proteins in the cell, but either fail to bind to the DNA of target genes altogether, or bind to target gene DNA but fail to transactivate these target genes. The expression of a gene encoding a Shell-like interfering polypeptide, provides an interfering polypeptide to bind with endogenous SEP-like proteins in the cell, forming dysfunctional dimers. This in turn can decrease the availability of endogenous SEP-like proteins which are able to form functional dimers with endogenous SHELL proteins, and in this way, expression of transgene encoding for an interfering polypeptide modulates the shell thickness and oil yield phenotypes of a plant. Alternatively, the expression of a gene encoding a SEP-like interfering polypeptide, provides an interfering polypeptide that binds with endogenous SHELL proteins in the cell, forming non-productive dimers. This in turn can decrease the availability of endogenous SHELL proteins which are able to form functional dimers with endogenous SEP-like proteins, and in this way, expression of a transgene encoding for the interfering polypeptide modulates the shell thickness and oil yield phenotypes of a plant.

The shell thickness and oil yield phenotypes of a plant, can also be optimized by introducing a mutation in the SHELL gene such that the mutation disrupts the binding interface in the encoded protein between SHELL:SEP-like protein dimers and DNA, thereby inhibiting DNA binding and target gene transactivation. The sh^{AVROS} allele is one example of such a mutation. It is observed that the protein encoded by the sh^{AVROS} allele does interact with OSMADS24, a rice SEP family member, in a yeast two hybrid screen. This is similar to the interaction of the protein encoded by the wild type Sh^{DeliDura} allele with OSMADS24. However, even though the protein encoded by the sh^{AVROS} allele can dimerize with a SEP-like protein, palms which are homozygous for the sh^{AVROS} allele are pisifera type and lack altogether a shell, while palms which are heterozygous for Sh^{DeliDura}/sh^{AVROS} alleles are tenera type and have an intermediate thickness shell. This suggests that the sh^{AVROS} encoded SHELL protein:SEP-like protein dimers are able to form, however they are dysfunctional as a complex and fail to transactivate target genes. The sh^{AVROS} mutation encodes for a LYS to ASN amino acid change in an alpha helix of the MADS box gene which has been shown in other plant systems to be critical for nuclear localization and DNA binding. Therefore, the protein encoded by the sh^{AVROS} allele is able to form a dimer with SEP-like proteins, but the dysfunctional dimers are likely unable to bind DNA and transactivate target genes. It follows therefore that introducing a mutation in a SEP-like gene in a plant, which does not disrupt the dimer formation of SHELL with its encoded SEP-like protein, but does inhibit DNA binding also modulates the shell thickness and oil yield phenotypes of a palm. It also follows that identifying naturally occurring mutations in a SEP-like gene in a plant or seed, which are analogous to the sh^{AVROS} mutation in the SHELL gene, will enable the selection of plants or seeds, which are able to form dimers between SHELL and its variant SEP-like protein, but unable to bind DNA, thereby identifying plants or seeds with altered shell thickness and oil yield phenotypes. Once seeds or plants are identified as having analogous mutation in SEP-like genes in this way, these plants or seed can be selected for planting or for breeding trials, or for destruction or removal from the field.

The shell thickness and oil yield phenotypes of a plant, can also be optimized by introducing a mutation in SHELL or a SEP-like gene such that the resulting encoding proteins in a SHELL:SEP-like protein complex is able to bind DNA but is incapable of transactivating target genes. To the extent that the dysfunctional mutant SHELL:SEP-like protein complex, or alternatively the dysfunctional SHELL:mutant SEP-like protein complex occupies the DNA binding site of the target gene, this bound dysfunctional complex will block functional complexes from binding to the site and prevent target gene transactivation. In this way, the expression of a gene encoding such a SHELL or SEP-like gene mutation will modulate the shell thickness and oil yield phenotypes of a palm.

The shell thickness and oil yield phenotypes of a plant, can also be optimized by expressing a gene encoding an interfering polypeptide which can bind to either SHELL or SEP-like gene products and form a complex that is able to bind target DNA but unable to transactivate target genes. To the extent that the dysfunctional interfering polypeptide:SHELL protein complex, or alternatively the dysfunctional interfering polypeptide:SEP-like protein complex, occupies the DNA binding site of the target gene, this bound dysfunctional complex will block functional complexes from binding to the site and successfully prevent target gene transactivation. In this way, the expression of a gene encoding such interfering polypeptides will modulate the shell thickness phenotype of a plant.

The term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety.

1. A method for sorting palm seeds by predicted shell thickness, the method comprising
obtaining a sample from a plurality of oil palm seeds or plants, thereby providing a plurality of samples;
detecting expression or genotype of a SEP-like gene in the samples; and
sorting the plurality of seeds, germinated seeds or plants based on the seed's or plant's predicted shell thickness, wherein the thickness of the shell is correlated to an expression level or mutation in the SEP-like gene.

2. A method for detecting a palm plant or seed with a reduced fruit shell thickness as compared to a plant or seed with a dura fruit form, the method comprising,
providing a sample from the plant; and
screening the sample for a mutation in a SEP-like gene, wherein the mutation in the SEP-like gene indicates that the plant or seed has a reduced fruit shell thickness as compared to a plant or seed with a dura fruit form.

3. The method of claim 2, wherein the SEP-like gene is at least 80% identical to a polynucleotide selected from the group consisting of SEQ ID NOs: 78-151.

4. The method of claim 2, the method further comprising determining a SHELL genotype of the plant or seed.

5. The method of claim 2, wherein the plant or seed is the product of a cross that included a parent with a wild-type SHELL genotype.

6. The method of claim 2, wherein the plant or seed is the product of a cross that included a parent with a wild-type SHELL allele.

7. The method of claim 2, wherein the plant or seed is heterozygous for a wild-type SHELL allele.

8. The method of claim 2, wherein the plant or seed is homozygous for a wild-type SHELL allele.

9. The method of claim 1, wherein the plant or seed is heterozygous for a wild-type SHELL allele.

10. The method of claim 2, wherein the plant or seed is homozygous for a mutant SHELL allele.

11. The method of claim 2, wherein the plant or seed is heterozygous for one mutant SHELL allele and heterozygous for another mutant SHELL allele.

12. The method of claim 2, wherein the plant is less than 5 years old.

13. The method of claim 2, wherein the plant is less than 1 year old.

14. The method of claim 2, further comprising:
providing a plurality of samples, each from a plurality of plants or seeds; and
screening for a mutation in a SEP-like gene in each of the plurality of samples.

15. The method of claim 2, wherein the SEP-like gene is 80% identical to a polynucleotide selected from the group consisting of SEQ ID NOs: 78-151.

16. The method of claim 2, further comprising selecting the plant for cultivation, breeding or destruction if the plant is heterozygous or homozygous for the mutation in the SEP-like gene.

17. The method of claim 2, further comprising selecting the plant or seed for cultivation, breeding or destruction if the plant is homozygous for the mutation in the SEP-like gene.

18. The method of claim 16, further comprising
selecting the plant for cultivation, breeding, or destruction if the plant is homozygous for the wild-type SHELL allele; or
selecting the plant for cultivation, breeding, or destruction if the plant is heterozygous for the wild-type SHELL allele.

19. A method for detecting a palm plant or seed with a reduced fruit shell thickness as compared to a plant with a dura fruit form, the method comprising,
providing a sample from the plant or seed; and
screening the sample for an increase or decrease in expression of a SEP-like gene as compared to a wild-type plant, wherein the increase or decrease in expression of the SEP-like gene indicates that the plant or seed has a reduced fruit shell thickness phenotype as compared to a plant or seed with a dura fruit form.

20. The method of claim 19, wherein the SEP-like gene is at least 80% identical to a polynucleotide selected from the group consisting of SEQ ID NOs: 78-151.

21. The method of claim 19, the method further comprising determining a SHELL genotype of the plant or seed.

22. The method of claim 19, wherein the plant or seed is heterozygous for a wild-type SHELL allele.

23. The method of claim 19, wherein the plant or seed is homozygous for a wild-type SHELL allele.

24. The method of claim 19, wherein the plant is less than 5 years old.

25. The method of claim 19, wherein the plant is less than 1 year old.

26. The method of claim 19, further comprising:
providing a plurality of samples, each from a plurality of plants or seeds; and
screening for an increase or decrease in expression of a SEP-like gene as compared to a wild-type plant in each of the plurality of samples.

27. The method of claim 26, wherein the SEP-like gene is at least 80% identical to a polynucleotide gene selected from the group consisting of SEQ ID NOs: 78-151.

28. The method of claim 19, further comprising selecting the plant or seed corresponding to the sample with increased expression of a SEP-like gene as compared to a wild-type plant for cultivation, breeding, or destruction.

29. The method of claim 19, further comprising selecting the plant or seed corresponding to the sample with decreased expression of a SEP-like gene as compared to a wild-type plant for cultivation, breeding, or destruction.

30. The method of claim 19, further comprising
selecting the plant or seed for cultivation, breeding, or destruction if the plant or seed is homozygous for the wild-type SHELL allele;
selecting the plant or seed for cultivation, breeding, or destruction if the plant is heterozygous for the wild-type SHELL allele;
selecting the plant or seed for cultivation, breeding, or destruction if the plant or seed is homozygous for the mutant SHELL allele; or
selecting the plant or seed for cultivation, breeding, or destruction if the plant or seed is heterozygous for one mutant SHELL allele and heterozygous for another mutant SHELL allele.

31-76. (canceled)


SEQUENCE LISTING (211 sequences)

SEQ ID NO: 1 - protein, 252 aa, Elaeis guineensis
  oil palm SEPALLATA (SEP)-like EG4P29517
MGRGRVELKRIENKINRQVTFAKRRNGLLKKAYELSVLCDAEVALIIFSNRGKLYEFCSSSRVKLDDKSAKEGNAKETHMVTITQIMMKTLERYQKCNYGAPETNIISRETQSSQQEYLKLKARAEALQRSQRNLLGEDLGPLSSKELEQLERQLDASLKQIRSTRTQYMLDQLADLQRKLEESNQAGQQQVWDPTAHAVGYGRQPPQPQSDGFYQQIDSEPTLQIGYPPEQITIAAAPGPSVNTYMPGWLA

SEQ ID NO: 2 - protein, 292 aa, Elaeis guineensis
  oil palm SEPALLATA (SEP)-like EG4P81074
MGRGRVELKRIENKINRQVTFAKRRNGLLKKAFELSVLCDAEVALIIFSSRGRLFEFCSSSRTNAGTITKKKGKLVTVQIFTREYLKNKWVPDFELEPYSTHLKLILQPFSQELFIMLKTLERYQRCNYSASEAAAPSSEIQNTYQEYVRLKARVEFLQHSQRNLLGEDLDPLSTNELDQLENQLEKSLKQIRSAKTQSMLDQLCDLKRRLREAASQNPLQLTWANGSGDHAAGSSNGPCNREAALSRGFFQPLACHPPEQIGTRAVLAKLKSTFINSLHFQLIEHWLKVFT

SEQ ID NO: 3 - protein, 283 aa, Elaeis guineensis
  oil palm SEPALLATA (SEP)-like EG4P15412
MGRGKVELKRIENKINRQVTFAKRRNGLLKKANELSVLCDAEVALIIFSSSGRRFEFCSCSSVLKTIERYQTYNYAASEVVAPPSETQQNTYQEYAKLKARVEFLQRSHRNLLGEDLDPLSTNELEQLENQVEKSLKQISSAKDSKWPYLKVSQITILPNFTLEGDQSCCHLTHLMLDQLYDLKRKLQEAIPYNPLQWSWINGGGNGAGGASDGPCNHESALSEEFFQPLACHPLQVGNSCDLVMGFKQNKDKFMQIFLATPRTHFPLYLEETTRCWVIDRAG

SEQ ID NO: 4 - protein, 186 aa, Elaeis guineensis
  oil palm SEPALLATA (SEP)-like EG4P57231
MGRGKIEIKRIENSTNRQVTFSKRRNGIIKKAREISVLCDAQVSVVIFSSSGKMSEYCSPSTTLSRILERYQHNSGKKLWDAKHESLSAEIDRIKKENDNMQIELRHLKGEDLNSLSPKELIPIEDALQNGLISVRDKQHQQELAMDANVRELELGYPSKDRDFASHMPLAFHNSVMERFTLRRET

SEQ ID NO: 5 - protein, 106 aa, Elaeis guineensis
  oil palm SEPALLATA (SEP)-like EG4P67349
MGRGRVELKRIENKINRQVTFAKRRNGLLKKAYELSVLCDAEVALIVFSNRGKLYEFCSSSSMLKTLERYQKCNYGAPETNIVSRETQEDRRPYLIYEMKENKSWT

SEQ ID NO: 6 - protein, 179 aa, Elaeis guineensis
  oil palm SEPALLATA (SEP)-like EG4P109263
MGRGKIEIKRIENSTNRQVTFSKRRNGIIKKAREISVLCDAQVSLVIFSSSGKMSEYCSPSTTLSRLLEKYQVNSGKKLWDVKHENLSVEIDRIKKENDNMQIELRHLKGGDLNSLNPKELILIEDVLQNGLTSVRGKQHHQELAMNGNVRELELGDPLKARDFACQIPIAFREWEEVA

SEQ ID NO: 7 - protein, 69 aa, Elaeis guineensis
  oil palm SEPALLATA (SEP)-like EG4P29529
MGRGRVELKRIENKINRQVTFAKRRNGLLKKAYELSVLCDAEVALIIFSNRGKLYEFCSSSRRNIELNV

SEQ ID NO: 8 - protein, 133 aa, Elaeis guineensis
  oil palm SEPALLATA (SEP)-like EG4P115489
MGRGKIEIKKIENPTNRQVTYSKRRTGIMKKAKELTVLCDAEVSLIMFSSTGKFSEYCSPLSEQRMGEDLDSLGIHELRGLEQNLDEALKVVRHRKILYPEGPLDLADIEYPFMEKEIHDTVRKVVMLGDEKI

SEQ ID NO: 9 - protein, 112 aa, Elaeis guineensis
  oil palm SEPALLATA (SEP)-like EG4P6889
MGRGKIEIKRIENTTNRQVTFCKRRNGLLKKAYELSVLCDAEVALIVFSSRGRLYEYANNRLLASTNLWREPFTRSPHVKATIERYKRACTDTSNSGSVSEADSQLNSSFLE

SEQ ID NO: 10 - protein, 66 aa, Elaeis guineensis
  oil palm SEPALLATA (SEP)-like EG4P39137
MGRGKVELKRIENKINRQVTFSKRRNGLLKKAYELSVLCDAEVALIIFSSRGKLYEFGSVGGSLVS

SEQ ID NO: 11 - protein, 117 aa, Elaeis guineensis
  oil palm SEPALLATA (SEP)-like EG4P44072
MGRGRVELKRIENKINRQVTFSKRRNGLVKKANELSVLCDAEVALIIFSNRGRITEFCSSSSGGTSQKLITSKAWKALELTTPYSIHEILSVVAIYPHLKSHTNLQQPEHSEFDDGS

SEQ ID NO: 12 - protein, 78 aa, Elaeis guineensis
  oil palm SEPALLATA (SEP)-like EG4P62915
MGRGKVELKRIENKINRQVTFSKRRNGLLKKAYELSILCDAEVALIIFSGRGKLYEFGSVGHLGNRIGVGRTPFRLSD

SEQ ID NO: 13 - protein, 83 aa, Elaeis guineensis
  oil palm SEPALLATA (SEP)-like EG4P64304
MGRGKIEIKRIENTTNRQVTFCKRRNGLLKKAYELSVLCDAEIALIIFSGRGRLYEYSNNRSVFIDLHPKDEGCFSQILYREL

SEQ ID NO: 14 - protein, 78 aa, Elaeis guineensis
  oil palm SEPALLATA (SEP)-like EG4P104954
MKKIVKSKEIMGRGKIEIKRIENTTNRQVTFCKRRNGLLKKAYELSVLCDAEIALIVFSSRGRLYEYSNNRCVYVDVR

SEQ ID NO: 15 - protein, 172 aa, Elaeis guineensis
  oil palm SEPALLATA (SEP)-like EG4P82414
MGRGRVELKRIENKINRQVTFSKRRSGLLKKAYELSVLCDAEIALIIFSSRGKLYEFGSVGSRANYNPAKETVTNVAINPLPPPPIKGEPIYTRDESQPFGKHTARKPILSRAFYLDLVPNIENKTSISRLEILLPYSKACPQRKSERSVKLIMDRIISNMIRFLLSDIPLS

SEQ ID NO: 16 - protein, 219 aa, Elaeis guineensis
  oil palm SEPALLATA (SEP)-like EG4P39130
MVRGKTEMKLIENATSRQVTFSKRRNGLLKKAFELSVLCDAEVAVIVFSPRGKLYEFSSTSLSMPDTQQKSGSSQEPCSELLEDEELEGVDNVCDGVVGSGWTYDPYAKGNPLQKEEHAKKLFFSLRLGKRNPTWVRSAVVTWNQLLEEQIATLKEQEQTLMEENALLREKCKLQSQLRPAAAPEETVPCSQDGENMEVETELYIGWPGRGRTNCRSQG

SEQ ID NO: 17 - protein, 133 aa, Elaeis guineensis
  oil palm SEPALLATA (SEP)-like EG4P44048
MGRGRVQLKRIENKINRQVTFSKRRSGLLKKAHEISVLCDAEVALIVFSTKGKLYEYSTNARLRSVFGGAGGGQPKSKLENGIFLQRTSKVSLWGYPPLLGQSRISAMLILGRGAFFAHGCLSLLESSLDRNK

SEQ ID NO: 18 - protein, 135 aa, Elaeis guineensis
  oil palm SEPALLATA (SEP)-like EG4P2672
MGRGRVQLKRIENEINRQVTFSKRRSGLLKKAHEISVLCDAEVAVVVFSTKGKLYEYSTDSRMDQGGLGGLASVRGGGLAGCPAVTVDDGEARDGWRQVKANERKAFNSQGKPKNKKWSAPSWRWHPNLDAPLWH

SEQ ID NO: 19 - protein, 250 aa, Elaeis guineensis
  oil palm SEPALLATA (SEP)-like EG4P15413
MGRGRVQLRRIENKINRQVTFSKRRSGLLKKAHEISVLCDAEVALIIFSTKGKLYEYATDSWLQAATTAWKTHWDLTISCWLADRQCNWHEATVGRRRGDPAARGRPSRWPVAATDAHTFKKARIPFSKKSDDSGRRRSCTRARGERRRREEGEEAHLRRRRGFSGEQKKDGTGTVSAVVFQRLPPTESRIFGERERGGFSLNRAGGGALSDSDWEPLLSSRTIELGRPDLHGSLVAITGISAELCDCNR

SEQ ID NO: 20 - protein, 141 aa, Elaeis guineensis
  oil palm SEPALLATA (SEP)-like EG4P155269
MEGIGELRGLIEKRTPAIWSKGRGHAAFPLSLPPLGIHGNGVPLKVRRKLEEKRVRISIWKWISGELEVIPPLLKSKEIMGRGKIEIKRIENTTNRQVTFCKRRNGLLKKAYELSVLCDAEIALIIFSGRGRLYEYSNNRN

SEQ ID NO: 21 - protein, 227 aa, Elaeis guineensis
  oil palm SEPALLATA (SEP)-like EG4P11519
MARGKVQMRRIENPVQRQVTFCKRRAGLLKKARELSVLCGADIGIIIFSTHGKLYELATNGDMQSLIERYKSIGAEAQIEGGEVNQPQVSEQEISMLKQEINLLQKGIRKCNLPESNSESHYYGEEEIEDNNKPRRLRHATGEGDERGREKVSREATGVEGRPSSGSAALALSPVSTDLRATDLGGVVANAAACVLGEAGWTSRPEGEVVAGRTLVEGLRKRNASKA

SEQ ID NO: 22 - protein, 259 aa, Elaeis guineensis
  oil palm SEPALLATA (SEP)-like EG4P14715
MLMHLTLKDKCVGDELELEVGDGLTFGEVCVHKISYAALYTSPGVASLVLERGRCICFWCCEKRTMVRGRREIKRIENPIQRQSTFYKRRDGLFKKARELSILCDADLLLLLFSSSGKLYEYHTPSVPSAEELVKRYEVATQNKIWRDLHLERNAEMEKVQKLCELLERDLRFMKVDASQHYSLPVLDVLEGNLEAAINKVRSEKDRKIVGEINHLENMVRDRQQERYDLGDKVARAQGLKDMAVPLNRLDLKLGTCVS

SEQ ID NO: 23 - protein, 146 aa, Elaeis guineensis
  oil palm SEPALLATA (SEP)-like EG4P82401
MVRGKTEIKRIENATSRQVTFSKRRNGLLKKAFELSVLCDAEVALIVFSPRGKLYEFSSTRYTGYLGKINVKIMQDKNKTLRACLVFVNILITLMPGNALSLQCHALLTPSQYNQNLSSTNDEGLRFKSDSSFNKMGEWPDSVLVK

SEQ ID NO: 24 - protein, 137 aa, Elaeis guineensis
  oil palm SEPALLATA (SEP)-like EG4P37080
MVRGKTEVRRIENATSRQVTFSKRRNGLLKKAFELSVLCDAEVALIVFSPRGKLYEFSSSSRLIVMAVTTSLADHVDRISENLNDRIVDNISEALRLLAPKPLHDFLHMCVSPRLDRGVLRGVSSCWRVEAVVNPMT

SEQ ID NO: 25 - protein, 243 aa, Elaeis guineensis
  oil palm SEPALLATA (SEP)-like EG4P63104
MRGPCEEHRAGRATRARLSLGRAPCAPAHWATCSQPSRMLPRAPAQAAYRKTQVRRIENATSRQVTFSKRRNGLLKKAFELSVLCDAEVALIVFSPRGKLYEFSSSRATVSFGSRKVWIIQATMDAEANDCGRASSTKMLSACNSCCVQAVGEWVYTAFNRGGSESKTREVSQDLGTESCAIEELHDLELQLEQSLSSIRNRKLNAEPRLQLCAPAVSDDYDSQNTDVETELVIGRPGTCKVK

SEQ ID NO: 26 - protein, 213 aa, Elaeis guineensis
  oil palm SEPALLATA (SEP)-like EG4P37079
MVRGKTEVRRIENATSRQVTFSKRRNGLLKKAFELSVLCDAEVALIVFSPKGKLYEFSSSSRDGVEDQYSGGERTYSSLVSFSKYMLRNCTEDPLGMMIKPKLYHLVTKSYAGTILLQYRIQKTVDRYLMHTKDVNINIRATEQNMQCKTEPPVQLITQASSNGDACQNMEVETELIIGRPGTCEAKQQDHVSLNKQWSQENGAFGMESRQNP

SEQ ID NO: 27 - protein, 172 aa, Elaeis guineensis
  oil palm SEPALLATA (SEP)-like EG4P29559
MVRGRVELRRIEDKTSRQVSFSKRRSGLLKKAHELAVLCDAEVGLIIFSAKGKLYDFASTSSVYRYNIIMDNRPELLEEKRIECYVALMHDLYIKIWCKIALSNVDYKLAAEFALLRCKPLTRPFNERHPTMSWKLLVEQRKAQTGYTPLNSTPHLYGGNWPGHSCTPLGSG

SEQ ID NO: 28 - protein, 135 aa, Elaeis guineensis
  oil palm SEPALLATA (SEP)-like EG4P43162
MGRGKIVIRRIENSTSRQVTFSKRRKGLLKKAKELAILCDAEVGFVIFSSTGRLYDFASSSEAELGHHKTKVYISATEWWQRIEFESDQIWVGSKNLQRPLHQYKDKTFFLRQHRGKTFGSSLLQWMEDADNLWG

SEQ ID NO: 29 - protein, 309 aa, Elaeis guineensis
  oil palm SEPALLATA (SEP)-like EG4P31052
MRLRLSSFTLHLPRPHPIIVYVASIVRVVFGFDGTKPSPLSDPDAPRATRPAPFAASPHRHPLSFSLTTPMNPSPCGFIATYTVPESQEGGTVQNGGTNFRRESVWCILGSMVREKIQIRKIDNATARQVTFSKRRRGLLKKAEELSILCDAEVALIVFSSTGKLYEYSSSSAPLPFAAPLPSPIVSPYRRPSHAGGLLVPAMLVASLCCGLPARQHQLPPLAVCPLFTWAGVGLPLDRPLPLPPLLSPIASIMKEIIEKHSMHSKNLQKPDQPPLDLNGEWLLHAIVTPKYLHQVLTSNDEYFSPDET

SEQ ID NO: 30 - protein, 64 aa, Elaeis guineensis
  oil palm SEPALLATA (SEP)-like EG4P86343
MVRGKVQMKRIENPVHRQVTFCKRRAGLLKKAKELSVLCDAEIGIIIFSTHGKLYELATKGSYN

SEQ ID NO: 31 - protein, 147 aa, Elaeis guineensis
  oil palm SEPALLATA (SEP)-like EG4P39902
MGRVKLQIKRIENNTNRQVTFSKRRNGLIKKAYELSVLCDIDIALIMFSPSGRLSHFSGRRRFFEPDPLSITSMDELESCEKFLMEALRRVAERKHGGSWVKLVQLPRGWYQNELPHLAVFTNDTKFLIPMLLKNTVICIVYRQKLL

SEQ ID NO: 32 - protein, 253 aa, Elaeis guineensis
  oil palm SEPALLATA (SEP)-like EG4P48307
MDKLEARSFRTRFIGYPKKIMRYYFYLPENHNRRSDLITFNLPWRRCASLMRRHGSGSHNTYLSCGQGMPLRAARVITRGSETITRTRKPNRPITTTPTCRVPRGEIRVPNGVWNPRWASPLPVHLPRSSRPPAHSNGLSLGFRRPTAAAMRRGKVQIRRIEDKASRQVTFSKRRGGLFKKARELAVLCDAEVGLIVFSPSGKPYEFCSSSRCVSILLLRLRSSDPSRSIDSLRDQPGSVRQTLRSSSFLRRW

SEQ ID NO: 33 - protein, 151 aa, Elaeis guineensis
  oil palm SEPALLATA (SEP)-like EG4P23857
MGRGKIEIKRIENPTNRQVTFSKRRGGLLKKANELAILCDVQASMRQYTGEDLSSMTMNDLNQLEQQLEYSVNKVRTRKLSEHQAAMEHQQAAMEHKVPDVPMLEPFGLFYQDEPSRNLLQLSPQLHAFRLQPAQPNLQEASLPGHSLQLW

SEQ ID NO: 34 - protein, 224 aa, Elaeis guineensis
  oil palm SEPALLATA (SEP)-like EG4P29533
MVTLLLAQSSQQEYLKLKARVEALQRSQRNLLGEDLGPLSSKELEQLERQLDASLKQIRSTRTQYMLDQLADLQRRLEESNQAGQQQVWDPTAHAVGYGRQPPQPQSDGFYQQIDGEPTLQISVEGEEDEGELVEEDMEKRASDVKEELEYTLVYVMRYPPEQITIAAAPGSSWAIISNKLDDEKEEEEGSFSDDDWRLTVVDSEWVISMRLVMGSFPCFVKED

SEQ ID NO: 35 - protein, 389 aa, Elaeis guineensis
  oil palm SEPALLATA (SEP)-like EG4P70708
MGEEHLSDGKTASPIQLSEESRRGMAREKIQIRKIDNATARQVTFSKRRRGLFKKAEELAILCDADVALIIFSSTGKLFEFSSSRVFMVIRVKLRTGLARWVLLQMITTLPKSGHSSVGIPLISFKAIVVEMARAGRRVLTDSENVMYEDGQSSESVTNASQLVVPPNYDDSSDTSLKLGSTDCGLTEVCVDYDLYVTTSCTLFEGYTAVRKQALSLFLYDRSTHAAQIDRKRRQQVRIQEWRRLSKLTGLLAGALNLFGAVSGPKYDGKFLHSKVKELLGDTKLHQTLTNIVIPAFDIKLLQPVIFSTFEDDTLEGDTASVDVSTSENLRKLVQVGQDLLKKPVSRVNLETGVSEACDVEGTNEDALIRFAKMLSNERKSRNAKMSAA

SEQ ID NO: 36 - protein, 135 aa, Elaeis guineensis
  oil palm SEPALLATA (SEP)-like EG4P67350
MDKFEIAIKTSQQEYLKLKARVEALQRSQRNLLGDDLGPLSSKELEQLERQLDASLKQIRSTRLEESNQATQQQVWDPNAPAVGYGRQPPQPQGDGFYQQIECDPTLHIGYPPEQITIAAAPGPSVSNYMPGWLA

SEQ ID NO: 37 - protein, 190 aa, Elaeis guineensis
  oil palm SEPALLATA (SEP)-like EG4P44069
MAEDRWRLAAGRRRAAQKWQRPAWVRRVRPSTCVRDAAQALAQACMRVQPRPTRARAGNLMLKTIERYQRCSYNATDAIVPPKETQDLGPLSVKELEQLENQIEISLKHIRSKKTQLMLDQLCDLERKEQMLQEANKALRRRLEEDTINSLQLSWQNGANVVGNAPCDGEPPQTEGFFQPLGCEPSLQIG

SEQ ID NO: 38 - protein, 64 aa, Elaeis guineensis
  oil palm SEPALLATA (SEP)-like EG4P67198
MSERGSREHWWWTEDVELKRIENKINRQVTFSKRCNGLLKKAYEVSILCDVEVALIIFSSRGKL

SEQ ID NO: 39 - protein, 226 aa, Elaeis guineensis
  oil palm SEPALLATA (SEP)-like EG4P130373
MVRKPSMGRQKIDIKRIESEEARQVCFSKRRAGLFKKANELSILCGAEIGVIVFSPAGKPFSFGHPSVDSIIDRFLFGSPSPTTLPSADPRMPVAREMMVVHEFNQQYTVLTALLETEKRKKAVLEEAVRVKQAGEAALWGANIEELSLGELESLHKSFERLRRDVAMRADQLVIEAAHTRSSSVAAAGSFVPPPPLGVNLGFGRGVEGSMALPPPTFFGYGRGPF

SEQ ID NO: 40 - protein, 301 aa, Elaeis guineensis
  oil palm SEPALLATA (SEP)-like EG4P128041
MDRGDVDLQKIDGKENLANPFTKALTIKEFDNHKKKEEEALRTTPTEDDDDMILLDEGVDIASSSKRDNSDHACNMVRKPSMGRQKIDIKRIESEEARQVCFSKRRAGLFKKANELSILCGAEIGVIVFSPAGKPFSFGHPSVDSIIDRFLSGSPSPMTLPSADPRMPAAREMMVVHEFNQQYTVLTALLETERRKKAVLEEAVRVKRAGEAALWGANIEELGLGELESLYNSFERLRRDVAMRADQLVIEAAHTRSSSVAAAGSTVPPPPPGVNLGFGRGVEGSMALPPPTSFGYGRGPF

SEQ ID NO: 41 - protein, 224 aa, Elaeis guineensis
  oil palm SEPALLATA (SEP)-like EG4P147209
MGRQKIEIKRIQNEEARQVCFSKRRTGLFKKASELSILCGAEIGVVVFSPAGKAFSFGHPSVDAVFDRFLTGNPHHGNSGGPAADSRRGAVVRELNRQYMELHGLVDAERKRREALEEAMKGEQGGRPYWWDNNVDSLALEDLEEYEKKLLELRNNVAKVADQLLHEAMARKQQQHHHHHHQQQQQQFPMVGAAVALPGPFAIKNEDAIHPSLGGGLGFGHGFF

SEQ ID NO: 42 - protein, 226 aa, Elaeis guineensis
  oil palm SEPALLATA (SEP)-like EG4P37712
MGRQKIEIKRIESEEARQVCFSKRRVGLFKKANELSILCGAEIGVIVFSPAGQPFSFGHPSVDSIIDRFLSGGPSPPTLASADRRMPAAREMMVVRELNRQYTELAALLETERRRKVVLEEAVRVKRAGEAALWGANVDELGLGELERLHKSLERLRRDVARCADQLVIEAAHARSSSIAAASRSTAPPPPPGIHLGFGRGLEGSMALILPPPPTPTAFGYGRGLF

SEQ ID NO: 43 - protein, 222 aa, Elaeis guineensis
  oil palm SEPALLATA (SEP)-like EG4P153108
MVKAEVELMGIVEDKTLERYQKCNYGAPETNIISRETQILELVEWIRYKWLDEDIDKNLLGEDLGPLSSKELEQLERQLDASLKQIRSTREQMLCEANKSLRRRLEESNQAGQQQVWDPTAHAVGYGRQPPQPQSDGFYQQIDGEPTLQISVEGEEDEGELVEEDMEKRASDVKEELEYTLVSSRTNNNRSSTRDTDESIEIKGLKLQKFDKDQGEGQHTAL

SEQ ID NO: 44 - protein, 219 aa, Elaeis guineensis
  oil palm SEPALLATA (SEP)-like EG4P108259
MGRQKIEIKRIESEEARQVCFSKRRAGLFKKAIELSILCGAEIGVIVFSPAGKPFSFGHPSVDSIIDRFISGSPSPTTIPSANPRMPAAREMMVVRELNRQYTDLAALLETERRKKVVLEEAVRVMRAGKAVSWEANIEELGLGELEGLQKSFERLRMDMAMRADQLVIEAAHAQSSSMAAASSAAPPPSGVSLGFGRELEGSMALPPPTFFGHGRGLF

SEQ ID NO: 45 - protein, 244 aa, Elaeis guineensis
  oil palm SEPALLATA (SEP)-like EG4P71703
MARRTSHGRRKIEIKRIEDEQTRQVTFSKRRGGLFKKASELSTLCGAQVGILVYSPGGRPYSFGQPGFVEVSDRFLPCVPTPIGSDPPPMPPPAYLSVSQPSKHYLEVVNVLEAARAKGAVLKERLAMVLEEEGRAYESENDDLTVEELGDLVARLEALKMRVFSRFSTILNQQQASSSSAALTVTPLNVINPYATNGPQAYPGGGFVLGNNGHGAGGFLGTGGHGTPSGFMGNDGNGPLGFIA

SEQ ID NO: 46 - protein, 217 aa, Elaeis guineensis
  oil palm SEPALLATA (SEP)-like EG4P2959
MVRKTSNGHRKIEIKRIENEQIRQVTFSKRRQGLFKKASELSTLCGAQVGILVYSPAGRPYSFGQPGFEVVSNQLIAHNSFMTSPNPIEGPQGNAIVQQLNCHCMEIMSLLDTAKTKGAVLKERLEITPKGKEKAFETELEGFGMDELERLVKSYNDLKLKADSRIYKIMSGGASSSGGPLPVNPKLARDRELLFQPNICLEIFSIIKDRSMQRGAE

SEQ ID NO: 47 - protein, 96 aa, Elaeis guineensis
  oil palm SEPALLATA (SEP)-like EG4P82416
MAKLKAKFESLQRSQRHLLGEDLGPLSVKELQQLERQLESALSQARQRKAQIMLDQMEELRKKVSMLDEGQGSEHLEARFPCSIEEIAIVGFSRVV

SEQ ID NO: 48 - protein, 361 aa, Elaeis guineensis
  oil palm SEPALLATA (SEP)-like EG4P14105
MGRVKLKIKKLENSSGRQVTYSKRRAGILKKAKELSILCDIDLVLLMFSPTGKPTLCVGDRSTIEEVVAKFAQLTPQERAKSYWTDPDKINNVDHIGAMEQSLQESLSRIQVHKENLGKQLMSLDCSGQVKALLGKQAEANDQLQEDSLHEFSQNACLRLQLGGQYPYQSYCQNLIGENAFKPDTENSLPESTIDYQVDHFEPPRPGYDASFQNWASTSGTCDVAIYDDQSYSRRSAFRHSIDPVAYRGSYDWCPSTCVPQCFPYPPTSAVPAPNHDRSFPKRRLINIHPVNLRDPLLKPHLFLGSLKNHVPKWRSQKDLARANPASGLPTRASRGTHTLTPPKREQIKSTHTCQRHNILL

SEQ ID NO: 49 - protein, 206 aa, Elaeis guineensis
  oil palm SEPALLATA (SEP)-like EG4P37867
MSKEIVGKKTPYPHEEALAGSQGQGVSKNSQQDCTLAKGTAISWKPWNAPPQSHHYSAIETARAQNSTATTSKLVKTSGRLSAEMARGKVQMRRIENPVHRQVTFCKRRAGLLKKAKELSVLTDADIGDISSKARDQHTTEVFEIVEQNGHFDVAPMMVQQNGHFGVSPMIVQQNEHFTAAPAMEDIPYPLTIQNDYSSFTSLDMG

SEQ ID NO: 50 - protein, 197 aa, Elaeis guineensis
  oil palm SEPALLATA (SEP)-like EG4P71708
MATMPKKTMGRQKVKLKRIENEDALYVTFSKRKSSLFQKAAELATLCGSEIALVVFSPAGRPYSLGLPTVDKVFHRVLSSGPAQMGSGHSVVSHSAKQCSEITKHLEQEKSRKAILVERLQKEAPPRWEDGLHGLGWDDLLILAKEVEELKSKVDSRVCEILLQGASSSTANADAWPVGSSEGSYGVGPRGPLDNNI

SEQ ID NO: 51 - protein, 253 aa, Elaeis guineensis
  oil palm SEPALLATA (SEP)-like EG4P37348
MPRKTRTTRGKQKIEIKRIEKEEARQICFSKRRSGVFTKASDLSTLCGPDVAVLAFSPRGKPFSFGSPAVNPVIDRFVLDISSSPGSGHHCGPPSNTVQQLSKLCLDLTNQLHACKAKSAVLEEKLSSPGYDILELDWFENVDDLELDKLGKLAEALKRVKVNADAHVDARLLHGRGALSSSTTPVMTANQVEGASSSNRVMAAASSKGVMAAGNVPVAFLTISMLAMFGNMIKKNHLDNVEVSPYWTRLDAK

SEQ ID NO: 52 - protein, 193 aa, Elaeis guineensis
  oil palm SEPALLATA (SEP)-like EG4P71707
MAERTFRGRQKIEIKKIEKKAARDVTFSKRRVGVFGKASELATLCGVDIGVVAFSPAGRPYTFGHPDANVVFNRFLGLVQPEGSSGSVGAMARHRAEMLRQLTLHCSQMMDRLAAEREKRAVLEERLRKVSEDPQERAWPEDLEGLGLERLARMVRGFEEQRAKARARLHQIRELGESSSGPSATVEFKKSVV

SEQ ID NO: 53 - protein, 291 aa, Elaeis guineensis
  oil palm SEPALLATA (SEP)-like EG4P104943
MNGENDAASRIIFSSLKERLVQSGVSYAKAVKKHPIPSPVVRKSTETVKDLMSSNSGNVHHHPRSRGHRVKLLSKGTCFRCGDRDHTRESCRNPIKCFLCKGYGHVQKSTASPFWKGVLSTHGLFQQLFSITIGNGKWVSCWTFIKSTIERYKKACANTSNSGSIVDVDSQQYYQQESAKLRHQIQILQNANRHLMGDSLGSLTVKELKQLENRLERGITRIRSKKIAETERAQQVSIIEAGHEFDALPGFDSRNYYHPHISQQKSMMALVNEKEQSQNQSQLLQELGQSE

SEQ ID NO: 54 - protein, 272 aa, Elaeis guineensis
  oil palm SEPALLATA (SEP)-like EG4P35645
MGRSKVKLKFIEEQHRRSATYRRRIAGLKKKASELAILCDIPVLVISFGPREQVETWPEDNQAARHIIDRYRELSIDIRNKNKLDLPGYMKAEIIRHQASFNRRCRDLADMPLLPLDGLFYALLKSLRELAHQLDSRMEVIKERIQLLKDRKHFNLGETMNMGSQLLEITPRDGMMGIQNTASAYDMMFSDPYLTMNASLQDPPQPTSFSSGQISPDAFLQYLYGPMGMDEVPLAMVPSIPSNMDEVPLAMMPSIPMNMNEPPGAQLAKLCD

SEQ ID NO: 55 - protein, 239 aa, Elaeis guineensis
  oil palm SEPALLATA (SEP)-like EG4P37749
MARKKVNLAWIANDSTRRATFKKRRKGLMKKVSELATLCDVKACVIVYGPQEPQPEVWPSVPEVTRVLARFKSMPEMEQCKKMMNQEGFLRQRVAKQQEQLRKQERENRELETMLLMYQGLAGRSLHSLRIEDATSLAWMVEMKVKAVQERMGLVRAQMASSSQQVVLEAPIEAPAPMAVMKEKTPLEAAMEALQRQNWLMEVMNPNDNLMFGGGEEMVQPYMDHTNNPWLDPCYFPLN

SEQ ID NO: 56 - protein, 251 aa, Elaeis guineensis
  oil palm SEPALLATA (SEP)-like EG4P154153
MARNKVKLAWIANDATRRATLKKRRKGLLKKVQELSILCGVEACAIVYGPNDRVPEVWPSPPEAARIVGRFKSMPEMEQTRKMVNQEGFLRQRAVKLLEQLRKQERENREMEMKLLIREGLKGRSFDNLGIEDVTCLSWMLERKIKEIYDKMDEIKNKVTVNQVAGGPSALPLQVMAPPPAAPIGPVVPKEKTTVEQAMEALQRQNWFMDMMSPWPEDFYQPAQPMDPYQPPPPAPLDHTIPWPDPSFPFN

SEQ ID NO: 57 - protein, 251 aa, Elaeis guineensis; identical to SEQ ID NO: 56
  oil palm SEPALLATA (SEP)-like EG4P45603

SEQ ID NO: 58 - protein, 189 aa, Elaeis guineensis
  oil palm SEPALLATA (SEP)-like EG4P140076
MARRRRRWQFIENQRQRLATYRKRRGGLRKKASQLSSLCGVPIAVISFGPNGRLDTWPDDQGAIHDLLLTYRSFDPEKRRKHDLDLPTLLEAQEGSQNLLWDPRLDAMPTESLRNLTNSLDSKVKAIDERIQQLLEENSKCSNQDNNNSSREQGVNSKCNDQDNNNTGSEQRDDSKSSNQAKQIKRVRK

SEQ ID NO: 59 - protein, 229 aa, Elaeis guineensis
  oil palm SEPALLATA (SEP)-like EG4P41944
MGKIEKKEALHICFTKRRQGIFKKAGELAVLCGAQITVITLSPGGKPFSFGQPSTDAVIARYLDPGRHQVPIPITTSLEIRLRYYLKYCKLGEQSGGGLWWWEAPIDGLDLEELVVMKGAIEELYKAILKKANQPTSAGEAVQGMPQKPSLAMLNGLDSCDWLIQLLANCSQWLRDLKRVCGSLLSIFPNITIKAEVRGSVDRRLATHIIRDEDKQQVHRSTAIMRINV

SEQ ID NO: 60 - protein, 462 aa, Elaeis guineensis
  oil palm SEPALLATA (SEP)-like EG4P3001
MRRSQVKRILLKCPVKKAKEGEEPLEAVANKIWPNDDLEFQSGKSMIQKVKGMLRVRSMDTAIYSSKVMYLPKITLPYQKFTNTWCLGWFGPIIQQLPIGSAPGTLTFVTCRSESQTHPRTWLTTSPTWDTSMKSVIERYNKTKEENHLVMNASSETKPIRFRLASTAKSHNSDGADERGKDSNLMLVDAHERQELLTDLGRNQPHKHHFYRNREADHIQPQGGAAISYEVKDVFVQEDGIFWQREAASLRQQLHNLQESHRQLLGEELSGLSVKDLQNLENQLEMSLRGIRMKKVYAMRGVNGIDKGPITPYGFNVTEDANISIHLELSQPQLQTDATLAQGQGNKEVDQGHSHQPTNEDIMPSGFTIEYVLAIEQVVAGAPTAPFPRGQRGPTLDPRRANLGRRHVGVVGGGNLFAKRYDFLEENVGFRRVTIISLQKYGTSTESISRLRSNLFQNNKKS

SEQ ID NO: 61 - protein, 219 aa, Elaeis guineensis
  oil palm SEPALLATA (SEP)-like EG4P60802
MTNRGRGLQLIENRTQCLVTYRKRRESLKKKANQLSSLCGVLIAVISFDPDGRLHTWPDDQGALPDLLLTYRSLDPKKRQKHDLDLPTLLGAMPAGSLRTGPAKGHLCLRKLANSLHSKVEAIDERIQQLLDKNSKCTNQDNNSTSREQDDDSKCNKKGKNNNTSSEKGDDDSKGSNQGNNNNNTSSEQGDYSKSNNEGNDKNKVCLLVVTRWSFIPSL

SEQ ID NO: 62 - protein, 231 aa, Elaeis guineensis
  oil palm SEPALLATA (SEP)-like EG4P14015
MSRSSMKLELIADDAARKTSLKKRKKGLLKKVQELSILCDVDACAIIYEPDDRHPELWPSSEEATRMLVRLRSMPEMEQKQKMMNQEEFLYQKMRKLVDQLHKQEFENKELEKKLKMYEALRTGDFSELDMEQAMNLSMMIEQMLKKIYEKMDAIKKHQAAMARVDGVVQEGGNAAGLNTPRENTPTEKDNEILQRQKQMLDMMIPRSSKTYQPSAGPTNPWPANSLFPFN

SEQ ID NO: 63 - protein, 245 aa, Elaeis guineensis
  oil palm SEPALLATA (SEP)-like EG4P21371
MTNPDDGEVGGGGGSERCVASEKVTGKKARRATFKKRKKGLMKKVSELSTLCDVKACLIVYGPNEPEAEVWPSVPDAMRVLTKLKKMPEMEQSKKMMNQEGFMRQRIMKLQEQLRKQDRENRELETILLMYQGLAGRSLHTVTIEDMTSLAWLIEMKVNKVQERIEHSKGEIASKMVEGMKEEKKKVEGPSNIKEKISLEVAMEELQRQEWFTEIMNPHDLMICGNEVVQPYIDHNNPWLDAYFP

SEQ ID NO: 64 - protein, 250 aa, Elaeis guineensis
  oil palm SEPALLATA (SEP)-like EG4P122402
MGRHKIPVKMIDKKDESNICFSKQKKGLFSKAKQIARAGSEVAIIVFSRVGNIFTFCHPSIESVASRFLSQQNIKHRSSNDDNFHGNADFVYPGSDAARGGLTGPSEEGETSNKGDNKLDGGNTIMQDKGFESDHEEEEVESKTSSKAEGSDVAGSSQEEHALMHDGEEHATGEKETSSDETLHSGRFWWNNRIDNRELHELLEFESALVELREKVRDQANQILVQKPVMGYYLDFSNYKFKFDEQASQD

SEQ ID NO: 65 - protein, 202 aa, Elaeis guineensis
  oil palm SEPALLATA (SEP)-like EG4P42750
MVPRAELWAVWAGIAYARLALTVDRLIIEGDSGTMVKWIQMRDTEDAAHPLLRDIAMLLRGATITAVTIRMENLSIRASSFSLTNGRSELSGLVCGGVPKIQSSIFTERVSSCISRVDSPFVPVCSNVPEKLMGEQLSGLNVKELQNLEIQLERSLHCVQKKKGYLLHNENIELYKKVNLIRQENMELRKKPRNILSRTDKA

SEQ ID NO: 66 - protein, 311 aa, Elaeis guineensis
  oil palm SEPALLATA (SEP)-like EG4P157194
MNGENDAASRIIFSSLKERLVQSGVSYAKAVKKHPIPSPVVRKSTETVKDLMSSNSGNVHHHPRSRGHRVKLLSKGTCFRCGDRDHTRESCRNPIKCFLCKGYGHVQKGFATLSTKIETGATSCPVSLVVLESKTSLPLSLCRFLRGPYWKVILGYIARDTSELSYDDCFERRERTFGWRGLFFGPSAITSLSSLWCRLPICNLRRPYLVLFSFRQNLNLVDKHLMGDSLGSLTVKELKQLENRLERGITRIRSKKIAETERAQQVSIIEAGHEFDALPGFDSRNYYHVSMLEAAPHYSHQQDQTALHLGI

SEQ ID NO: 67 - protein, 162 aa, Elaeis guineensis
  oil palm SEPALLATA (SEP)-like EG4P6887
MGLRNKPPNQRRYGISYERNFKGIPRNLMGESLGSMSPRDLKQLEGRLEKGINKIRTKKIAENERAQQQMNMLPQTTEYEVMAPYDSRNFLQVNLMQSNQHYSHQQQTTLQLGKKIVDRVASSTDRSDVGIIQDLPNQRGPEGRRPWSDGLQQHGRWFGSGD

SEQ ID NO: 68 - protein, 60 aa, Elaeis guineensis
  oil palm SEPALLATA (SEP)-like EG4P91665
MSIVDNSDMSMASCRLQLIESRRQRLATYRKRRESLKKKANQLSSLCGVPIAVISFGPNG

SEQ ID NO: 69 - protein, 224 aa, Elaeis guineensis
  oil palm SEPALLATA (SEP)-like EG4P126213
MEVLPIIDLHPTVILGSVLELPQREGKPQRRIEEAKKNWFFHPWMDDRRSRRALLFPLRDANDPTPAHDSDLSQQGLWQPPTATPSQPRSVTDIWLCKWIESDFRNSFGSWEELFFLKINFQPVFSRHLMGDALSSLSVKELKQLENRLERGITRIRSKKIAENEQAALQVSIAQEGPQFDALPAFDSRNYYHVNLLEAATHYSHQQDQTALHLGYEARSDHAA

SEQ ID NO: 70 - protein, 82 aa, Elaeis guineensis
  oil palm SEPALLATA (SEP)-like EG4P36286
MPRRKVVLEPHPTEQARMQCYLTRRNGIKKKVRELSILCDADIAHLSIPPAGEPSLFLGAHTSCGGLVVLAGSVYSTIALHP

SEQ ID NO: 71 - protein, 252 aa, Elaeis guineensis
  oil palm SEPALLATA (SEP)-like EG4P3542
MAPPLGSGAATSGGNGDGRGERYRWKSIEKRTWGLCKKAYELATLCDVDVALICYLPSVDTPTIWPPYRHKVEQVVHRYVDIPADKKLPKNQITLHIPNSTAGNTKDAGEAAAVADADRIRVPFPYDEDKLIAIVRYLDSKIVEVRRMIAARRMERRSEPALAVASGGDGDPGTADWDRGKRVARDCGPVWGRGRPDFSALAAAAAAAARGGGSGGAPNSSRSCLCCYCPHHGHWFTGFDGRNASRDGSDGI

SEQ ID NO: 72 - protein, 613 aa, Elaeis guineensis
  oil palm SEPALLATA (SEP)-like EG4P71936
MAPPRGDGRSDKSLRLSIKNRTKGLCKKAYELATLCDVELALVSYPSDGAEPTTWPPDRSKIEDAFHRYFETPAHKKLPKNQITLDNPNPGAVEKKDAAKAAASKAPKETDRLRIPFPDDEDKLIALRGILDSRLEAVRKMIAIRRAEERRDPRPSARDTEKELAVAVANAGGGDPTPSAGDPGKRLAQGQGGPLPAAAAVAAASAGREDPRPSVRDVEKMVAGDCGPVSGRGNPDCSAAPAAAGSGGGGAPNSWLQPSAHGGRSHWSYRLQTEPTFSPQKEAAGNGRYPPGTRESVAYPVIQPKLQWHSSSLAPPQRHLLREAASPITPPFTVTWHRRRFTHFLRRRNATYDTVHGKWKHHDIKVKDSKTLLFGEKQVTVFGIRNPEEIPWGETGAEYVVESTGVFTDKEKASAHLKGGAKKVIISAASKDVPMFVVGVNEHEYKSDIDIVSNASCTTNCLAVLAKVINDKFGIIEGLMSTVHSITATQKTVDGPSSKDWRGGRAASFNIIPSSTGAAKVGRSFGVLTTTYKDAAEDKADRCRNQTVRGEEEADVWDRTLTTAEETLNSSADRRRIGGRSVGAGNCTFGSDSASGRAASGGSGRRNIGDFTD

SEQ ID NO: 73 - protein, 72 aa, Elaeis guineensis
  oil palm SEPALLATA (SEP)-like EG4P29531
MEGVEKIEEIIARELNMMKTLERYQKCNYGAPETNIISRETQEDVDALYGQVCDIFLKYPNELAVEWSEGLD

SEQ ID NO: 74 - protein, 396 aa, Elaeis guineensis
  oil palm SEPALLATA (SEP)-like EG4P44436
MREAIGGSQPRAQGGERRSRDRGDGRRSRARGGRLGGQGGRRQAGARGRELEEVGGSQGLEEASRGLREAEGGRGLTVGGSESRETAWILGRRSDAHSRGLEEVRDGRMLTIGGSRRRRQRKEGVGKNKGGWQGTGLGLSSTAINKASYPSQEPEAWSKPMVGKKLNVEFIKHRKKRLATYRRRKEALKQAAYELSTLCGTPTAVIYFGPDGQPESWPEDEGAVRDIIGRHPGLGAKKRSTRPFDLRDLPPFDDTSEEFLREMLCSMESGMEAVKERIQLLKKDSRCNQGDFHGDTGGVQQQGCQCNNPAFMEECFDVPMVSKAAMDDGPGQGHGAFAPMELKQVEGVAADAFLPCSSNASMDFNDELAAFSMPLIFMPPPFTGATSEHDIACIWQ

SEQ ID NO: 75 - protein, 238 aa, Elaeis guineensis
  oil palm MADS-box type transcription factor SHELL DeliDura allele, ShDeliDura, Sh+, EG4P37875
MGRGKIEIKRIENTTSRQVTFCKRRNGLLKKAYELSVLCDAEVALIVFSSRGRLYEYANNSIRSTIDRYKKACANSSNSGATIEINSQQYYQQESAKLRHQIQILQNANRHLMGEALSTLTVKELKQLENRLERGITRIRSKKHELLFAEIEYMQKREVELQNDNMYLRAKIAENERAQQAGIVPAGPDFDALPTFDTRNYYHVNMLEAAQHYSHHQDQTTLHLGYEMKADPAAKNLL

SEQ ID NO: 76 - protein, 238 aa, Elaeis guineensis
  oil palm MADS-box type transcription factor SHELL MPOB allele, shMPOB, sh
MGRGKIEIKRIENTTSRQVTFCKRRNGLPKKAYELSVLCDAEVALIVFSSRGRLYEYANNSIRSTIDRYKKACANSSNSGATIEINSQQYYQQESAKLRHQIQILQNANRHLMGEALSTLTVKELKQLENRLERGITRIRSKKHELLFAEIEYMQKREVELQNDNMYLRAKIAENERAQQAGIVPAGPDFDALPTFDTRNYYHVNMLEAAQHYSHHQDQTTLHLGYEMKADPAAKNLL

SEQ ID NO: 77 - protein, 238 aa, Elaeis guineensis
  oil palm MADS-box type transcription factor SHELL AVROS allele, shAVROS, sh-
MGRGKIEIKRIENTTSRQVTFCKRRNGLLKNAYELSVLCDAEVALIVFSSRGRLYEYANNSIRSTIDRYKKACANSSNSGATIEINSQQYYQQESAKLRHQIQILQNANRHLMGEALSTLTVKELKQLENRLERGITRIRSKKHELLFAEIEYMQKREVELQNDNMYLRAKIAENERAQQAGIVPAGPDFDALPTFDTRNYYHVNMLEAAQHYSHHQDQTTLHLGYEMKADPAAKNLL

SEQ ID NO: 78 - DNA, 759 bp, Elaeis guineensis
  oil palm SEPALLATA (SEP)-like EG4P29517
atggggaggggaagggtggagctgaagagaatcgagaacaagatcaatcgccaggtgaccttcgcgaagcggcggaatgggctcctcaagaaggcctacgagctctccgtgctctgcgacgccgaggttgctctcatcatcttctccaaccgcgggaagctttacgagttctgcagcagctccagagttaagcttgatgataagagtgccaaagaaggtaatgcaaaagagacacatatggtcaccatcactcaaattatgatgaagacacttgaaaggtatcaaaaatgcaactatggtgctccggagactaatattatatcaagagagactcagagtagtcagcaggagtacttgaaactaaaagcacgtgctgaagccttacagagatcgcaaagaaatctcctcggtgaggacttgggcccactcagcagcaaggagcttgagcagcttgagcggcaacttgatgcatcgttaaagcaaatcagatcaacacggacccaatacatgcttgatcagcttgcagatcttcaacgaaagttggaggaaagtaaccaggctggtcagcagcaagtttgggatcccactgctcatgcagtaggctatggccggcagccacctcaaccacagagcgatggattctaccaacagatagatagtgaacctactctccaaatcgggtatcctccagaacaaataacaatcgcagcagcacccgggccaagtgtgaatacttatatgccaggatggcttgcataa

SEQ ID NO: 79 - DNA, 879 bp, Elaeis guineensis
  oil palm SEPALLATA (SEP)-like EG4N81074
atggggaggggaagggtggagctgaagaggatcgagaacaagataaacaggcaggtgacgttcgccaagcggcggaacgggttgctgaagaaggccttcgagctctccgtcctctgcgacgccgaggtcgccctcatcattttctccagccgcggccgcctcttcgaattctgcagcagctccaggaccaatgcgggaacaataactaaaaagaagggaaaacttgtaactgttcaaatctttactcgagaatatctgaaaaataagtgggtgcccgacttcgaactcgagccatatagtacacacctgaagctgattctccaacctttctctcaagaacttttcatcatgcttaagacactcgaaaggtaccaaagatgcaattatagtgcatcagaagctgctgctccgtcaagtgagatacagaacacttaccaagagtacgtgaggctgaaggcaagagttgagtttctgcagcactcacagagaaatctccttggtgaggacttggacccactaagtacaaatgaacttgatcaacttgagaatcaactagagaaatctttaaagcagatcagatcagcaaagacacaatcaatgctcgatcagctttgtgatcttaaaagaaggttgcgagaagcagcttcacaaaatcccctccaattgacatgggcaaatggtagtggtgatcatgctgctggttcatcaaatggcccttgtaatcgtgaggctgctctatcaaggggattcttccagccattggcatgtcaccctcctgagcaaattggaacacgggctgtactcgccaagctgaagtccactttcatcaacagcctccattttcagttaatagagcattggctcaaggtgttcacatga

SEQ ID NO: 80 - DNA, 852 bp, Elaeis guineensis
  oil palm SEPALLATA (SEP)-like EG4N15412
atggggagggggaaggtggagctgaaaaggattgagaacaagataaacaggcaggttacctttgcaaagcgacggaacggattgctgaagaaggctaacgagctctctgtcctctgcgacgccgaggtcgccctcatcatcttctccagcagcggccgccgcttcgagttctgcagctgctccagcgtgcttaagacaatcgagaggtaccaaacatacaactatgctgcatcagaagttgttgccccaccaagcgagacacagcagaacacttatcaggaatatgcgaagctgaaggcaagagttgagtttctgcaacgttcgcatagaaatctcctaggtgaggacttggacccattaagtacaaatgaacttgagcaacttgagaatcaagtagagaagtctttaaagcagatcagttcagcaaaggattccaaatggccatatctcaaggtgtctcagatcaccattcttcccaacttcaccttagagggtgaccaatcatgctgtcatcttacgcatttaatgcttgatcaactttatgatcttaagagaaagttacaagaagccattccatataatcccctccagtggtcatggataaatggtggtggcaatggtgctggtggtgcatccgatggcccttgtaatcacgagtctgctctatcagaggaattcttccagccattggcatgccaccctctacaagttggtaatagttgtgatctggttatgggattcaagcagaataaggataaatttatgcagatttttcttgcaacgcctcgtacacatttcccgctttacctggaggagactacgagatgttgggtgattgaccgggccgggtag

SEQ ID NO: 81 - DNA, 561 bp, Elaeis guineensis
  oil palm SEPALLATA (SEP)-like EG4N57231
atggggcgagggaagattgagattaagcggatcgagaactccaccaaccggcaagtgaccttctccaagcggcggaatgggatcatcaagaaggcacgggagatcagcgtcctctgcgatgcccaggtctccgtcgtcatcttctccagctccggcaagatgtccgagtactgcagcccctccaccacgctgtcgaggattctcgagaggtaccagcataactctggcaagaagctctgggatgccaagcacgagagtcttagtgctgagatcgaccggatcaagaaagagaatgacaacatgcagatcgagctgaggcatttgaagggtgaggatctgaactcactgagcccaaaggaactcattccaattgaagatgccctccagaatggtctcatcagtgttcgggacaagcagcaccagcaggaattggcaatggatgcaaatgtaagggaactggagcttggatatccttcgaaagatagggattttgcttcccacatgccactagccttccataactccgtaatggaaaggttcacactcaggcgggagacttag

SEQ ID NO: 82 - DNA, 321 bp, Elaeis guineensis
  oil palm SEPALLATA (SEP)-like EG4N67349
atggggagaggaagggtggagctgaagaggatcgagaacaagatcaatcgccaggtaaccttcgcgaagcggcggaacgggcttctcaagaaagcctacgagctctccgtgctctgcgacgccgaggtcgcccttatcgtcttctccaaccgcgggaagctctatgagttctgcagcagctccagtatgttgaagacactagaaaggtaccaaaaatgcaactatggtgcaccagagactaatattgtgtcaagggaaactcaggaggacagaagaccctacttaatctatgagatgaaggagaacaaatcatggacataa

SEQ ID NO: 83 - DNA, 540 bp, Elaeis guineensis
  oil palm SEPALLATA (SEP)-like EG4N109263
atggggcgagggaagattgagatcaagcggatcgagaactccaccaaccggcaggtaaccttctccaagcggcggaatgggatcatcaagaaggcccgggagataagcgtgctctgcgatgcccaggtctccctcgtcatcttctccagctccgggaagatgtccgagtactgcagcccctccaccacgttgtcgaggttgctggagaagtaccaggtgaactctggcaagaagctctgggatgtcaagcacgagaatctgagtgttgagattgaccgaatcaagaaggagaatgacaacatgcagattgagctgaggcatttgaagggtggcgatctgaactcgctgaacccaaaggaactcattctaattgaggatgtcctccagaatggtctcaccagtgttaggggcaagcagcatcaccaggaattggcaatgaatggaaatgtaagggaattggagcttggggatcctctgaaagctagggattttgcatgccagattccaatagccttccgtgagtgggaggaagttgcttag

SEQ ID NO: 84 - DNA, 210 bp, Elaeis guineensis
  oil palm SEPALLATA (SEP)-like EG4N29529
atggggaggggaagggtggagctgaagagaatcgagaacaagatcaatcgccaggtgactttcgcgaagcggcggaatgggctcctcaagaaggcctacgagctctccgtcctctgcgacgccgaggtcgctctcatcatcttctccaaccgcgggaagctttacgagttctgcagcagctccaggaggaacatcgaactaaatgtctag

SEQ ID NO: 85 - DNA, 402 bp, Elaeis guineensis
  oil palm SEPALLATA (SEP)-like EG4N115489
atggggagggggaagatagagatcaagaagatagagaatcctaccaacaggcaggtgacctactccaagaggaggacggggatcatgaagaaggctaaggagctgacggtgctttgcgatgctgaggtctcgcttatcatgttctccagcaccggcaagttctccgagtattgcagccccctttccgagcagcggatgggtgaagatctcgacagtttgggcatccatgaactgcgcggtcttgagcaaaatttagatgaggctttgaaggttgttcgtcacagaaaaattctttatccagaaggacctctggatcttgctgacattgagtatccatttatggagaaagaaatccatgatacagtgcggaaagtggtgatgcttggcgatgagaagatttga

SEQ ID NO: 86 - DNA, 339 bp, Elaeis guineensis
  oil palm SEPALLATA (SEP)-like EG4N6889
atgggtcgaggaaagatcgagatcaagaggatagagaacacgaccaaccggcaggtgaccttctgcaagcgccgcaacggcctgctcaaaaaggcctacgagttgtccgtgctctgcgacgcggaggtcgccctcatcgtcttctcgagccgcggccgcctctacgaatacgccaacaacaggttgctagcttctacgaatctttggagggaaccgttcacgagatctccccatgtgaaagctaccatcgagaggtataaaagagcatgcactgatacctccaactctggatctgtttctgaagctgattctcagcttaattcttcctttcttgagtga

SEQ ID NO: 87 - DNA, 201 bp, Elaeis guineensis
  oil palm SEPALLATA (SEP)-like EG4N39137
atggggaggggaaaagttgagctgaagaggatcgagaacaagatcaaccgccaggttaccttctccaagcgccgcaacggcctgctcaagaaggcctacgaactctccgtcctctgcgatgccgaggttgcactcatcatcttctccagccgcggcaagctctacgagttcggcagcgttgggggttctctagttagttag

SEQ ID NO: 88 - DNA, 354 bp, Elaeis guineensis
  oil palm SEPALLATA (SEP)-like EG4N44072
atggggagggggagggtggagctgaagaggatcgagaacaagataaaccggcaggtgacgttctccaagcggaggaacgggctggtgaagaaggcgaacgagctgtcggtgctctgcgatgcggaggtcgccctcatcatcttctccaaccgcggcaggatcaccgagttctgcagcagctccagcggaggaacttcccagaaattgataacttcaaaggcgtggaaggctttagagctgaccaccccctattccatacatgagatcctatcggtggtagcaatttatccccacctcaagagtcacaccaacctccaacagcctgagcatagcgagtttgacgacggcagctag

SEQ ID NO: 89 - DNA, 237 bp, Elaeis guineensis
  oil palm SEPALLATA (SEP)-like EG4N62915
atggggagggggaaagtggagctgaagaggattgagaacaagatcaaccgccaggtgaccttctccaagagaagaaatgggctcctaaagaaggcttatgagttgtcgattctttgcgatgccgaggtcgccctcatcatcttctccggtcgtggaaagctctatgagttcggcagcgtcggccacttgggcaatagaataggcgttggacgcactccattcaggctgtctgactga

SEQ ID NO: 90 - DNA, 252 bp, Elaeis guineensis
  oil palm SEPALLATA (SEP)-like EG4N64304
atggggagggggaagattgagatcaagagaattgagaacactacaaaccgccaagtgaccttctgcaagcggaggaatggtttgctgaagaaagcctatgaattatcggttctttgtgatgcagagatcgcgctcatcatcttctcaggccgtggccggctctatgagtactccaataacagatctgtctttatagatcttcatcccaaggatgaaggatgcttctcccaaatcctttatagagaactgtga

SEQ ID NO: 91 - DNA, 237 bp, Elaeis guineensis
  oil palm SEPALLATA (SEP)-like EG4N104954
atgaaaaagatagtgaagagtaaggagatcatggggaggggtaagattgagatcaagagaattgagaacactacaaatcgccaagtgaccttctgcaagcggaggaatggtttgctgaagaaagcctatgaactttcggttctttgtgatgcagagatcgccctcatcgtcttctcaagccgtggccgcctctacgagtactccaataacaggtgtgtttatgtggatgtgaggtga

SEQ ID NO: 92 - DNA, 519 bp, Elaeis guineensis
  oil palm SEPALLATA (SEP)-like EG4N82414
atggggagggggagagttgaactgaagaggatcgaaaacaagatcaaccgccaggtaaccttctccaagcgccgcagcggcctgctcaagaaggcctatgagctctccgtcctctgcgacgccgagattgcactcatcatcttctccagccgcggcaagctctacgagttcggcagcgttgggtccagagcaaattataatcctgccaaagaaacggttacaaacgtcgccatcaatccattacctcctccacctataaaaggagaacccatatacaccagagatgaatcccagccttttgggaagcacacagctcggaagcctatcttaagcagggcattctatttggatttggtccccaatatcgagaacaagacatcaatctctcgcttggaaattcttcttccttacagcaaagcatgtcctcaaagaaagtcagaaagatctgtgaagctcatcatggatcgaatcatatccaatatgattcgattccttctctcggatatcccattaagttga

SEQ ID NO: 93 - DNA, 660 bp, Elaeis guineensis
  oil palm SEPALLATA (SEP)-like EG4N39130
atggtgagggggaagacggagatgaagctgatagagaacgcgacgagcaggcaggtgacgttctcgaagcggaggaatgggcttctgaagaaggcgttcgagctctcggtcctttgcgacgccgaggtcgccgtcatcgtcttctctccccgtggaaagctctacgagttctccagcaccagcttgtcaatgccagatacacaacagaaaagtggatcttctcaggaaccttgttcagagctacttgaagatgaagaactggaaggagttgataatgtttgtgatggagtcgttggcagtggatggacatatgacccatatgccaaggggaatccacttcaaaaagaagagcatgcaaagaaattattcttttccttaagattaggcaagagaaatcctacatgggtgaggtcagctgtggtgacatggaatcagttacttgaagagcaaattgcaacgctcaaagaacaggagcagacacttatggaggagaatgcattactacgagagaagtgcaagctacaatctcaactacggccagccgctgctccagaggaaactgttccatgcagccaggacggtgagaatatggaggtagagacagagctgtacattggatggccaggaaggggaaggaccaattgcaggtcgcaaggttga

SEQ ID NO: 94 - DNA, 402 bp, Elaeis guineensis
  oil palm SEPALLATA (SEP)-like EG4N44048
atggggagaggtagggtgcagctgaagaggatcgagaacaagataaaccggcaggtgacgttctccaagcggcggtcggggctgttgaagaaggcgcacgagatctcggtgctctgcgacgcggaggtcgctctcatcgtcttctccaccaagggcaagctctacgagtactccaccaacgccaggttgaggtcagtgtttggcggagctggaggtggtcagccaaaatccaaactagagaatggcatcttccttcaaaggacttcaaaggtttccttatggggttatcccccacttctcggacaatcaaggatttctgctatgctcatcttgggacgaggggcattctttgctcatggttgtttgagtcttcttgaatcatctctcgatcggaacaagtaa

SEQ ID NO: 95 - DNA, 408 bp, Elaeis guineensis
  oil palm SEPALLATA (SEP)-like EG4N2672
atggggagagggagggtgcagctgaagaggatcgagaacgagataaacaggcaggtgacgttctcgaaacgccggtcggggctgctgaagaaggcgcacgagatctcggtgctctgtgacgccgaggtcgccgtcgtcgtcttctctaccaagggcaagctctacgagtactccaccgactccaggatggaccaagggggacttggtggcttggcttcggtgaggggcggcggcttggccggatgtccggcagtgacggtcgacgatggtgaggcaagggatggctggcggcaagtaaaagcaaatgagagaaaagctttcaatagtcaaggtaaaccaaagaataaaaagtggagcgccccttcgtggaggtggcatcctaacttggatgcccctctttggcactag

SEQ ID NO: 96 - DNA, 753 bp, Elaeis guineensis
  oil palm SEPALLATA (SEP)-like EG4N15413
atggggagagggagggtgcagctgaggcggatcgagaacaagataaaccggcaggtgacgttctcgaagcgccggtcggggctcctgaagaaagcccacgagatctccgtcctctgcgacgccgaggtcgccctcatcatcttctcgaccaagggcaagctctacgagtacgccaccgactcctggctccaagcagctacaactgcttggaaaacccattgggatctcacaatctcctgttggctggccgaccgacagtgcaactggcatgaggcgactgtcggcaggaggaggggtgacccagcggcaagaggaaggccaagccggtggccggtggcggccaccgacgcccacacattcaaaaaggcccgaatccctttctcaaagaaatccgacgactccggtcgccggcgatcgtgcacacgggcacggggagaaaggaggagaagagaggaaggggaggaggctcaccttcgacgtcggcgaggcttttccggcgagcaaaaaaaagatggcacagggacggtctccgcggtggttttccaacgattgccgccgactgagtctcgaatcttcggtgagagggagagaggaggattctccttaaatagagccggagggggggctctttccgactccgattgggagccgcttctatcatcaaggactattgagcttgggagacccgacctccatggctctttggtggccattacaggcatctccgctgagctatgtgattgcaatcgctga

SEQ ID NO: 97 - DNA, 426 bp, Elaeis guineensis
  oil palm SEPALLATA (SEP)-like EG4N155269
atggaagggataggagagcttcgggggctcattgaaaagagaacaccggccatctggtccaagggccgcggccatgcagcttttcctctctcacttcctcccctcggaatccacggaaatggagttcctctgaaagttagaaggaaactagaagaaaaaagggtgagaatctcgatttggaagtggatttccggggagttggaggtcattcctccacttctaaagagcaaggagatcatggggagggggaagattgagatcaagagaattgagaacactacaaaccgccaagtgaccttctgcaagcggaggaatggtttgctgaagaaagcctatgaattatcggttctttgtgatgcagagatcgcgctcatcatcttctcaggccgtggccggctctatgagtactccaataacaggaactga

SEQ ID NO: 98 - DNA, 684 bp, Elaeis guineensis
  oil palm SEPALLATA (SEP)-like EG4N11519
atggcacgcggaaaggtgcagatgagacggattgagaaccctgtccagcggcaggtcaccttctgcaagcgccgagccggactgctcaaaaaggctagggagttgtcagtgttgtgtggtgctgatattggcatcattatattctccacccatggcaagctttatgagctagccactaacggggacatgcaaagtttgattgagagatacaagagcattggtgcagaagctcaaattgaaggtggtgaagtgaatcaacctcaggtctcagaacaggagatatccatgttgaagcaagagatcaatctgctgcagaagggcataaggaagtgcaaccttcccgaatcaaacagtgagagtcactactatggagaagaggagatcgaagacaacaacaaaccaaggaggctccggcatgcgacgggagaaggcgacgagagggggcgcgagaaggtctccagagaggccactggggtggaggggaggccgtcaagcggcagcgccgccttggccttgtcacccgtctccacggacttgagagccacggatttgggaggagtggtggcaaacgccgccgcctgcgtgttaggggaggccggctggacgtcgaggcccgaaggcgaggtcgtggccggacggactctcgtcgagggactgcgaaaaagaaatgcttcaaaggcctag

SEQ ID NO: 99 - DNA, 780 bp, Elaeis guineensis
  oil palm SEPALLATA (SEP)-like EG4N14715
atgttgatgcatttgacactgaaggacaaatgtgttggagatgagcttgagcttgaagttggtgatggacttacatttggagaagtttgtgtacataagatctcttatgcagctctttatacaagcccaggggtggcaagccttgttttggagagggggcggtgcatttgtttctggtgttgtgagaagagaacgatggtgagaggaagaagggagataaaaagaatcgagaaccccatccagaggcagtccactttctataaaagaagggatggcttgtttaaaaaagccagggagctctccattctctgcgacgccgacctcctcctcctcctcttttcctcttccggaaagctctacgagtatcacaccccttctgtgcccagtgccgaggagcttgtcaagaggtacgaggttgccacccaaaataagatttggagggacctccacttggaacgaaatgctgagatggagaaggtccagaagttgtgcgagctcttagaaagagatctaagattcatgaaggttgacgcaagccaacactactcgctgccagttctcgacgttttagagggcaatctggaggcagccatcaacaaggtccggtcggagaaggatcggaagatagtaggagagatcaaccacttggaaaacatggtaagagatcgccagcaagagaggtacgatttgggcgacaaggttgcccgtgcacagggtcttaaagacatggcagtaccactcaaccgactggatctgaaattgggtacttgtgtttcctaa

SEQ ID NO: 100 - DNA, 441 bp, Elaeis guineensis
  oil palm SEPALLATA (SEP)-like EG4N82401
atggtgaggggaaagacggagataaagcggatagagaacgcgacgagcaggcaggtgacgttctcgaagcggaggaatgggcttctgaagaaggcgttcgagctttcggtcctctgcgacgccgaggtcgccctcatcgtcttctccccccgggggaagctctacgaattctccagcaccagatatactggctatttgggaaaaatcaatgtcaaaataatgcaggacaagaacaagactttgagagcttgtttggtgtttgtcaacatcttaatcaccttgatgccagggaacgcattatcattgcaatgccatgctctactcaccccttcgcaatacaaccagaatctttcgagtacgaatgatgaaggccttcgtttcaaatcagattcatcttttaacaaaatgggggagtggcccgattcagttttggtgaaatga

SEQ ID NO: 101 - DNA, 414 bp, Elaeis guineensis
  oil palm SEPALLATA (SEP)-like EG4N37080
atggttcgagggaagacggaggtgagacggatcgagaacgcgaccagccggcaggtaacgttctccaagcgccggaatggtctcctgaagaaggccttcgagctctccgtcctctgcgacgccgaggtggctctcatcgtcttctctccccgaggaaaattgtacgagttctcgagctccagcagacttattgtgatggctgtgaccacaagcttagctgatcacgtagataggatctcagagaatctcaacgatcgtatcgtggacaatatctcagaagctttaaggttgctggctccaaagcctctgcatgacttcctccacatgtgcgttagcccacgtttggatcgtggagtcttgagaggagtatcgagttgctggagggtcgaagctgtggtgaatcctatgacctag

SEQ ID NO: 102 - DNA, 732 bp, Elaeis guineensis
  oil palm SEPALLATA (SEP)-like EG4N63104
atgcgtggaccgtgtgaggagcatcgcgctggccgtgcaacgcgcgcccgcctgagcctgggccgcgcaccttgtgcgcccgcacattgggccacatgctcacagccatcccgcatgctgccacgtgcacccgctcaggcggcctacaggaagacacaggtgagacggatcgagaacgccaccagccggcaggtaacgttttccaagcgccggaatgggcttcttaagaaggccttcgagctctccgtcctctgcgacgccgaggtcgcccttatcgtcttctcccctagagggaagctctacgagttctccagctccagagctactgtgagttttggttccaggaaggtatggattattcaagctacaatggatgcagaagccaatgactgtggtagagcatcctccacgaagatgctctctgcatgcaactcttgctgtgtgcaggctgtaggggagtgggtctatactgccttcaatagaggaggttctgagagtaaaactcgagaggtttcccaagatctgggcacagaatcatgtgcaattgaggaactgcatgatctagagctccagttagagcaaagcctaagcagcatcagaaatcggaaattaaatgcagaacctcggctacagctatgtgctcctgctgtttctgatgattatgatagtcagaatacagatgtagagacagagctggtaattggtaggccagggacttgcaaggtcaagtga

SEQ ID NO: 103 - DNA, 642 bp, Elaeis guineensis
  oil palm SEPALLATA (SEP)-like EG4N37079
atggttcgggggaagacggaggtgagacggatcgagaacgcgaccagccggcaggtgacgttctccaagcgccggaatggtctcctgaagaaggccttcgagctctccgtcctctgcgacgccgaggtggctcttatcgtcttctcccccaagggaaagctctacgagttctccagctccagcagggatggagtcgaagatcaatactcaggaggtgagcgaacctatagctccttagtctcgttttccaaatatatgttaagaaactgtactgaggatccattaggaatgatgattaagcccaagctttaccatctcgttaccaaatcctatgcgggtactatcttattacagtatcgcattcaaaagacagttgatcgttatttaatgcacacaaaagatgtcaacatcaacatcagagcaacggaacaaaatatgcagtgcaagacagaacctccagtacaactgataactcaggcatcttcaaatggtgatgcttgtcaaaatatggaggtagagactgagctgattattggaaggccaggaacctgtgaggctaaacaacaggatcatgttagcctcaacaagcagtggtcgcaggaaaatggggcattcggaatggagagcagacaaaacccataa

SEQ ID NO: 104 - DNA, 519 bp, Elaeis guineensis
  oil palm SEPALLATA (SEP)-like EG4N29559
atggtgagggggagggtggagctccggcggatcgaggacaagacgagccgccaggtgagcttctccaagcggcggagtggcctactcaagaaggcgcacgagctcgccgtcctctgcgacgccgaggtcggcctcatcatcttctctgccaagggcaagctctacgacttcgcgagcacctccagtgtgtacagatacaacatcatcatggacaataggccagaattgttggaagaaaaaaggatcgaatgttatgtggccctgatgcatgatttgtacataaagatttggtgcaaaattgcactgagtaatgtggattataaacttgctgccgagtttgcccttctaagatgcaagcctttaacacgtcctttcaatgaaaggcatccaacaatgtcttggaagcttcttgtggagcaaaggaaggcccaaacaggctatacacccttgaacagcacccctcacctctatggaggaaattggccaggccattcctgcactccgcttggaagtggttga

SEQ ID NO: 105 - DNA, 408 bp, Elaeis guineensis
  oil palm SEPALLATA (SEP)-like EG4N43162
atgggcagagggaagatcgtgatccgaaggattgagaactcgaccagccggcaggtgaccttctctaagcggcgcaagggtctgttgaagaaggccaaggagctcgccatcctttgcgatgccgaggtcggctttgtcatcttctccagcactggcaggctctacgattttgccagctccagcgaggctgaacttgggcatcacaaaaccaaagtctatataagcgcaacggaatggtggcaaaggattgagtttgagtcggatcaaatatgggttgggtcaaagaatcttcaacgaccactccatcaatataaagataagacctttttcttaaggcaacatagaggcaagactttcggctcaagtctcctccaatggatggaggatgctgataacttgtggggataa

SEQ ID NO: 106 - DNA, 930 bp, Elaeis guineensis
  oil palm SEPALLATA (SEP)-like EG4N31052
atgaggctcaggttgtcgtcgttcacactacacctaccgcggccccaccctattattgtctacgtcgcatccatcgttcgtgtagtattcggctttgacggcaccaagccttctcccctttccgatcctgatgcaccccgtgcgacccgccccgcaccctttgcggcctcgccccaccgccatcccctttccttctctcttacgaccccgatgaatccgagcccttgtggctttatagcgacatacacggttcccgagagccaggaaggcggaaccgtccaaaacgggggcaccaactttcgacgagaaagcgtctggtgcatattaggatcaatggtgagggagaaaatccagataaggaagatagacaacgcgacagcgaggcaggtgacgttttccaagaggaggaggggactgctgaagaaggcggaggagctctcgatcctctgcgatgccgaggtcgcccttatcgtcttctcgtccaccggcaagctctacgagtactcgagctccagtgccccacttccattcgccgcccccctcccctcgcccatagtatctccataccggcggccttcccacgccggcggcctccttgtgccggcaatgctggtagcgtccctgtgctgtggcctccctgcgaggcagcatcagctgccccctcttgctgtctgtcccctcttcacgtgggcaggcgttggccttccacttgatcgccccctccctttgccccccctcctctcacccatagcatccatcatgaaggagatcattgaaaagcacagcatgcattcaaagaacctacagaaaccagaccaacccccccttgacttaaatggagaatggcttctacatgcaattgtaaccccgaagtatttacatcaagttctaacatcaaatgatgaatacttctcccctgatgaaacttaa

SEQ ID NO: 107 - DNA, 195 bp, Elaeis guineensis
  oil palm SEPALLATA (SEP)-like EG4N86343
atggtgcgtggcaaggtgcagatgaagaggatcgagaaccccgtccaccggcaagtcaccttctgcaaacgccgggcagggctgctgaagaaggccaaggagctgtctgtgttgtgtgatgccgaaatcggaatcataatcttctccacgcatggcaagttgtatgagctagctactaaggggtcttacaactga

SEQ ID NO: 108 - DNA, 444 bp, Elaeis guineensis
  oil palm SEPALLATA (SEP)-like EG4N39902
atggggcgtgttaagctccagataaagagaatagagaacaacaccaatcgccaggtgaccttctccaagcgtcgcaatgggctcatcaagaaagcctacgagctctcggttctttgtgacattgatatcgccctcatcatgttctctccctccgggaggctcagccatttctccggcagacggagattttttgagccagaccccctcagcatcacttctatggatgagcttgaatcatgtgagaaatttctcatggaggccttaaggcgcgtggcagagagaaagcatggaggatcatgggtcaaattagtacaattaccgcgaggatggtaccaaaatgaactgccacatctagcggtattcaccaacgacacaaagttcttaattcccatgctgctgaagaacaccgtgatttgtattgtgtatcgccaaaagcttttgtga

SEQ ID NO: 109 - DNA, 762 bp, Elaeis guineensis
  oil palm SEPALLATA (SEP)-like EG4N48307
atggataaattagaggctagatcctttaggactcgctttatagggtatcctaagaaaatcatgagatactacttctatcttcctgagaatcacaataggcgatcagacttgataactttcaatttgccatggagaagatgtgctagtttgatgagacggcatggcagtggctcacacaacacctacctgagttgtggtcaaggcatgcctttgcgggccgctagggtgataactagaggaagcgaaaccatcactcggacgcgaaaaccgaaccgccccatcaccaccacgccaacgtgtcgcgtcccgagaggggagattcgggtgccgaatggagtctggaatcctcggtgggcctcccctctccccgttcatcttcctcggtcctcaagaccgccagcccactctaacggcttaagcttggggttccggcgtccaacggcggcggcgatgagaagggggaaggtccagattcggcgaatcgaggacaaggccagccgccaggtgaccttttccaagcggcggggcggcctcttcaagaaagcccgcgagctcgccgtcctctgcgacgcggaggtcggcctgatcgtcttctcccccagcggcaagccctacgaattctgcagctcctccaggtgcgtttccattctcctccttcggcttaggtcgtcggatccctcgagatccatcgattccctcagagaccagcccggctcagttcgtcaaacacttcgctcgtcttcgttcttgagacggtggtga

SEQ ID NO: 110 - DNA, 456 bp, Elaeis guineensis
  oil palm SEPALLATA (SEP)-like EG4N23857
atgggtcgtggaaagatagagatcaagaggatcgagaacccaactaaccgtcaggtcaccttctccaagaggcggggagggctcctcaagaaggcaaatgagcttgcgatactgtgtgatgtgcaggctagcatgaggcagtacactggggaagacttgagctctatgaccatgaatgacttgaatcagctcgaacaacagctggagtactcggttaacaaggttcgaacaaggaagctatcagagcaccaggcagcaatggagcatcagcaggctgccatggagcacaaggtgccggacgtgcccatgctggagccattcgggttgttctatcaggatgagccatcgaggaatttgctgcagctttcgccccaactgcatgcattccgtctccagccggcgcaacccaatctgcaagaggccagcctcccaggtcatagtctgcagctgtggtaa

SEQ ID NO: 111 - DNA, 675 bp, Elaeis guineensis
  oil palm SEPALLATA (SEP)-like EG4N29533
atggttactcttttgctagcacagagtagtcagcaagagtacttgaaattaaaagcacgtgttgaagccttacagagatcgcaaagaaatctcctcggtgaggacttgggtccactcagcagcaaggagcttgagcagctcgagcggcaacttgatgcatcgttaaagcaaatcagatcaacacggacccaatacatgcttgatcagcttgcagatcttcaacgaaggttggaagaaagtaaccaggctggtcagcagcaagtttgggatcccactgctcatgcagtaggctatggccggcagccacctcaaccacagagcgatggattctaccaacagatagatggtgaacctactctccaaatcagtgttgaaggagaggaggatgagggtgaattagtagaggaggacatggagaaaagagcaagtgatgtaaaagaggaattggagtacacccttgtatatgtgatgaggtatcctccagaacaaataacaatcgcagcagcacccgggtcaagttgggccataatttctaacaaactcgatgatgaaaaagaagaagaagaggggtccttttccgatgatgattggaggctgacggtggttgattcggagtgggtcatatcgatgaggttggtgatgggttcttttccatgctttgtcaaggaagactaa

SEQ ID NO: 112 - DNA, 1170 bp, Elaeis guineensis
  oil palm SEPALLATA (SEP)-like EG4N70708
atgggggaggaacatctttccgacggaaagactgcctcgccgatccagttgagtgaggagtctaggagagggatggcgagggagaagattcagataaggaagatagacaacgcgacggcgaggcaggtgaccttctccaagaggaggagggggctcttcaagaaggccgaggaactcgccatcctctgcgacgccgacgtcgccctcatcatcttctcctccaccggcaagctttttgagttctcgagctcaagggtttttatggtgatcagagtgaagctccgtacgggtttagctaggtgggttttgttgcagatgattacaactctaccaaaatctggacactcaagtgttggaattccattgattagcttcaaggctattgtggtggagatggccagagcagggagacgtgtgctgactgattcggaaaatgttatgtatgaggatgggcagtcatcggagtcggttactaatgcttcacaattggtagtgccaccgaactatgacgacagctccgacacatccctcaaattggggtccactgattgtgggctcactgaggtctgtgtggattatgatctgtatgtcacaacctcctgcactttgtttgagggatatactgctgtgagaaaacaggcactgtctttgttcttatatgatcggagtacgcatgcagcacaaattgatagaaaacggcgccagcaagtacggatccaggaatggcgccggttgagcaaattgactggtctcttagctggagcacttaatttgtttggcgccgtatcagggccaaaatatgatggcaaatttctgcactctaaagtgaaagaactgcttggtgatacaaagcttcatcaaactttaactaacattgtgattcccgctttcgacatcaagcttcttcaacctgtcatattctcaacctttgaggatgacaccttggaaggagacacggcatccgtggacgtctcgacgagtgagaacttgcgaaagttggtgcaagttggccaggatctccttaagaagccggtatcgagggtcaatctagagactggcgtgtctgaggcctgcgatgttgaaggaaccaacgaagatgccctcatccgctttgcgaagatgctctccaacgaaagaaagtctaggaatgcaaaaatgtcagctgcttga

SEQ ID NO: 113 - DNA, 408 bp, Elaeis guineensis
  oil palm SEPALLATA (SEP)-like EG4N67350
atggacaaatttgaaatagctatcaagactagtcagcaagagtacttaaaacttaaagcacgtgttgaagcattacagagatcacagagaaatctccttggtgatgacttagggccactcagcagcaaggagcttgagcagcttgagcggcaactagatgcatcattgaagcaaatcagatccacaaggttggaggaaagcaaccaggctactcagcagcaagtttgggatcccaatgctcctgcagtgggctatggccggcagccacctcaaccacagggagatggattctaccaacagatagagtgcgatccaactctccatatcgggtatcctccagaacaaataacgattgctgcagcgcctgggcctagcgtgagtaattacatgccaggatggcttgcgtga

SEQ ID NO: 114 - DNA, 573 bp, Elaeis guineensis
  oil palm SEPALLATA (SEP)-like EG4N44069
atggcggaggaccgctggcggcttgcggcgggccggcggcgcgcggcccagaagtggcagcgcccggcttgggtgcgcagggtgcggcctagtacatgcgtgcgggatgcggcccaggccctggcccaggcgtgcatgcgggtgcagcctaggcccacgcgagcccgtgctggaaacctcatgctcaagacaatcgagaggtaccagaggtgcagctataatgcaacagatgcaatagttcctccaaaggagacacaggaccttggtccattaagtgtaaaggagctcgagcaacttgagaatcaaatagagatatctctcaagcacatcagatcaaaaaagacccaattaatgcttgatcagctatgtgatcttgagcgcaaggaacaaatgttgcaggaagctaacaaagccttgagaagaaggttggaagaagatacaattaattccctccaactttcatggcaaaatggagccaatgttgtggggaatgccccatgtgatggtgaacctcctcaaacagagggattctttcaaccgctgggatgtgaaccttctctgcaaattgggtaa

SEQ ID NO: 115 - DNA, 195 bp, Elaeis guineensis
  oil palm SEPALLATA (SEP)-like EG4N67198
atgagtgagcgggggagcagggagcattggtggtggacggaagacgttgagctgaagaggatcgagaacaagatcaaccgccaggttaccttctccaagcgctgcaacggcctgctcaagaaggcctacgaggtctccatcctttgcgatgtcgaggttgcactcatcatcttctccagccgtggcaagctctag

SEQ ID NO: 116 - DNA, 681 bp, Elaeis guineensis
  oil palm SEPALLATA (SEP)-like EG4N130373
atggtgaggaagccgagcatgggccgtcagaagatcgacatcaaaaggattgagagtgaggaggcccgccaggtgtgcttctcgaagcgccgcgccgggctcttcaagaaggccaacgagctgtccatcttgtgtggcgccgagatcggtgtcatcgtcttttcccccgcaggcaagccgttctccttcggccacccctccgtcgactccatcatcgaccgcttcctctttggcagcccctcccctacgactctgccgtccgccgacccccgcatgccggtggcgcgcgagatgatggtcgtccacgagttcaatcaacagtacacggtgctcacggccttgctggagaccgagaagaggaagaaagcggtgctcgaggaggccgtgagggtgaagcaggctggggaggccgccttgtggggcgcaaacattgaggaactcagcctgggggagctcgaaagtctgcacaagtcctttgagaggctgaggagggacgtggcgatgcgcgccgaccagctcgtcatagaggccgcgcatactcgcagctccagcgtcgcagcggcaggtagttttgttcctcctcctccccttggtgtcaatctaggctttggtcgtggggtggaggggagcatggcgcttcctcctcccactttctttggttatggccgtgggcccttttag

SEQ ID NO: 117 - DNA, 906 bp, Elaeis guineensis
  oil palm SEPALLATA (SEP)-like EG4N128041
atggatcgaggtgacgtcgaccttcaaaagatcgatggaaaggagaacctggctaaccccttcactaaagccctgacgataaaggagttcgacaaccacaagaagaaggaagaagaggcattaaggaccacacccacggaagatgatgatgatatgatattgttggatgaaggtgttgatatagcatcctctagtaagagagataatagtgatcatgcgtgcaatatggtgaggaagccgagcatgggccgtcagaagatcgacatcaaaaggattgagagtgaggaggcccgccaggtgtgcttctcgaagcgccgcgccgggctcttcaagaaggccaacgagctgtccatcttgtgtggcgccgagatcggtgtcatcgtcttttcccccgcgggtaagccgttctccttcggccacccctccgtcgactccatcatcgaccgcttcctctctggcagcccctcccctatgactctgccgtccgccgacccccgcatgccggcggcgcgtgagatgatggtcgtccacgagttcaaccaacagtacacggtgctcacggccttgctggagaccgagaggaggaagaaagctgtgctcgaggaggccgtgagggtgaagcgggctggggaggccgccttgtggggcgcaaacattgaggaactcggcctgggggagctcgaaagtctgtacaattcctttgagaggctgaggagggacgtggcgatgcgcgccgaccagctcgtcatagaggccgcgcatactcgcagctccagcgtcgctgcggcaggtagtactgttcctcctcctcctcctggtgtcaatctaggctttggtcgtggggtggaggggagcatggcgcttcctcctcccacttcctttggttatggccgtgggcccttttag

SEQ ID NO: 118 - DNA, 675 bp, Elaeis guineensis
  oil palm SEPALLATA (SEP)-like EG4N147209
atgggtcgccagaagatcgagatcaagcggatccagaacgaggaggcccgccaggtgtgcttctcgaagcgccggaccggccttttcaagaaggcgagcgagctgtccatcctctgcggcgccgagatcggggtcgtcgtattctcccccgccggcaaggccttctccttcggccacccgtcggtcgacgcggtcttcgaccgcttcctcacgggcaacccccaccacggcaacagcggggggcccgcggcggactcgcggcgcggggcggtcgtgcgcgagctgaaccgccagtacatggagctgcatgggctggtggacgcggagaggaagcggcgggaggccctggaggaggccatgaagggggagcaggggggccgcccctactggtgggacaacaacgtggactccctcgccctggaggatctggaggagtacgagaagaagctgctggagctgaggaacaatgtcgccaaggttgctgatcagctgctgcatgaggccatggctcgcaagcagcagcagcaccatcaccaccaccaccagcagcagcagcagcagtttccgatggtcggcgctgccgtcgctctccctgggcccttcgccattaagaacgaggatgccatccatccttctcttggtggcgggttgggtttcgggcatggcttcttctga

SEQ ID NO: 119 - DNA, 681 bp, Elaeis guineensis
  oil palm SEPALLATA (SEP)-like EG4N37712
atgggccgtcagaagattgagatcaagcgaatcgagagcgaggaagcccgccaggtgtgcttctcgaagcgccgcgtcgggctcttcaagaaggccaacgagctctccatcctgtgcggcgccgagatcggcgtcatcgtcttctcccccgccggccagcctttctccttcggccacccctccgtcgactccatcatcgaccgcttcctctccggcggcccctcccctccgactctagcctccgccgaccgccgcatgccggcggcgcgcgagatgatggtcgtccgcgagctcaaccgccagtacacggagctcgcggccttgctggagacggagaggaggaggaaggtggtgctggaggaggccgtgagggtgaagcgggcgggggaggccgccttgtggggtgcgaacgtggacgagctcggcctgggggagctcgagaggctgcacaagtccttggagaggctgaggagggacgtggcgaggtgcgccgaccagctcgtcatcgaggccgcgcatgctcggagctccagcatcgcagcggcgagtcgcagtactgctcctcctcctcctcctggtatccatctgggctttggtcgtggattggaggggagcatggcgttaattcttcctcctcctcccactcccactgcctttggttacggccgtgggctcttttag

SEQ ID NO: 120 - DNA, 669 bp, Elaeis guineensis
  oil palm SEPALLATA (SEP)-like EG4N153108
atggtcaaagctgaagtggagctaatgggcatagtcgaggataagacactcgaaaggtaccaaaaatgtaactatggtgctccggagactaatattatatcaagagagactcagattcttgagcttgtagaatggatccgctataagtggcttgatgaagatatcgacaaaaatctcctcggtgaggacttgggtccactcagcagcaaggagcttgagcagctcgagcggcaacttgatgcatcgttaaagcaaatcagatcaacacgggaacaaatgctatgtgaggccaacaaaagtctaaggcgaaggttggaagaaagtaaccaggctggtcagcagcaagtttgggatcccactgctcatgcagtaggctatggccggcagccacctcaaccacagagcgatggattctaccaacagatagatggtgaacctactctccaaatcagtgttgaaggagaggaggatgagggtgaattagtagaggaggacatggagaaaagagcaagtgatgtaaaagaggaattggagtacacccttgtatcctccagaacaaataacaatcgcagcagcacccgggatacagatgagtcaatagaaatcaaggggctcaaacttcaaaagttcgacaaggaccaaggggagggccagcacactgccctataa

SEQ ID NO: 121 - DNA, 660 bp, Elaeis guineensis
  oil palm SEPALLATA (SEP)-like EG4N108259
atgggccgtcagaagatcgaaatcaagaggatcgagagtgaagaggcccgccaggtatgcttctcgaagcgccgcgccgggctgttcaagaaggccatcgagctgtccatcctgtgcggcgccgagatcggtgtcatcgtcttctcccccgccggcaagccgttctccttcggccacccctcggtcgactccatcatcgaccgcttcatctctggcagcccctcccctacgactattccatccgccaacccccgcatgccggcggcgcgcgagatgatggtcgtccgcgagctcaaccgccaatacacggatctcgcggccttgctggagactgaaaggaggaagaaggtggtgctcgaggaggccgtgagggtgatgcgggcggggaaggccgtctcgtgggaagcgaacatcgaggagctcggcctgggggagctcgaaggactgcagaagtcctttgagaggctgaggatggacatggcgatgcgcgccgaccagctcgtcatcgaggccgcgcatgctcagagctccagcatggcagcggcaagcagtgctgctcctcctccttctggtgtcagtctaggctttggtcgtgaattggaggggagcatggcgcttcctcctcccactttctttggtcatggccgtgggctcttttag

SEQ ID NO: 122 - DNA, 735 bp, Elaeis guineensis
  oil palm SEPALLATA (SEP)-like EG4N71703
atggccaggagaaccagccacggccggcgaaagatcgagatcaagaggatagaagatgaacaaactcggcaagtgacgttctcaaaacgtcgaggtgggttgttcaagaaggccagcgagctttccaccctgtgtggggctcaggtcgggatcttggtgtactccccaggaggaaggccctactccttcggccaacctggcttcgtggaggtctctgatcgattcctcccatgcgtccccacgccgatcggctcagaccctcctcctatgccacctccagcctacttgtcggtgtcccagcccagcaagcactacctggaggtcgtgaacgtgctggaggccgcgcgggccaagggtgcagtgcttaaggagagacttgccatggttctcgaggaggaggggcgggcctatgagtctgaaaatgatgacctcaccgtggaggagcttggagacctcgtcgcgcgattggaggcgcttaaaatgcgggtgttttccagattctctacgatcctgaatcaacaacaagcttcttcatcgagtgctgctttgactgtcaccccgctgaatgtgatcaacccttatgccaccaatggaccccaggcttatccaggtggtgggttcgtcctggggaataatggccatggtgccggtgggttcctgggaaccggtggccatggtactcccagtggattcatggggaacgatggtaatggtcctcttgggttcattgcttga

SEQ ID NO: 123 - DNA, 654 bp, Elaeis guineensis
  oil palm SEPALLATA (SEP)-like EG4N2959
atggttagaaagacaagcaatggtcaccggaaaattgagatcaagaggatagaaaatgaacaaatccggcaagtcacattctcaaagcgacgacagggcctgttcaagaaggccagcgagctttcaaccctatgtggtgctcaagttggaattttggtctattctcctgctggaaggccctattcattcggccaacctgggttcgaagtggtatcgaatcaattaatcgctcacaactccttcatgaccagcccaaaccctatagagggacctcagggcaatgcaattgtgcaacaactgaattgtcactgtatggagatcatgagtctactcgacaccgcgaagaccaaaggtgcagtgctgaaagaaagacttgaaataactccaaaggggaaggagaaggctttcgagaccgagcttgaaggctttggtatggatgagcttgaaaggttggtgaagtcctacaatgatttgaaactaaaggcggattcaagaatttataagataatgagtggaggagcttcttcatcaggtggccctttgcccgttaaccctaagcttgctagagatagagagttactcttccaacctaatatctgcttggagatcttttcaatcataaaagaccgatctatgcagcgaggagcggagtga

SEQ ID NO: 124 - DNA, 291 bp, Elaeis guineensis
  oil palm SEPALLATA (SEP)-like EG4N82416
atggcgaagttgaaggcaaagtttgagtctctgcagcgctcccagaggcatttgctgggggaagaccttggaccattgagtgtgaaagaactgcaacaacttgaacgtcaacttgagtctgctctgtcacaagctaggcaaagaaaggctcagataatgctggaccagatggaagaacttcggaaaaaagtaagcatgctggatgaaggccaaggttcagaacatttggaggcacgatttccatgttcgatagaagagattgccatcgttggcttcagcagagtggtgtag

SEQ ID NO: 125 - DNA, 1086 bp, Elaeis guineensis
  oil palm SEPALLATA (SEP)-like EG4N14105
atggggagggtgaagctaaagatcaagaaattggagaatagcagtggtcggcaggtcacctactcgaaacggagggctggaatattgaaaaaggctaaggagctatccatattgtgtgacatagatctcgtccttctcatgttctcacccactggaaagccgacattatgcgttggagaccggagcaccattgaggaggttgttgcaaagtttgcccaactaactccacaagaaagagcaaaaagttattggaccgatcctgataagattaataacgtagaccatattggggctatggaacaatctctccaggaatctctcagccgcattcaggtgcataaggaaaaccttggaaaacaacttatgtctctagattgcagtggccaggtaaaagcacttcttggtaagcaagcagaggccaatgaccaattacaagaggattctttgcatgagtttagccaaaacgcatgcttgaggttgcagctaggaggccagtacccttaccagtcctattgtcagaatttaattggcgagaatgcattcaagcctgatacagagaatagcttaccggaaagcactatagattaccaagttgaccactttgagccacctagacctggatacgatgcaagctttcagaattgggcttcgacatctgggacatgtgatgttgctatatatgatgaccagtcgtactcccgacgctccgcgttccgtcattccatcgaccctgtagcataccgtggatcttacgattggtgtccgtcaacctgtgttccccaatgcttcccctatccacccacatctgctgtaccagcaccgaatcatgaccgttccttccccaaacgtaggctcattaatattcatccagtcaacctacgcgacccgttgcttaagccccaccttttccttggatcactcaaaaaccatgttccaaaatggagaagtcagaaggatctcgcacgtgccaacccggcctcgggcctcccaacacgtgccagtcgcggtacccacacgttgacgccacccaaaagggaacaaataaaaagtactcacacgtgtcagcgtcataacatcctcctgtaa

SEQ ID NO: 126 - DNA, 621 bp, Elaeis guineensis
  oil palm SEPALLATA (SEP)-like EG4N37867
atgtcgaaagaaatagtggggaaaaaaactccttatcctcatgaagaagccttggcaggttctcaaggccaaggagtgtccaaaaattctcaacaagactgcacattagctaaaggaacagcaattagttggaagccatggaatgcccctccccagagtcatcactatagtgcaatagagacagctagagctcagaacagtactgcaacaacctcgaagctagtcaaaactagtgggaggttgtctgcggagatggcacgcggcaaggtgcagatgaggaggattgagaaccccgtccaccggcaggtcacgttctgcaaacgccgggcagggctgctcaagaaggcgaaggagctatcagtgttaaccgatgccgatattggagatatcagttctaaagcaagagatcaacatactacagaagtgtttgagatagtggagcaaaatgggcattttgatgtagctccaatgatggtacaacaaaatgggcattttggtgtatccccaatgatagtacagcaaaatgagcattttactgcagctccagcgatggaagacattccatatccactaaccatacagaatgactattccagttttacgagcttagacatgggctaa

SEQ ID NO: 127 - DNA, 594 bp, Elaeis guineensis
  oil palm SEPALLATA (SEP)-like EG4N71708
atggccaccatgcccaagaagaccatgggccgtcaaaaggttaagctcaagaggatagaaaatgaggatgctctctatgtgaccttctccaagagaaagtcgagtctcttccagaaagctgccgagcttgccaccctgtgcgggtccgagattgcactggtggtgttctccccggcaggccggccgtactctctcggcctccccaccgtcgacaaggtcttccaccgagtcctctcgagtggacctgcccaaatgggctccggccacagcgtggtgagccactccgccaagcagtgctccgagataaccaaacacttggaacaagagaagagcaggaaggccattctcgtggagaggctccagaaggaggcaccacccaggtgggaggatgggctccatggactcgggtgggacgacctcctgatactggctaaagaggtggaggagctcaagtccaaggtggattccagggtctgcgagatccttctccaaggggcttcatcatccacggctaatgctgatgcttggcccgtcggaagctctgagggttcgtatggggttggaccacgggggccgctggataataacatctaa

SEQ ID NO: 128 - DNA, 762 bp, Elaeis guineensis
  oil palm SEPALLATA (SEP)-like EG4N37348
atgcctaggaaaaccaggaccacgcggggcaaacaaaagatagagatcaagaggatcgagaaggaggaagctcgccaaatttgcttctccaaaagaagatctggcgtctttacgaaggctagcgatctctccaccctctgtggcccggatgttgcagtgctggcattctcccctcgaggtaagcctttttcttttggcagcccggccgtcaacccggtgatcgaccggttcgtgttggatatttcttcctcccccggttcaggccaccattgtggaccgccgagcaatacggtccaacaactcagcaagctatgcctggacctcaccaatcagctacatgcttgtaaggccaagagtgcagtgctggaggagaagctcagctcccccggttatgatatcttggagctcgattggttcgagaacgtggatgacttggagctggacaaactggggaagctggcagaggctctgaagcgagtgaaggtgaacgctgatgcacacgttgacgcacgcctcctgcatggtaggggggccttgtcctcctctactactcctgttatgaccgccaaccaagttgagggagcttcgtcttctaatagggtgatggctgctgcatcttctaaaggggtcatggctgcaggaaatgtgccggtggcattcttgacgatctccatgttagcgatgttcgggaatatgatcaagaagaaccacttggataatgtggaggttagtccatattggacaaggttggatgccaagtga

SEQ ID NO: 129 - DNA, 582 bp, Elaeis guineensis
  oil palm SEPALLATA (SEP)-like EG4N71707
atggctgagaggaccttcagaggccgccagaagatcgagataaaaaagatagagaaaaaggctgctcgagatgtgacattctccaagcgtagggttggggtgttcggcaaggcgagcgagctggcaaccctgtgcggtgtggacattggggtggtggccttctcgcccgctggccggccatatacgttcggccatccggatgccaatgtggtgttcaatcgttttctcgggctggtccaaccagaaggctctagcggctccgtaggcgcgatggcaaggcatcgggctgagatgcttcgccagctgaccctacactgctcgcagatgatggaccgcctcgcggcggaaagagagaagagagctgtcctggaagagaggcttcgcaaggtgagcgaagatccccaggaacgcgcatggcccgaggacctcgaggggttggggctcgagagacttgccaggatggtgaggggcttcgaggagcagagggcgaaggctcgagcgaggctgcatcagatacgggagttgggggaatcatcttcggggccttcggccactgtggaatttaagaagagtgttgtatga

SEQ ID NO: 130 - DNA, 876 bp, Elaeis guineensis
  oil palm SEPALLATA (SEP)-like EG4N104943
atgaacggcgagaacgacgctgctagcaggatcatcttttcttctctgaaagaacggctggtacaatccggtgtttcctatgcaaaagcggtcaaaaagcaccccatcccatccccagtggtcaggaaatctaccgaaacagtcaaggatctcatgagttccaattcaggaaatgtacatcatcatccccgttctcgagggcaccgggtgaagctcttgagtaaaggaacttgttttcgctgtggagatcgtgatcacacccgagaatcttgcagaaatccgattaaatgctttctttgcaagggttatgggcatgttcaaaagagcacagcatcacccttctggaaaggtgtcttaagcacgcatggactttttcagcagctcttctcaatcaccataggcaatggaaaatgggtctcatgctggactttcatcaaatcaaccattgagagatacaagaaggcatgtgctaatacttcaaattcaggttctattgttgacgttgattctcaacaatattatcagcaagaatcagcaaaactgcgccaccagatccaaatattacaaaatgcaaatcggcacttaatgggtgattctctgggttctttgactgtgaaggagcttaagcaactcgaaaaccgacttgaaagaggcatcacaaggatcagatcaaagaagattgcagagactgagcgagcacagcaagtaagcatcattgaagcaggacatgagtttgatgctcttccaggatttgattctaggaactactaccatccgcatatatcgcaacaaaaatctatgatggctcttgtaaatgaaaaagaacagtcacaaaatcaatcacagctcctccaagagcttggtcagtcagaatga

SEQ ID NO: 131 - DNA, 819 bp, Elaeis guineensis
  oil palm SEPALLATA (SEP)-like EG4N35645
atgggccggtccaaggtgaaactaaagttcattgaagaacagcatcgacgttcggcaacctataggagaagaatagcagggctaaagaagaaggctagtgaattggccattctttgtgacatcccggtcttggtgataagctttggaccccgagaacaagtagagacatggcctgaggacaatcaagcagctcgacacattattgacaggtatcgagagcttagtatcgatatccgaaacaagaacaaacttgacttaccaggttacatgaaggctgaaatcatcagacatcaagcatcattcaataggaggtgcagggatttagctgatatgccattgttgcctttggatggtttgttttatgccctgctcaagtcactaagggagcttgctcatcaactggactcaagaatggaggtgatcaaagagagaatccaattgcttaaagatagaaagcacttcaatttaggagagaccatgaacatgggaagccaattgctagaaatcactccccgtgatgggatgatgggtattcaaaatacagcttctgcttatgatatgatgttttcggatccatatctcaccatgaacgcttctttgcaagaccctccacagccaacgagcttcagtagcggacagatttctccagatgctttcttgcagtatctttatgggccaatgggcatggatgaggtacccttagctatggtgccttcaattccatcgaacatggatgaggtacccttggctatgatgccttcgattccaatgaacatgaatgagcctccaggggcacaattggcaaaattatgtgactaa

SEQ ID NO: 132 - DNA, 720 bp, Elaeis guineensis
  oil palm SEPALLATA (SEP)-like EG4N37749
atggcaaggaagaaggtgaacctggcatggatcgccaacgactcgacgaggagggcgacgttcaagaagaggaggaaggggttgatgaagaaggtgagcgagctggcgacgctgtgcgacgtgaaggcgtgcgtgatcgtgtacggccctcaggagccgcagccggaggtgtggccgtcggtgccggaggtgacgagggtgctggcgcggttcaagagcatgccggagatggagcagtgcaagaagatgatgaaccaggaaggattcctccgccagcgcgtcgccaagcagcaggagcagctgcggaagcaggagcgcgagaaccgggagttggagacgatgctgctcatgtaccaaggcctggcggggaggagcctgcacagcctccgcatcgaggatgcgaccagcctggcgtggatggtggagatgaaggtgaaggcggtgcaggagaggatggggctggtgagggcacagatggcgtccagcagccagcaggtggtgctggaggcgccgatcgaggcaccggcaccgatggcggtgatgaaggagaagacgccgctggaggcggccatggaggcgctccagaggcagaactggctcatggaggtgatgaaccccaatgacaacttgatgtttggtggtggagaggagatggtgcagccctacatggaccataccaacaacccatggcttgacccctgctacttccctttgaactga

SEQ ID NO: 133 - DNA, 756 bp, Elaeis guineensis
  oil palm SEPALLATA (SEP)-like EG4N154153
atggcccgtaacaaggtgaagctcgcctggatcgccaacgacgctacccgccgcgcgaccctgaagaagagacgaaagggtctgctgaagaaggtgcaggagctgagcatcctgtgcggtgttgaagcatgcgcgatcgtgtacgggccgaacgaccgggtgccggaggtgtggccgtcgcccccggaggcggctcggatcgtggggcggttcaagagcatgccggagatggagcagacgcgcaagatggtcaaccaggaagggttcctccgccagcgcgccgtgaagctgttggagcagctccgcaagcaggagcgcgagaatagagagatggaaatgaagctgctgatccgcgaggggctcaagggacggagcttcgacaacctcggcatcgaggatgtcacctgcctctcctggatgcttgaacgaaaaataaaagaaatttatgataaaatggatgagataaagaataaggtgactgttaaccaagtcgccggcggcccgtcggcactgccactgcaggtcatggctcctcctcctgctgctccgatcgggccggtcgtgcccaaggagaagactacagtggagcaggcgatggaggccctccaaaggcagaactggttcatggatatgatgagtccatggcctgaggacttctaccagcctgctcagccgatggatccttaccagcctcctcctcctgcacctctggaccacaccatcccatggccggatccatcgttcccgttcaactga

SEQ ID NO: 134 - DNA, 756 bp, Elaeis guineensis; identical to SEQ ID NO: 133
  oil palm SEPALLATA (SEP)-like EG4N45603

SEQ ID NO: 135 - DNA, 570 bp, Elaeis guineensis
  oil palm SEPALLATA (SEP)-like EG4N140076
atggcccgtcgtcggcgtcgatggcagttcatagaaaaccagagacaacgtttggccacctacaggaagaggagaggaggcctcaggaagaaggccagccagctctcctccctctgcggcgtccccatcgccgtcatctctttcggtcccaacggccggctcgacacatggccggacgaccaaggagccatccacgacctcctcctcacctatcgaagcttcgaccccgagaagcggcggaagcacgacctcgacctaccgaccctcctcgaagcccaagaaggcagccaaaacctcctgtgggatcctcgcctcgacgccatgcccacggagtcccttcgaaacctcaccaactcactcgactccaaggtgaaggctatcgacgagagaatccaacagctgctcgaggaaaattccaagtgcagcaaccaagacaacaataattccagcagagaacaaggtgttaattccaagtgcaacgaccaggataacaataacaccggcagtgaacagcgtgatgattccaagagcagcaaccaagctaagcagataaaaagggtgagaaaataa

SEQ ID NO: 136 - DNA, 690 bp, Elaeis guineensis
  oil palm SEPALLATA (SEP)-like EG4N41944
atgggcaagatcgaaaagaaggaagcactccatatttgtttcaccaagcgccgccaggggatcttcaaaaaggccggagagctcgccgtcctctgcggtgcccagattaccgtcatcacactctctcctggtgggaagcccttctccttcggccaaccctccactgatgccgtcatcgcccgataccttgacccaggacgccaccaggtcccaatccccatcactacttcacttgagatccgactgagatattatctaaagtactgcaaactgggggagcagtccggcggtgggttatggtggtgggaagcgcccatagatgggctcgacctcgaagaacttgtggtgatgaaaggtgcaatagaggagctctacaaggccatcctgaagaaggccaaccagcctacgagtgcaggcgaagcagtacaaggcatgccacaaaaaccatcgctagcaatgctgaatggattagacagttgtgattggcttatccagcttttggccaactgctcccagtggttgcgtgatttgaaaagagtgtgtgggagtctgctgtcaatctttccgaatataacgatcaaagcggaagtcagaggaagtgtggatcgacggcttgccacgcatattattagagatgaggataaacagcaggtgcacaggtcgacagccatcatgaggatcaatgtttga

SEQ ID NO: 137 - DNA, 1389 bp, Elaeis guineensis
  oil palm SEPALLATA (SEP)-like EG4N3001
atgagaaggtctcaagtcaagcggatacttttaaaatgtcctgtaaagaaagctaaggagggcgaggagcctttggaggctgttgccaacaaaatctggcctaatgatgatctggagtttcaaagtggaaagtcgatgattcagaaagtgaaggggatgctgagggttagaagcatggatacggctatatattcttccaaagttatgtaccttccaaaaattactcttccttatcaaaaattcacaaacacttggtgcttggggtggtttggaccaattatccagcagctgccaatcggttcagcaccaggaacacttacttttgtgacttgtcgctcagagtcacaaacccatcctaggacttggttgaccaccagcccgacctgggacactagcatgaagtcagtgatagaacgctacaacaagaccaaagaggagaatcatctagttatgaatgcaagttcagagactaagcctatcaggttccgcctagcttcaactgccaaaagtcataattctgatggggcagatgaaaggggaaaggactcaaatttaatgcttgtagatgctcatgagcgacaagaattactgacagatttaggacggaatcaacctcacaaacatcacttctacagaaatagagaggcagatcacattcagcctcaaggtggagcagcaatttcctatgaggtgaaggatgtttttgtccaagaggatggaattttttggcaaagggaggcagcaagcttgaggcagcaactgcataacttgcaagaaagtcaccggcagttgttgggagaagagctttctggcctaagtgtgaaagatctacaaaatctagagaaccaacttgagatgagcttacgtggtatccgaatgaagaaggtttatgcaatgaggggtgtaaatggcattgataaaggtccgattactccatatggttttaatgtcaccgaggatgcaaacatatccattcatcttgaactcagccagccacaactgcaaacagatgcaacgcttgctcaaggccaaggaaacaaggaagttgaccaaggtcattctcatcaacctaccaatgaagatataatgccttccgggttcaccatagaatacgtgttggccattgaacaggtagtagcgggtgcccccactgctccctttccacgtggacagagaggcccgacgctggacccccgacgtgccaacttaggtcgtcgacacgtgggtgttgtcggcggtgggaacctctttgcgaagagatatgactttttggaagagaatgttggtttccgaagagttacaatcatatctcttcaaaaatatggcacttcgacagagtctataagtaggcttcgatccaatttgtttcaaaataataaaaaatcttaa

SEQ ID NO: 138 - DNA, 660 bp, Elaeis guineensis
  oil palm SEPALLATA (SEP)-like EG4N60802
atgacaaatcgtgggcgtggattgcagttgatagaaaatcggacacaatgtttggtcacctacaggaagaggagagaaagcctcaagaagaaggccaaccagctttcctccctctgtggcgtcctcatcgccgtcatctctttcgatcccgatggccggctccacacatggccagatgaccaaggagctctccccgacctcctcctcacctatcgaagcctcgaccccaagaagcggcagaaacacgacctcgacctaccgaccctcctcggtgccatgcccgcgggatcccttcgaacaggaccggctaaaggccatctctgccttcgaaagctcgccaactcactccactccaaggtggaggctatcgacgagagaatccaacaactgctcgacaagaattccaagtgcaccaaccaagacaataatagtaccagcagagaacaagacgatgattccaagtgtaacaagaaaggtaaaaataataataccagcagtgaaaaaggtgatgatgactccaagggcagcaaccaaggtaataataacaataataccagcagtgaacaaggtgattattccaagagtaacaacgagggtaatgataagaacaaggtttgcctccttgtagtaacccggtggtctttcatcccttccctataa

SEQ ID NO: 139 - DNA, 696 bp, Elaeis guineensis
  oil palm SEPALLATA (SEP)-like EG4N14015
atgtcgaggagcagcatgaagctcgagttgattgccgatgatgctgctcggaagacatccctgaagaagagaaagaagggcttgttgaagaaggtgcaggaactcagcatcctatgcgatgtcgatgcatgtgcgataatttacgagccagatgatcgccacccagagttatggccctcatccgaagaggctacccggatgctcgtgcggctccgaagcatgccagaaatggaacagaagcagaagatgatgaaccaagaggagttcctctaccagaagatgaggaaattggtagaccaacttcataagcaggagttcgagaataaggagctggagaagaagctaaagatgtatgaggcactgaggacgggggacttcagtgaattggacatggagcaagccatgaacctgtcgatgatgatcgagcagatgttgaagaaaatctatgagaagatggacgcgatcaagaagcatcaagcagcaatggcacgggttgacggagtagtgcaagagggtgggaatgcggctggactgaacactccgagggagaacaccccaacggagaaggataacgagatactccagaggcagaagcagatgctggatatgatgatcccgaggtcaagtaaaacctatcagccttctgcgggtccgaccaacccatggccggctaattccttgttccccttcaattga

SEQ ID NO: 140 - DNA, 738 bp, Elaeis guineensis
  oil palm SEPALLATA (SEP)-like EG4N21371
atgacgaatccggacgatggagaggtgggcggaggaggaggaagcgagcgatgtgtagcatcagagaaagttacagggaagaaggctaggagagctacatttaagaagagaaagaagggtttgatgaagaaggtaagtgaattgagcactttatgtgatgtcaaagcatgtttgattgtctatgggccaaatgaaccagaagcggaggtatggccatcagtgccagatgctatgcgtgtgcttacaaagctaaagaaaatgcccgagatggagcaaagcaaaaaaatgatgaaccaagaaggcttcatgcgtcagaggatcatgaagctacaagaacaactcaggaagcaagatagagagaacagagagctcgagacaatcctattgatgtatcaaggcttggcagggaggagcttacacaccgtgactattgaagatatgacaagcctcgcatggcttattgagatgaaggtaaataaagtacaagagaggatagagcattcaaaaggagagatcgcatcaaagatggtggaggggatgaaagaggagaagaagaaagtcgaagggccatcaaatatcaaagaaaaaatatctttggaggttgccatggaggaacttcagaggcaagaatggttcactgaaataatgaatccacatgacctaatgatttgtggaaatgaagtcgtgcaaccctacatagatcataataacccatggttggatgcttactttccttga

SEQ ID NO: 141 - DNA, 753 bp, Elaeis guineensis
  oil palm SEPALLATA (SEP)-like EG4N122402
atgggtcgccacaagatccccgtcaagatgatcgacaaaaaagacgagagcaacatctgcttctcgaagcaaaagaagggtctcttctccaaggcgaagcaaatcgctcgtgcaggcagtgaagtcgccatcatcgtcttctcccgtgtcggtaacatattcactttctgccaccctagcatagaatctgttgctagtcgcttcctcagccagcaaaacatcaaacacagatcatccaatgatgataattttcatggcaatgccgacttcgtgtatccggggtccgacgctgcaagaggaggtcttaccggaccatccgaagaaggtgaaacatcaaataaaggagataataaattagatggaggaaacaccatcatgcaggataaggggttcgagtctgaccatgaagaagaagaagtggaaagtaagaccagctcgaaggctgaagggtcggacgtcgccggcagttcgcaagaggaacatgcattgatgcatgatggagaagaacatgcaacaggagaaaaagagacttcttctgacgagacactgcatagcggtcgattttggtggaacaaccgaattgataatcgtgagttacatgagctgttagagtttgagagcgcgctcgtggagctgcgggagaaggtgcgagaccaagcaaatcagatcctggttcagaaaccagtgatgggatattatttagattttagtaattacaagttcaagtttgatgagcaggcgtcacaggattag

SEQ ID NO: 142 - DNA, 609 bp, Elaeis guineensis
  oil palm SEPALLATA (SEP)-like EG4N42750
atggtcccgagggcagagctgtgggcagtgtgggctggtattgcctatgcgaggctggctcttacagtagaccgactcatcattgagggtgactcaggcactatggttaaatggattcaaatgcgggatacagaggatgctgctcacccacttctgagggatatcgcgatgctgctgaggggggccaccatcactgcagtcacaatccggatggaaaatctctcaataagagcatcctcgttcagtctaacaaatggtcgatctgagctctctggactagtctgtggaggggtgccaaaaattcagtcttctatcttcactgagagagtcagctcttgcatctcaagagtcgactcgccattcgtgccagtgtgttccaatgtgccagagaaattgatgggcgaacagttgtctggcttaaatgtcaaagaactgcaaaatctagagatccaacttgaaaggagtcttcattgtgtccaaaagaagaaggggtaccttcttcacaatgaaaatattgaactctacaagaaggtaaaccttatacgtcaagaaaacatggagttgcgtaagaagcctcgcaatatactcagtcgcactgacaaagcatag

SEQ ID NO: 143 - DNA, 936 bp, Elaeis guineensis
  oil palm SEPALLATA (SEP)-like EG4N157194
atgaacggcgagaacgacgctgctagcaggatcatcttttcttctctgaaagaacggctggtacaatccggtgtttcctatgcaaaagcggtcaaaaagcaccccatcccatccccagtggtcaggaaatctaccgaaacagtcaaggatctcatgagttccaattcaggaaatgtacatcatcatccccgttctcgagggcaccgggtgaagctcttgagtaaaggaacttgttttcgctgtggagatcgtgatcacacccgagaatcttgcagaaatccgattaaatgctttctttgcaagggttatgggcatgttcaaaagggtttcgccactcttagcaccaagatagaaactggggccacctcctgcccggtttcccttgtggtgctagagtctaaaacctctctccctctctccctttgtcgtttcctccggggcccttattggaaagtaatattgggttacattgctcgtgacacatctgagcttagttatgatgattgctttgaacggagagagagaacttttggctggcgtggattgttttttggaccgagcgccatcacgtcgctttcaagcttgtggtgtcgtctgcccatttgtaatctccgaaggccgtaccttgtcttgttttcctttcgccagaaccttaacctcgtcgataagcacttaatgggtgattctctgggttctttgactgtgaaggagcttaagcaactcgaaaaccgacttgaaagaggcatcacaaggatcagatcaaagaagattgcagagactgagcgagcacagcaagtaagcatcattgaagcaggacatgagtttgatgctcttccaggatttgattctaggaactactaccatgtcagtatgttggaggcagcaccccactactcacaccaacaagatcagacagcccttcatctcggtatataa

SEQ ID NO: 144 - DNA, 489 bp, Elaeis guineensis
  oil palm SEPALLATA (SEP)-like EG4N6887
atgggtctacgaaacaagccaccaaatcaaaggagatatgggatatcttacgagagaaatttcaagggaataccaaggaatttgatgggagagtctcttggctctatgagccctagggacctgaagcaactggagggtaggttggaaaagggcataaacaaaataaggacaaaaaagattgctgagaatgagagagcacagcaacagatgaatatgttaccccagacaactgaatatgaggtcatggctccgtacgattcaaggaacttccttcaagtgaatctcatgcaaagcaatcagcattactctcatcagcagcagacgactctccaactaggaaagaagatcgtagatcgggtggctagttcaactgacagatcggatgttgggataattcaggatcttcctaaccaaaggggaccagaggggcgtcgcccgtggtccgacgggctacagcagcatggtcgctggttcggcagtggtgactga

SEQ ID NO: 145 - DNA, 183 bp, Elaeis guineensis
  oil palm SEPALLATA (SEP)-like EG4N91665
atgagcatcgtcgataactctgatatgtcgatggcatcgtgtcgattgcaattgatagaaagccggagacaacgtttggccacctacaggaagaggagggaaagcctcaagaagaaggccaaccagctctcctccctctgcggcgtccccatcgccgtcatctctttcggtcccaatggttga

SEQ ID NO: 146 - DNA, 675 bp, Elaeis guineensis
  oil palm SEPALLATA (SEP)-like EG4N126213
atggaagtcctcccgatcattgacctccacccgactgttatcttgggatcagttcttgaattgccccagcgagaaggaaagccccaaagaagaatagaagaagcaaaaaagaactggttcttccacccatggatggatgatagaagatcgaggagagctcttctctttccgcttcgagatgccaatgacccaacaccagcacacgacagtgacctctcgcagcaggggctgtggcaacctcctacggcaaccccatcacagccacgttcagtgacagatatttggttgtgcaagtggattgaaagtgactttcggaactcgtttggttcatgggaagaacttttcttcctaaaaattaactttcaaccagttttttccaggcacttgatgggtgatgctctgagttctttgagtgtgaaggaacttaagcaacttgaaaaccgacttgaaagaggcatcacaaggatcagatcaaagaagattgcagagaatgagcaagcagcactgcaggtaagcattgcacaagaaggacctcagtttgatgctcttccagcatttgattctagaaactactaccatgtcaatctgttggaggctgcaacccattactcccaccaacaagatcaaacagctctccatcttgggtatgaagcaagatctgatcatgctgcatag

SEQ ID NO: 147 - DNA, 249 bp, Elaeis guineensis
  oil palm SEPALLATA (SEP)-like EG4N36286
atgccacggaggaaggtcgtgttagagccccaccccaccgagcaagctcggatgcagtgctacttgactcgaaggaatggtattaagaagaaggtgagggagctctccatcctctgcgatgccgatattgcccacctctccatccctcctgcaggagagccttcgctgttcctcggcgcccacacgtcatgtggaggccttgtggtgctcgctggctcggtgtactccaccatagccttgcacccctag

SEQ ID NO: 148 - DNA, 759 bp, Elaeis guineensis
  oil palm SEPALLATA (SEP)-like EG4N3542
atggctcctcctctcggaagcggcgccgccacctccggcggcaacggcgacggtcgcggcgagagataccggtggaaatccatcgagaagcggacgtggggcctctgcaagaaagcgtacgagctcgccaccctctgcgacgtcgacgtcgccctcatctgctacctccccagcgtcgacacgcccaccatctggccgccgtaccgccataaagtcgaacaagtcgtccaccgctacgtcgacatccccgccgacaagaagctccccaagaaccagatcaccctccacatccccaactccacggccgggaacacgaaggacgcaggcgaggcggcggcagtggcggacgccgaccgcatccgtgtccccttcccctacgatgaagacaagctgatagctatcgtgaggtatttggattcgaagatcgtggaggtgcggaggatgatcgcggcccgtcggatggagcggaggagcgagccggcgctggcggtggcgagcggcggtgatggggatcctgggacggccgattgggatagggggaagagggtagcccgggattgcggtccggtttggggacgggggcgtccggatttctcggctctggcggcggcggcggcggcggcggcgaggggcggtggcagcgggggagcaccgaattcttcgcgctcctgcctgtgctgttactgcccccatcacgggcactggttcactggattcgacggtagaaatgcttcgagagatggatcggacggcatttga

SEQ ID NO: 149 - DNA, 1842 bp, Elaeis guineensis
  oil palm SEPALLATA (SEP)-like EG4N71936
atggctcctccccgaggcgacggtcgaagcgataaatccctccgcctatccatcaagaatcggacgaagggcctctgcaagaaggcgtacgagctcgccactctctgcgacgtcgagctcgccctcgtctcctacccctccgacggcgccgaacccaccacatggccgcccgaccgatccaagatcgaagacgccttccaccgctacttcgaaacccccgcccacaagaagctccccaagaaccagatcaccctcgacaaccccaaccccggtgccgtcgagaagaaagacgccgccaaagcggccgcgtcgaaggcgccgaaggagaccgaccgcctccgcatcccctttcctgacgacgaggacaagctgatagcgctgcgagggatcttggattcgaggctcgaggcggtgcggaagatgatcgcgatccgtcgggcggaggagaggagggatccgagaccgtccgctcgggatacggagaaggagcttgccgtcgcagtggcgaatgccggtggtggtgatccgacgccgtccgctggagatccggggaaaaggcttgcccagggtcaaggtgggccgctgccagcagcggcggcggtcgcggcggcgagcgccggtcgagaggatccgcggccgtccgttcgagatgtggagaagatggtggccggggattgcggtccggtttctggacgggggaatccggattgctcggccgcgccggctgcggcgggcagcggaggcggcggggcaccaaattcttggcttcaaccatctgctcatggtggaagaagccattggagctacaggctccaaaccgaacccaccttctcaccccagaaagaagccgccggaaacggaagatacccccccggaacgcgggaatcagtggcatatcccgtaattcaacccaaactccagtggcattcttcttccctggccccacctcaacgtcacctcttgcgtgaagcggcgtcaccgatcacgccccccttcacggtgacgtggcaccggcggcggtttacccatttcctgcgccgccggaacgccacttatgataccgtgcatgggaagtggaagcaccacgatatcaaggtcaaggactcgaagacccttctctttggcgagaagcaagtcactgtctttggcattaggaaccctgaggagatcccatggggtgaaactggtgcagagtatgttgtggagtctactggtgtctttactgacaaggagaaggcttctgctcacctgaagggtggtgccaagaaggtcatcatctctgctgctagcaaagatgttcctatgtttgtggtgggtgtgaacgagcatgaatacaagtctgacattgatatcgtctccaatgctagctgcaccacaaactgtctagctgttctggccaaggtcatcaatgataaatttggcatcattgagggtttgatgagcacagtgcattccatcactgctactcagaagactgttgatgggccatccagcaaggactggaggggtggacgagctgccagctttaacatcattcctagcagcactggtgctgccaaggttggaaggagttttggggtacttaccactacgtacaaggatgccgctgaggataaggccgaccgatgccgaaatcagacagtacgcggcgaggaagaggccgacgtctgggaccggaccctcacgaccgccgaagaaaccctcaacagcagtgccgaccgtcgtcgcatcggcggccgatcagtcggagccggtaattgcactttcggctccgacagcgcctccggaagagcggccagcggaggaagtggccgaaggaacatcggtgatttcaccgattga

SEQ ID NO: 150 - DNA, 219 bp, Elaeis guineensis
  oil palm SEPALLATA (SEP)-like EG4N29531
atggaaggggtggaaaaaattgaggaaataattgctcgtgagctaaatatgatgaagacactcgaaaggtaccaaaaatgtaactatggtgctccggagactaatattatatcaagagagactcaggaagatgtggatgctttgtatggccaagtttgtgatatttttcttaaatatcctaacgaactagcagttgaatggtctgaaggtctagattag

SEQ ID NO: 151 - DNA, 1191 bp, Elaeis guineensis
  oil palm SEPALLATA (SEP)-like EG4N44436
atgcgggaggcgatcgggggctcgcagccaagggctcagggaggcgagaggcggtcaagggatcgaggagatgggaggcgatcgagggctaggggaggcagattggggggtcagggaggtaggaggcaggcaggggctcgcggtcgggagctcgaggaggtgggaggcagccaggggctcgaggaggcaagccgggggcttagagaggcggaaggcggtcgggggctcacagtcgggggctcggagagtcgggagacagcctggatcttagggaggcggtcggatgctcatagtcgagggcttgaggaggtcagagacggtcggatgcttacgatcgggggctcgaggaggcggaggcagaggaaagagggggtggggaaaaataagggggggtggcagggcacgggactgggactctcctcaaccgctataaataaagcaagctacccctcacaagaaccagaagcttggagcaaaccaatggttggtaaaaaattgaacgtagaattcataaaacaccggaaaaagcgtttggccacctaccggaggaggaaagaagccctcaagcaggcggcctacgagctctcgacgctctgcggcacccccaccgccgtcatatacttcggtcccgatggccagcccgaatcatggccggaggacgaaggagccgtccgcgacatcatcggaaggcatccaggcctcggcgcaaagaagcggagcacgcgtcccttcgacttacgggatcttcctccgtttgacgacacgtcggaggagtttttgagagagatgctttgttcaatggagtcgggtatggaggctgtcaaggagaggatccaacttctcaaaaaggattccaggtgcaaccaaggcgacttccatggtgatactggcggtgtacaacaacaaggttgccaatgtaataatcctgctttcatggaggagtgctttgatgtgccaatggtgtccaaggcagccatggatgatggaccaggccaaggccatggtgctttcgcgccgatggagctaaaacaagtggaaggagttgctgccgatgctttcttgccatgttcttctaatgcatcgatggacttcaatgatgaactggcggcgttctccatgccgttaattttcatgccaccaccattcaccggagctacttcagagcatgacattgcatgcatctggcagtga

SEQ ID NO: 152 - DNA, 733 bp, Elaeis guineensis
  oil palm MADS-box type transcription factor SHELL DeliDura allele, ShDeliDura, Sh+, EG4N37875
atgggtagaggaaagattgagatcaagaggatcgagaacaccacaagccggcaggtcactttctgcaaacgccgaaatggactgctgaagaaagcttatgagttgtctgtcctttgtgatgctgaggttgcccttattgtcttctccagccggggccgcctctatgagtacgccaataacagcataagatcaacaattgataggtacaagaaggcatgtgccaacagttcaaactcaggtgccaccatagagattaattctcaacaatactatcagcaggaatcagcaaagttgcgccaccagatacagattttacaaaatgcaaacaggcacttaatgggtgaagctttgagcactctgactgtaaaggagctcaagcaactcgaaaacagacttgaaagaggtatcacacggatcagatcgaagaagcatgagctgttgtttgcagagatcgagtatatgcagaaaagggaagtagaactccaaaatgacaatatgtacctcagagctaagatagcagagaatgagcgagcacagcaagcaggtattgtgccggcagggcctgattttgatgctcttccaacgtttgataccagaaactattaccatgtcaatatgctggaggcagcacaacactattcacaccatcaagaccagacaacccttcatcttggatatgaaatgaaagctgatccagctgcaaaaaatttactttaagtatgtcgctgcttgt

SEQ ID NO: 153 - DNA, 733 bp, Elaeis guineensis
  oil palm MADS-box type transcription factor SHELL MPOB allele, shMPOB, sh-
atgggtagaggaaagattgagatcaagaggatcgagaacaccacaagccggcaggtcactttctgcaaacgccgaaatggactgccgaagaaagcttatgagttgtctgtcctttgtgatgctgaggttgcccttattgtcttctccagccggggccgcctctatgagtacgccaataacagcataagatcaacaattgataggtacaagaaggcatgtgccaacagttcaaactcaggtgccaccatagagattaattctcaacaatactatcagcaggaatcagcaaagttgcgccaccagatacagattttacaaaatgcaaacaggcacttaatgggtgaagctttgagcactctgactgtaaaggagctcaagcaactcgaaaacagacttgaaagaggtatcacacggatcagatcgaagaagcatgagctgttgtttgcagagatcgagtatatgcagaaaagggaagtagaactccaaaatgacaatatgtacctcagagctaagatagcagagaatgagcgagcacagcaagcaggtattgtgccggcagggcctgattttgatgctcttccaacgtttgataccagaaactattaccatgtcaatatgctggaggcagcacaacactattcacaccatcaagaccagacaacccttcatcttggatatgaaatgaaagctgatccagctgcaaaaaatttactttaagtatgtcgctgcttgt

SEQ ID NO: 154 - DNA, 733 bp, Elaeis guineensis
  oil palm MADS-box type transcription factor SHELL AVROS allele, shAVROS, sh-
atgggtagaggaaagattgagatcaagaggatcgagaacaccacaagccggcaggtcactttctgcaaacgccgaaatggactgctgaagaatgcttatgagttgtctgtcctttgtgatgctgaggttgcccttattgtcttctccagccggggccgcctctatgagtacgccaataacagcataagatcaacaattgataggtacaagaaggcatgtgccaacagttcaaactcaggtgccaccatagagattaattctcaacaatactatcagcaggaatcagcaaagttgcgccaccagatacagattttacaaaatgcaaacaggcacttaatgggtgaagctttgagcactctgactgtaaaggagctcaagcaactcgaaaacagacttgaaagaggtatcacacggatcagatcgaagaagcatgagctgttgtttgcagagatcgagtatatgcagaaaagggaagtagaactccaaaatgacaatatgtacctcagagctaagatagcagagaatgagcgagcacagcaagcaggtattgtgccggcagggcctgattttgatgctcttccaacgtttgataccagaaactattaccatgtcaatatgctggaggcagcacaacactattcacaccatcaagaccagacaacccttcatcttggatatgaaatgaaagctgatccagctgcaaaaaatttactttaagtatgtcgctgcttgt

SEQ ID NO: 155 - protein, 174 aa, Artificial Sequence
  synthetic Elaeis guineensis oil palm SHELL Sh-DeliDura allele MADS-box, I and K domains
GRGKIEIKRIENTTSRQVTFCKRRNGLLKKAYELSVLCDAEVALIVFSSRGRLYEYANNSIRSTIDRYKKACANSSNSGATIEINSQQYYQQESAKLRHQIQILQNANRHLMGEALSTLTVKELKQLENRLERGITRIRSKKHELLFAEIEYMQKREVELQNDNMYLRAKIAEN

SEQ ID NO: 156 - protein, 176 aa, Artificial Sequence
  synthetic Oryza sativa rice OsMADS24 MADS-box, I and K domains
GRGRVELKRIENKINRQVTFAKRRNGLLKKAYELSVLCDAEVALIIFSNRGKLYEFCSGQSMTRTLERYQKFSYGGPDTAIQNKENELVQSSRNEYLKLKARVENLQRTQRNLLGEDLGTLGIKELEQLEKQLDSSLRHIRSTRTQHMLDQLTDLQRREQMLCEANKCLRRKLEES

SEQ ID NO: 157 - protein, 177 aa, Oryza sativa
  rice SEPALLATA (SEP)-like OsMADS13
MGRGRIEIKRIENTTSRQVTFCKRRNGLLKKAYELSVLCDAEVALIVFSSRGRLYEYSNNNNVKATIDRYKKAHACGSTSGAPLIEVNAQQYYQQESAKLRHQIQMLQNTNKHLVGDNVSNLSLKELKQLESRLEKGISKIRARKNELLASEINYMAKREIELQNDNMDLRTKIAEE

SEQ ID NO: 158 - protein, 167 aa, Oryza sativa
  rice SEPALLATA (SEP)-like OsMADS2
MGRGKIEIKRIENSTNRQVTFSKRRSGILKKAREISVLCDAEVGVVIFSSAGKLYDYCSPKTSLSRILEKYQTNSGKILWDEKHKSLSAEIDRIKKENDNMQIELRHLKGEDLNSLQPKELIMIEEALDNGIVNVNDKLMDHWERHVRTDKMLEDENKLLAFKLHQQ

SEQ ID NO: 159 - protein, 175 aa, Oryza sativa
  rice SEPALLATA (SEP)-like OsMADS3
MGRGKIEIKRIENTTNRQVTFCKRRNGLLKKAYELSVLCDAEVALIVFSSRGRLYEYANNSVKSTVERYKKANSDTSNSGTVAEVNAQHYQQESSKLRQQISSLQNANSRTIVGDSINTMSLRDLKQVENRLEKGIAKIRARKNELLYAEVEYMQKREVELQNDNMYLRSKVVEN

SEQ ID NO: 160 - protein, 174 aa, Elaeis guineensis
  oil palm SEPALLATA (SEP)-like EgAGL21
MGRGRVELKRIENKINRQVTFAKRRNGLLKKAYELSVLCDAEVALIIFSNRGKLYEFCSSSSMMKTLERYQKCNYGAPETNIISRETQSSQQEYLKLKARAEALQRSQRNLLGEDLGPLSSKELEQLERQLDASLKQIRSTRTQYMLDQLADLQRKEQMLCEANKSLRRRLEES

SEQ ID NO: 161 - protein, 173 aa, Elaeis guineensis
  oil palm SEPALLATA (SEP)-like EgAGL61
MGRGRVELKRIENKINRQVTFSKRRSGLLKKAYELSVLCDAEIALIIFSSRGKLYEFGSVGTSKTLERYQRCCYTSQDSNFADQETQNWYQEMAKLKAKFESLQRSQRHLLGEDLGPLTVKELQQLERQLESALSQARQRKAQIMLDQMEELRKKERHLGEINKQLKDRLDAE

SEQ ID NO: 162 - protein, 174 aa, Oryza sativa
  rice SEPALLATA (SEP)-like OsMADS33
MVRGKVQMRRIENPVHRQVTFCKRRGGLLKKARELSVLCDADVGVIIFSSQGKLHELATNGNMHNLVERYQSNVAGGQMEPGALQRQQVAEQGIFLLREEIDLLQRGLRSTYGGGAGEMTLDKLHALEKGLELWIYQIRTTKMQMMQQEIQFLRNKEGILKEANEMLQEKVKEQ

SEQ ID NO: 163 - protein, 176 aa, Oryza sativa
  rice SEPALLATA (SEP)-like OsMADS5
MGRGKVELKRIENKISRQVTFAKRRNGLLKKAYELSVLCDAEVALIIFSTRGRLFEFSTSSCMYKTLERYRSCNYNLNSCEASAALETELSNYQEYLKLKTRVEFLQTTQRNLLGEDLVPLSLKELEQLENQIEISLMNIRSSKNQQLLDQVFELKRKEQQLQDANKDLKRKIQET

SEQ ID NO: 164 - protein, 177 aa, Oryza sativa
  rice SEPALLATA (SEP)-like OsMADS7
MGRGRVELKRIENKINRQVTFAKRRNGLLKKAYELSVLCDAEVALIIFSNRGKLYEFCSTQSMTKTLEKYQKCSYAGPETAVQNRESEQLKASRNEYLKLKARVENLQRTQRNLLGEDLDSLGIKELESLEKQLDSSLKHVRTTRTKHLVDQLTELQRKEQMVSEANRCLRRKLEES

SEQ ID NO: 165 - protein, 173 aa, Oryza sativa
  rice SEPALLATA (SEP)-like OsMADS26
MARGKVQLRRIENPVHRQVTFCKRRAGLLKKARELSILCEADIGIIIFSAHGKLYDLATTGTMEELIERYKSASGEQANACGDQRMDPKQEAMVLKQEINLLQKGLRYIYGNRANEHMTVEELNALERYLEIWMYNIRSAKMQIMIQEIQALKSKEGMLKAANEILQEKIVEQ

SEQ ID NO: 166 - protein, 175 aa, Elaeis guineensis
  oil palm SEPALLATA (SEP)-like EgSQUA3
MGRGRVQLKRIENEINRQVTFSKRRSGLLKKAHEISVLCDAEVAVVVFSTKGKLYEYSTDSSMEKILERYRQYSNAEKALAQGDPGPQGSWLHEFGELKSKVEALQKCQRHLMGEQLDSLALKELQQLEQRLESALRHIRSRKNQLLFDSIAELRRKEKSLQEQNCILEKRLVES

SEQ ID NO: 167 - protein, 175 aa, Oryza sativa
  rice SEPALLATA (SEP)-like OsMADS47
TGKRERIAIRRIDNLAARQVTFSKRRRGLFKKAEELSILCDAEVGLVVFSATGKLFQFASTSMEQIIDRYNSHSKTLQRAEPSQLDLQGEDSSTCARLKEELAETSLRLRQMRGEELHRLNVEQLQELEKSLESGLGSVLKTKSKKILDEIDGLERKRMQLIEENLRLKEQLQVS

SEQ ID NO: 168 - protein, 174 aa, Elaeis guineensis
  oil palm SEPALLATA (SEP)-like EgMADS12
  VARIANT (62)...(126): Xaa = any amino acid
MGRGKIEIKRIENTTNRQVTFCKRRNGLLKKAYELSVLCDAEVALIIFSSRGRLYEYANNSXKATIERYKKHVLDTSNSGSVSEADSQYYQQESLKLRQQITSLQNSNRNLMGDSLGSMSLRDLKXLEGRLEKGINKIRTKKNELLFAEIEYMQKRETELQNANMYLRNKIAEN

SEQ ID NO: 169 - protein, 151 aa, Elaeis guineensis
  oil palm SEPALLATA (SEP)-like EgSQUA2
MGRGRVQLKRIENKINRQVTFSKRRSGLLKKAHEISVLCDAEVALIVFSTKGKLYEYSTNASMEKILERYQCYSYAERALVEEDPKSQGRWCHDYGELRSKVEALQKSQRHLMGEQLESLTFKELQQLELHLDGALRHIRSRRVNQSFSFH

SEQ ID NO: 170 - protein, 173 aa, Oryza sativa
  rice SEPALLATA (SEP)-like OsMADS25
MGRGKIAIKRIDNTMNRQVTFSKRRGGLMKKARELAILCDADVGLIVFSCTGRLYDFSSSSMKSIIERYQEAGEEHCRLLNPMSEAKFWQREVTTLRQQVQNLHHNNRQLLGEEISNFTVRDLQLLQNQVEMSLHSIRNKKDQLLAEEILKLNEKGSLVQKENSELRKKFNIA

SEQ ID NO: 171 - protein, 159 aa, Oryza sativa
  rice SEPALLATA (SEP)-like OsMADS23
MGRGKIEIKRIDNATSRQVTFSKRRSGLFKKARELSILCDAEVGLLVFSSTSRLYDFASSSMKSIIERYNETKEDPHQTMNASSEAKLWQQEAASLRQQLHNLQEYHRQLLGQQLSGLDVEDLQNLESKLEMSLKNIRLRKDNVMMDQIQELSRKVVTT

SEQ ID NO: 172 - protein, 174 aa, Oryza sativa
  rice SEPALLATA (SEP)-like OsMADS30
MGQGKIEMKRIEDATRRQVTFSKRRAGFLKKANELAVLCDAQVGVVVFSDKGKLFDFCSPPVILMELFHRYEITTRNTRLQETNRDDEQMVMEITRLRNEIDQLEASLRRQTGEDLSSVSTVDELSQLQLQLESSLSKVHARKDELMSQQLEDMRRMHQTVHEQNNFLCRMVTK

SEQ ID NO: 173 - protein, 175 aa, Oryza sativa
  rice SEPALLATA (SEP)-like OsMADS58
IGSRGKIEIKRIENTTNRQVTFCKRRSGLLKKAYELSVLCDAEVALVVFSSRGRLYEYSNNSVKETIERYKKANSDTSNASTVAEINAQHYQQEAAKLKQQITNLQNSNRTLVGDNITTMNHRELKQLEGRLDKGLGKIRARKNELLCAEIEYMQRRETELQNDNMYLKSKVAES

SEQ ID NO: 174 - protein, 172 aa, Oryza sativa
  rice SEPALLATA (SEP)-like OsMADS57
MGRGKIVIRRIDNSTSRQVTFSKRRNGLLKKAKELSILCDAEVGLVVFSSTGRLYEFSSTNMKTVIDRYTNAKEELLGGNATSEIKIWQREAASLRQQLHNLQESHKQLMGEELSGLGVRDLQGLENRLEISLRNIRMRKDNLLKSEIEELHVKGSLIHQENIELSRSLNVM

SEQ ID NO: 175 - protein, 175 aa, Oryza sativa
  rice SEPALLATA (SEP)-like OsMADS15
MGRGKVQLKRIENKINRQVTFSKRRNGLLKKAHEISVLCDAEVAAIVFSPKGKLYEYATDSRMDKILERYERYSYAEKALISAESESEGNWCHEYRKLKAKIETIQKCHKHLMGEDLESLNLKELQQLEQQLESSLKHIISRKSHLMLESISELQKKERSLQEENKALQKELVER

SEQ ID NO: 176 - protein, 168 aa, Oryza sativa
  rice SEPALLATA (SEP)-like OsMADS31
MGRGRVELKKIENPTNRQVTFSKRRMGLLKKANELAILCDAQIGVIVFSGTGKMYEYSSPPWRIANIFDRYLKAPSTRFEEMDVQQRIIQEMTRMKDENNRLRIIMRQYMGDDLASLTLQDVSNLEQQIEFSLYKVRLRKQQLLDQQLLEMHSRVCNKRIYRFSILPY

SEQ ID NO: 177 - protein, 173 aa, Oryza sativa
  rice SEPALLATA (SEP)-like OsMADS27
MGRGKIVIRRIDNSTSRQVTFSKRRNGIFKKAKELAILCDAEVGLMIFSSTGRLYEYSSTSMKSVIDRYGKSKDEQQAVANPNSELKFWQREAASLRQQLHNLQENHRQLMGEDLSGLNVKELQSLENQLEISLRSVRTKKDHVLIDEIHELNRKGSLVHQENMELYKKISLI

SEQ ID NO: 178 - protein, 177 aa, Oryza sativa
  rice SEPALLATA (SEP)-like OsMADS24
MGRGRVELKRIENKINRQVTFAKRRNGLLKKAYELSVLCDAEVALIIFSNRGKLYEFCSGQSMTRTLERYQKFSYGGPDTAIQNKENELVQSSRNEYLKLKARVENLQRTQRNLLGEDLGTLGIKELEQLEKQLDSSLRHIRSTRTQHMLDQLTDLQRREQMLCEANKCLRRKLEES

SEQ ID NO: 179 - protein, 175 aa, Elaeis guineensis
  oil palm SEPALLATA (SEP)-like Eg_SHELL
MGRGKIEIKRIENTTSRQVTFCKRRNGLLKNAYELSVLCDAEVALIVFSSRGRLYEYANNSIRSTIDRYKKACANSSNSGATIEINSQQYYQQESAKLRHQIQILQNANRHLMGEALSTLTVKELKQLENRLERGITRIRSKKHELLFAEIEYMQKREVELQNDNMYLRAKIAEN

SEQ ID NO: 180 - protein, 173 aa, Oryza sativa
  rice SEPALLATA (SEP)-like OsMADS50
MVRGKTQMKRIENPTSRQVTFSKRRNGLLKKAFELSVLCDAEVALIVFSPRGKLYEFASASTQKTIERYRTYTKENIGNKTVQQDIEQVKADADGLAKKLEALETYKRKLLGEKLDECSIEELHSLEVKLERSLISIRGRKTKLLEEQVAKLREKEMKLRKDNEELREKCKNQ

SEQ ID NO: 181 - protein, 172 aa, Oryza sativa
  rice SEPALLATA (SEP)-like OsMADS29
MGRGKIEIKRIENATNRQVTFSKRRGGLLKKANELAVLCDARVGVVIFSSTGKMFEYCSPTCSLRELIEHYQTVTNTHFEEINHDQQIFVEMTRMRNEMEKLDGGIRRFTGDDLSNLTLADINDLEQQLEFSVTKVRARKHQLLNQQLDNLRRKEHILEDQNSFLCRMINEN

SEQ ID NO: 182 - protein, 171 aa, Oryza sativa
  rice SEPALLATA (SEP)-like OsMADS16
MGRGKIEIKRIENATNRQVTYSKRRTGIMKKARELTVLCDAQVAIIMFSSTGKYHEFCSPSTDIKGIFDRYQQAIGTSLWIEQYENMQRTLSHLKDINRNLRTEIRQRMGEDLDGLEFDELRGLEQNVDAALKEVRHRKYHVITTQTETYKKKVKHSYEAYETLQQELGLR

SEQ ID NO: 183 - protein, 176 aa, Oryza sativa
  rice SEPALLATA (SEP)-like OsMADS21
MGRGKIEIKRIENKTSRQVTFCKRRNGLLKKAYELAILCDAEIALIVFSSRGRLYEFSNVNSTRSTIERYKKASASTSGSAPVIDVNSHQYFQQEAAKMRHQIQTLQNANRHLIGESIGNMTAKELKSLENRLEKGISRIRSKKHELLFSEIEYMQKREADLQNENMFLRAKVAEA

SEQ ID NO: 184 - protein, 171 aa, Oryza sativa
  rice SEPALLATA (SEP)-like OsMADS22
MARERREIKRIESAAARQVTFSKRRRGLFKKAEELSVLCDADVALIVFSSTGKLSHFASSSMNEIIDKYNTHSNNLGKAEQPSLDLNLEHSKYAHLNEQLAEASLRLRQMRGEELEGLSIDELQQLEKNLEAGLHRVMLTKDQQFMEQISELQRKSSQLAEENMQLRNQVS

SEQ ID NO: 185 - protein, 175 aa, Elaeis guineensis
  oil palm SEPALLATA (SEP)-like EgFULlike
MGRGRVQLRRIENKINRQVTFSKRRSGLPKKAHEISVLCDAEVALIIFSTKGKLYEYATDSCMERILERYERYTYAEKALISSGPELQGNWCHEFGKLKAKVEALQKSQRHLMGEQLEPLNLKELQQLEQQLESSLKHIRTRKCQLMFESISELQKKEKSLQGAEQDAGEGAHGR

SEQ ID NO: 186 - protein, 194 aa, Oryza sativa
  rice SEPALLATA (SEP)-like OsMADS55
MARERREIRRIESAAARQVTFSKRRRGLFKKAEELAVLCDADVALVVFSSTGKLSQFASSNMNEIIDKYTTHSKNLGKTDKQPSIDLNFFLIILLRTYTNSYAYIHLLLQLEHSKCSSLNEQLAEASLQLRQMRGEELEGLSVEELQQMEKNLEAGLQRVLCTKDQQFMQEISELQRKGIQLAEENMRLRDQMP

SEQ ID NO: 187 - protein, 178 aa, Oryza sativa
  rice SEPALLATA (SEP)-like OsMADS20
MGRGKVQVRRIENEVSRQVTFSKRRPGLLKKAHEIAVLCDVDVAAIVFSAKGNLFHYASSHTTMERILEKYDRHELLSEGNNVIEEFPELEGSMSYDHIKLRGRIEALKKSQRNLMGQELDSLTLQDIQQLENQIDTSLNNIRSRKEKLLMEKNTILEKKITELETLHTCIRASPTKA

SEQ ID NO: 188 - protein, 167 aa, Elaeis guineensis
  oil palm SEPALLATA (SEP)-like EgGLO1
MGRGKIEIKRIENSTNRQVTFSKRRNGIIKKAREISVLCDAQVSVVIFSSSGKMSEYCSPSTTLSRILERYQHNSGKKLWDAKHESLSAEIDRIKKENDNMQIELRHLKGEDLNSLSPKELIPIEDALQNGLISVRDKQMEFLKKLKKNERLLEEENKHLTYLLHQQ

SEQ ID NO: 189 - protein, 167 aa, Elaeis guineensis
  oil palm SEPALLATA (SEP)-like EgGLO2
MGRGKIEIKRIENSTNRQVTFSKRRNGIIKKAREISVLCDAQVSLVIFSSSGKMSEYCSPSTTLSRLLEKYQVNSGKKLWDVKHENLSVEIDRIKKENDNMQIELRHLKGGDLNSLNPKELILIEDVLQNGLTSVRGKQMDFLKKLKKNERLLEEENKHLTFLLHHQ

SEQ ID NO: 190 - protein, 168 aa, Elaeis guineensis
  oil palm SEPALLATA (SEP)-like Eg_OPMADS1
MVRGKTEMKLIENATSRQVTFSKRRNGLLKKAFELSVLCDAEVAVIVFSPRGKLYEFSSTSMEKTIDRYRRHAKSGINNNEVTQQWKFEAASMSRKIESLEVSKRKLLGENLESCSAEELHEIEGKIEQSLCHVRGKKNQLLEEQIATLKEQEQTLMEENALLREKCK

SEQ ID NO: 191 - protein, 171 aa, Elaeis guineensis
  oil palm SEPALLATA (SEP)-like EgAGL20
  VARIANT (136)...(136): Xaa = any amino acid
MVRGKTQVRRIENATSRQVTFSKRRNGLLKKAFELSVLCDAEVALIVFSPRGKLYEFSSSSSSMEKTIDRYIMHAKDVNINKKSREHNAQQLKSDAASLMKKIERLEASKRKLLGENLESCAIEELHDLELQLEQXLSSIRNRKYQMLEEQICRLKEKEKILTASIQEKLN

SEQ ID NO: 192 - protein, 174 aa, Oryza sativa
  rice SEPALLATA (SEP)-like OsMADS56
MVRGRTELKRIENPTSRQVTFSKRRNGLLKKAFELSVLCDAEVALIVFSPRGRLYEFASAPSLQKTIDRYKAYTKDHVNNKTIQQDIQQVKDDTLGLAKKLEALDESRRKILGENLEGFSIEELRGLEMKLEKSLHKIRLKKTELLEQQIAKLKEKERTLLKDNENLRGKHRNL

SEQ ID NO: 193 - protein, 172 aa, Oryza sativa
  rice SEPALLATA (SEP)-like OsMADS4
MGRGKIEIKRIENSTNRQVTFSKRRAGILKKAREIGVLCDAEVGVVIFSSAGKLSDYCTPKTTSVFPPLSRILEKYQTNSGKILWDEKHKSLSAEIDRVKKENDNMQIELRHMKGEDLNSLQPKELIAIEEALNNGQANLRDKMMDHWRMHKRNEKMLEDEHKMLAFRVHQQ

SEQ ID NO: 194 - protein, 171 aa, Elaeis guineensis
  oil palm SEPALLATA (SEP)-like EgDEF1
MGRGKIEIKKIENPTNRQVTYSKRRTGIMKKAKELTVLCDAEVSLIMFSSTGKFSEYCSPLSDTKTIFDRYQQVSGINLWSAQYEKMQNTLNHLREINQNLRREIRQRMGEDLDSLGIHELRGLEQNLDEALKVVRHRKYHVITTQTDTYKKKLKNSNEAHKNLLHELEMK

SEQ ID NO: 195 - protein, 172 aa, Oryza sativa
  rice SEPALLATA (SEP)-like OsMADS32
MGRGRSEIKRIENPTQRQSTFYKRRDGLFKKARELAVLCDADLLLLLFSASGKLYHFLSPTVPSVREFVERYEATTHTKVWADIRQERRAELEKVGSMCDLLEKQLRFMTVDDGEEYTVPSLEALEHNLEAAMRKVRSEKDRKIGGEICYLQNIIRGRQEERYGLCDKIAHA

SEQ ID NO: 196 - protein, 174 aa, Elaeis guineensis
  oil palm SEPALLATA (SEP)-like EgAG2
MGRGKIEIKRIENTTNRQVTFCKRRNGLLKKAYELSVLCDAEVALIIFSSRGRLYEYANNSVKATIERYKKACTDTSNSGSVSEADSQYYQQESLKLRQQITSLQNSNRNLMGDSLGSMSLRDLKQLEGRLEKGINKIRTKKNELLFAEIEYMQKRETELQNANMYLRNKIAEN

SEQ ID NO: 197 - protein, 174 aa, Elaeis guineensis
  oil palm SEPALLATA (SEP)-like EgMEF2
MGRGKIEIKRIENTTNRQVTFCKRRNGLLKKAYELSVLCDAEVALIVFSSRGRLYEYANNSVKATIERYKRACTDTSNSGSVSEADSQYYQQESTKLRQQIISLQNSNRNLMGESLGSMSPRDLKQLEGRLEKGINKIRTKKNELLFAEIEYMQKREVELQNANMYLRNKIAEN

SEQ ID NO: 198 - protein, 171 aa, Elaeis guineensis
  oil palm SEPALLATA (SEP)-like EgSTMADS111
MAREKIQIRKIDNATARQVTFSKRRRGLFKKAEELAILCDADVALIIFSSTGKLFEFSSSSMKEILEKHSLHSKNLQKVEPPSLDLNLENSNYTRLDKQVAEASLQLRQMRGEELQGLTMQELQQLEKNLETGLSCVLERKGQQIMEQINHLQQKGMHLMEENERLRKQVV

SEQ ID NO: 199 - protein, 160 aa, Elaeis guineensis
  oil palm SEPALLATA (SEP)-like EgAGL25
MGRGKVELKRIENKINRQVTFAKRRNGLLKKANELSVLCDAEVALIIFSSSGRRFEFCSCSSVLKTIERYQTYNYAASEVVAPPSETQNTYQEYAKLKARVEFLQRSHRNLLGEDLDPLSTNELEQLENQVEKSLKQISSAKEQVLQEVNRELKRKLQEA

SEQ ID NO: 200 - protein, 175 aa, Elaeis guineensis
  oil palm SEPALLATA (SEP)-like EgSQUA1
MGRGRVQLRRIENKINRQVTFSKRRSGLLKKAHEISVLCDAEVALIIFSTKGKLYEYATDSCMERILERYERYTYAEKALISSGPELQGNWCHEFGKLKAKVEALQKSQRHLMGEQLEPLNLKELQQLEQQLESSLKHIRTRKCQLMFESISELQKKEKSLQEQNKMLEKELMEK

SEQ ID NO: 201 - protein, 175 aa, Oryza sativa
  rice SEPALLATA (SEP)-like OsMADS14
MGRGKVQLKRIENKINRQVTFSKRRSGLLKKANEISVLCDAEVALIIFSTKGKLYEYATDSCMDKILERYERYSYAEKVLISAESDTQGNWCHEYRKLKAKVETIQKCQKHLMGEDLESLNLKELQQLEQQLENSLKHIRSRKSQLMLESINELQRKEKSLQEENKVLQKELVEK

SEQ ID NO: 202 - protein, 175 aa, Oryza sativa
  rice SEPALLATA (SEP)-like OsMADS18
MGRGPVQLRRIENKINRQVTFSKRRNGLLKKAHEISVLCDADVALIVFSTKGKLYEFSSHSSMEGILERYQRYSFDERAVLEPNTEDQENWGDEYGILKSKLDALQKSQRQLLGEQLDTLTIKELQQLEHQLEYSLKHIRSKKNQLLFESISELQKKEKSLKNQNNVLQKLMETE

SEQ ID NO: 203 - protein, 174 aa, Elaeis guineensis
  oil palm SEPALLATA (SEP)-like EgAGL24
MGRGRVELKRIENKINRQVTFAKRRNGLLKKAFELSVLCDAEVALIIFSSRGRLFEFCSSSSMLKTLERYQRCNYSASEAAAPSSEIQNTYQEYVRLKARVEFLQHSQRNLLGEDLDPLSTNELDQLENQLEKSLKQIRSAKTQSMLDQLCDLKRREQEMQETNRSLNRKLREA

SEQ ID NO: 204 - protein, 174 aa, Elaeis guineensis
  oil palm SEPALLATA (SEP)-like EgMADS8
MGRGRVELKRIENKINRQVTFAKRRNGLLKKAYELSVLCDAEVALIIFSNRGKLYEFCSSSSMMKTLERYQKCNYGAPETNIISRETQSSQQEYLKLKARAEALQRSQRNLLGEDLGPLSSKELEQLEGQLDASLKQIRSTRTQYMLDQLADLQRKEQMLCEANKSLRRRLEES

SEQ ID NO: 205 - protein, 174 aa, Elaeis guineensis
  oil palm SEPALLATA (SEP)-like EgMADS2
MVRGKVELKRIENKINRQVTFAKRRNGLLKKAYELSVLCDAEVALIIFSNRGKLYEFCSSSSMMKTLERYQKCNYGAPETNIISRETQSSQQEYLKLKARAEALQRSQRNLLGEDLGPLSSKELEQLERQLDASLKQIRSTRTQYMLDQLADLQRKEQMLCEANKSLRRRLEES

SEQ ID NO: 206 - protein, 174 aa, Elaeis guineensis
  oil palm SEPALLATA (SEP)-like EgAGL22
MGRGRVELKRIENKINRQVTFAKRRNGLLKKAYELSVLCDAEVALIIFSNRGKLYEFCSSSSMMKTLERYQKCNYGAPETNIISRETQSSQQEYLKLKARVEALQRSQRNLLGEDLGPLSSKELEQLERQLDASLKQIRSTRTQYMLDQLADLQRREQMLCEANKSLRRRLEES

SEQ ID NO: 207 - protein, 175 aa, Oryza sativa
  rice SEPALLATA (SEP)-like OsMADS34
MGRGKVVLQRIENKISRQVTFAKRRNGLLKKAYELSILCDAEVALVLFSHAGRLYQFSSSSNMLKTLERYQRYIYASQDAAAPTSDEMQNNYQEYVNLKAHVEILQQSQRNLLGEDLAPLATNELEQLESQVVRTLKQIRSRKTQVLLDELCDLKRKEQMLQDANRVLKRKLDEI

SEQ ID NO: 208 - protein, 172 aa, Oryza sativa
  rice SEPALLATA (SEP)-like OsMADS1
MGRGKVELKRIENKISRQVTFAKRRNGLLKKAYELSLLCDAEVALIIFSGRGRLFEFSSSSCMYKTLERYRSCNYNSQDAAAPENEINYQEYLKLKTRVEFLQTTQRNILGEDLGPLSMKELEQLENQIEVSLKQIRSRKNQALLDQLFDLKSKEQQLQDLNKDLRKKLQET

SEQ ID NO: 209 - protein, 174 aa, Elaeis guineensis
  oil palm SEPALLATA (SEP)-like EgAGL23
MGRGRVELKRIENKINRQVTFAKRRNGLLKKAYELSVLCDAEVALIVFSNRGKLYEFCSSSSMLKTLERYQKCNYGAPETNIVSRETQTSQQEYLKLKARVEALQRSQRNLLGDDLGPLSSKELEQLERQLDASLKQIRSTRTQYMLDQLADLQRREQMLCEANKSLKQRLEES

SEQ ID NO: 210 - protein, 175 aa, Oryza sativa
  rice SEPALLATA (SEP)-like OsMADS17
MGRGRVELKRIENKINRQVTFSKRRNGLLKKAYELSVLCDAEVALIIFSSRGKLYEFGSAGINKTLEKYNSCCYNAQGSNSALAGGEHQSWYQEMSRLKTKLECLQRSQRHMLGEDLGPLSIKELQQLEKQLEYSLSQARQRKTQIMMEQVDDLRRKERQLGELNKQLKNKLEAE

SEQ ID NO: 211 - protein, 173 aa, Oryza sativa
  rice SEPALLATA (SEP)-like OsMADS6
MGRGRVELKRIENKINRQVTFSKRRNGLLKKAYELSVLCDAEVALIIFSSRGKLYEFGSAGITKTLERYQHCCYNAQDSNNALSETQSWYHEMSKLKAKFEALQRTQRHLLGEDLGPLSVKELQQLEKQLECALSQARQRKTQLMMEQVEELRRKERQLGEINRQLKHKLEVE